(19) United States
(12) Patent Application Publication
(10) Pub. No.: US 2013/0280775 A1
(43) Pub. Date: Oct. 24, 2013

(54) Recombinant N-propanol and Isopropanol Production

(71) Applicants: Thomas Grotkjaer, Frederiksberg (DK); Bjarke Christensen, Lyngby (DK); Torsten Bak Regueira, Copenhagen (DK); Steen Troels Joergensen, Alleroed (DK); Alan Berry, Granite Bay, CA (US)

(72) Inventors: Thomas Grotkjaer, Frederiksberg (DK); Bjarke Christensen, Lyngby (DK); Torsten Bak Regueira, Copenhagen (DK); Steen Troels Joergensen, Alleroed (DK); Alan Berry, Granite Bay, CA (US)

(73) Assignees: Novozymes, Inc., Davis, CA (US); Novozymes A/S, Bagsvaerd (DK)

(21) Appl. No.: 13/882,336

(22) PCT Filed: Oct. 28, 2011
(86) PCT No.: PCT/US2011/058405

§ 371 Date: Jul. 15, 2013

Related U.S. Application Data
(60) Provisional application No. 61/408,138, filed on Oct. 29, 2010.
(60) Provisional application No. 61/408,146, filed on Oct. 29, 2010.
(60) Provisional application No. 61/408,154, filed on Oct. 29, 2010.

(51) Int. Cl. C12P 7/04

(52) U.S. Cl. CPC C12P 7/04
USPC 435/157; 435/167; 435/252.3

(57) ABSTRACT

The present invention relates to methods of producing n-propanol, isopropanol, and coproducing n-propanol with isopropanol. The present invention also relates to methods for producing propylene, as well as host cells capable of n-propanol and isopropanol production.

### CROSS REFERENCE TO RELATED APPLICATIONS

This application claims priority benefit of U.S. Provisional Application No. 61/408,154, filed Oct. 29, 2010; U.S. Provisional Application No. 61/408,146, filed Oct. 29, 2010; and U.S. Provisional Application No. 61/408,138, filed Oct. 29, 2010. The content of these applications is hereby incorporated by reference as if it was set forth in full below.

### REFERENCE TO A SEQUENCE LISTING

This application contains a Sequence Listing in computer readable form, which is incorporated herein by reference.

### REFERENCE TO A DEPOSIT OF BIOLOGICAL MATERIAL

This application contains a reference to a deposit of biological material, which deposit is incorporated herein by reference.

### BACKGROUND OF THE INVENTION

1. Field of the Invention

The present invention relates to methods for the recombinant production of n-propanol and isopropanol.

2. Description of the Related Art

Concerns related to future supply of oil have prompted research in the area of renewable energy and renewable sources of other raw materials. Biofuels, such as ethanol and bioplastics (e.g., particularly polylactic acid) are examples of products that can be made directly from agricultural sources using microorganisms. Additional desired products may then be derived using non-enzymatic chemical conversions, e.g., dehydration of ethanol to ethylene.

Polymerization of ethylene provides polyethylene, a type of plastic with a wide range of useful applications. Ethylene is traditionally produced by refined non-renewable fossil fuels. However, dehydration of biologically-derived ethanol to ethylene offers an alternative route to ethylene from renewable carbon sources, i.e., ethanol from fermentation of fermentable sugars. This process has been utilized for the production of “Green Polyethylene” that—save for minute differences in the carbon isotope distribution—is identical to polyethylene produced from oil.

Similarly, isopropanol and n-propanol can be dehydrated to propylene, which in turn can be polymerized to polypropylene. As with polyethylene, using biologically-derived starting material (i.e., isopropanol or n-propanol) would result in “Green Polypropylene.” However, unlike polyethylene, the production of the polyethylene starting material from renewable sources has proved challenging. Proposed efforts at propanol production have been reported in WO 2009/049274, WO 2009/103026, WO 2009/131286, WO 2010/071697, WO 2011/031897, WO 2011/029166, and WO 2011/022651. It is clear that the successful development of a process for the biological production of propanol requires careful selection of enzymes in the metabolic pathways as well as an efficient overall metabolic engineering strategy.

It would be advantageous in the art to provide methods of producing recombinant n-propanol and isopropanol. The present invention provides such methods as well as recombinant host cells used in the methods.

### SUMMARY OF THE INVENTION

The present invention relates to, inter alia, recombinant host cells for the production of n-propanol and/or isopropanol. In one aspect, the host cells comprise thiolase activity, CoA-transferase activity, acetoacetate decarboxylase activity, and/or isopropanol dehydrogenase activity, wherein the host cell produces (or is capable of producing) isopropanol. In one aspect, the host cells comprises aldehyde dehydrogenase activity, wherein the host cell produces (or is capable of producing) n-propanol. In one aspect, the host cell comprises thiolase activity, CoA-transferase activity, acetoacetate decarboxylase activity, isopropanol dehydrogenase activity, and/or aldehyde dehydrogenase activity, wherein the host cell produces (or is capable of producing) n-propanol and isopropanol. In some of these aspects, the host cells optionally further comprise methylmalonyl-CoA mutase activity, methylmalonyl-CoA decarboxylase activity, methylmalonyl-CoA epimerase activity and/or n-propanol dehydrogenase activity.

In one aspect, the recombinant host cells comprise a heterologous polynucleotide encoding a thiolase; one or more (several) heterologous polynucleotides encoding a CoA-transferase (e.g., one or more (several) heterologous polynucleotides encoding a succinyl-CoA:acetoacetate transferase); a heterologous polynucleotide encoding an acetoacetate decarboxylase; a heterologous polynucleotide encoding an isopropanol dehydrogenase; and/or a heterologous polynucleotide encoding an aldehyde dehydrogenase. The host cells may optionally further comprise a heterologous polynucleotide encoding methylmalonyl-CoA mutase, a heterologous polynucleotide encoding a methylmalonyl-CoA decarboxylase, a heterologous polynucleotide encoding a methylmalonyl-CoA epimerase, and/or a heterologous polynucleotide encoding an n-propanol dehydrogenase.

The present invention also relates to methods of using recombinant host cells for the production of n-propanol, the production of isopropanol, or the coproduction of n-propanol and isopropanol.

In one aspect, the invention related to methods of producing isopropanol, comprising: (a) cultivating a recombinant host cell having thiolase activity, CoA-transferase activity, acetoacetate decarboxylase activity, and isopropanol dehydrogenase activity in a medium under suitable conditions to produce isopropanol; and (b) recovering the isopropanol. In some embodiments of the methods, the recombinant host cells comprise a heterologous polynucleotide encoding a thiolase; one or more (several) heterologous polynucleotides encoding a CoA-transferase; a heterologous polynucleotide encoding an acetoacetate decarboxylase; and/or a heterologous polynucleotide encoding an isopropanol dehydrogenase.

In another aspect, the invention related to methods of producing n-propanol, comprising: (a) cultivating a recombinant host cell having aldehyde dehydrogenase activity in a medium under suitable conditions to produce n-propanol; and (b) recovering the n-propanol. In some embodiments of the methods, the recombinant host cell comprises a heterologous polynucleotide encoding an aldehyde dehydrogenase. In embodiments of the methods, the recombinant host cell further comprises one or more (several) heterologous polynucleotides encoding a methylmalonyl-CoA mutase; a heterologous polynucleotide encoding a methylmalonyl-CoA decarboxylase; and/or a heterologous polynucleotide encoding an n-propanol dehydrogenase.

In another aspect, the invention related to methods of coproducing n-propanol and isopropanol, comprising: (a) cultivating a recombinant host cell having thiolase activity, CoA-transferase activity, acetoacetate decarboxylase activity, isopropanol dehydrogenase activity, and aldehyde dehydrogenase activity in a medium under suitable conditions to produce n-propanol and isopropanol; and (b) recovering the n-propanol and isopropanol. In some embodiments of the methods, the recombinant host cells comprise a heterologous polynucleotide encoding a thiolase; one or more (several) heterologous polynucleotides encoding a CoA-transferase (e.g., one or more (several) heterologous polynucleotides encoding a succinyl-CoA:acetoacetate transferase); a heterologous polynucleotide encoding an acetoacetate decarboxylase; a heterologous polynucleotide encoding an isopropanol dehydrogenase; and/or a heterologous polynucleotide encoding an aldehyde dehydrogenase. The host cells of the methods may optionally further comprise a heterologous polynucleotide encoding methylmalonyl-CoA mutase, a heterologous polynucleotide encoding a methylmalonyl-CoA decarboxylase, a heterologous polynucleotide encoding a methylmalonyl-CoA epimerase, and/or a heterologous polynucleotide encoding an n-propanol dehydrogenase.

The present invention also relates to methods of producing propylene, comprising: (a) cultivating a recombinant host cell described herein in a medium under suitable conditions to produce n-propanol and/or isopropanol; (b) recovering the n-propanol and/or isopropanol; (c) dehydrating the n-propanol and/or isopropanol under suitable conditions to produce propylene; and (d) recovering the propylene.

In some aspects, the host cell is a Lactobacillus host cell (e.g., a L. plantarum or L. reuteri host cell). In other aspects, the host cell is a Propionibacterium (e.g., Propionibacterium acidipropionici host cell).

### BRIEF DESCRIPTION OF THE FIGURES

FIG. 1 shows a metabolic pathway from glucose for the production of isopropanol.

FIG. 2 shows a metabolic pathway from glucose for the production of n-propanol.

FIG. 3 shows a metabolic pathway from glucose for the coproduction of isopropanol and n-propanol.

FIG. 4 shows a restriction map of pTRGU88.

FIG. 5 shows a restriction map of pSJ10600.

FIG. 6 shows a restriction map of pSJ10603.

### DEFINITIONS

Thiolase: The term “thiolase” is defined herein as an acyltransferase that catalyzes the chemical reaction of two molecules of acetyl-CoA to acetoacetyl-CoA and CoA (EC 2.3.1.9). For the purpose of the inventions described herein, thiolase activity may be determined according to the procedure described by D. P. Wiesenborn et al., 1988, Appl. Environ. Microbiol. 54:2717-2722, the content of which is hereby incorporated by reference in its entirety. For example, thiolase activity may be measured spectrophotometrically by monitoring the condensation reaction coupled to the oxidation of NADH using 3-hydroxyacyl-CoA dehydrogenase in 100 mM Tris hydrochloride (pH 7.4), 1.0 mM acetyl-CoA, 0.2 mM NADH, 1 mM dithiothreitol, and 2 U of 3-hydroxyacyl-CoA dehydrogenase. After equilibration of the cuvette contents at 30° C. for 2 min, the reaction is initiated by the addition of about 125 ng of thiolase in 10 μL. The absorbance decrease at 340 nm due to oxidation of NADH is measured, and an extinction coefficient of 6.22 mM⁻¹ cm⁻¹ used. One unit of thiolase activity equals the amount of enzyme capable of releasing 1 micromole of acetoacetyl-CoA per minute at pH 7.4, 30° C.

A thiolase may have at least 20%, e.g., at least 40%, at least 50%, at least 60%, at least 70%, at least 80%, at least 90%, at least 95%, at least 96%, at least 97%, at least 98%, at least 99%, or 100% of the thiolase activity of the mature polypeptide of SEQ ID NO: 3, 35, 114, or 116.

CoA-transferase: As used herein, the term “CoA-transferase” is defined as any enzyme that catalyzes the removal of coenzyme A from acetoacetyl-CoA to generate acetoacetate. In some aspects, the CoA-transferase is an acetoacetyl-CoA:acetate/butyrate CoA transferase of EC 2.8.3.9. In some aspects, the CoA-transferase is an acetoacetyl-CoA hydrolase of EC 3.1.2.11. In some aspects, the CoA-transferase is an acetoacetyl-CoA transferase that converts acetoacetyl-CoA and acetate to acetoacetate and acetyl-CoA.

A Co-A transferase may have at least 20%, e.g., at least 40%, at least 50%, at least 60%, at least 70%, at least 80%, at least 90%, at least 95%, at least 96%, at least 97%, at least 98%, at least 99%, or 100% of the Co-A transferase activity of a protein complex comprising the mature polypeptide of SEQ ID NO: 37 and the mature polypeptide of SEQ ID NO: 39; or a protein complex comprising the mature polypeptide of SEQ ID NO: 41 and the mature polypeptide of SEQ ID NO: 43.

In some aspects, the CoA-transferase is a succinyl-CoA:acetoacetate transferase. As used herein, “succinyl-CoA:acetoacetate transferase” is an acetotransferase that catalyzes the chemical reaction of acetoacetyl-CoA and succinate to acetoacetate and succinyl-CoA (EC 2.8.3.5). The succinyl-CoA:acetoacetate transferase may be in the form of a protein complex comprising one or more (several) subunits (e.g., two heteromeric subunits) as described herein. For the purpose of the inventions described herein, succinyl-CoA:acetoacetate transferase activity may be determined according to the procedure described by L. Stols et al., 1989, Protein Expression and Purification 53:396-403, the content of which is hereby incorporated by reference in its entirety. For example, succinyl-CoA:acetoacetate transferase activity may be measured spectrophotometrically by monitoring the formation of the enolate anion of acetoacetyl-CoA, wherein absorbance is measured at 310 nm/30° C. over 4 minutes in an assay buffer of 67 mM lithium acetoacetate, 300 μM succinyl-CoA, and 15 mM MgCl₂ in 50 mM Tris, pH 9.1. One unit of succinyl-CoA:acetoacetate transferase activity equals the amount of enzyme capable of releasing 1 micromole of acetoacetate per minute at pH 9.1, 30° C.

A succinyl-CoA:acetoacetate transferase may have at least 20%, e.g., at least 40%, at least 50%, at least 60%, at least 70%, at least 80%, at least 90%, at least 95%, at least 96%, at least 97%, at least 98%, at least 99%, or 100% of the succinyl-CoA:acetoacetate transferase activity of a protein complex comprising the mature polypeptide of SEQ ID NO: 6 and the mature polypeptide of SEQ ID NO: 9; or a protein complex comprising the mature polypeptide of SEQ ID NO: 12 and the mature polypeptide of SEQ ID NO: 15.

Acetoacetate decarboxylase: The term “acetoacetate decarboxylase” is defined herein as an enzyme that catalyzes the chemical reaction of acetoacetate to carbon dioxide and acetone (EC 4.1.1.4). For the purpose of the inventions described herein, acetoacetate decarboxylase activity may be determined according to the procedure described by D. J. Petersen, et al., 1990, Appl. Environ. Microbiol. 56, 3491-3498, the content of which is hereby incorporated by reference in its entirety. For example, acetoacetate decarboxylase activity may be measured spectrophotometrically by monitoring the depletion of acetoacetate at 270 nm in 5 nM acetoacetate, 0.1 M KPO₄, pH 5.9 at 26° C. One unit of acetoacetate decarboxylase activity equals the amount of enzyme capable of consuming 1 micromole of acetoacetate per minute at pH 5.9, 26° C.

An acetoacetate decarboxylase may have at least 20%, e.g., at least 40%, at least 50%, at least 60%, at least 70%, at least 80%, at least 90%, at least 95%, at least 96%, at least 97%, at least 98%, at least 99%, or 100% of the acetoacetate decarboxylase activity of the mature polypeptide of SEQ ID NO: 18, 45, 118, or 120.

Isopropanol dehydrogenase: The term “isopropanol dehydrogenase” is defined herein as any suitable oxidoreductase that catalyzes the reduction of acetone to isopropanol (e.g., any suitable enzyme of EC1.1.1.1 or EC 1.1.1.80). For the purpose of the inventions described herein, isopropanol dehydrogenase activity may be determined spectrophotometrically by decrease in absorbance at 340 nm in an assay containing 200 μM NADPH and 10 mM acetone in 25 mM potassium phosphate, pH 7.2 at 25° C. One unit of isopropanol dehydrogenase activity may be defined as the amount of enzyme releasing 1 micromole of NADP+ per minute using a molar extinction coefficient of NADPH of 6220 M⁻¹*cm⁻¹.

An isopropanol dehydrogenase may have at least 20%, e.g., at least 40%, at least 50%, at least 60%, at least 70%, at least 80%, at least 90%, at least 95%, at least 96%, at least 97%, at least 98%, at least 99%, or 100% of the isopropanol dehydrogenase activity of the mature polypeptide of SEQ ID NO: 21, 24, 47, or 122.

Aldehyde dehydrogenase: The term “aldehyde dehydrogenase” is defined herein as an enzyme that catalyzes the oxidation of an aldehyde (EC 1.2.1.3). The aldehyde dehydrogenase may be reversible, e.g., and may catalyze the chemical reaction of propionyl-CoA to propanal. For the purpose of the inventions described herein, aldehyde dehydrogenase activity may be determined according to the procedure described by N. Hosoi et al., 1979, J. Ferment. Technol., 57:418-427, the content of which is hereby incorporated by reference in its entirety. For example, aldehyde dehydrogenase activity may be measured spectrophotometrically by monitoring the reduction of NAD+ by an increase in absorbance at 340 nm at 30° C. using a 3 mL solution containing 100 μmol propionaldehyde, 3 μmol NAD+, 0.3 μmol CoA, 30 μmol GSH, 100 μg bovine serum albumin, 120 μmol veronal-HCl buffer (pH 8.6). One unit of aldehyde dehydrogenase transferase activity equals the amount of enzyme capable of releasing 1 micromole of propionyl-CoA per minute at pH 8.6, 30° C.

An aldehyde dehydrogenase may have at least 20%, e.g., at least 40%, at least 50%, at least 60%, at least 70%, at least 80%, at least 90%, at least 95%, at least 96%, at least 97%, at least 98%, at least 99%, or 100% of the aldehyde dehydrogenase activity of the mature polypeptide of SEQ ID NO: 27, 30, 33, 51, 54, 57, 60, or 63.

In one aspect, the aldehyde dehydrogenase has an initial reaction rate (v₀) for a acetyl-CoA substrate that is less than 95%, 90%, 80%, 70%, 60%, 50%, 40%, 30%, 20%, 15%, 10%, 7.5%, 5%, 2.5%, or 1% of the initial reaction rate (v₀) for an propionyl-CoA substrate under the same conditions.

Methylmalonyl-CoA Mutase:

The term “methylmalonyl-CoA mutase” is defined herein as an enzyme that catalyzes the reversible isomerization of methylmalonyl-CoA to succinyl-CoA (EC 5.4.99.2). In some aspects, the methylmalonyl-CoA mutase requires vitamin B12 for methylmalonyl-CoA mutase activity. For the purpose of the inventions described herein, methylmalonyl-CoA mutase activity may be determined according to the procedure described by T. Haller et al., 2000, Biochemistry, 39:4622-4629, the content of which is hereby incorporated by reference in its entirety. For example, methylmalonyl-CoA mutase activity may be measured by HPLC analysis to measure the depletion of succinyl-CoA at 37° C. in a 500 μL solution of Sodium Tris-HCl (50 mM) containing succinyl-CoA (2-43 μM), methylmalonyl-CoA mutase (8 nM), KCl (30 mM) and a kinetic excess of methylmalonyl-CoA decarboxylase (ygfG, T. Haller et al., 2000, supra) at pH 7.5. One unit of methylmalonyl-CoA mutase activity equals the amount of enzyme capable of consuming 1 micromole of succinyl-CoA per minute at pH 7.5, 37° C.

A methylmalonyl-CoA mutase may have at least 20%, e.g., at least 40%, at least 50%, at least 60%, at least 70%, at least 80%, at least 90%, at least 95%, at least 96%, at least 97%, at least 98%, at least 99%, or 100% of the methylmalonyl-CoA mutase activity of the mature polypeptide sequence of SEQ ID NO: 93; or a protein complex containing a first subunit having the mature polypeptide sequence of SEQ ID NO: 66 and a second subunit having the mature polypeptide sequence of SEQ ID NO: 69.

Methylmalonyl-CoA decarboxylase: The term “methylmalonyl-CoA decarboxylase” is defined herein as an enzyme that catalyzes the chemical reaction of methylmalonyl-CoA to propionyl-CoA and carbon dioxide (e.g., EC 4.1.1.41). The methylmalonyl-CoA decarboxylase may catalyzes the conversion of either (2R)-methylmalonyl-CoA, (2S)-methylmalonyl-CoA, or both. In one aspect, the methylmalonyl-CoA decarboxylase has a greater specificity for (2R)-methylmalonyl-CoA over (2S)-methylmalonyl-CoA under the same conditions. In another aspect, the methylmalonyl-CoA decarboxylase has a greater specificity for (2S)-methylmalonyl-CoA over (2R)-methylmalonyl-CoA under the same conditions.

For the purpose of the inventions described herein, methylmalonyl-CoA decarboxylase activity may be determined according to the procedure described by T. Haller et al., 2000, supra. For example, methylmalonyl-CoA decarboxylase activity may be measured by continuous spectrophotometric analysis to determine the conversion of methylmalonyl-CoA to propionyl-CoA by monitoring the oxidation of NADH in the presence of oxalacetate, transcarboxylase, and lactate dehydrogenase at 37° C. In this example, a 1.2 mL solution of potassium phosphate (16.7 mM) contains methylmalonyl-CoA decarboxylase (0.6 μM), methylmalonyl-CoA (3-45 μM), oxalacetate (8.3 mM), NADH (0.33 mM), transcarboxylase (5 mU) and lactate dehydrogenase (4 mU) at pH 7.2. One unit of methylmalonyl-CoA decarboxylase activity equals the amount of enzyme capable of decarboxylating 1 micromole of methylmalonyl-CoA per minute at pH 7.2, 37° C.

A methylmalonyl-CoA decarboxylase may have at least 20%, e.g., at least 40%, at least 50%, at least 60%, at least 70%, at least 80%, at least 90%, at least 95%, at least 96%, at least 97%, at least 98%, at least 99%, or 100% of the methylmalonyl-CoA decarboxylase activity of the mature polypeptide sequence of SEQ ID NO: 103.

Methylmalonyl-CoA epimerase: The term “methylmalonyl-CoA epimerase” is defined herein as an enzyme that catalyzes the chemical epimerization of methylmalonyl-CoA (e.g., R-methylmalonyl-CoA to S-methylmalonyl-CoA and/or S-methylmalonyl-CoA to R-methylmalonyl-CoA; see EC 5.1.99.1). For the purpose of the inventions described herein, methylmalonyl-CoA epimerase activity may be determined according to the procedure described by Dayem et al., 2002, Biochemistry, 41:5193-5201, the content of which is hereby incorporated by reference in its entirety.

A methylmalonyl-CoA epimerase may have at least 20%, e.g., at least 40%, at least 50%, at least 60%, at least 70%, at least 80%, at least 90%, at least 95%, at least 96%, at least 97%, at least 98%, at least 99%, or 100% of the methylmalonyl-CoA epimerase activity of the mature polypeptide sequence of SEQ ID NO: 75.

n-Propanol dehydrogenase: The term “n-propanol dehydrogenase” is defined herein as any alcohol dehydrogenase (EC 1.1.1.1) that catalyzes the reduction of propanal to n-propanol. For the purpose of the inventions described herein, n-propanol dehydrogenase activity may be determined according to the procedure described by C. Drewke and M. Ciriacy, 1988, Biochemica et Biophysica Acta, 950:54-60, the content of which is hereby incorporated by reference in its entirety. For example, n-propanol dehydrogenase activity may be measured spectrophotometrically following the kinetics of NAD⁺ reduction of NADH oxidation at pH 8.3. One unit of n-propanol dehydrogenase activity equals the amount of enzyme capable of converting 1 micromole of propanal per minute to n-propanol at pH 8.3, 30° C.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide whose expression is quantitatively altered by the introduction of one or more (several) extra copies of the polynucleotide into the host cell.

Isolated/purified: The terms “isolated” or “purified” mean a polypeptide or polynucleotide that is removed from at least one component with which it is naturally associated.

For example, a polypeptide may be at least 1% pure, e.g., at least 5% pure, at least 10% pure, at least 20% pure, at least 40% pure, at least 60% pure, at least 80% pure, at least 90% pure, at least 93% pure, at least 95% pure, at least 97%, at least 98% pure, or at least 99% pure, as determined by SDS-PAGE and a polynucleotide may be at least 1% pure, e.g., at least 5% pure, at least 10% pure, at least 20% pure, at least 40% pure, at least 60% pure, at least 80% pure, at least 90%, at least 93% pure, at least 95% pure, at least 97%, at least 98% pure, or at least 99% pure, as determined by agarose electrophoresis.

Mature polypeptide sequence: The term “mature polypeptide sequence” means the portion of the referenced polypeptide sequence after any post-translational sequence modifications (such as N-terminal processing and/or C-terminal truncation). The mature polypeptide sequence may be predicted, e.g., based on the SignalP program (Nielsen et al., 1997, Protein Engineering 10:1-6) or the InterProScan program (The European Bioinformatics Institute). In some instances, the mature polypeptide sequence may be identical to the entire referenced polypeptide sequence. It is known in the art that a host cell may produce a mixture of two of more different mature polypeptide sequences (i.e., with a different C-terminal and/or N-terminal amino acid) expressed by the same polynucleotide.

Mature polypeptide coding sequence: The term “mature polypeptide coding sequence” means a polynucleotide that encodes the referenced mature polypeptide.

Sequence Identity The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Fragment: The term “fragment” means a polypeptide having one or more (e.g., two, several) amino acids deleted from the amino and/or carboxyl terminus of a referenced polypeptide sequence. In one aspect, the fragment has thiolase activity, CoA-transferase activity (e.g., succinyl-CoA:acetoacetate transferase activity), acetoacetate decarboxylase activity, isopropanol dehydrogenase activity, methylmalonyl-CoA mutase activity, methylmalonyl-CoA decarboxylase activity, aldehyde dehydrogenase activity, or n-propanol dehydrogenase activity. In another aspect, the number of amino acid residues in the fragment is at least 75%, e.g., at least 80%, 85%, 90%, or 95% of the number of amino acid residues of any amino acid sequence referenced herein.

Subsequence: The term “subsequence” means a polynucleotide having one or more (e.g., two, several) nucleotides deleted from the 5′ and/or 3′ end of the referenced nucleotide sequence. In one aspect, the subsequence encodes a fragment having thiolase activity, CoA-transferase activity (e.g., succinyl-CoA:acetoacetate transferase activity), acetoacetate decarboxylase activity, isopropanol dehydrogenase activity, methylmalonyl-CoA mutase activity, methylmalonyl-CoA decarboxylase activity, aldehyde dehydrogenase activity, or n-propanol dehydrogenase activity. In another aspect, the number of nucleotides residues in the subsequence is at least 75%, e.g., at least 80%, 85%, 90%, or 95% of the number of nucleotide residues in any polynucleotide sequence referenced herein.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be genomic DNA, cDNA, a synthetic polynucleotide, and/or a recombinant polynucleotide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA. In some instances, a cDNA sequence may be identical to a genomic DNA sequence.

Nucleic acid construct: The term “nucleic acid construct” means a nucleic acid molecule, either single-stranded or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” means all components necessary for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Variant: The term “variant” means a polypeptide having the referenced enzyme activity, or a polypeptide of a protein complex having the referenced enzyme activity, wherein the polypeptide comprises an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding one or more (several), e.g., 1-3 amino acids, adjacent to an amino acid occupying a position.

Volumetric productivity: The term “volumetric productivity” refers to the amount of referenced product produced (e.g., the amount of n-propanol and/or isopropanol produced) per volume of the system used (e.g., the total volume of media and contents therein) per unit of time.

Fermentable medium: The term “fermentable medium” refers to a medium comprising one or more (several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as propanol. In some instances, the fermentation medium is derived from a natural source, such as sugar cane, starch, or cellulose, and may be the result of pretreating the source by enzymatic hydrolysis (saccharification). In one aspect, the fermentable medium does not comprise 1,2-propanediol.

Sugar cane juice: The term “sugar cane juice” refers to the liquid extract from pressed Saccharum grass (sugarcane), such as pressed Saccharum officinarum or Saccharum robustom.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

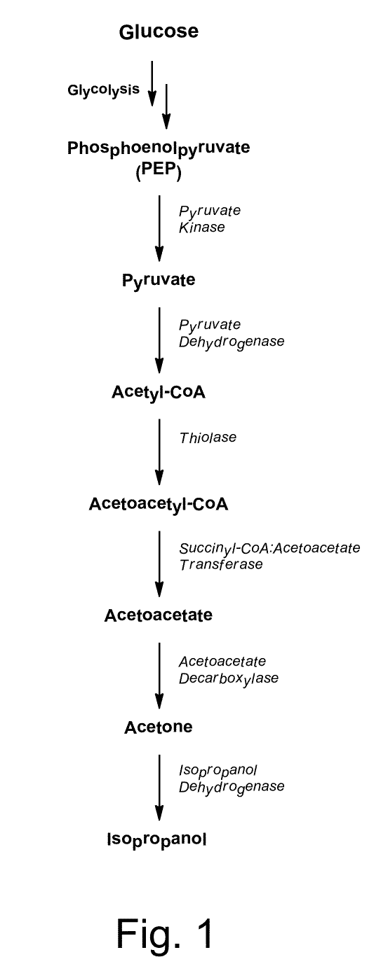

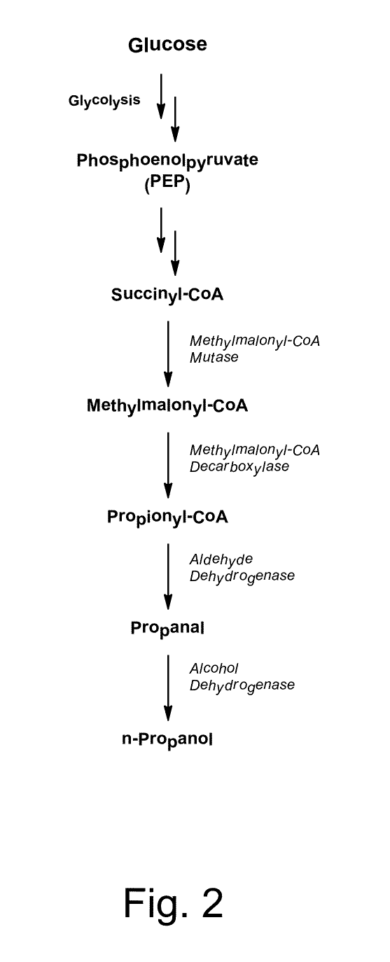

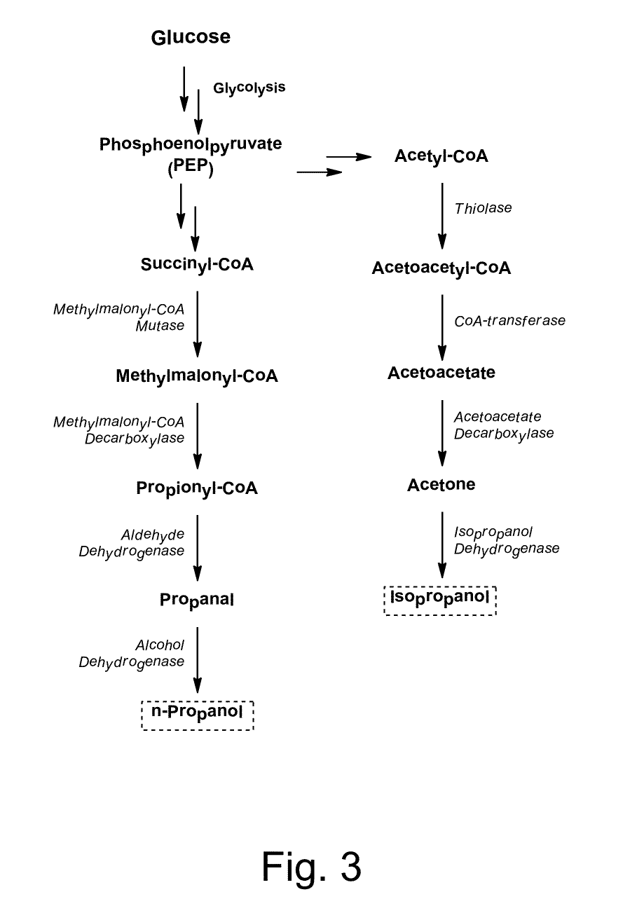

The present invention describes, inter alia, the overexpression of specific genes in a host cell (e.g., a prokaryotic host cell) to produce n-propanol or isopropanol (e.g., as depicted in FIGS. 1 and 2) or to coproduce n-propanol or isopropanol (e.g., as depicted in FIG. 3). The invention encompasses the use of heterologous genes for acetylation of acetyl-CoA to acetoacetyl-CoA by a thiolase, conversion of acetoacetyl-CoA to acetoacetate by a CoA-transferase, decarboxylation of acetoacetate to acetone by an acetoacetate decarboxylase, reduction of acetone to isopropanol by an isopropanol dehydrogenase, the isomerization of succinyl-CoA to methylmalonyl-CoA by a methylmalonyl-CoA mutase, decarboxylation of methylmalonyl-CoA to propionyl-CoA by a methylmalonyl-CoA decarboxylase, reduction of propionyl-CoA to propanal by an aldehyde dehydrogenase, and/or reduction of propanal to n-propanol by an n-propanol dehydrogenase. Any suitable thiolase, CoA transferase, acetoacetate decarboxylase, isopropanol dehydrogenase, methylmalonyl-CoA mutase, methylmalonyl-CoA decarboxylase, aldehyde dehydrogenase, and/or n-propanol dehydrogenase may be used to produce n-propanol and/or isopropanol.

In one aspect, the present invention relates to a recombinant host cell comprising thiolase activity, succinyl-CoA:acetoacetate transferase activity, acetoacetate decarboxylase activity and/or isopropanol dehydrogenase activity, wherein the recombinant host cell produces (or is capable of producing) isopropanol. The recombinant host cell may comprise one or more (several) heterologous polynucleotides, such as a heterologous polynucleotide encoding a thiolase; one or more (several) heterologous polynucleotides encoding a CoA-transferase (e.g., succinyl-CoA:acetoacetate transferase); a heterologous polynucleotide encoding an acetoacetate decarboxylase; and/or a heterologous polynucleotide encoding an isopropanol dehydrogenase.

In one aspect, the present invention relates to a recombinant host cell comprising aldehyde dehydrogenase activity, wherein the recombinant host cell produces (or is capable of producing) propanal or n-propanol. In some aspects, the recombinant host cell produces (or is capable of producing) propanal or n-propanol from propionyl-CoA. The recombinant host cell may comprise a heterologous polynucleotide encoding an aldehyde dehydrogenase. In some aspects, the recombinant host cell further comprises one or more (several) heterologous polynucleotides encoding a methylmalonyl-CoA mutase; a heterologous polynucleotide encoding a methylmalonyl-CoA decarboxylase; a heterologous polynucleotide encoding a methylmalonyl-CoA epimerase; and/or a heterologous polynucleotide encoding an n-propanol dehydrogenase.

In one aspect, the present invention relates to a recombinant host cell comprising thiolase activity, CoA-transferase activity (e.g., succinyl-CoA:acetoacetate transferase activity), acetoacetate decarboxylase activity, isopropanol dehydrogenase activity, and aldehyde dehydrogenase activity wherein the recombinant host cell produces (or is capable of producing) both n-propanol and isopropanol. The recombinant host cell may comprise one or more (several) heterologous polynucleotides, such as a heterologous polynucleotide encoding a thiolase; one or more (several) heterologous polynucleotides encoding a CoA-transferase (e.g., a succinyl-CoA:acetoacetate transferase); a heterologous polynucleotide encoding an acetoacetate decarboxylase; a heterologous polynucleotide encoding an isopropanol dehydrogenase; and/or a heterologous polynucleotide encoding an aldehyde dehydrogenase. The host cell may optionally further comprise a heterologous polynucleotide encoding methylmalonyl-CoA mutase, a heterologous polynucleotide encoding a methylmalonyl-CoA decarboxylase, and/or a heterologous polynucleotide encoding an n-propanol dehydrogenase.

### Thiolase and Polynucleotides Encoding Thiolase

In the present invention, the thiolase can be any thiolase that is suitable for practicing the invention. In one aspect, the thiolase is a thiolase that is overexpressed under culture conditions wherein an increased amount of acetoacetyl-CoA is produced.

In one aspect of the recombinant host cells and methods described herein, the thiolase is selected from: (a) a thiolase having at least 60% sequence identity to the mature polypeptide of SEQ ID NO: 3, 35, 114, or 116; (b) a thiolase encoded by a polynucleotide that hybridizes under at least low stringency conditions with the mature polypeptide coding sequence of SEQ ID NO: 1, 2, 34, 113, or 115, or the full-length complementary strand thereof; and (c) a thiolase encoded by a polynucleotide having at least 60% sequence identity to the mature polypeptide coding sequence of SEQ ID NO: 1, 2, 34, 113, or 115. As can be appreciated by one of skill in the art, in some instances the thiolase may qualify under more than one of the selections (a), (b) and (c) noted above.

In one aspect, the thiolase comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 3, 35, 114, or 116m and having thiolase activity.

In one aspect, the thiolase comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 3, and having thiolase activity.

In one aspect, the thiolase comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 35, and having thiolase activity.

In one aspect, the thiolase comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 114, and having thiolase activity.

In one aspect, the thiolase comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 116, and having thiolase activity.

In one aspect, the thiolase comprises an amino acid sequence that differs by no more than ten amino acids, e.g., by no more than five amino acids, by no more than four amino acids, by no more than three amino acids, by no more than two amino acids, or by one amino acid from SEQ ID NO: 3, 35, 114, or 116.

In one aspect, the thiolase comprises the amino acid sequence of SEQ ID NO: 3 or an allelic variant thereof; or a fragment of the foregoing, having thiolase activity. In another aspect, the thiolase comprises or consists of the mature polypeptide of SEQ ID NO: 3. In another aspect, the thiolase comprises the amino acid sequence of SEQ ID NO: 3. In another aspect, the thiolase comprises or consists of amino acids 1 to 392 of SEQ ID NO: 3. In one aspect, the thiolase comprises the amino acid sequence of SEQ ID NO: 35 or an allelic variant thereof; or a fragment of the foregoing, having thiolase activity. In another aspect, the thiolase comprises or consists of the mature polypeptide of SEQ ID NO: 35. In another aspect, the thiolase comprises the amino acid sequence of SEQ ID NO: 35. In another aspect, the thiolase comprises or consists of amino acids 1 to 392 of SEQ ID NO: 35. In another aspect, the thiolase comprises or consists of the mature polypeptide of SEQ ID NO: 114. In another aspect, the thiolase comprises the amino acid sequence of SEQ ID NO: 114. In another aspect, the thiolase comprises or consists of the mature polypeptide of SEQ ID NO: 116. In another aspect, the thiolase comprises the amino acid sequence of SEQ ID NO: 116.

In one aspect, the thiolase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 1, 2, 34, 113, or 115 or the full-length complementary strand thereof (see, e.g., J. Sambrook, E. F. Fritsch, and T. Maniatus, 1989, Molecular Cloning, A Laboratory Manual, 2d edition, Cold Spring Harbor, N.Y.).

In one aspect, the thiolase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 1 or 2, or the full-length complementary strand thereof.

In one aspect, the thiolase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 34, or the full-length complementary strand thereof.

In one aspect, the thiolase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 113, or the full-length complementary strand thereof.

In one aspect, the thiolase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 115, or the full-length complementary strand thereof.

In one aspect, the thiolase is encoded by a subsequence of SEQ ID NO: 1, 2, 34, 113, or 115; wherein the subsequence encodes a polypeptide having thiolase activity.

The polynucleotide of SEQ ID NO: 1, 2, 34, 113, or 115, or a subsequence thereof; as well as the amino acid sequence of SEQ ID NO: 3, 35, 114, or 116; or a fragment thereof; may be used to design nucleic acid probes to identify and clone DNA encoding a thiolase from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 700 nucleotides, even more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having thiolase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, 2, 34, 113, or 115, or a subsequence thereof, the carrier material is preferably used in a Southern blot.

For purposes of the present invention, hybridization indicates that the polynucleotide hybridizes to a labeled nucleic acid probe corresponding to the mature polypeptide coding sequence of SEQ ID NO: 1, 2, 34, 113, or 115, or a full-length complementary strand thereof; or a subsequence of the foregoing; under very low to very high stringency conditions. Molecules to which the nucleic acid probe hybridizes under these conditions can be detected using, for example, X-ray film.

In one aspect, the nucleic acid probe is the mature polypeptide coding sequence of SEQ ID NO: 1, 2, 34, 113, or 115. In another aspect, the nucleic acid probe is the mature polypeptide coding sequence of SEQ ID NO: 1. In another aspect, the nucleic acid probe is SEQ ID NO: 1. In another aspect, the nucleic acid probe is the mature polypeptide coding sequence of SEQ ID NO: 2. In another aspect, the nucleic acid probe is SEQ ID NO: 2. In another aspect, the nucleic acid probe is a polynucleotide that encodes the polypeptide of SEQ ID NO: 3, or a fragment thereof. In another aspect, the nucleic acid probe is the mature polypeptide coding sequence of SEQ ID NO: 34. In another aspect, the nucleic acid probe is SEQ ID NO: 34. In another aspect, the nucleic acid probe is a polynucleotide that encodes the polypeptide of SEQ ID NO: 35, or a fragment thereof. In another aspect, the nucleic acid probe is the mature polypeptide coding sequence of SEQ ID NO: 113. In another aspect, the nucleic acid probe is SEQ ID NO: 113. In another aspect, the nucleic acid probe is a polynucleotide that encodes the polypeptide of SEQ ID NO: 114, or a fragment thereof. In another aspect, the nucleic acid probe is the mature polypeptide coding sequence of SEQ ID NO: 115. In another aspect, the nucleic acid probe is SEQ ID NO: 115. In another aspect, the nucleic acid probe is a polynucleotide that encodes the polypeptide of SEQ ID NO: 116, or a fragment thereof.

For long probes of at least 100 nucleotides in length, very low to very high stringency conditions are defined as prehybridization and hybridization at 42° C. in 5×SSPE, 0.3% SDS, 200 micrograms/mL sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₙ, using the calculation according to Bolton and McCarthy (1962, Proc. Natl. Acad. Sci. USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per mL following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 1, 2, 34, 113, or 115. In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 1. In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 2. In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 34. In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 113. In one aspect, the thiolase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 115.

In one aspect, the thiolase is a variant comprising a substitution, deletion, and/or insertion of one or more (several) amino acids of the mature polypeptide of SEQ ID NO: 3, 35, 114, or 116. Preferably, amino acid changes are of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for thiolase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some aspects, the total number of amino acid substitutions, deletions and/or insertions of the mature polypeptide of SEQ ID NO: 3, 35, 114, or 116 is not more than 10, e.g., 1, 2, 3, 4, 5, 6, 7, 8 or 9. In some aspects, the total number of amino acid substitutions, deletions and/or insertions of the mature polypeptide of SEQ ID NO: 3, 35, 114, or 116 is 1, 2, 3, 4, 5, 6, 7, 8, 9, or 10.

In another aspect, the thiolase is a fragment of SEQ ID NO: 3, 35, 114, or 116, wherein the fragment has thiolase activity. In another aspect, the fragment has thiolase activity and contains at least 75%, e.g., at least 80%, 85%, 90%, or 95% of the number of amino acid residues in SEQ ID NO: 3, 35, 114, or 116.

The thiolase may be a fused polypeptide or cleavable fusion polypeptide in which another polypeptide is fused at the N-terminus or the C-terminus of the polypeptide of the present invention. A fused polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Techniques used to isolate or clone a polynucleotide encoding a thiolase, as well as any other polypeptide used in any of the aspects mentioned herein are known in the art and include isolation from genomic DNA, preparation from cDNA, or a combination thereof. The cloning of the polynucleotides from such genomic DNA can be effected, e.g., by using the well known polymerase chain reaction (PCR) or antibody screening of expression libraries to detect cloned DNA fragments with shares structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Schizosaccharomyces, or another or related organism and thus, for example, may be an allelic or species variant of the polypeptide encoding region of the nucleotide sequence.

The thiolase may be obtained from microorganisms of any genus. For purposes of the present invention, the term “obtained from” as used herein in connection with a given source shall mean that the thiolase encoded by a polynucleotide is produced by the source or by a cell in which the polynucleotide from the source has been inserted.

The thiolase may be a bacterial thiolase. For example, the thiolase may be a Gram positive bacterial polypeptide such as a Bacillus, Streptococcus, Streptomyces, Staphylococcus, Enterococcus, Lactobacillus, Lactococcus, Clostridium, Geobacillus, or Oceanobacillus thiolase, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma thiolase.

In one aspect, the thiolase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis thiolase.

In another aspect, the thiolase is a Streptococcus equisimilis, Streptococcus pyogenes, Streptococcus uberis, or Streptococcus equi subsp. Zooepidemicus thiolase. In another aspect, the thiolase is a Streptomyces achromogenes, Streptomyces avermitilis, Streptomyces coelicolor, Streptomyces griseus, or Streptomyces lividans thiolase.

In another aspect, the thiolase is a Clostridium thiolase, such as a Clostridium acetobutylicum thiolase (e.g., Clostridium acetobutylicum thiolase of SEQ ID NO: 3). In another aspect, the thiolase is a Lactobacillus thiolase, such as a Lactobacillus reuteri thiolase (e.g., Lactobacillus reuteri thiolase of SEQ ID NO: 35) or a Lactobacillus brevis thiolase (e.g., Lactobacillus brevis thiolase of SEQ ID NO: 114). In another aspect, the thiolase is a Propionibacterium thiolase, such as a Propionibacterium freudenreichii thiolase (e.g., Propionibacterium freudenreichii of SEQ ID NO: 114).

The thiolase may be a fungal thiolase. In one aspect, the fungal thiolase is a yeast thiolase such as a Candida, Kluyveromyces, Pichia, Saccharomyces, Schizosaccharomyces, or Yarrowia thiolase.

In another aspect, the fungal thiolase is a filamentous fungal thiolase such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria thiolase.

In another aspect, the thiolase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis thiolase.

In another aspect, the thiolase is an Acremonium cellulolyticus, Aspergillus aculeatus, Aspergillus awamori, Aspergillus flavus, Aspergillus fumigatus, Aspergillus foetidus, Aspergillus japonicus, Aspergillus nidulans, Aspergillus niger, Aspergillus oryzae, Aspergillus sojae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonaturn, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride thiolase.

Other thiolase polypeptides that can be used to practice the invention include, e.g., a E. coli thiolase (NP_{—}416728, Martin et al., Nat. Biotechnology 21:796-802 (2003)), and a S. cerevisiae thiolase (NP_{—}015297, Hiser et al., J. Biol. Chem. 269:31383-31389 (1994)), a C. pasteurianum thiolase (e.g., protein ID ABAI8857.I), a C. beijerinckii thiolase (e.g., protein ID EAP59904.1 or EAP59331.1), a Clostridium perfringens thiolase (e.g., protein ID ABG86544.I, ABG83108.I), a Clostridium difficile thiolase (e.g., protein ID CAJ67900.1 or ZP_{—}01231975.1), a Thermoanaerobacterium thermosaccharolyticum thiolase (e.g., protein ID CAB07500.1), a Thermoanaerobacter tengcongensis thiolase (e.g., A.L\.M23825.1), a Carboxydothermus hydrogenoformans thiolase (e.g., protein ID ABB13995.1), a Desulfotomaculum reducens MI-I thiolase (e.g., protein ID EAR45123.1), or a Candida tropicalis thiolase (e.g., protein ID BAA02716.1 or BAA02715.1).

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The thiolase may also be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. The polynucleotide encoding a thiolase may then be derived by similarly screening a genomic or cDNA library of another microorganism or mixed DNA sample. Once a polynucleotide encoding a thiolase has been detected with suitable probe(s) as described herein, the sequence may be isolated or cloned by utilizing techniques that are known to those of ordinary skill in the art (see, e.g., J. Sambrook, E. F. Fritsch, and T. Maniatus, 1989, Molecular Cloning, A Laboratory Manual, 2d edition, Cold Spring Harbor, N.Y.).

### CoA-Transferase and Polynucleotides Encoding a CoA-Transferase

In the present invention, the CoA-transferase can be any CoA-transferase that is suitable for practicing the invention. In some aspects, the CoA-transferase is an acetoacetyl-CoA:acetate/butyrate CoA transferase of EC 2.8.3.9. In some aspects, the CoA-transferase is an acetoacetyl-CoA hydrolase of EC 3.1.2.11. In some aspects, the CoA-transferase is an acetoacetyl-CoA transferase that converts acetoacetyl-CoA and acetate to acetoacetate and acetyl-CoA. In some aspects, the CoA-transferase is a succinyl-CoA:acetoacetate transferase.

In one aspect, the CoA-transferase is a CoA-transferase that is overexpressed under culture conditions wherein an increased amount of acetoacetate is produced.

In one aspect of the recombinant host cells and methods described herein, the CoA-transferase is a protein complex having CoA-transferase activity wherein the one or more (several) heterologous polynucleotides encoding the CoA-transferase complex comprises a first heterologous polynucleotide encoding a first polypeptide subunit and a second polynucleotide encoding a second polypeptide subunit. In one aspect, protein complex is a heteromeric protein complex wherein the first polypeptide subunit and the second polypeptide subunit comprise different amino acid sequences.

In one aspect, the heterologous polynucleotide encoding the first polypeptide subunit, and the heterologous polynucleotide encoding the second polypeptide subunit are contained in a single heterologous polynucleotide. In another aspect, the heterologous polynucleotide encoding the first polypeptide subunit, and the heterologous polynucleotide encoding the second polypeptide are contained in separate heterologous polynucleotides. An expanded discussion of nucleic acid constructs related to CoA-transferase and other polypeptides is described herein.

In one aspect of the recombinant host cells and methods described herein, the CoA-transferase is a protein complex having CoA-transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6, 12, 37, or 41; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 4, 5, 10, 11, 36, or 40, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4, 5, 10, 11, 36, or 40;

and the second polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9, 15, 39, or 43; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 7, 8, 13, 14, 38, or 42, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7, 8, 13, 14, 38, or 42. As can be appreciated by one of skill in the art, in some instances the first and second polypeptide subunits may qualify under more than one of the selections (a), (b) and (c) noted above.

In one aspect of the recombinant host cells and methods described herein, the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 4 or 5, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4 or 5;

and the second polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 7 or 8, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7 or 8.

In one aspect of the recombinant host cells and methods described herein, the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 12; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 10 or 11, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 10 or 11;

and the second polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 15; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 13 or 14, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13 or 14.

In one aspect of the recombinant host cells and methods described herein, the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 37; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 36, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 36;

and the second polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 39; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 38, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 38.

In one aspect of the recombinant host cells and methods described herein, the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 41; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 40, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 40;

and the second polypeptide subunit is selected from: (a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 43; (b) a polypeptide encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 42, or the full-length complementary strand thereof; and (c) a polypeptide encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 42.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6, 12, 37, or 41, and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9, 15, 39, or 43. In one aspect, the first polypeptide subunit comprises an amino acid sequence that differs by no more than ten amino acids, e.g., by no more than five amino acids, by no more than four amino acids, by no more than three amino acids, by no more than two amino acids, or by one amino acid from SEQ ID NO: 6, 12, 37, or 41, and the second polypeptide subunit comprises an amino acid sequence that differs by no more than ten amino acids, e.g., by no more than five amino acids, by no more than four amino acids, by no more than three amino acids, by no more than two amino acids, or by one amino acid from SEQ ID NO: 9, 15, 39, or 43.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6, and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 12, and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 15.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 37, and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 39.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 41, and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 43.

In one aspect, the first polypeptide subunit comprises or consists of the amino acid sequence of SEQ ID NO: 6, 12 37, 41, an allelic variant thereof, or a fragment of the foregoing; and the second polypeptide subunit comprises or consists of the amino acid sequence of SEQ ID NO: 9, 15, 39, 43, an allelic variant thereof, or a fragment of the foregoing. In another aspect, the first polypeptide subunit comprises the amino acid sequence of SEQ ID NO: 6; and the second polypeptide subunit comprises the amino acid sequence of SEQ ID NO: 12. In another aspect, the first polypeptide subunit comprises the amino acid sequence of SEQ ID NO: 9; and the second polypeptide subunit comprises the amino acid sequence of SEQ ID NO: 15. In some aspects of SEQ ID NO: 9 described herein, amino acid 1 of SEQ ID NO: 9 may be a valine or a methionine. In another aspect, the first polypeptide subunit comprises the amino acid sequence of SEQ ID NO: 37; and the second polypeptide subunit comprises the amino acid sequence of SEQ ID NO: 39. In another aspect, the first polypeptide subunit comprises the amino acid sequence of SEQ ID NO: 41; and the second polypeptide subunit comprises the amino acid sequence of SEQ ID NO: 43.

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 4, 5, 10, 11, 36, 40, or the full-length complementary strand thereof, and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 7, 8, 13, 14, 38, 42, or the full-length complementary strand thereof (J. Sambrook, E. F. Fritsch, and T. Maniatis, 1989, supra).

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 4 or 5, or the full-length complementary strand thereof, and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 7 or 8, or the full-length complementary strand thereof.

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 10 or 11, or the full-length complementary strand thereof, and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 13 or 14, or the full-length complementary strand thereof.

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 36, or the full-length complementary strand thereof, and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 38, or the full-length complementary strand thereof.

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 40, or the full-length complementary strand thereof, and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 42, or the full-length complementary strand thereof.

In one aspect, the first polypeptide subunit is encoded by a subsequence of SEQ ID NO: 4, 5, 10, 11, 36, or 40; and/or the second polypeptide subunit is encoded by a subsequence of SEQ ID NO: 7, 8, 13, 14, 38, or 42; wherein the first polypeptide subunit together with the second polypeptide subunit forms a protein complex having CoA-transferase activity (e.g., succinyl-CoA:acetoacetate transferase activity or acetoacetyl-CoA transferase activity).

The polynucleotide of SEQ ID NO: 4, 5, 7, 8, 10, 11, 13, 14, 36, 38, 40, or 42; or a subsequence thereof; as well as the encoded amino acid sequence of SEQ ID NO: 6, 9, 12, 15, 37, 39, 41, 43; or a fragment thereof; may be used to design nucleic acid probes to identify and clone DNA encoding the polypeptide subunits from strains of different genera or species, as described supra. Such probes are encompassed by the present invention. A genomic DNA or cDNA library prepared from such other organisms may be screened for DNA that hybridizes with the probes described above and encodes a polypeptide subunit, as described supra.

In one aspect, the nucleic acid probe is SEQ ID NO: 4, 5, 7, 8, 10, 11, 13, 14, 36, 38, 40, or 42. In another aspect, the nucleic acid probe is a polynucleotide sequence that encodes SEQ ID NO: 6, 9, 12, 15, 37, 39, 41, 43, or a subsequence thereof. In another aspect, the nucleic acid probe is the mature polypeptide coding sequence contained in plasmid pTRGU60 within E. coli DSM 24122, wherein the mature polypeptide coding sequence encodes a polypeptide subunit of a protein complex having succinyl-CoA:acetoacetate transferase activity. In another aspect, the nucleic acid probe is the mature polypeptide coding sequence contained in plasmid pTRGU61 within E. coli DSM 24123, wherein the mature polypeptide coding sequence encodes a polypeptide subunit of a protein complex having succinyl-CoA:acetoacetate transferase activity.

For long probes of at least 100 nucleotides in length, very low to very high stringency and washing conditions are defined as described supra. For short probes of about 15 nucleotides to about 70 nucleotides in length, stringency and washing conditions are defined as described supra.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4, 5, 10, 11, 36, or 40; and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7, 8, 13, 14, 38, or 42.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4 or 5, and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7 or 8.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 10 or 11, and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13 or 14.

In another aspect, the first polypeptide subunit is encoded by the mature polypeptide coding sequence contained in plasmid pTRGU60 within E. coli DSM 24122; and/or the second polypeptide subunit is encoded by the mature polypeptide coding sequence contained in plasmid pTRGU61 within E. coli DSM 24123.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 36, and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 38.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 40, and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 42.

In another aspect, the first polypeptide subunit is a variant comprising a substitution, deletion, and/or insertion of one or more (several) amino acids of the mature polypeptide of SEQ ID NO: 6, 12, 37, 41; and/or the second polypeptide subunit is a variant comprising a substitution, deletion, and/or insertion of one or more (several) amino acids of the mature polypeptide of SEQ ID NO: 9, 15, 39, or 43, as described supra. In some aspects, the total number of amino acid substitutions, deletions and/or insertions of the mature polypeptide of SEQ ID NO: 6, 9, 12, 15, 37, 39, 41, or 43 is not more than 10, e.g., not more than 1, 2, 3, 4, 5, 6, 7, 8 or 9. In another aspect, the total number of amino acid substitutions, deletions and/or insertions of the mature polypeptide of SEQ ID NO: 6, 9, 12, 15, 37, 39, 41, or 43, is 1, 2, 3, 4, 5, 6, 7, 8, 9, or 10.

In another aspect, the first polypeptide subunit is a fragment of SEQ ID NO: 6, 12, 37, or 41, and/or the second polypeptide subunit is a fragment of SEQ ID NO: 9, 15, 39, or 43, wherein the first and second polypeptide subunits together form a protein complex having CoA-transferase activity (e.g., succinyl-CoA:acetoacetate transferase activity or acetoacetyl-CoA transferase activity).

The CoA-transferases (and polypeptide subunits thereof) can also include fused polypeptides or cleavable fusion polypeptides, as described supra.

Techniques used to isolate or clone a polynucleotide encoding a CoA-transferase, and polypeptide subunits thereof, are described supra.

The CoA-transferase (and polypeptide subunits thereof) may be obtained from microorganisms of any genus. In one aspect, the CoA-transferase may be a bacterial, yeast, or fungal CoA-transferase transferase obtained from any microorganism described herein. In one aspect, the CoA-transferase is a Bacillus succinyl-CoA:acetoacetate transferase, e.g., a Bacillus subtilis succinyl-CoA:acetoacetate transferase with a first polypeptide subunit of SEQ ID NO: 6 and a second polypeptide subunit of SEQ ID NO: 9; or a Bacillus mojavensis succinyl-CoA:acetoacetate transferase with a first polypeptide subunit of SEQ ID NO: 12 and a second polypeptide subunit of SEQ ID NO: 15. In another aspect, the CoA-transferase is an E. coli acetoacetyl-CoA transferase, e.g., an E. coli acetoacetyl-CoA transferase with a first polypeptide subunit of SEQ ID NO: 37 and a second polypeptide subunit of SEQ ID NO: 37. In another aspect, the CoA-transferase is a C. acetobutylicum acetoacetyl-CoA transferase, e.g., a C. acetobutylicum acetoacetyl-CoA transferase with a first polypeptide subunit of SEQ ID NO: 41 and a second polypeptide subunit of SEQ ID NO: 43.

Other succinyl-CoA:acetoacetate transferases that can be used to practice the invention include, e.g., a Helicobacter pylori succinyl-CoA:acetoacetate transferase (YP_{—}627417, YP_{—}627418, Corthesy-Theulaz, et al., J Biol Chem 272:25659-25667 (1997)), and Homo sapiens succinyl-CoA:acetoacetate transferase (NP_{—}000427, NP071403, Fukao, T., et al., Genomics 68:144-151 (2000); Tanaka, H., et al., Mol Hum Reprod 8:16-23 (2002)).

The CoA-transferases (and polypeptide subunits thereof) may also be identified and obtained from other sources including microorganisms isolated from nature (e.g., soil, composts, water, etc.) or DNA samples obtained directly from natural materials (e.g., soil, composts, water, etc,) as described supra.

### Acetoacetate Decarboxylase and Polynucleotides Encoding Acetoacetate Decarboxylase

In the present invention, the acetoacetate decarboxylase can be any acetoacetate decarboxylase that is suitable for practicing the invention. In one aspect, the acetoacetate decarboxylase is an acetoacetate decarboxylase that is overexpressed under culture conditions wherein an increased amount of acetone is produced.

In one aspect of the recombinant host cells and methods described herein, the heterologous polynucleotide encoding the acetoacetate decarboxylase is selected from: (a) an acetoacetate decarboxylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 18, 45, 118, or 120; (b) an acetoacetate decarboxylase encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 16, 17, 44, 117, or 119, or the full-length complementary strand thereof; and (c) an acetoacetate decarboxylase encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 16, 17, 44, 117, or 119. As can be appreciated by one of skill in the art, in some instances the acetoacetate decarboxylase may qualify under more than one of the selections (a), (b) and (c) noted above.

In one aspect, the acetoacetate decarboxylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 18. In one aspect, the acetoacetate decarboxylase comprises an amino acid sequence that differs by no more than ten amino acids, e.g., by no more than five amino acids, by no more than four amino acids, by no more than three amino acids, by no more than two amino acids, or by one amino acid from SEQ ID NO: 18.

In one aspect, the acetoacetate decarboxylase comprises or consists of the amino acid sequence of SEQ ID NO: 18, an allelic variant thereof, or a fragment of the foregoing. In another aspect, the acetoacetate decarboxylase comprises the mature polypeptide of SEQ ID NO: 18. In one aspect, the mature polypeptide of SEQ ID NO: 18 is amino acids 1 to 246 of SEQ ID NO: 18.

In one aspect, the acetoacetate decarboxylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 45. In one aspect, the acetoacetate decarboxylase comprises an amino acid sequence that differs by no more than ten amino acids, e.g., by no more than five amino acids, by no more than four amino acids, by no more than three amino acids, by no more than two amino acids, or by one amino acid from SEQ ID NO: 45.

In one aspect, the acetoacetate decarboxylase comprises or consists of the amino acid sequence of SEQ ID NO: 45, an allelic variant thereof, or a fragment of the foregoing. In another aspect, the acetoacetate decarboxylase comprises the mature polypeptide of SEQ ID NO: 45. In one aspect, the mature polypeptide of SEQ ID NO: 45 is amino acids 1 to 259 of SEQ ID NO: 45.

In one aspect, the acetoacetate decarboxylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 118. In one aspect, the acetoacetate decarboxylase comprises an amino acid sequence that differs by no more than ten amino acids, e.g., by no more than five amino acids, by no more than four amino acids, by no more than three amino acids, by no more than two amino acids, or by one amino acid from SEQ ID NO: 118.

In one aspect, the acetoacetate decarboxylase comprises or consists of the amino acid sequence of SEQ ID NO: 118, an allelic variant thereof, or a fragment of the foregoing. In another aspect, the acetoacetate decarboxylase comprises the mature polypeptide of SEQ ID NO: 118.

In one aspect, the acetoacetate decarboxylase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 120. In one aspect, the acetoacetate decarboxylase comprises an amino acid sequence that differs by no more than ten amino acids, e.g., by no more than five amino acids, by no more than four amino acids, by no more than three amino acids, by no more than two amino acids, or by one amino acid from SEQ ID NO: 120.

In one aspect, the acetoacetate decarboxylase comprises or consists of the amino acid sequence of SEQ ID NO: 120, an allelic variant thereof, or a fragment of the foregoing. In another aspect, the acetoacetate decarboxylase comprises the mature polypeptide of SEQ ID NO: 120.

In one aspect, the acetoacetate decarboxylase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 16 or 17, or the full-length complementary strand thereof (J. Sambrook, E. F. Fritsch, and T. Maniatis, 1989, supra). In one aspect, the acetoacetate decarboxylase is encoded by a subsequence of SEQ ID NO: 16 or 17, wherein the acetoacetate decarboxylase has acetoacetate decarboxylase activity.

In one aspect, the acetoacetate decarboxylase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 44, or the full-length complementary strand thereof. In one aspect, the acetoacetate decarboxylase is encoded by a subsequence of SEQ ID NO: 44, wherein the acetoacetate decarboxylase has acetoacetate decarboxylase activity.

In one aspect, the acetoacetate decarboxylase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 117, or the full-length complementary strand thereof. In one aspect, the acetoacetate decarboxylase is encoded by a subsequence of SEQ ID NO: 117, wherein the acetoacetate decarboxylase has acetoacetate decarboxylase activity.

In one aspect, the acetoacetate decarboxylase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 119, or the full-length complementary strand thereof. In one aspect, the acetoacetate decarboxylase is encoded by a subsequence of SEQ ID NO: 119, wherein the acetoacetate decarboxylase has acetoacetate decarboxylase activity.

The polynucleotide of SEQ ID NO: 16, 17, 44, 117, or 119; or a subsequence thereof; as well as the amino acid sequence of SEQ ID NO: 18, 45, 118, or 120; or a fragment thereof; may be used to design nucleic acid probes to identify and clone DNA encoding acetoacetate decarboxylases from strains of different genera or species, as described supra. Such probes are encompassed by the present invention. A genomic DNA or cDNA library prepared from such other organisms may be screened for DNA that hybridizes with the probes described above and encodes a acetoacetate decarboxylase, as described supra.

In one aspect, the nucleic acid probe is SEQ ID NO: 16, 17, 44, 117, or 119. In one aspect, the nucleic acid probe is SEQ ID NO: 16. In one aspect, the nucleic acid probe is SEQ ID NO: 17. In one aspect, the nucleic acid probe is SEQ ID NO: 44. In one aspect, the nucleic acid probe is SEQ ID NO: 17. In one aspect, the nucleic acid probe is SEQ ID NO: 117. In one aspect, the nucleic acid probe is SEQ ID NO: 17. In one aspect, the nucleic acid probe is SEQ ID NO: 119. In another aspect, the nucleic acid probe is a polynucleotide sequence that encodes SEQ ID NO: 18, or a subsequence thereof. In another aspect, the nucleic acid probe is a polynucleotide sequence that encodes SEQ ID NO: 45, or a subsequence thereof. In another aspect, the nucleic acid probe is a polynucleotide sequence that encodes SEQ ID NO: 118, or a subsequence thereof. In another aspect, the nucleic acid probe is a polynucleotide sequence that encodes SEQ ID NO: 120, or a subsequence thereof.

For long probes of at least 100 nucleotides in length, very low to very high stringency and washing conditions are defined as described supra. For short probes of about 15 nucleotides to about 70 nucleotides in length, stringency and washing conditions are defined as described supra.

In another aspect, the acetoacetate decarboxylase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 16 or 17, which encodes a polypeptide having acetoacetate decarboxylase activity.

In another aspect, the acetoacetate decarboxylase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 44, which encodes a polypeptide having acetoacetate decarboxylase activity.

In another aspect, the acetoacetate decarboxylase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 117, which encodes a polypeptide having acetoacetate decarboxylase activity.

In another aspect, the acetoacetate decarboxylase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 119, which encodes a polypeptide having acetoacetate decarboxylase activity.

In another aspect, the acetoacetate decarboxylase is a variant comprising a substitution, deletion, and/or insertion of one or more (several) amino acids of the mature polypeptide of SEQ ID NO: 18, 45, 118, or 120 as described supra. In some aspects, the total number of amino acid substitutions, deletions and/or insertions of the mature polypeptide of SEQ ID NO: 18 or 45 is not more than 10, e.g., not more than 1, 2, 3, 4, 5, 6, 7, 8 or 9. In another aspect, the total number of amino acid substitutions, deletions and/or insertions of the mature polypeptide of SEQ ID NO: 18, 45, 118, or 120 is 1, 2, 3, 4, 5, 6, 7, 8, 9, or 10.

In another aspect, the acetoacetate decarboxylase is a fragment of SEQ ID NO: 18, 45, 118, or 120, wherein the fragment has acetoacetate decarboxylase activity. In one aspect, the number of amino acid residues in the fragment is at least 75%, e.g., at least 80%, 85%, 90%, or 95% of the number of amino acid residues in SEQ ID NO: 18, 45, 118, or 120.

The acetoacetate decarboxylase can also include fused polypeptides or cleavable fusion polypeptides, as described supra.

Techniques used to isolate or clone a polynucleotide encoding an acetoacetate decarboxylase are described supra.

The acetoacetate decarboxylase may be obtained from microorganisms of any genus. In one aspect, the acetoacetate decarboxylase may be a bacterial, yeast, or fungal acetoacetate decarboxylase obtained from any microorganism described herein. In another aspect, the acetoacetate decarboxylase is a Clostridium acetoacetate decarboxylase, e.g., a Clostridium beijerinckii acetoacetate decarboxylase of SEQ ID NO: 18 or a Clostridium acetobutylicum acetoacetate decarboxylase of SEQ ID NO: 45. In another aspect, the acetoacetate decarboxylase is a Lactobacillus acetoacetate decarboxylase, e.g., a Lactobacillus salvarius acetoacetate decarboxylase of SEQ ID NO: 118 or a Lactobacillus plantarum acetoacetate decarboxylase of SEQ ID NO: 120.

Other acetoacetate decarboxylases that can be used to practice the invention include, e.g., a Clostridium saccharoperbutylacetonicum acetoacetate decarboxylase (AAP42566.1, Kosaka, et al., Biosci. Biotechnol Biochem. 71:58-68 (2007)).

The acetoacetate decarboxylases may also be identified and obtained from other sources including microorganisms isolated from nature (e.g., soil, composts, water, etc.) or DNA samples obtained directly from natural materials (e.g., soil, composts, water, etc,) as described supra.

### Isopropanol Dehydrogenase and Polynucleotides Encoding Isopropanol Dehydrogenase

In the present invention, the isopropanol dehydrogenase can be any isopropanol dehydrogenase that is suitable for practicing the invention. In one aspect, the isopropanol dehydrogenase is an isopropanol dehydrogenase that is overexpressed under culture conditions wherein an increased amount of isopropanol is produced.

In one aspect of the recombinant host cells and methods described herein, the heterologous polynucleotide encoding the isopropanol dehydrogenase is selected from: (a) an isopropanol dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 21, 24, 47, or 122; (b) an isopropanol dehydrogenase encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 19, 20, 22, 23, 46, or 121, or the full-length complementary strand thereof; and (c) an isopropanol dehydrogenase encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 19, 20, 22, 23, 46, or 121. As can be appreciated by one of skill in the art, in some instances the isopropanol dehyrogenase may qualify under more than one of the selections (a), (b) and (c) noted above.

In one aspect, the isopropanol dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 21. In another aspect, the isopropanol dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 24. In another aspect, the isopropanol dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 47. In another aspect, the isopropanol dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 122.

In one aspect, the isopropanol dehydrogenase comprises or consists of the amino acid sequence of SEQ ID NO: 21, 24, 47, 122, an allelic variant thereof, or a fragment of the foregoing. In another aspect, the isopropanol dehydrogenase comprises the mature polypeptide of SEQ ID NO: 21. In one aspect, the mature polypeptide of SEQ ID NO: 21 is amino acids 1 to 351 of SEQ ID NO: 21. In another aspect, the isopropanol dehydrogenase comprises the mature polypeptide of SEQ ID NO: 24. In one aspect, the mature polypeptide of SEQ ID NO: 24 is amino acids 1 to 352 of SEQ ID NO: 24. In another aspect, the isopropanol dehydrogenase comprises the mature polypeptide of SEQ ID NO: 47. In one aspect, the mature polypeptide of SEQ ID NO: 47 is amino acids 1 to 356 of SEQ ID NO: 47. In another aspect, the isopropanol dehydrogenase comprises the mature polypeptide of SEQ ID NO: 122.

In one aspect, the isopropanol dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 19, 20, 22, 23, 46, or 121, or the full-length complementary strand thereof (J. Sambrook, E. F. Fritsch, and T. Maniatis, 1989, supra). In one aspect, the isopropanol dehydrogenase is encoded by a subsequence of SEQ ID NO: 19, 20, 22, 23, 46, or 121 wherein the isopropanol dehydrogenase has isopropanol dehydrogenase activity.

In one aspect, the isopropanol dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 19 or 20, or the full-length complementary strand thereof. In one aspect, the isopropanol dehydrogenase is encoded by a subsequence of SEQ ID NO: 19 or 20, wherein the isopropanol dehydrogenase has isopropanol dehydrogenase activity.

In one aspect, the isopropanol dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 22, or 23 or the full-length complementary strand thereof. In one aspect, the isopropanol dehydrogenase is encoded by a subsequence of SEQ ID NO: 22 or 23, wherein the isopropanol dehydrogenase has isopropanol dehydrogenase activity.

In one aspect, the isopropanol dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 46, or the full-length complementary strand thereof. In one aspect, the isopropanol dehydrogenase is encoded by a subsequence of SEQ ID NO: 46, wherein the isopropanol dehydrogenase has isopropanol dehydrogenase activity.

In one aspect, the isopropanol dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 121, or the full-length complementary strand thereof. In one aspect, the isopropanol dehydrogenase is encoded by a subsequence of SEQ ID NO: 121, wherein the isopropanol dehydrogenase has isopropanol dehydrogenase activity.

The polynucleotide of SEQ ID NO: 19, 20, 22, 23, 46, or 121; or a subsequence thereof; as well as the amino acid sequence of SEQ ID NO: 21, 24, 47, or 122; or a fragment thereof; may be used to design nucleic acid probes to identify and clone DNA encoding isopropanol dehydrogenases from strains of different genera or species, as described supra. Such probes are encompassed by the present invention. A genomic DNA or cDNA library prepared from such other organisms may be screened for DNA that hybridizes with the probes described above and encodes an isopropanol dehydrogenase, as described supra.

In one aspect, the nucleic acid probe is the mature polypeptide coding sequence of SEQ ID NO: 19, 20, 22, 23, 46, or 121. In one aspect, the nucleic acid probe is the mature polypeptide coding sequence of SEQ ID NO: 19 or 20. In another aspect, the nucleic acid probe is SEQ ID NO: 19 or 20. In another aspect, the nucleic acid probe is the mature polypeptide coding sequence of SEQ ID NO: 22 or 23. In one aspect, the nucleic acid probe is the mature polypeptide coding sequence of SEQ ID NO: 22 or 23. In one aspect, the nucleic acid probe is the mature polypeptide coding sequence of SEQ ID NO: 46. In another aspect, the nucleic acid probe is SEQ ID NO: 46. In one aspect, the nucleic acid probe is the mature polypeptide coding sequence of SEQ ID NO: 121. In another aspect, the nucleic acid probe is SEQ ID NO: 121. In another aspect, the nucleic acid probe is a polynucleotide sequence that encodes SEQ ID NO: 21, 24, 47, 122, or a subsequence thereof. In another aspect, the nucleic acid probe is a polynucleotide sequence that encodes SEQ ID NO: 21, or a subsequence thereof. In another aspect, the nucleic acid probe is a polynucleotide sequence that encodes SEQ ID NO: 24, or a subsequence thereof. In another aspect, the nucleic acid probe is a polynucleotide sequence that encodes SEQ ID NO: 47, or a subsequence thereof. In another aspect, the nucleic acid probe is a polynucleotide sequence that encodes SEQ ID NO: 122, or a subsequence thereof.

For long probes of at least 100 nucleotides in length, very low to very high stringency and washing conditions are defined as described supra. For short probes of about 15 nucleotides to about 70 nucleotides in length, stringency and washing conditions are defined as described supra.

In another aspect, the isopropanol dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 19, 20, 22, 23, 46, or 121. In one aspect, the isopropanol dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 19 or 20. In another aspect, the isopropanol dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 22 or 23. In another aspect, the isopropanol dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 46. In another aspect, the isopropanol dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 121.

In another aspect, the isopropanol dehydrogenase is a variant comprising a substitution, deletion, and/or insertion of one or more (several) amino acids of the mature polypeptide of SEQ ID NO: 21, 24, 47, or 122, as described supra. In one aspect, the isopropanol dehydrogenase is a variant comprising a substitution, deletion, and/or insertion of one or more (several) amino acids of the mature polypeptide of SEQ ID NO: 21. In another aspect, the isopropanol dehydrogenase is a variant comprising a substitution, deletion, and/or insertion of one or more (several) amino acids of the mature polypeptide of SEQ ID NO: 24. In another aspect, the isopropanol dehydrogenase is a variant comprising a substitution, deletion, and/or insertion of one or more (several) amino acids of the mature polypeptide of SEQ ID NO: 47. In another aspect, the isopropanol dehydrogenase is a variant comprising a substitution, deletion, and/or insertion of one or more (several) amino acids of the mature polypeptide of SEQ ID NO: 122. In some aspects, the total number of amino acid substitutions, deletions and/or insertions of the mature polypeptide of SEQ ID NO: 21, 24, 47 or 122 is not more than 10, e.g., not more than 1, 2, 3, 4, 5, 6, 7, 8 or 9. In another aspect, the total number of amino acid substitutions, deletions and/or insertions of the mature polypeptide of SEQ ID NO: 21, 24, 47, or 122 is 1, 2, 3, 4, 5, 6, 7, 8, 9, or 10.

In another aspect, the isopropanol dehydrogenase is a fragment of SEQ ID NO: 21, 24, 47, or 122, wherein the fragment has isopropanol dehydrogenase activity. In one aspect, the number of amino acid residues in the fragment is at least 75%, e.g., at least 80%, 85%, 90%, or 95% of the number of amino acid residues in SEQ ID NO: 21, 24, 47, or 122.

The isopropanol dehydrogenase can also include fused polypeptides or cleavable fusion polypeptides, as described supra.

Techniques used to isolate or clone a polynucleotide encoding an isopropanol dehydrogenase are described supra.

The isopropanol dehydrogenase may be obtained from microorganisms of any genus. In one aspect, the isopropanol dehydrogenase may be a bacterial, yeast, or fungal isopropanol dehydrogenase obtained from any microorganism described herein. In another aspect, the isopropanol dehydrogenase is a Clostridium isopropanol dehydrogenase, e.g., a Clostridium beijerinckii isopropanol dehydrogenase of SEQ ID NO: 21. In another aspect, the isopropanol dehydrogenase is a Thermoanaerobacter isopropanol dehydrogenase, e.g., a Thermoanaerobacter ethanolicus isopropanol dehydrogenase of SEQ ID NO: 24. In another aspect, the isopropanol dehydrogenase is a Lactobacillus isopropanol dehydrogenase, e.g., a Lactobacillus antri isopropanol dehydrogenase of SEQ ID NO: 47 or a Lactobacillus fermentum isopropanol dehydrogenase of SEQ ID NO: 122.

Other dehydrogenases that can be used to practice the invention include, e.g., a Thermoanaerobacter brockii dehydrogenase (P14941.1, Hanai et al., Appl. Environ. Microbiol. 73:7814-7818 (2007); Peretz et al., Anaerobe 3:259-270 (1997)), a Ralstonia eutropha dehydrogenase (formerly Alcaligenes eutrophus) (YP_{—}299391.1, Steinbuchel and Schlegel et al., Eur. J. Biochem. 141:555-564 (1984)), a Burkholderia sp. AIU 652 dehydrogenase, and a Phytomonas species dehydrogenase (AAP39869.1, Uttaro and Opperdoes et al., Mol. Biochem. Parasitol. 85:213-219 (1997)).

The isopropanol dehydrogenases may also be identified and obtained from other sources including microorganisms isolated from nature (e.g., soil, composts, water, etc.) or DNA samples obtained directly from natural materials (e.g., soil, composts, water, etc,) as described supra.

### Aldehyde Dehydrogenase and Polynucleotides Encoding Aldehyde Dehydrogenase

In the present invention, the aldehyde dehydrogenase can be any aldehyde dehydrogenase that is suitable for practicing the invention. In one aspect, the aldehyde dehydrogenase is an aldehyde dehydrogenase that is overexpressed under culture conditions wherein an increased amount of propanal is produced.

In one aspect of the recombinant host cells and methods described herein, the aldehyde dehydrogenase is selected from: (a) an aldehyde dehydrogenase having at least 60% sequence identity to the mature polypeptide of SEQ ID NO: 27, 30, 33, 51, 54, 57, 60, or 63; (b) an aldehyde dehydrogenase encoded by a polynucleotide that hybridizes under at least low stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof; and (c) an aldehyde dehydrogenase encoded by a polynucleotide having at least 60% sequence identity to the mature polypeptide coding sequence of SEQ ID NO: 25, 26, 28, 29, 31, 32, 48, 49, 50, 52, 53, 55, 56, 58, 59, 61, or 62. As can be appreciated by one of skill in the art, in some instances the aldehyde dehyrogenase may qualify under more than one of the selections (a), (b) and (c) noted above.

In one aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 27.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 30.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 33.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 51.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 54.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 57.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 60.

In another aspect, the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 63.

In one aspect, the aldehyde dehydrogenase comprises or consists of the amino acid sequence of SEQ ID NO: 27, 30, 33, 51, 54, 57, 60, or 63, an allelic variant thereof, or a fragment of the foregoing.

In one aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof (see, e.g., J. Sambrook, E. F. Fritsch, and T. Maniatus, supra).

In one aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 25 or 26, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 28 or 29, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 31 or 32, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 48, 49, or 50, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 52 or 53, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 55 or 56, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 58 or 59, or the full-length complementary strand thereof.

In another aspect, the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 61 or 62, or the full-length complementary strand thereof.

In one aspect, the aldehyde dehydrogenase is encoded by a subsequence of SEQ ID NO: 25, 26, 28, 29, 31, 32, 48, 49, 50, 52, 53, 55, 56, 58, 59, 61, or 62; wherein the subsequence encodes a polypeptide having aldehyde dehydrogenase activity.

The polynucleotide of SEQ ID NO: 25, 26, 28, 29, 31, 32, 48, 49, 50, 52, 53, 55, 56, 58, 59, 61, or 62; or a subsequence thereof; as well as the encoded amino acid sequence of SEQ ID NO: 27, 30, 33, 51, 54, 57, 60, or 63; or a fragment thereof; may be used to design nucleic acid probes to identify and clone DNA encoding aldehyde dehydrogenases from strains of different genera or species, as described supra. Such probes are encompassed by the present invention. A genomic DNA or cDNA library prepared from such other organisms may be screened for DNA that hybridizes with the probes described above and encodes an aldehyde dehydrogenase, as described supra.

For long probes of at least 100 nucleotides in length, very low to very high stringency and washing conditions are defined as described supra. For short probes of about 15 nucleotides to about 70 nucleotides in length, stringency and washing conditions are defined as described supra.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 25, 26, 28, 29, 31, 32, 48, 49, 50, 52, 53, 55, 56, 58, 59, 61, or 62.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 25 or 26.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 28 or 29.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 31 or 32.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 48, 49, or 50.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 52 or 53.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 55 or 56.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 58 or 59.

In one aspect, the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 61 or 62.

In one aspect, the aldehyde dehydrogenase is a variant comprising a substitution, deletion, and/or insertion of one or more (several) amino acids of the mature polypeptide of SEQ ID NO: 27, 30, 33, 51, 54, 57, 60, or 63 as described supra. In some aspects, the total number of amino acid substitutions, deletions and/or insertions of the mature polypeptide of SEQ ID NO: 27, 30, 33, 51, 54, 57, 60, or 63 is not more than 10, e.g., not more than 1, 2, 3, 4, 5, 6, 7, 8 or 9. In another aspect, the total number of amino acid substitutions, deletions and/or insertions of the mature polypeptide of SEQ ID NO: 27, 30, 33, 51, 54, 57, 60, or 63 is 1, 2, 3, 4, 5, 6, 7, 8, 9, or 10.

In another aspect, the aldehyde dehydrogenase is a fragment of SEQ ID NO: 27, 30, 33, 51, 54, 57, 60, or 63, wherein the fragment has aldehyde dehydrogenase activity. In one aspect, the number of amino acid residues in the fragment is at least 75%, e.g., at least 80%, 85%, 90%, or 95% of the number of amino acid residues in SEQ ID NO: 27, 30, 33, 51, 54, 57, 60, or 63.

The aldehyde dehydrogenase can also include fused polypeptides or cleavable fusion polypeptides, as described supra.

Techniques used to isolate or clone a polynucleotide encoding an aldehyde dehydrogenase are described supra.

The aldehyde dehydrogenase may be obtained from microorganisms of any genus. In one aspect, the aldehyde dehydrogenase may be a bacterial, yeast, or fungal aldehyde dehydrogenase obtained from any microorganism described herein.

In one aspect, the aldehyde dehydrogenase is a bacterial aldehyde dehydrogenase. For example, the aldehyde dehydrogenase may be a Gram positive bacterial polypeptide such as a Bacillus, Streptococcus, Streptomyces, Staphylococcus, Enterococcus, Lactobacillus, Lactococcus, Clostridium, Geobacillus, Oceanobacillus, or Propionibacterium aldehyde dehydrogenase, or a Gram negative bacterial polypeptide such as an E. coli (Dawes et al., 1956, Biochim. Biophys. Acta, 22: 253, the content of which is incorporated herein by reference), Pseudomonas, Salmonella, Campylobacter, Helicobacter, Flavobacterium, Fusobacterium, Ilyobacter, Neisseria, or Ureaplasma aldehyde dehydrogenase.

In one aspect, the aldehyde dehydrogenase is a Bacillus aldehyde dehydrogenase, such as a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis aldehyde dehydrogenase.

In another aspect, the aldehyde dehydrogenase is a Lactobacillus aldehyde dehydrogenase, such as a Lactobacillus coffinoides aldehyde dehydrogenase (e.g., the Lactobacillus collinoides aldehyde dehydrogenase of SEQ ID NO: 30)

In another aspect, the aldehyde dehydrogenase is a Propionibacterium aldehyde dehydrogenase, such as a Propionibacterium freudenreichii aldehyde dehydrogenase (e.g., the Propionibacterium freudenreichii aldehyde dehydrogenase of SEQ ID NO: 27 or 51).

In another aspect, the aldehyde dehydrogenase is a Rhodopseudomonas aldehyde dehydrogenase, such as a Rhodopseudomonas palustris aldehyde dehydrogenase (e.g., the Rhodopseudomonas palustris aldehyde dehydrogenase of SEQ ID NO: 54),

In another aspect, the aldehyde dehydrogenase is a Rhodobacter aldehyde dehydrogenase, such as a Rhodobacter capsulatus aldehyde dehydrogenase (e.g., the Rhodobacter capsulatus aldehyde dehydrogenase of SEQ ID NO: 57)

In another aspect, the aldehyde dehydrogenase is a Rhodospirillum aldehyde dehydrogenase, such as a Rhodospirillum rubrum aldehyde dehydrogenase (e.g., the Rhodospirillum rubrum aldehyde dehydrogenase of SEQ ID NO: 60)

In another aspect, the aldehyde dehydrogenase is a Eubacterium aldehyde dehydrogenase, such as a Eubacterium hallii aldehyde dehydrogenase (e.g., the Eubacterium hallii aldehyde dehydrogenase of SEQ ID NO: 63)

In another aspect, the aldehyde dehydrogenase is a Streptococcus aldehyde dehydrogenase, such as a Streptococcus equisimilis, Streptococcus pyogenes, Streptococcus uberis, or Streptococcus equi subsp. Zooepidemicus aldehyde dehydrogenase. In another aspect, the aldehyde dehydrogenase is a Streptomyces aldehyde dehydrogenase, such as a Streptomyces achromogenes, Streptomyces avermitilis, Streptomyces coelicolor, Streptomyces griseus, or Streptomyces lividans aldehyde dehydrogenase.

In another aspect, the aldehyde dehydrogenase is a Clostridium aldehyde dehydrogenase, such as a Clostridium beijerinckii aldehyde dehydrogenase (e.g., the Clostridium beijerinckii aldehyde dehydrogenase of SEQ ID NO: 33), or a Clostridium kluyveri aldehyde dehydrogenase (Burton et al., 1953, J. Biol. Chem., 202: 873, the content of which is incorporated herein by reference).

Other aldehyde dehydrogenases that can be used to practice the present invention include, but are not limited to Rhodococcus opacus (GenBank Accession No. AP011115.1), Entamoeba dispar (GenBank Accession No. DS548207.1) and Lactobacillus reuteri (GenBank Accession No. ACHG01000187.1).

The aldehyde dehydrogenase may also contain n-propanol dehydrogenase activity wherein the enzyme is capable of converting propionyl-CoA to propanal and further reducing propanal to n-propanol. Examples of such multifunctional enzymes having alcohol dehydrogenase activity and aldehyde dehydrogenase activity include, but are not limited to, Lactobacillus sakei (Gen Bank Accession No. CR936503.1), Giardia intestinalis (Gen Bank Accession No. U93353.1), Shewanella amazonensis (GenBank Accession No. CP000507.1), The rmosynechococcus elongatus (GenBank Accession No. BA000039.2), Clostridium acetobutylicum (GenBank Accession No. AE001438.3) and Clostridium carboxidivorans ATCC No. BAA-624T (GenBank Accession No. ACVI01000101.1).

The aldehyde dehydrogenases may also be identified and obtained from other sources including microorganisms isolated from nature (e.g., soil, composts, water, etc.) or DNA samples obtained directly from natural materials (e.g., soil, composts, water, etc,) as described supra.

### Methylmalonyl-CoA Mutase and Polynucleotides Encoding Methylmalonyl-CoA Mutase

In some aspects of the recombinant host cells and methods of use thereof, the host cells have methylmalonyl-CoA mutase activity. In some aspects, the host cells comprise one or more (several) heterologous polynucleotides encoding a methylmalonyl-CoA mutase. The methylmalonyl-CoA mutase can be any methylmalonyl-CoA mutase that is suitable for practicing the invention. In one aspect, the methylmalonyl-CoA mutase is a methylmalonyl-CoA mutase that is overexpressed under culture conditions wherein an increased amount of R-methylmalonyl-CoA is produced.

In one aspect, the methylmalonyl-CoA mutase is selected from (a) a methylmalonyl-CoA mutase having at least 60% sequence identity to the mature polypeptide of SEQ ID NO: 93; (b) a methylmalonyl-CoA mutase encoded by a polynucleotide that hybridizes under low stringency conditions with mature polypeptide coding sequence of SEQ ID NO: 79 or 80, or the full-length complementary strand thereof; and (c) a methylmalonyl-CoA mutase encoded by a polynucleotide having at least 60% sequence identity to mature polypeptide coding sequence of SEQ ID NO: 79 or 80. As can be appreciated by one of skill in the art, in some instances the methylmalonyl-CoA mutase may qualify under more than one of the selections (a), (b) and (c) noted above.

In one aspect, the methylmalonyl-CoA mutase comprises or consists of an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to mature polypeptide of SEQ ID NO: 93. In one aspect, the methylmalonyl-CoA mutase comprises an amino acid sequence that differs by no more than ten amino acids, e.g., by no more than five amino acids, by no more than four amino acids, by no more than three amino acids, by no more than two amino acids, or by one amino acid from mature polypeptide of SEQ ID NO: 93.

In one aspect, the methylmalonyl-CoA mutase comprises or consists of the amino acid sequence of mature polypeptide of SEQ ID NO: 93, an allelic variant thereof, or a fragment of the foregoing, having methylmalonyl-CoA mutase activity. In another aspect, the methylmalonyl-CoA mutase comprises or consists of the amino acid sequence of SEQ ID NO: 93. In another aspect, the methylmalonyl-CoA mutase comprises or consists of the mature polypeptide of SEQ ID NO: 93.

In one aspect, the methylmalonyl-CoA mutase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 79 or 80, or the full-length complementary strand thereof (J. Sambrook, E. F. Fritsch, and T. Maniatis, 1989, supra).

In one aspect, the methylmalonyl-CoA mutase is encoded by a polynucleotide having at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 79 or 80.

In one aspect, the methylmalonyl-CoA mutase is encoded by SEQ ID NO: 79 or 80, the mature polypeptide coding sequence thereof, or a degenerate coding sequence of the foregoing. In one aspect, the methylmalonyl-CoA mutase is encoded by SEQ ID NO: 79 or 80, or a degenerate coding sequence thereof. In one aspect, the methylmalonyl-CoA mutase is encoded by the mature polypeptide coding sequence of SEQ ID NO: 79 or 80, or a degenerate coding sequence of the foregoing. In one aspect, the methylmalonyl-CoA mutase is encoded by a subsequence of SEQ ID NO: 79 or 80 or a degenerate coding thereof, wherein the subsequence encodes a polypeptide having methylmalonyl-CoA mutase activity.

In one aspect, the methylmalonyl-CoA mutase is a variant comprising a substitution, deletion, and/or insertion of one or more (several) amino acids of the mature polypeptide of SEQ ID NO: 93, as described supra. In one aspect, the methylmalonyl-CoA mutase is a variant comprising a substitution, deletion, and/or insertion of one or more (several) amino acids of SEQ ID NO: 93. In one aspect, the methylmalonyl-CoA mutase is a variant comprising a substitution, deletion, and/or insertion of one or more (several) amino acids of the mature polypeptide sequence of SEQ ID NO: 93. In some aspects, the total number of amino acid substitutions, deletions and/or insertions of the mature polypeptide of SEQ ID NO: 93 is not more than 10, e.g., not more than 1, 2, 3, 4, 5, 6, 7, 8 or 9.

In another aspect, the methylmalonyl-CoA mutase is a fragment of the mature polypeptide of SEQ ID NO: 93, wherein the fragment has methylmalonyl-CoA mutase activity. In one aspect, the number of amino acid residues in the fragment is at least 75%, e.g., at least 80%, 85%, 90%, or 95% of the number of amino acid residues in SEQ ID NO: 93.

In one aspect of the recombinant host cells and methods described herein, the methylmalonyl-CoA mutase is a protein complex having methylmalonyl-CoA mutase activity wherein the one or more (several) heterologous polynucleotides encoding the methylmalonyl-CoA mutase complex comprises a first heterologous polynucleotide encoding a first polypeptide subunit and a second heterologous polynucleotide encoding a second polypeptide subunit. In one aspect, the first polypeptide subunit and the second polypeptide subunit comprise different amino acid sequences.

In one aspect, the heterologous polynucleotide encoding the first polypeptide subunit and the heterologous polynucleotide encoding the second polypeptide subunit are contained in a single heterologous polynucleotide. In another aspect, the heterologous polynucleotide encoding the first polypeptide subunit and the heterologous polynucleotide encoding the second polypeptide are contained in separate heterologous polynucleotides. An expanded discussion of nucleic acid constructs related to methylmalonyl-CoA mutases and other polypeptides is described herein.

In one aspect of the methylmalonyl-CoA mutase protein complex, the first polypeptide subunit is selected from: (a) a polypeptide having at least 60% sequence identity to the mature polypeptide SEQ ID NO: 66; (b) a polypeptide encoded by a polynucleotide that hybridizes under at least low stringency conditions with the mature polypeptide coding sequence of SEQ ID NO: 64 or 65, or the full-length complementary strand thereof; and (c) a polypeptide encoded by a polynucleotide having at least 60% sequence identity to the mature polypeptide coding sequence of SEQ ID NO: 64 or 65;

and the second polypeptide subunit is selected from: (a) a polypeptide having at least 60% sequence identity to the mature polypeptide of SEQ ID NO: 69; (b) a polypeptide encoded by a polynucleotide that hybridizes under at least low stringency conditions with the mature polypeptide coding sequence of SEQ ID NO: 67 or 68, or the full-length complementary strand thereof; and (c) a polypeptide encoded by a polynucleotide having at least 60% sequence identity the mature polypeptide coding sequence of SEQ ID NO: 67 or 68.

In one aspect, the first polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 66; and the second polypeptide subunit comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 69. In one aspect, the first polypeptide subunit comprises an amino acid sequence that differs by no more than ten amino acids, e.g., by no more than five amino acids, by no more than four amino acids, by no more than three amino acids, by no more than two amino acids, or by one amino acid from the mature polypeptide of SEQ ID NO: 66; and the second polypeptide subunit comprises an amino acid sequence that differs by no more than ten amino acids, e.g., by no more than five amino acids, by no more than four amino acids, by no more than three amino acids, by no more than two amino acids, or by one amino acid from the mature polypeptide of SEQ ID NO:69.

In one aspect, the first polypeptide subunit comprises or consists of the amino acid sequence of SEQ ID NO: 66, the mature polypeptide of SEQ ID NO: 66, an allelic variant thereof, or a fragment of the foregoing; and the second polypeptide subunit comprises or consists of the amino acid sequence of SEQ ID NO: 69, the mature polypeptide of SEQ ID NO: 69; an allelic variant thereof, or a fragment of the foregoing. In another aspect, the first polypeptide subunit comprises the amino acid sequence of SEQ ID NO: 66; and the second polypeptide subunit comprises the amino acid sequence of SEQ ID NO: 69. In another aspect, the first polypeptide subunit comprises the mature polypeptide of SEQ ID NO: 66; and the second polypeptide subunit comprises the mature polypeptide of SEQ ID NO: 69.

In one aspect, the first polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence SEQ ID NO: 66, or the full-length complementary strand thereof; and the second polypeptide subunit is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 69, or the full-length complementary strand thereof (see, e.g., J. Sambrook, E. F. Fritsch, and T. Maniatus, 1989, supra).

In one aspect, the first polypeptide subunit is encoded by a subsequence of SEQ ID NO: 66; and/or the second polypeptide subunit is encoded by a subsequence of SEQ ID NO: 69; wherein the first polypeptide subunit together with the second polypeptide subunit forms a protein complex having methylmalonyl-CoA mutase activity.

In another aspect, the first polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 66; and the second polypeptide subunit is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 69.

In one aspect, the first polypeptide subunit is encoded by SEQ ID NO: 66, the mature polypeptide coding sequence thereof, or a degenerate coding sequence of the foregoing; and the second polypeptide subunit is encoded by SEQ ID NO: 69, the mature polypeptide coding sequence thereof, or a degenerate coding sequence of the foregoing. In one aspect, the first polypeptide subunit is encoded by SEQ ID NO: 66, or a degenerate coding sequence thereof. In one aspect, the second polypeptide subunit is encoded by SEQ ID NO: 69, or a degenerate coding sequence thereof. In one aspect, the first polypeptide subunit is encoded by the mature polypeptide coding sequence of SEQ ID NO: 66, or a degenerate coding sequence of the foregoing. In one aspect, the second polypeptide subunit is encoded by the mature polypeptide coding sequence of SEQ ID NO: 69, or a degenerate coding sequence of the foregoing.

In one aspect, the first polypeptide subunit is encoded by a subsequence of SEQ ID NO: 66; and/or the second polypeptide subunit is encoded by a subsequence of SEQ ID NO: 69; wherein the first polypeptide subunit together with the second polypeptide subunit forms a protein complex having methylmalonyl-CoA mutase activity.

In another aspect, the first polypeptide subunit is a variant comprising a substitution, deletion, and/or insertion of one or more (several) amino acids of SEQ ID NO: 66 or the mature polypeptide thereof; and/or the second polypeptide subunit is a variant comprising a substitution, deletion, and/or insertion of one or more (several) amino acids of SEQ ID NO: 69 or the mature polypeptide thereof, as described supra. In some aspects, the total number of amino acid substitutions, deletions and/or insertions of SEQ ID NO: 66 or the mature polypeptide sequence thereof; or the total number of amino acid substitutions, deletions and/or insertions of SEQ ID NO: 69 or the mature polypeptide sequence thereof, is not more than 10, e.g., not more than 1, 2, 3, 4, 5, 6, 7, 8 or 9.

In another aspect, the first polypeptide subunit is a fragment of SEQ ID NO: 66, and/or the second polypeptide subunit is a fragment of SEQ ID NO: 69, wherein the first and second polypeptide subunits together form a protein complex having methylmalonyl-CoA mutase activity. In one aspect, the number of amino acid residues in the fragment(s) is at least 75%, e.g., at least 80%, 85%, 90%, or 95% of the number of amino acid residues in SEQ ID NO: 66 or 69.

The methylmalonyl-CoA mutase (or subunits thereof) may also be an allelic variant or artificial variant of a methylmalonyl-CoA mutase.

The methylmalonyl-CoA mutase (or subunits thereof) can also include fused polypeptides or cleavable fusion polypeptides, as described supra.

Techniques used to isolate or clone a polynucleotide encoding a methylmalonyl-CoA mutase (and subunits thereof) are described supra.

The polynucleotide sequences of SEQ ID NO: 79, 80, 64, 65, 67, and 68, or a subsequences thereof; as well as the amino acid sequences of SEQ ID NO: 93, 66, and 69 or a fragment thereof; may be used to design nucleic acid probes to identify and clone DNA encoding methylmalonyl-CoA mutase from strains of different genera or species, as described supra. Such probes are encompassed by the present invention. A genomic DNA or cDNA library prepared from such other organisms may be screened for DNA that hybridizes with the probes described above and encodes a methylmalonyl-CoA mutase, as described supra.

For long probes of at least 100 nucleotides in length, very low to very high stringency and washing conditions are defined as described supra. For short probes of about 15 nucleotides to about 70 nucleotides in length, stringency and washing conditions are defined as described supra.

The methylmalonyl-CoA mutase, and subunits thereof, may be obtained from microorganisms of any genus. In one aspect, the methylmalonyl-CoA mutase may be a bacterial, yeast, or fungal methylmalonyl-CoA mutase obtained from any microorganism described herein.

In one aspect, the methylmalonyl-CoA mutase is an E. coli methylmalonyl-CoA mutase, such as an E. coli methylmalonyl-CoA mutase of SEQ ID NO: 93.

In another aspect, the methylmalonyl-CoA mutase is a Propionibacterium methylmalonyl-CoA mutase, such as a Propionibacterium freudenreichii methylmalonyl-CoA mutase protein complex comprising a first subunit of SEQ ID NO: 66 and a second subunit of SEQ ID NO: 69.

Other methylmalonyl-CoA mutases that can be used to practice the present invention include, but are not limited to the Homo sapiens methylmalonyl-CoA mutase (GenBank ID P22033.3; see Padovani, Biochemistry 45:9300-9306 (2006)), and the Methylobacterium extorquens methylmalonyl-CoA mutase (mcmA subunit, GenBank ID Q84FZ1 and mcmB subunit, GenBank ID Q6TMA2; see Korotkova, J Biol. Chem. 279:13652-13658 (2004)), as well as Shigella flexneri sbm (GenBank ID NP_{—}838397.1), Salmonella enteric SARI 04585 (GenBank ID ABX24358.1), and Yersinia frederiksenii YfreA_{—}01000861 (GenBank ID ZP_{—}00830776.1).

The methylmalonyl-CoA mutase, and subunits thereof, may also be identified and obtained from other sources including microorganisms isolated from nature (e.g., soil, composts, water, etc.) or DNA samples obtained directly from natural materials (e.g., soil, composts, water, etc,) as described supra.

In some aspects of the recombinant host cells and methods of use thereof, the host cells further comprise a heterologous polynucleotide encoding a polypeptide that associates or complexes with the methylmalonyl-CoA mutase. Such polypeptides may increase activity of the methylmalonyl-CoA mutase and may be expressed, e.g., from genes originating adjacent to the methylmalonyl-CoA mutase source genes.

In one aspect, the polypeptide that associates or complexes with the methylmalonyl-CoA mutase is selected from (a) a polypeptide having at least 60% sequence identity to the mature polypeptide of SEQ ID NO: 72 or 94; (b) a polypeptide encoded by a polynucleotide that hybridizes under low stringency conditions with mature polypeptide coding sequence of SEQ ID NO: 70, 71, 81, or 82, or the full-length complementary strand thereof; and (c) a polypeptide encoded by a polynucleotide having at least 60% sequence identity to mature polypeptide coding sequence of SEQ ID NO: 70, 71, 81, or 82.

In one aspect, the polypeptide that associates or complexes with the methylmalonyl-CoA mutase comprises or consists of an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to mature polypeptide of SEQ ID NO: 72. In one aspect, the polypeptide that associates or complexes with the methylmalonyl-CoA mutase comprises an amino acid sequence that differs by no more than ten amino acids, e.g., by no more than five amino acids, by no more than four amino acids, by no more than three amino acids, by no more than two amino acids, or by one amino acid from mature polypeptide of SEQ ID NO: 72.

In one aspect, the polypeptide that associates or complexes with the methylmalonyl-CoA mutase comprises or consists of an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to mature polypeptide of SEQ ID NO: 94. In one aspect, the polypeptide that associates or complexes with the methylmalonyl-CoA mutase comprises an amino acid sequence that differs by no more than ten amino acids, e.g., by no more than five amino acids, by no more than four amino acids, by no more than three amino acids, by no more than two amino acids, or by one amino acid from mature polypeptide of SEQ ID NO: 94.

In one aspect, the polypeptide that associates or complexes with the methylmalonyl-CoA mutase comprises or consists of the amino acid sequence of mature polypeptide of SEQ ID NO: 72 or 94, an allelic variant thereof, or a fragment of the foregoing, having methylmalonyl-CoA mutase activity.

In one aspect, the polypeptide that associates or complexes with the methylmalonyl-CoA mutase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 70 or 71, or the full-length complementary strand thereof (J. Sambrook, E. F. Fritsch, and T. Maniatis, 1989, supra).

In one aspect, the polypeptide that associates or complexes with the methylmalonyl-CoA mutase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 81 or 82, or the full-length complementary strand thereof.

In one aspect, the polypeptide that associates or complexes with the methylmalonyl-CoA mutase is encoded by a polynucleotide having at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 70 or 71.

In one aspect, the polypeptide that associates or complexes with the methylmalonyl-CoA mutase is encoded by a polynucleotide having at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 81 or 82.

In one aspect, the polypeptide that associates or complexes with the methylmalonyl-CoA mutase is encoded by SEQ ID NO: 70, 71, 81, 82, the mature polypeptide coding sequence thereof, or a degenerate coding sequence of the foregoing.

In one aspect, the polypeptide that associates or complexes with the methylmalonyl-CoA mutase is a variant comprising a substitution, deletion, and/or insertion of one or more (several) amino acids of the mature polypeptide of SEQ ID NO: 72 or 94, as described supra. In some aspects, the total number of amino acid substitutions, deletions and/or insertions of the mature polypeptide of SEQ ID NO: 72 or 94 is not more than 10, e.g., not more than 1, 2, 3, 4, 5, 6, 7, 8 or 9. In another aspect, the polypeptide that associates or complexes with the methylmalonyl-CoA mutase is a fragment of the mature polypeptide of SEQ ID NO: 72 or 94.

Other polypeptides that associate or complex with the methylmalonyl-CoA mutase that can be used to practice the present invention include, but are not limited polypeptides from Propionibacterium acnes KPAI71202 (GenBank ID YP_{—}055310.1) and Methylobacterium extorquens meaB (GenBank ID 2QM8—B; see Korotkova, J Biol. Chem. 279: 13652-13658 (2004)).

### Methylmalonyl-CoA Decarboxylase and Polynucleotides Encoding Methylmalonyl-CoA Decarboxylase

In some aspects of the recombinant host cells and methods of use thereof, the host cells have methylmalonyl-CoA decarboxylase activity. In some aspects, the host cells comprise a heterologous polynucleotide encoding a methylmalonyl-CoA decarboxylase. The methylmalonyl-CoA decarboxylase can be any methylmalonyl-CoA decarboxylase that is suitable for practicing the invention. In one aspect, the methylmalonyl-CoA decarboxylase is a methylmalonyl-CoA decarboxylase that is overexpressed under culture conditions wherein an increased amount of propionyl-CoA is produced.

In one aspect, the methylmalonyl-CoA decarboxylase is selected from (a) a methylmalonyl-CoA decarboxylase having at least 60% sequence identity to the mature polypeptide of SEQ ID NO: 103; (b) a methylmalonyl-CoA decarboxylase encoded by a polynucleotide that hybridizes under low stringency conditions with the mature polypeptide coding sequence of SEQ ID NO: 102, or the full-length complementary strand thereof; and (c) a methylmalonyl-CoA decarboxylase encoded by a polynucleotide having at least 60% sequence identity to the mature polypeptide coding sequence of SEQ ID NO: 102. As can be appreciated by one of skill in the art, in some instances the methylmalonyl-CoA decarboxylase may qualify under more than one of the selections (a), (b) and (c) noted above.

In one aspect, the methylmalonyl-CoA decarboxylase comprises or consists of an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the mature polypeptide of SEQ ID NO: 103. In one aspect, the methylmalonyl-CoA decarboxylase comprises an amino acid sequence that differs by no more than ten amino acids, e.g., by no more than five amino acids, by no more than four amino acids, by no more than three amino acids, by no more than two amino acids, or by one amino acid from the mature polypeptide of SEQ ID NO: 103.

In one aspect, the methylmalonyl-CoA decarboxylase comprises or consists of the amino acid sequence of SEQ ID NO: 103, the mature polypeptide sequence of SEQ ID NO: 103, an allelic variant thereof, or a fragment of the foregoing, having methylmalonyl-CoA decarboxylase activity. In another aspect, the methylmalonyl-CoA decarboxylase comprises or consists of the amino acid sequence of SEQ ID NO: 103. In another aspect, the methylmalonyl-CoA decarboxylase comprises or consists of the mature polypeptide sequence of SEQ ID NO: 103.

In one aspect, the methylmalonyl-CoA decarboxylase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 102, or the full-length complementary strand thereof (J. Sambrook, E. F. Fritsch, and T. Maniatis, 1989, supra).

In one aspect, the methylmalonyl-CoA decarboxylase is encoded by a polynucleotide having at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 102.

In one aspect, the methylmalonyl-CoA decarboxylase is encoded by SEQ ID NO: 102, the mature polypeptide coding sequence thereof, or a degenerate coding sequence of the foregoing. In one aspect, the methylmalonyl-CoA decarboxylase is encoded by SEQ ID NO: 102, or a degenerate coding sequence thereof. In one aspect, the methylmalonyl-CoA decarboxylase is encoded by the mature polypeptide coding sequence of SEQ ID NO: 102, or a degenerate coding sequence of the foregoing. In one aspect, the methylmalonyl-CoA decarboxylase is encoded by a subsequence of SEQ ID NO: 102 or a degenerate coding thereof, wherein the subsequence encodes a polypeptide having methylmalonyl-CoA decarboxylase activity.

In one aspect, the methylmalonyl-CoA decarboxylase is a variant comprising a substitution, deletion, and/or insertion of one or more (several) amino acids of the mature polypeptide of SEQ ID NO: 103, as described supra. In one aspect, the methylmalonyl-CoA decarboxylase is a variant comprising a substitution, deletion, and/or insertion of one or more (several) amino acids of SEQ ID NO: 103. In some aspects, the total number of amino acid substitutions, deletions and/or insertions of SEQ ID NO: 103 or the mature polypeptide sequence thereof is not more than 10, e.g., not more than 1, 2, 3, 4, 5, 6, 7, 8 or 9.

In another aspect, the methylmalonyl-CoA decarboxylase is a fragment of SEQ ID NO: 103 or the mature polypeptide sequence thereof, wherein the fragment has methylmalonyl-CoA decarboxylase activity. In one aspect, the number of amino acid residues in the fragment is at least 75%, e.g., at least 80%, 85%, 90%, or 95% of the number of amino acid residues in SEQ ID NO: 103.

The methylmalonyl-CoA decarboxylase may also be an allelic variant or artificial variant of a methylmalonyl-CoA decarboxylase.

The methylmalonyl-CoA decarboxylase can also include fused polypeptides or cleavable fusion polypeptides, as described supra.

Techniques used to isolate or clone a polynucleotide encoding a methylmalonyl-CoA decarboxylase are described supra.

The polynucleotide sequence of SEQ ID NO: 102 or a subsequence thereof; as well as the amino acid sequence of SEQ ID NO: 103 or a fragment thereof; may be used to design nucleic acid probes to identify and clone DNA encoding methylmalonyl-CoA decarboxylase from strains of different genera or species, as described supra. Such probes are encompassed by the present invention. A genomic DNA or cDNA library prepared from such other organisms may be screened for DNA that hybridizes with the probes described above and encodes a methylmalonyl-CoA decarboxylase, as described supra.

In one aspect, the nucleic acid probe is SEQ ID NO: 102 or a degenerate coding sequence thereof. In another aspect, the nucleic acid probe is the mature polypeptide sequence of SEQ ID NO: 102 or a degenerate coding sequence thereof. In another aspect, the nucleic acid probe is a polynucleotide sequence that encodes SEQ ID NO: 103, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

The methylmalonyl-CoA decarboxylase may be obtained from microorganisms of any genus. In one aspect, the methylmalonyl-CoA decarboxylase may be a bacterial, yeast, or fungal methylmalonyl-CoA decarboxylase obtained from any microorganism described herein.

In one aspect, the methylmalonyl-CoA decarboxylase is an E. coli methylmalonyl-CoA decarboxylase, such as the E. coli methylmalonyl-CoA decarboxylase of SEQ ID NO: 103.

Other methylmalonyl-CoA decarboxylases that can be used to practice the present invention include, but are not limited to the Propionigenium modestum (mmdA subunit, GenBank ID CAA05137; mmdB subunit, GenBank ID CAA05140; mmdC subunit, GenBank ID CAA05139; mmdD subunit, GenBank ID CAA05138; see Bott et al., Eur. J. Biochem. 250:590-599 (1997) and Veillonella parvula (mmdA subunit, GenBank ID CAA80872; mmdB subunit, GenBank ID CAA80876; mmdC subunit, GenBank ID CAA80873; mmdD subunit, GenBank ID CAA80875; mmdE subunit, GenBank ID CAA80874; see Huder, J. Biol. Chem. 268:24564-24571 (1993).

The methylmalonyl-CoA decarboxylase may also be identified and obtained from other sources including microorganisms isolated from nature (e.g., soil, composts, water, etc.) or DNA samples obtained directly from natural materials (e.g., soil, composts, water, etc,) as described supra.

### Methylmalonyl-CoA Epimerase and Polynucleotides Encoding Methylmalonyl-CoA Epimerase

In some aspects of the recombinant host cells and methods of use thereof, the host cells have methylmalonyl-CoA epimerase activity. In some aspects, the host cells comprise a heterologous polynucleotide encoding a methylmalonyl-CoA epimerase. The methylmalonyl-CoA epimerase can be any methylmalonyl-CoA epimerase that is suitable for practicing the invention. In one aspect, the methylmalonyl-CoA epimerase is a methylmalonyl-CoA epimerase that is overexpressed under culture conditions wherein an increased amount of S-methylmalonyl-CoA is produced.

In one aspect, the methylmalonyl-CoA epimerase is selected from (a) a methylmalonyl-CoA epimerase having at least 60% sequence identity to the mature polypeptide of SEQ ID NO: 75; (b) a methylmalonyl-CoA epimerase encoded by a polynucleotide that hybridizes under low stringency conditions with the mature polypeptide coding sequence of SEQ ID NO: 73 or 74, or the full-length complementary strand thereof; and (c) a methylmalonyl-CoA epimerase encoded by a polynucleotide having at least 60% sequence identity to the mature polypeptide coding sequence of SEQ ID NO: 73 or 74. As can be appreciated by one of skill in the art, in some instances the methylmalonyl-CoA epimerase may qualify under more than one of the selections (a), (b) and (c) noted above.

In one aspect, the methylmalonyl-CoA epimerase comprises or consists of an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the mature polypeptide of SEQ ID NO: 75. In one aspect, the methylmalonyl-CoA epimerase comprises an amino acid sequence that differs by no more than ten amino acids, e.g., by no more than five amino acids, by no more than four amino acids, by no more than three amino acids, by no more than two amino acids, or by one amino acid from the mature polypeptide of SEQ ID NO: 75.

In one aspect, the methylmalonyl-CoA epimerase comprises or consists of the amino acid sequence of SEQ ID NO: 75, the mature polypeptide sequence of SEQ ID NO: 75, an allelic variant thereof, or a fragment of the foregoing, having methylmalonyl-CoA epimerase activity. In another aspect, the methylmalonyl-CoA epimerase comprises or consists of the amino acid sequence of SEQ ID NO: 75. In another aspect, the methylmalonyl-CoA epimerase comprises or consists of the mature polypeptide sequence of SEQ ID NO: 75.

In one aspect, the methylmalonyl-CoA epimerase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 73 or 74, or the full-length complementary strand thereof (J. Sambrook, E. F. Fritsch, and T. Maniatis, 1989, supra).

In one aspect, the methylmalonyl-CoA epimerase is encoded by a polynucleotide having at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 73 or 74.

In one aspect, the methylmalonyl-CoA epimerase is encoded by SEQ ID NO: 73 or 74, the mature polypeptide coding sequence thereof, or a degenerate coding sequence of the foregoing. In one aspect, the methylmalonyl-CoA epimerase is encoded by SEQ ID NO: 73 or 74, or a degenerate coding sequence thereof. In one aspect, the methylmalonyl-CoA epimerase is encoded by the mature polypeptide coding sequence of SEQ ID NO: 73 or 74, or a degenerate coding sequence thereof. In one aspect, the methylmalonyl-CoA epimerase is encoded by a subsequence of SEQ ID NO: 73 or 74 or a degenerate coding thereof, wherein the subsequence encodes a polypeptide having methylmalonyl-CoA epimerase activity.

In one aspect, the methylmalonyl-CoA epimerase is a variant comprising a substitution, deletion, and/or insertion of one or more (several) amino acids of the mature polypeptide of SEQ ID NO: 75, as described supra. In one aspect, the methylmalonyl-CoA epimerase is a variant comprising a substitution, deletion, and/or insertion of one or more (several) amino acids of SEQ ID NO: 75. In one aspect, the methylmalonyl-CoA epimerase is a variant comprising a substitution, deletion, and/or insertion of one or more (several) amino acids of the mature polypeptide sequence of SEQ ID NO: 75. In some aspects, the total number of amino acid substitutions, deletions and/or insertions of the mature polypeptide of SEQ ID NO: 75 is not more than 10, e.g., not more than 1, 2, 3, 4, 5, 6, 7, 8 or 9.

In another aspect, the methylmalonyl-CoA epimerase is a fragment of SEQ ID NO: 75, wherein the fragment has methylmalonyl-CoA epimerase activity. In one aspect, the number of amino acid residues in the fragment is at least 75%, e.g., at least 80%, 85%, 90%, or 95% of the number of amino acid residues in SEQ ID NO: 75.

The methylmalonyl-CoA epimerase may also be an allelic variant or artificial variant of a methylmalonyl-CoA epimerase.

The methylmalonyl-CoA epimerase can also include fused polypeptides or cleavable fusion polypeptides, as described supra.

Techniques used to isolate or clone a polynucleotide encoding a methylmalonyl-CoA epimerase are described supra.

The polynucleotide sequence of SEQ ID NO: 75 or a subsequence thereof; as well as the amino acid sequence of SEQ ID NO: 73 or 74 or a fragment thereof; may be used to design nucleic acid probes to identify and clone DNA encoding methylmalonyl-CoA epimerases from strains of different genera or species, as described supra. Such probes are encompassed by the present invention. A genomic DNA or cDNA library prepared from such other organisms may be screened for DNA that hybridizes with the probes described above and encodes a methylmalonyl-CoA epimerase, as described supra.

In one aspect, the nucleic acid probe is SEQ ID NO: 73 or 74, or a degenerate coding sequence thereof. In another aspect, the nucleic acid probe is the mature polypeptide coding sequence of SEQ ID NO: 75 or a degenerate coding sequence thereof. In another aspect, the nucleic acid probe is a polynucleotide sequence that encodes SEQ ID NO: 75, the mature polypeptide sequence thereof, or a fragment of the foregoing.

For long probes of at least 100 nucleotides in length, very low to very high stringency and washing conditions are defined as described supra. For short probes of about 15 nucleotides to about 70 nucleotides in length, stringency and washing conditions are defined as described supra.

The methylmalonyl-CoA epimerase may be obtained from microorganisms of any genus. In one aspect, the methylmalonyl-CoA epimerase may be a bacterial, yeast, or fungal methylmalonyl-CoA epimerase obtained from any microorganism described herein.

In one aspect, the methylmalonyl-CoA epimerase is an Propionibacterium methylmalonyl-CoA epimerase, such as a Propionibacterium freudenreichii methylmalonyl-CoA epimerase, e.g., the Propionibacterium freudenreichii methylmalonyl-CoA epimerase of SEQ ID NO: 75.

Other methylmalonyl-CoA epimerases that can be used to practice the present invention include, but are not limited to the Bacillus subtilis YqjC (GenBank ID NP_{—}390273; see Haller, Biochemistry, 39:4622-4629 (2000)), Homo sapiens MCEE (Gen Bank ID Q96PE7.1; see (Fuller, Biochemistry, 1213:643-650 (1983)), Rattus norvegicus Mcee (GenBank ID NP 001099811.1; see Bobik, Biol. Chem. 276:37194-37198 (2001)), Propionibacterium shermanii AF454511 (GenBank ID AAL57846.1; see Haller, Biochemistry 39:4622-9 (2000); McCarthy, Structure 9:637-46 (2001) and Fuller, Biochemistry, 1213:643-650 (1983)), Caenorhabditis elegans mmce (GenBank ID AAT92095.1; see Kuhnl et al., FEBS J 272: 1465-1477 (2005)), and Bacillus cereus AE016877 (GenBank ID AAP08811.1).

The methylmalonyl-CoA epimerase may also be identified and obtained from other sources including microorganisms isolated from nature (e.g., soil, composts, water, etc.) or DNA samples obtained directly from natural materials (e.g., soil, composts, water, etc,) as described supra.

### N-Propanol Dehydrogenase and Polynucleotides Encoding N-Propanol Dehydrogenase

In the present invention, the n-propanol dehydrogenase can be any alcohol dehydrogenase that is suitable for practicing the invention. In one aspect, the n-propanol dehydrogenase is a n-propanol dehydrogenase that is overexpressed under culture conditions wherein an increased amount of n-propanol is produced.

Techniques used to isolate or clone a polynucleotide encoding a n-propanol dehydrogenase are described supra.

The n-propanol dehydrogenase may be obtained from microorganisms of any genus. In one aspect, the n-propanol dehydrogenase may be a bacterial, yeast, or fungal n-propanol dehydrogenase obtained from any microorganism described herein. In another aspect, the n-propanol dehydrogenase is a P. shermanii n-propanol dehydrogenase. In another aspect, the n-propanol dehydrogenase is a S. cerevisiae n-propanol dehydrogenase.

The n-propanol dehydrogenase may also be identified and obtained from other sources including microorganisms isolated from nature (e.g., soil, composts, water, etc.) or DNA samples obtained directly from natural materials (e.g., soil, composts, water, etc,) as described supra.

### Nucleic Acid Constructs

The present invention also relates to nucleic acid constructs comprising a heterologous polynucleotide encoding a thiolase, one or more (several) heterologous polynucleotide(s) encoding CoA-transferase (such as a succinyl-CoA:acetoacetate transferase described herein), a heterologous polynucleotide encoding an acetoacetate decarboxylase, a heterologous polynucleotide encoding an isopropanol dehydrogenase, a heterologous polynucleotide encoding an aldehyde dehydrogenase (and optionally a heterologous polynucleotide encoding methylmalonyl-CoA mutase, a heterologous polynucleotide encoding a methylmalonyl-CoA decarboxylase, a heterologous polynucleotide encoding a methylmalonyl-CoA epimerase and/or a heterologous polynucleotide encoding an n-propanol dehydrogenase) linked to one or more (several) control sequences that direct the expression of the coding sequence(s) in a suitable host cell under conditions compatible with the control sequence(s). Such nucleic acid constructs may be used in any of the host cells and methods describe herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, a polynucleotide that is recognized by a host cell for expression of a polynucleotide encoding any polypeptide described herein. The promoter sequence contains transcriptional control sequences that mediate the expression of the polypeptide. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Each polynucleotide described herein may be operably linked to a promoter that is foreign to the polynucleotide. For example, in one aspect, the heterologous polynucleotide encoding a thiolase is operably linked to a promoter that is foreign to the polynucleotide. In another aspect, the heterologous polynucleotide encoding an acetoacetate decarboxylase is operably linked to promoter foreign to the polynucleotide. In another aspect, the heterologous polynucleotide encoding an isopropanol dehydrogenase is operably linked to promoter foreign to the polynucleotide. In another aspect, the heterologous polynucleotide encoding an aldehyde dehydrogenase is operably linked to a promoter that is foreign to the polynucleotide. In another aspect, the heterologous polynucleotide encoding a CoA-transferase is operably linked to a promoter that is foreign to the polynucleotide. In another aspect, the heterologous polynucleotide encoding a methylmalonyl-CoA mutase is operably linked to a promoter that is foreign to the polynucleotide. In another aspect, the heterologous polynucleotide encoding a methylmalonyl-CoA decarboxylase is operably linked to promoter foreign to the polynucleotide. In another aspect, the heterologous polynucleotide encoding an n-propanol dehydrogenase is operably linked to promoter foreign to the polynucleotide.

As described supra, for a protein complex (e.g., CoA-transferase protein complex) encoded by a heterologous polynucleotide encoding a first polypeptide subunit and a heterologous polynucleotide encoding a second polypeptide subunit, each polynucleotide may be contained in a single heterologous polynucleotide (e.g., a single plasmid), or alternatively contained in separate heterologous polynucleotides (e.g., on separate plasmids). In one aspect, the heterologous polynucleotide encoding the first polypeptide subunit, and the heterologous polynucleotide encoding the second polypeptide subunit are contained in a single heterologous polynucleotide operably linked to a promoter that is foreign to both the both the heterologous polynucleotide encoding the first polypeptide subunit, and the heterologous polynucleotide encoding the second polypeptide subunit. In one aspect, the heterologous polynucleotide encoding the first polypeptide subunit and the heterologous polynucleotide encoding the second polypeptide subunit are contained in separate heterologous polynucleotides wherein the heterologous polynucleotide encoding the first polypeptide subunit is operably linked to a foreign promoter, and the heterologous polynucleotide encoding the second polypeptide subunit is operably linked to a foreign promoter. The promoters in the foregoing may be the same or different.

Examples of suitable promoters for directing the transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from a gene encoding a neutral alpha-amylase in Aspergilli in which the untranslated leader has been replaced by an untranslated leader from a gene encoding triose phosphate isomerase in Aspergilli; non-limiting examples include modified promoters from the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the host cell of choice may be used in the present invention.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present at the N-terminus of a polypeptide, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a heterologous polynucleotide encoding a thiolase, one or more (several) heterologous polynucleotide(s) encoding a CoA-transferase (such as the succinyl-CoA:acetoacetate transferase described herein), a heterologous polynucleotide encoding an acetoacetate decarboxylase, a heterologous polynucleotide encoding an isopropanol dehydrogenase, and/or a heterologous polynucleotide encoding an aldehyde dehydrogenase (and optionally a heterologous polynucleotide encoding a methylmalonyl-CoA mutase, heterologous polynucleotide encoding a methylmalonyl-CoA decarboxylase, a heterologous polynucleotide encoding a methylmalonyl-CoA epimerase, and/or heterologous polynucleotide encoding an n-propanol dehydrogenase); as well as a promoter; and transcriptional and translational stop signals. Such recombinant expression vectors may be used in any of the host cells and methods described herein. The various nucleotide and control sequences may be joined together to produce a recombinant expression vector that may include one or more (several) convenient restriction sites to allow for insertion or substitution of the polynucleotide encoding the polypeptide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

In one aspect, each polynucleotide encoding a thiolase, a CoA-transferase, an acetoacetate decarboxylase, an isopropanol dehydrogenase, a methylmalonyl-CoA mutase, a methylmalonyl-CoA decarboxylase, an aldehyde dehydrogenase, and/or an n-propanol dehydrogenase described herein is contained on an independent vector. In one aspect, at least two of the polynucleotides are contained on a single vector. In one aspect, all the polynucleotides encoding the thiolase, the CoA-transferase, the acetoacetate decarboxylase, the isopropanol dehydrogenase, and the aldehyde dehydrogenase are contained on a single vector.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

As described herein, the present invention relates to, inter alia, recombinant host cells comprising one or more (several) polynucleotide(s) described herein which may be operably linked to one or more (several) control sequences that direct the expression of the polypeptides herein for the recombinant coproduction of n-propanol, isopropanol, or for the coproduction of both n-propanol and isopropanol. The invention also embraces methods of using such host cells for the production of n-propanol, isopropanol, or for the coproduction of both n-propanol and isopropanol.

The host cell may comprise any one or combination of a plurality of the polynucleotides described. For example, a host cell (e.g., a Lactobacillus host cell) designed for the coproduction of both n-propanol and isopropanol may comprise a heterologous polynucleotide encoding a thiolase; one or more (several) heterologous polynucleotides encoding a CoA-transferase (such as a succinyl-CoA:acetoacetate transferase); a heterologous polynucleotide encoding an acetoacetate decarboxylase; a heterologous polynucleotide encoding an isopropanol dehydrogenase; and a heterologous polynucleotide encoding an aldehyde dehydrogenase, wherein the cell produces (or is capable of producing) both n-propanol and isopropanol.

In one exemplary aspect, the recombinant host cell (e.g., Lactobacillus host cell) for the coproduction of n-propanol and isopropanol comprises:

(1) a heterologous polynucleotide encoding a thiolase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 3, 35, 114, or 116;

(2) one or more (several) heterologous polynucleotides encoding a CoA-transferase protein complex comprising a first polypeptide subunit and a second polypeptide subunit, wherein the first polypeptide subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6, 12, 37, or 41, and wherein the second polypeptide subunit has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 9, 15, 39, or 43;

(3) a heterologous polynucleotide encoding an acetoacetate decarboxylase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 18, 45, 118, or 120;

(4) a heterologous polynucleotide encoding an isopropanol dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 21, 24, 47, or 122; and

(5) a heterologous polynucleotide encoding an aldehyde dehydrogenase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 27, 30, 33, 51, 54, 57, 60, or 63;

wherein the recombinant host cell is capable of producing n-propanol and isopropanol.

In some aspects, the recombinant host cell further comprises a heterologous polynucleotide encoding a methylmalonyl-CoA mutase, a heterologous polynucleotide encoding a methylmalonyl-CoA decarboxylase, a heterologous polynucleotide encoding a methylmalonyl-CoA decarboxylase, and/or a heterologous polynucleotide encoding an n-propanol dehydrogenase.

A construct or vector (or multiple constructs or vectors) comprising one or more (several) polynucleotide(s) is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source. The aspects described below apply to the host cells, per se, as well as methods using the host cells.

The host cell may be any cell capable of the recombinant production of a polypeptide of the present invention, e.g., a prokaryote or a eukaryote, and/or any cell capable of the recombinant production of n-propanol, isopropanol, or both n-propanol and isopropanol.

The prokaryotic host cell may be any gram-positive or gram-negative bacterium. Gram-positive bacteria include, but not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The bacterial host cell may also be any Lactobacillus cell including, but not limited to, L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aquaticus, L. arizonensis, L. aviarius, L. bavaricus, L. bifermentans, L. bobalius, L. brevis, L. buchneri, L. bulgaricus, L. cacaonum, L. camelliae, L. capillatus, L. carni, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cypricasei, L. delbrueckii, L. dextrinicus, L. diolivorans, L. divergens, L. durianis, L. equi, L. equicursoris, L. equigenerosi, L. fabifermentans, L. farciminis, L. farraginis, L. ferintoshensis, L. fermentum, L. formicalis, L. fructivorans, L. fructosus, L. frumenti, L. fuchuensis, L. gallinarum, L. gasseri, L. gastricus, L. ghanensis, L. graminis, L. halotolerans, L. hammesii, L. hamsteri, L. harbinensis, L. hayakitensis, L. helveticus, L. heterohiochii, L. hilgardii, L. homohiochii, L. hordei, L. iners, L. ingluviei, L. intestinalis, L. jensenii, L. johnsonii, L. kalixensis, L. kandleri, L. kefiranofaciens, L. kefiranofaciens, L. kefirgranum, L. kefiri, L. kimchii, L. kisonensis, L. kitasatonis, L. kunkeei, L. lactis, L. leichmannii, L. lindneri, L. malefermentans, L. mali, L. maltaromicus, L. manihotivorans, L. mindensis, L. minor, L. minutus, L. mucosae, L. murinus, L. nagelii, L. namurensis, L. nantensis, L. nodensis, L. oeni, L. oligofermentans, L. oris, L. otakiensis, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. piscicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. senmaizukei, L. sharpeae, L. siliginis, L. similis, L. sobrius, L. spicheri, L. sucicola, L. suebicus, L. sunkii, L. suntoryeus, L. taiwanensis, L. thailandensis, L. thermotolerans, L. trichodes, L. tucceti, L. uli, L. ultunensis, L. uvarum, L. vaccinostercus, L. vaginalis, L. versmoldensis, L. viridescens, L. vitulinus, L. xylosus, L. yamanashiensis, L. zeae, and L. zymae. In one aspect, the bacterial host cell is L. plantarum, L. fructivorans, or L. reuteri.

In one aspect, the host cell is a member of a genus selected from Escherichia (e.g., Escherichia coli), Lactobacillus (e.g., Lactobacillus plantarum, Lactobacillus fructivorans, or Lactobacillus reuteri), and Propionibacterium (e.g., Propionibacterium freudenreichii). In one preferred aspect, the host cell is a Lactobacillus host cell.

The introduction of DNA into a Bacillus cell may, for instance, be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios 68: 189-207, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus aculeatus, Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In one aspect, the host cell is an Aspergillus host cell. In another aspect, the host cell is Aspergillus oryzae.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023 and Yelton et al., 1984, Proc. Natl. Acad. Sci. USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In some aspects, the host cell comprises one or more (several) polynucleotide(s) described herein, wherein the host cell secretes (and/or is capable of secreting) an increased level of isopropanol and/or n-propanol compared to the host cell without the one or more (several) polynucleotide(s) when cultivated under the same conditions. In some aspects, the host cell secretes and/or is capable of secreting an increased level of isopropanol and/or n-propanol of at least 25%, e.g., at least 50%, at least 100%, at least 150%, at least 200%, at least 300%, or at 500% compared to the host cell without the one or more (several) polynucleotide(s), when cultivated under the same conditions.

In any of these aspects, the host cell produces (and/or is capable of producing) n-propanol and/or isopropanol at a yield of at least than 10%, e.g., at least than 20%, at least than 30%, at least than 40%, at least than 50%, at least than 60%, at least than 70%, at least than 80%, or at least than 90%, of theoretical.

In any of these aspects, the recombinant host has an n-propanol and/or isopropanol volumetric productivity (or a combined n-propanol and isopropanol volumetric productivity) greater than about 0.1 g/L per hour, e.g., greater than about 0.2 g/L per hour, 0.5 g/L per hour, 0.75 g/L per hour, 1.0 g/L per hour, 1.25 g/L per hour, 1.5 g/L per hour, 1.75 g/L per hour, 2.0 g/L per hour, 2.25 g/L per hour, 2.5 g/L per hour, or 3.0 g/L per hour.

The recombinant host cells may be cultivated in a nutrient medium suitable for production of the enzymes described herein using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the desired polypeptide to be expressed and/or isolated. The cultivation takes place in a suitable nutrient medium comprising carbon and nitrogen sources and inorganic salts, using procedures known in the art. Suitable media are available from commercial suppliers, may be prepared according to published compositions (e.g., in catalogues of the American Type Culture Collection), or may be prepared from commercially available ingredients.

The enzymes herein and activities thereof can be detected using methods known in the art and/or described above. These detection methods may include use of specific antibodies, formation of an enzyme product, or disappearance of an enzyme substrate. See, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999); and Hanai et al., Appl. Environ. Microbiol. 73:7814-7818 (2007)).

### Methods

The present invention also relates to methods of using the recombinant host cells described herein for the production of n-propanol, isopropanol, or the coproduction of n-propanol and isopropanol.

In one aspect, the invention embraces a method of producing n-propanol, comprising: (a) cultivating any one of the recombinant host cells described herein (e.g., any host cell with methylmalonyl-CoA mutase activity, methylmalonyl-CoA decarboxylase activity, methylmalonyl-CoA epimerase activity, aldehyde dehydrogenase activity, and/or n-propanol dehydrogenase activity) in a medium under suitable conditions to produce n-propanol; and (b) recovering the n-propanol. In one aspect, the recombinant host cell comprises aldehyde dehydrogenase activity. In one aspect, the invention embraces a method of producing n-propanol, comprising: (a) cultivating in a medium any one of the recombinant host cells described herein, wherein the host cell comprises a heterologous polynucleotide encoding an aldehyde dehydrogenase (and optionally comprising one or more heterologous polynucleotides encoding a methylmalonyl-CoA mutase; a heterologous polynucleotide encoding a methylmalonyl-CoA decarboxylase; a heterologous polynucleotide encoding a methylmalonyl-CoA epimerase; and/or a heterologous polynucleotide encoding an n-propanol dehydrogenase) under suitable conditions to produce n-propanol; and (b) recovering the n-propanol. In one aspect, the medium is a fermentable medium.

In one aspect, the invention embraces a method of producing n-propanol described herein from, e.g., glucose, succinate, succinyl-CoA, or propoionyl-CoA. In one aspect, the invention embraces a method of producing propanal from a recombinant host cell described herein from, e.g., glucose, succinate, succinyl-CoA, or propoionyl-CoA.

In one aspect, the invention embraces a method of producing isopropanol, comprising: (a) cultivating any one of the recombinant host cells described herein (e.g., any host cell with thiolase activity, succinyl-CoA:acetoacetate transferase activity, acetoacetate decarboxylase activity, and isopropanol dehydrogenase activity) in a medium under suitable conditions to produce isopropanol; and (b) recovering the isopropanol. In one aspect, the invention embraces a method of producing isopropanol, comprising: (a) cultivating in a medium any one of the recombinant host cells described herein, wherein the host cell comprises a heterologous polynucleotide encoding a thiolase; one or more (several) heterologous polynucleotides encoding a succinyl-CoA:acetoacetate transferase; a heterologous polynucleotide encoding an acetoacetate decarboxylase; and/or a heterologous polynucleotide encoding an isopropanol dehydrogenase under suitable conditions to produce isopropanol; and (b) recovering the isopropanol. In one aspect, the medium is a fermentable medium. In another aspect, the medium is a fermentable medium comprising sugarcane juice (e.g., non-sterilized sugarcane juice).

In one aspect, the invention embraces a method of coproducing n-propanol and isopropanol, comprising: (a) cultivating any one of the recombinant host cells described herein (e.g., any host cell with thiolase activity, CoA-transferase activity, acetoacetate decarboxylase activity, isopropanol dehydrogenase activity, methylmalonyl-CoA mutase activity, methylmalonyl-CoA decarboxylase activity, aldehyde dehydrogenase activity, and/or n-propanol dehydrogenase activity) in a medium under suitable conditions to produce n-propanol and isopropanol; and (b) recovering the n-propanol and isopropanol. In one aspect, the invention embraces a method of producing n-propanol and isopropanol, comprising: (a) cultivating in a medium any one of the recombinant host cells described herein, wherein the host cell comprises a heterologous polynucleotide encoding a thiolase; one or more (several) heterologous polynucleotides encoding a CoA-transferase (e.g., succinyl-CoA:acetoacetate transferase); a heterologous polynucleotide encoding an acetoacetate decarboxylase; a heterologous polynucleotide encoding an isopropanol dehydrogenase; a heterologous polynucleotide encoding a methylmalonyl-CoA mutase; a heterologous polynucleotide encoding a methylmalonyl-CoA decarboxylase; a heterologous polynucleotide encoding an aldehyde dehydrogenase; and/or a heterologous polynucleotide encoding an n-propanol dehydrogenase under suitable conditions to produce n-propanol and isopropanol; and (b) recovering the n-propanol and isopropanol. In one aspect, the medium is a fermentable medium. In another aspect, the medium is a fermentable medium comprising sugarcane juice (e.g., non-sterilized sugarcane juice).

The methods may be performed in a fermentable medium comprising any one or more (several) sugars, such as glucose, fructose, sucrose, cellobiose, xylose, xylulose, arabinose, mannose, galactose, and/or soluble oligosaccharides. In some instances, the fermentation medium is derived from a natural source, such as sugar cane, starch, or cellulose, and may be the result of pretreating the source by enzymatic hydrolysis (saccharification). In one aspect, the medium is a fermentable medium comprising sugarcane juice (e.g., non-sterilized sugarcane juice).

In addition to the appropriate carbon sources from one or more (several) sugar(s), the fermentable medium may contain other nutrients or stimulators known to those skilled in the art, such as macronutrients (e.g., nitrogen sources) and micronutrients (e.g., vitamins, mineral salts, and metallic cofactors). In some aspects, the carbon source can be preferentially supplied with at least one nitrogen source, such as yeast extract, N₂ or peptone (e.g., Bacto™ Peptone). Nonlimiting examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of mineral salts and metallic cofactors include, but are not limited to Na, P, K, Mg, S, Ca, Fe, Zn, Mn, and Cu.

Suitable conditions used for the methods of production may be determined by one skilled in the art in light of the teachings herein. In some aspects of the methods, the host cells are cultivated for about 12 to about 216 hours, such as about 24 to about 144 hours, about 36 to about 96 hours. The temperature is typically between about 26° C. to about 60° C., in particular about 34° C. or 50° C., and at about pH 3 to about pH 8, such as around pH 4-5, 6, or 7.

Cultivation may be performed under anaerobic, substantially anaerobic (microaerobic), or aerobic conditions, as appropriate. Briefly, anaerobic refers to an environment devoid of oxygen, substantially anaerobic (microaerobic) refers to an environment in which the concentration of oxygen is less than air, and aerobic refers to an environment wherein the oxygen concentration is approximately equal to or greater than that of the air. Substantially anaerobic conditions include, for example, a culture, batch fermentation or continuous fermentation such that the dissolved oxygen concentration in the medium remains less than 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. In some embodiments, the cultivation is performed under anaerobic conditions or substantially anaerobic conditions.

The methods of the present invention can employ any suitable fermentation operation mode. For example, a batch mode fermentation may be used with a close system where culture media and host microorganism, set at the beginning of fermentation, have no additional input except for the reagents certain reagents, e.g. for pH control, foam control or others required for process sustenance. The process described in the present invention can also be employed in Fed-batch or continuous mode.

The methods of the present invention may be practiced in several bioreactor configurations, such as stirred tank, bubble column, airlift reactor and others known to those skilled in the art.

The methods may be performed in free cell culture or in immobilized cell culture as appropriate. Any material support for immobilized cell culture may be used, such as alginates, fibrous bed, or argyle materials such as chrysotile, montmorillonite KSF and montmorillonite K-10.

In one aspect of the methods, the product (e.g., n-propanol and/or isopropanol) is produced at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L. In one aspect of the methods, the product (e.g., n-propanol) is produced at a titer greater than about 0.01 gram per gram of carbohydrate, e.g., greater than about 0.02, 0.05, 0.75, 0.1, 0.2, 0.3, 0.4, 0.5, 0.6, 0.7, 0.8, 0.9, or 1.0 gram per gram of carbohydrate.

In one aspect of the methods, the amount of product (e.g., isopropanol and/or n-propanol) is at least 5%, e.g., at least 10%, at least 15%, at least 20%, at least 25%, at least 30%, at least 50%, at least 75%, or at least 100% greater compared to cultivating the host cell without the heterologous polynucleotide(s) under the same conditions.

The recombinant n-propanol and isopropanol can be optionally recovered from the fermentation medium using any procedure known in the art including, but not limited to, chromatography (e.g., size exclusion chromatography, adsorption chromatography, ion exchange chromatography), electrophoretic procedures, differential solubility, osmosis, distillation, extraction (e.g., liquid-liquid extraction), pervaporation, extractive filtration, membrane filtration, membrane separation, reverse, or ultrafiltration. In one example, the isopropanol is separated from other fermented material and purified by conventional methods of distillation. Accordingly, in one aspect, the method further comprises purifying the recovered n-propanol and isopropanol by distillation.

The recombinant n-propanol and isopropanol may also be purified by the chemical conversion of impurities (contaminants) to products more easily removed from isopropanol by the procedures described above (e.g., chromatography, electrophoretic procedures, differential solubility, distillation, or extraction) and/or by direct chemical conversion of one or more (several) of the impurities to n-propanol or isopropanol. For example, in one aspect, the method further comprises purifying the recovered isopropanol by converting acetone contaminant to isopropanol, or further comprises purifying the recovered n-propanol by converting propanal contaminant to n-propanol. Conversion of acetone to isopropanol or propanal to n-propanol may be accomplished using any suitable reducing agent known in the art (e.g., lithium aluminium hydride (LiAlH₄), a sodium species (such as sodium amalgam or sodium borohydride (NaBH₄)), tin species (such as tin(II) chloride), hydrazine, zinc-mercury amalgam (Zn(Hg)), diisobutylaluminum hydride (DIBAH), oxalic acid (C₂H₂O₄), formic acid (HCOOH), ascorbic acid, iron species (such as iron(II) sulfate), or the like).

In some aspects of the methods, the recombinant n-propanol and isopropanol before and/or after being optionally purified is substantially pure. With respect to the methods of producing isopropanol, “substantially pure” intends a recovered preparation of n-propanol and isopropanol that contains no more than 15% impurity, wherein impurity intends compounds other than propanol but does not include the other propanol isomer. In one variation, a preparation of substantially pure isopropanol is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

N-propanol and isopropanol produced by any of the methods described herein may be converted to propylene. Propylene can be produced by the chemical dehydration of n-propanol and/or isopropanol using acidic catalysts known in the art, such as acidic alumina and zeolites, acidic organic-sulfonic acid resins, mineral acids such as phosphoric and sulfuric acids, and Lewis acids such as boron trifluoride and aluminum compounds (March, Jerry. Advanced Organic Chemistry. New York: John Wiley and Sons, 1992). Suitable temperatures for dehydration of n-propanol and/or isopropanol to propylene typically range from about 180° C. to about 600° C., e.g., 300° C. to about 500° C., or 350° C. to about 450° C.

The dehydration reaction of n-propanol and/or iso-propanol is typically conduced in an adiabatic or isothermal reactor, which can also be a fixed or a fluidized bed reactor; and can be optimized using residence time ranging from about 0.1 to about 60 seconds, e.g., from about 1 to about 30 seconds. Non-converted alcohol can be recycled to the dehydration reactor.

In one aspect, the invention embraces a method of producing propylene, comprising: (a) cultivating a recombinant host cell described herein in a medium under suitable conditions to produce n-propanol and/or isopropanol; (b) recovering the n-propanol and isopropanol; (c) dehydrating the n-propanol and isopropanol under suitable conditions to produce propylene; and (d) recovering the propylene. In one aspect, the medium is a fermentable medium. In another aspect, the medium is a fermentable medium comprising sugarcane juice (e.g., non-sterilized sugarcane juice). In one aspect, the amount of n-propanol and/or isopropanol (or total amount of n-propanol and isopropanol) produced prior to dehydrating the n-propanol and isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L. In one aspect, dehydrating the n-propanol and isopropanol under suitable conditions to produce propylene comprises contacting or treating the n-propanol and isopropanol with an acid catalyst, as known in the art.

Contaminants that may be generated during dehydration may be removed through purification using techniques known in the art. For example, propylene can be washed with water or a caustic solution to remove acidic compounds like carbon dioxide and/or fed into beds to absorb polar compounds like water or for the removal of, e.g., carbon monoxide. Alternatively, a distillation column can be used to separate higher hydrocarbons such as propane, butane, butylene and higher compounds. The separation of propylene from contaminants like ethylene may be carried out by methods known in the art, such as cryogenic distillation.

Suitable assays to test for the production of n-propanol, isopropanol and propylene for the methods of production and host cells described herein can be performed using methods known in the art. For example, final n-propanol and isopropanol product, as well as intermediates (e.g., acetone) and other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of n-propanol and isopropanol in the fermentation broth can also be tested with the culture supernatant. Byproducts and residual sugar in the fermentation medium (e.g., glucose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

The propylene produced from n-propanol may be further converted to polypropylene or polypropylene copolymers by polymerization processes known in the art. Suitable temperatures typically range from about 105° C. to about 300° C. for bulk polymerization, or from about 50° C. to about 100° C. for polymerization in suspension. Alternatively, polypropylene can be produced in a gas phase reactor in the presence of a polymerization catalyst such as Ziegler-Natta or metalocene catalysts with temperatures ranging from about 60° C. to about 80° C.

The present invention is further described by the following examples that should not be construed as limiting the scope of the invention.

### EXAMPLES

Chemicals used as buffers and substrates were commercial products of at least reagent grade.

### Media

LB plates were composed of 37 g LB agar (Sigma cat no. L3027) and double distilled water to 1 L.

LBPGS plates were composed of 37 g LB agar (Sigma cat no. L3027), 0.5% starch (Merck cat. no. 101252), 0.01 M K₂PO₄, 0.4% glucose, and double distilled water to 1 L.

TY bouillon medium was composed of 20 g tryptone (Difco cat no. 211699), 5 g yeast extract (Difco cat no. 212750), 7*10⁻³ g ferrochloride, 1*10⁻³ g manganese(II)-chloride, 1.5*10⁻³ g magnesium sulfate, and double distilled water to 1 L.

Minimal medium (MM) was composed of 20 g glucose, 1.1 g KH₂PO₄, 8.9 g K₂HPO₄; 1.0 g (NH₄)₂SO₄; 0.5 g Na-citrate; 5.0 g MgSO₄.7H₂O; 4.8 mg MnSO₄.H₂O; 2 mg thiamine; 0.4 mg/L biotin; 0.135 g FeCl₃.6H₂O; 10 mg ZnCl₂.4H₂O; 10 mg CaCl₂.6H₂O; 10 mg Na₂MoO₄.2H₂O; 9.5 mg CuSO₄.5H₂O; 2.5 mg H₃BO₃; and double distilled water to 1 L, pH adjusted to 7 with HCl.

MRS medium was obtained from Difco™, as either Difco™ Lactobacilli MRS Agar or Difco™ Lactobacilli MRS Broth, having the following compositions—Difco™ Lactobacilli MRS Agar: Proteose Peptone No. 3 (10.0 g), Beef Extract (10.0 g), Yeast Extract (5.0 g), Dextrose (20.0 g), Polysorbate 80 (1.0 g), Ammonium Citrate (2.0 g), Sodium Acetate (5.0 g), Magnesium Sulfate (0.1 g), Manganese Sulfate (0.05 g), Dipotassium Phosphate (2.0 g), Agar (15.0 g) and water to 1 L. Difco™ Lactobacilli MRS Broth: Consists of the same ingredients without the agar.

LC (Lactobacillus Carrying) medium was composed of Trypticase (10 g), Tryptose (3 g), Yeast extract (5 g), KH₂PO₄ (3 g), Tween 80 (1 ml), sodium-acetate (1 g), ammonium citrate (1.5 g), Cystein-HCl (0.2 g), MgSO₄.7H₂O (12 mg), FeSO₄.7H₂O (0.68 mg), MnSO₄.2H₂O (25 mg), and double distilled water to 1 L, pH adjusted to 7.0. Stearile glucose is added after autoclaving, to 1% (5 ml of a 20% glucose stock solution/100 ml medium).

### Host Strains

Lactobacillus plantarum SJ10656 (O4ZY1):

Lactobacillus plantarum strain NC8 (Aukrust, T., and Blom, H. (1992) Transformation of Lactobacillus strains used in meat and vegetable fermentations. Food Research International, 25, 253-261) containing plasmid pVS2 (von Wright, A., Tynkkynen, S., Suominen, M. (1987) Cloning of a Streptococcus lactis subsp. Lactis chromosomal fragment associated with the ability to grow in milk. Applied and Environmental Microbiology, 53, 1584-1588) was received on a MRS agar plate with 5 microgram/ml erythromycin, and frozen as SJ10491. SJ10491 was cured for pVS2 by plating to single colonies from a culture propagated in MRS medium containing novobiocin at 0.125 microgram/ml, essentially as described by Ruiz-Barba et al. (Ruiz-Barba, J. L., Plard, J. C., Jiménez-Diaz, R. (1991) Plasmid profiles and curing of plasmids in Lactobacillus plantarum strains isolated from green olive fermentations. Journal of Applied Bacteriology, 71, 417-421). Erythromycin sensitive colonies were identified, absence of pVS2 was confirmed by plasmid preparation and PCR amplification using plasmid specific primers, and a plasmid-free derivative frozen as SJ10511.

SJ10511 was inoculated into MRS medium, propagated without shaking for one day at 37° C., and spread on MRS agar plates to obtain single colonies. After overnight growth at 37° C., a single colony was reisolated on MRS agar plates to obtain single colonies. After two days growth at 37° C., a single colony was again reisolated on a MRS agar plate, the plate incubated at 37° C. for three days, and the cell growth on the plate was scraped off and stored in the strain collection as SJ10656 (alternative name: O4ZY1).

Lactobacillus reuteri SJ10655 (O4ZXV):

A strain described as Lactobacillus reuteri DSM20016 was obtained from a public strain collection and kept in a Novozymes strain collection as NN016599. This strain was subcultured in MRS medium, and an aliquot frozen as SJ10468. SJ10468 was inoculated into MRS medium, propagated without shaking for one day at 37° C., and spread on MRS agar plates to obtain single colonies. After two days growth at 37° C., a single colony was reisolated on a MRS agar plate, the plate incubated at 37° C. for three days, and the cell growth on the plate was scraped off and stored in the strain collection as SJ10655 (alternative name: O4ZXV).

The same cell growth was used to inoculate a 10 ml MRS culture, which was incubated without shaking at 37° C. for 3 days, whereafter cells were harvested by centrifugation and genomic DNA was prepared (using a QIAamp DNA Blood Kit from QIAGEN) and sent for genome sequencing.

The genome sequence revealed that the isolate SJ1655 (O4ZXV) has a genome essentially identical to that of JCM1112, rather than to that of the closely related strain DSM20016. JCM1112 and DSM20016 are derived from the same original isolate, L. reuteri F275 (Morita, H, Toh, H., Fukuda, S., Horikawa, H., Oshima, K., Suzuki, T., Murakami, M., Hisamatsu, S., Kato, Y., Takizawa, T., Fukuoka, H., Yoshimura, T., Itoh, K., O'Sullivan, D. J., McKay, L., Ohno, H., Kikuchi, J., Masaoka, T., Hattori, M. (2008) Comparative genome analysis of Lactobacillus reuteri and Lactobacillus fermentum reveal a genomic island for reuterin and cobalamin production. DNA research, 15, 151-161.)

Lactobacillus reuteri SJ11044:

L. reuteri SJ11044 was obtained from SJ10655 (O4ZXV) by the following procedure: SJ10655 was transformed with pSJ10769 (described below), a pVS2-based plasmid containing an alcohol-dehydrogenase expression construct, resulting in SJ11016 (described below).

SJ11016 was propagated in MRS medium with 0.25 microgram/ml novobiocin, to cure the strain for the plasmid, plated on MRS agar plates, and erythromycin sensitive colonies identified by replica plating. One such strain was kept as SJ11044. Strain SJ11044 was prepared for electroporation, along with the original strain SJ10655, and no difference in electroporation frequency, using pSJ10600 (described below) as a test plasmid, was observed.

SJ11044 electrocompetent cells such manufactured were subsequently used for certain experiments, as an (identical) substitute for SJ10655.

Bacillus subtilis DN1885 has been described in (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis. Journal of Bacteriology, 172, 4315-4321). Bacillus subtilis JA1343, is a sporulation negative derivative of PL1801. Part of the gene SpoIIAC has been deleted to obtain the sporulation negative phenotype.

Escherichia coli:

SJ2: (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis. Journal of Bacteriology, 172, 4315-4321).

MG1655: (Blattner, F. R., Plunkett, G. 3rd, Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B., Shao, Y. (1997). The complete genome sequence of Escherichia coli K-12. Science, 277, 1453-1462).

TG1: TG1 is a commonly used cloning strain and was obtained from a commercial supplier having the following genotype: F′[traD36 lacIq Δ(lacZ) M15 proA+B+] glnV (supE) thi-1 Δ(mcrB-hsdSM)₅ (rK-mK-McrB-) thi Δ(lac-proAB).

### Example 1

### Electroporation Protocol for Lactobacillus Strains

Plasmid DNA was introduced into Lactobacillus strains by electroporation.

Lactobacillus plantarum strains were prepared for electroporation as follows: The strain was inoculated from a frozen stock culture into MRS medium with glycine added to 1%, and incubated without shaking at 37° C. overnight. It was then diluted 1:100 into fresh MRS+1% glycine, and incubated without shaking at 37° C. until OD₆₀₀ reached 0.6. The cells were harvested by centrifugation at 4000 rpm. for 10 minutes at 30° C. The cell pellet was subsequently resuspended in the original volume of 1 mM MgCl₂, and pelleted by centrifugation as above. The cell pellet was then resuspended in the original volume of 30% PEG1500, and pelleted by centrifugation as above. They cells were finally gently resuspended in 1/100 the original volume of 30% PEG1500, and 50 microliter aliquots were quickly frozen in an alcohol/dry ice bath, and kept at −80° C. until use.

For electroporation of plantarum, the frozen cells were thawed on ice, and 2 microliter of a DNA suspension in TE buffer was added. 40 microliters of the mixture was transferred to an ice-cold 2 mm electroporation cuvette, and electroporation carried out in a BioRad Gene Pulser™ with a setting of 1.5 kV; 25 microFarad; 400 Ohms. 500 microliter of a MRS-sucrose-MgCl₂ mixture (MRS: 6.5 ml; 2 M sucrose: 2.5 ml; 1 M MgCl₂: 1 ml) was added, and the mixture incubated without shaking at 30° C. for 2 hours before plating.

Lactobacillus reuteri strains were prepared for electroporation as follows: The strain was inoculated from a frozen stock culture into LCM medium, and incubated without shaking at 37° C. overnight. A 5 ml aliquot was transferred into 500 ml LCM and incubated at 37° C. without shaking until OD₆₀₀ reached approximately 0.8. The cells were harvested by centrifugation as above, resuspended and washed 2 times in 50 ml of ion-exchanged stearile water at room temperature, and harvested by centrifugation. The cells were finally gently resuspended in 2.5 ml of 30% PEG1500, and 50 microliter aliquots were quickly frozen in an alcohol/dry ice bath, and stored at −80° C. until use.

For electroporation of reuteri, the frozen cells were thawed on ice, and 2 microliter of a DNA suspension in TE buffer was added. 40 microliters of the mixture was transferred to an ice-cold 2 mm electroporation cuvette, kept on ice for 1-3 minutes, and electroporation carried out in a BioRad Gene Pulser™ with a setting of 1.5 kV; 25 microFarad; 400 Ohms. 500 microliter of LCM was added, and the mixture incubated without shaking for 2 hours at 37° C. before plating. Cells were plated on either LCM agar plates (LCM medium solidified with % agar) or MRS agar plates, supplemented with the required antibiotics, and incubated in an anaerobic chamber (Oxoid; equipped with Anaerogen sachet).

### Example 2

### Construction of Expression Vector pTRGU88

A 2349 bp fragment containing the LacI^{q} repressor, the trc promoter, and a multiple cloning site (MCS) was amplified from pTrc99A (E. Amann and J. Brosius, 1985, Gene 40(2-3), 183-190) using primers pTrcBglIItop and pTrcScaIbot shown below.

Primer pTrcBgIIItop:
(SEQ ID NO: 83)
5′-GAAGATCTATGGTGCAAAACCTTTCGCGG-3′
Primer pTrcScaIbot:
(SEQ ID NO: 84)
5′-AAAAGTACTCAACCAAGTCATTCTGAG-3′

PCR was carried out using Platinum Pfx DNA polymerase (Invitrogen, UK) and the amplification reaction was programmed for 25 cycles each at 95° C. for 2 minutes; 95° C. for 30 seconds, 42° C. for 30 seconds, and 72° C. for 2 minute; then one cycle at 72° C. for 3 minutes. The resulting PCR product was purified with a PCR Purification Kit (Qiagen, Hilden, Germany) according to manufacturer's instructions and digested overnight at 37° C. with 5 units each of Bg/II (New England Biolabs, Ipswich, Mass., USA) and ScaI (New England Biolabs) (restriction sites are underlined in the above primers). The digested fragment was then purified with a PCR Purification Kit (Qiagen) according to manufacturer's instructions.

Plasmid pACYC177 (Y. K. Mok, et al., 1988, Nucleic Acids Res. 16(1), 356) containing a p15A origin of replication was digested at 37° C. with 5 units ScaI (New England Biolabs) and 10 units BamHI (New England Biolabs) for two hours. 10 units of calf intestine phosphatase (CIP) (New England Biolabs) were added to the digest and incubation was continued for an additional hour, resulting in a 3256 bp fragment and a 685 bp fragment. The digest mixture was run on a 1% agarose gel and the 3256 bp fragment was excised from the gel and purified using a QIAquick Gel Extraction Kit (Qiagen) according to the manufacturer's instructions.

The purified 2349 bp PCR/restriction fragment was ligated into the 3256 bp restriction fragment using a Rapid Ligation Kit (F. Hoffmann-La Roche Ltd, Basel Switzerland) according to the manufacturer's instructions, resulting in pMIBa2. Plasmid pMIBa2 was digested with PstI using the standard buffer 3 and BSA as suggested by New England Biolabs, resulting in a 1078 bp PstI fragment containing the first 547 bp of blaTEM-1 (including the blaTEM-1 promoter and RBS) and a 4524 bp fragment containing the p15A origin of replication, the LacI^{q} repressor, the trc promoter, a multiple cloning site (MCS), and aminoglycoside 3′-phosphotransferase gene.

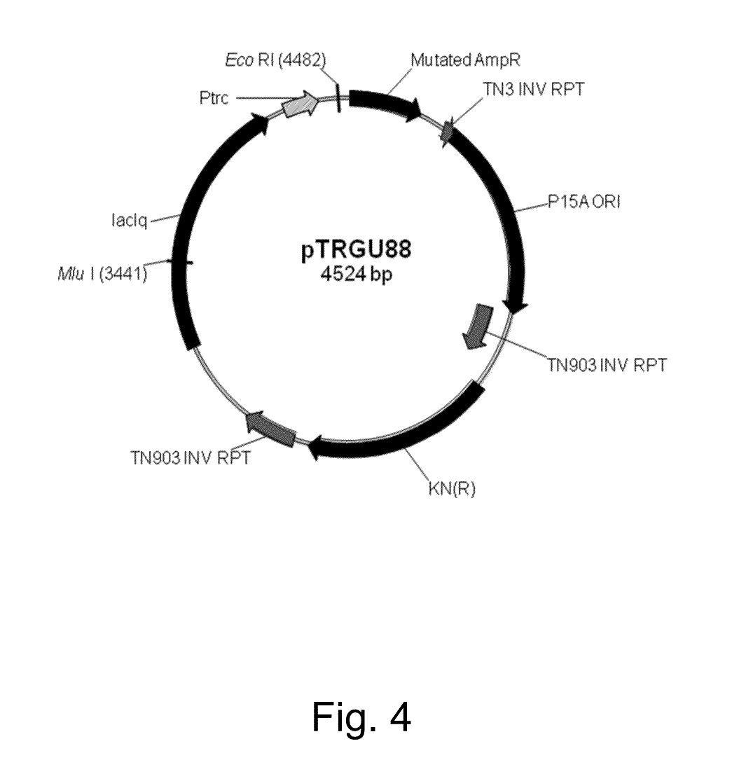

The 4524 bp fragment was ligated overnight at 16° C. using T4 DNA ligase in T4 DNA ligase buffer containing 10 mM ATP (F. Hoffmann-La Roche Ltd). A 1 μL aliquot of the ligation mixture was transformed into E. coli SJ2 cells using electroporation. Transformants were plated onto LBPGS plates containing 20 μg/ml kanamycin and incubated at 37° C. overnight. Selected colonies were then streaked on LB plates with 200 μg/mL ampicillin and on LB plates with 20 μg/mL kanamycin. Eight transformants that were ampicillin sensitive and kanamycin resistant were isolated and streak purified on LB plates with 20 μg/mL kanamycin. Each of eight colonies was inoculated in liquid TY bouillon medium and incubated overnight at 37° C. The plasmid from each colony was isolated using a Qiaprep®Spin Miniprep Kit (Qiagen) then double digested with EcoRI and MluI. Each plasmid resulted in a correct restriction pattern of 1041 bp and 3483 bp when analyzed using the electrophoresis system “FlashGel® System” from Lonza (Basel, Switzerland). The liquid overnight culture of one transformant designated E. coli TRGU88 was stored in 30% glycerol at −80° C. The corresponding plasmid pTRGU88 (FIG. 4) was isolated from E. coli TRGU88 with a Qiaprep® Spin Miniprep Kit (Qiagen) using the manufacturer's instructions and stored at −20° C.

### Example 3

### Design of Synthetic Aminoglycoside 3′-Phosphotransferase Gene with a Silent Mutation in the HindIII Restriction Site and Construction of Vector pTRGU186

The 971 bp nucleotide sequence ranging from 1524 to 2494 bp in vector pTRGU88 above includes the coding sequence of an aminoglycoside 3′-phosphotransferase gene with a HindlII restriction site, which was eliminated using a silent mutation described below.

bla gene with silent mutation
5′-CAT AAA CTT TTG-3′
Wild type bla gene:
5′-CAT AAG CTT TTG-3′

The 971 bp DNA fragment with the silent mutation was synthetically constructed into pTRGU186. The resulting sequence was then submitted to and synthesized by Geneart AG (Regenburg, Germany) and delivered in the pMA backbone vector containing the β-lactamase encoding gene blaTEM-1. When synthesized, the DNA fragment was flanked by StuI restriction sites to facilitate subsequent cloning steps.

The wild-type nucleotide sequence (WT), the sequence containing the silent mutation, and deduced amino acid sequence of the aminoglycoside 3′-phosphotransferase gene are listed as SEQ ID NO: 76, 77, and 78, respectively. The coding sequence is 816 bp including the stop codon and the encoded predicted protein is 271 amino acids.

### Example 4

### Removal of HindIII Site in the Aminoglycoside 3′-Phosphotransferase Gene of Vector pTRGU88 and Construction of Vector pTRGU187

Vectors pTRGU88 and pTRGU186 were chemically transformed into dam⁻/dcm⁻ E. coli from NEB (Cat. no. C2925H), and each re-isolated using a Qiaprep®Spin Miniprep Kit (Qiagen) from 5×4 ml of an overnight culture of 50 ml in LB medium.

The aminoglycoside 3′-phosphotransferase gene in pTRGU88 is flanked by StuI restriction sites which were used to excise the DNA fragment ranging from 1336 bp to 2675 bp. This fragment includes 284 bp upstream and 243 bp downstream of the coding sequence. The StuI fragment of pTRGU186 ranging from 400 bp to 1376 bp contains the coding sequence without the HindIII site as well as 99 bp upstream and 65 bp downstream of the coding sequence.

Both pTRGU88 and pTRGU186 were digested overnight at 37° C. with StuI (NEB). The enzyme was heat inactivated at 65° C. for 20 minutes and the pTRGU88 reaction mixture was dephosphorylated with 1 U Calf intestine phosphatase (CIP) (NEB) for 30 minutes at 37° C. The digested pTRGU88 and pTRGU186 were run on a 1% agarose gel, and bands of the expected sizes (pTRGU88: 1340 bp; pTRGU186:977 bp) were then purified using a QIAquick Gel Extraction Kit (Qiagen, Hilden, Germany) according to manufacturer's instructions.

The isolated DNA fragments were ligated overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation. Transformants were plated onto LB plates containing 20 μg/mL kanamycin and incubated at 37° C. overnight. Selected colonies were then streaked on LB plates with 20 μg/mL kanamycin. One colony, E. coli TRGU187, was inoculated in liquid TY bouillon medium with 10 μg/mL kanamycin and incubated overnight at 37° C. The corresponding plasmid pTRGU187 was isolated using a Qiaprep® Spin Miniprep Kit (Qiagen) and subjected to restriction analysis with BamHI and ClaI, which resulted in the bands BamHI-ClaI: 1764 bp and ClaI-BamHI: 2760 bp which confirmed a clockwise orientation of the gene in pTRGU187. E. coli TRGU187 from the liquid overnight culture containing pTRGU187 was stored in 30% glycerol at −80° C.

### Example 5

### Peptide-Inducible pSIP Expression Vectors

The peptide-inducible expression vectors pSIP409, pSIP410, and pSIP411 (Sørvig, E., Mathiesen, G., Naterstad, K., Eijsink, V. G. H., Axelsson, L. (2005). High-level, inducible gene expression in Lactobacillus sakei and Lactobacillus plantarum using versatile expression vectors. Microbiology, 151, 2439-2449.) were received from Lars Axelsson, Nofima Mat AS, Norway. pSIP409 and pSIP410 were transformed into E. coli SJ2 by electroporation, selecting erythromycin resistance (150 microgram/ml) on LB agar plates at 37° C. Two transformants containing pSIP409 were kept as SJ10517 and SJ10518, and two transformants containing pSIP410 were kept as SJ10519 and SJ10520.

pSIP411 was transformed into naturally competent Bacillus subtilis DN1885 cells, essentially as described (Yasbin, R. E., Wilson, G. A., Young, F. E. (1975). Transformation and transfection in lysogenic strains of Bacillus subtilis: Evidence for selective induction of prophage in competent cells. Journal of Bacteriology, 121, 296-304), selecting for erythromycin resistance (5 microgram/ml) on LBPGS plates at 37° C. Two such transformants were kept as SJ10513 and SJ10514.

pSIP411 was in addition transformed into E. coli MG1655 by electroporation, selecting erythromycin resistance (200 microgram/ml) on LB agar plates at 37° C., and two transformants kept as SJ10542 and SJ10543.

For use in induction of gene expression from these vectors in Lactobacillus, the inducing peptide, here named M-19-R and having the following amino acid sequence: “Met-Ala-Gly-Asn-Ser-Ser-Asn-Phe-Ile-His-Lys-Ile-Lys-Gln-Ile-Phe-Thr-His-Arg”, was obtained from “Polypeptide Laboratories France, 7 rue de Boulogne, 67100 Strasbourg, France”.

### Example 6

### Construction of pVS2-Based Vectors pSJ10600 and pSJ10603 for Constitutive Expression

A set of constitutive expression vectors were constructed based on the plasmid pVS2 (von Wright, A., Tynkkynen, S., Suominen, M. (1987) Cloning of a Streptococcus lactis subsp. Lactis chromosomal fragment associated with the ability to grow in milk. Applied and Environmental Microbiology, 53, 1584-1588) and promoters described by Rud et al. (Rud, I., Jensen, P. R., Naterstad, K., Axelsson, L. (2006) A synthetic promoter library for constitutive gene expression in Lactobacillus plantarum. Microbiology, 152, 1011-1019). A DNA fragment containing the P11 promoter with a selection of flanking restriction sites, and another fragment containing P27 with a selection of flanking restriction sites, was chemically synthesized by Geneart AG (Regenburg, Germany).

The DNA fragment containing P11 with flanking restriction sites, and the DNA fragment containing P27 with flanking restriction sites are shown in SEQ ID NOs: 85 and 86, respectively. Both DNA fragments were obtained in the form of DNA preparations, where the fragments had been inserted into the standard Geneart vector, pMA. The vector containing P11 was transformed into E. coli SJ2 cells, and a transformant kept as SJ10560, containing plasmid pSJ10560. The vector containing P27 was transformed into E. coli SJ2 cells, and a transformant kept as SJ10561, containing plasmid pSJ10561.

The promoter-containing fragments, in the form of 176 bp HindIII fragments, were excised from the Geneart vectors and ligated to HindIII-digested pUC19. The P11-containing fragment was excised from the vector prepared from SJ10560, ligated to pUC19, and correct transformants of E. coli SJ2 were kept as SJ10585 and SJ10586, containing pSJ10585 and pSJ10586, respectively. The P27 containing fragment was excised from the vector prepared from SJ10561, ligated to pUC19, and correct transformants of E. coli SJ2 were kept as SJ10587 and SJ10588, containing pSJ10587 and pSJ10588, respectively.

Plasmid pVS2 was obtained in Lactobacillus plantarum NC8, a strain kept as SJ10491, extracted from this strain by standard plasmid preparation procedures known in the art, and transformed into E. coli MG1655 selecting erythromycin resistance (200 microgram/ml) on LB agar plates at 37° C. Two such transformants were kept as SJ10583 and SJ10584.

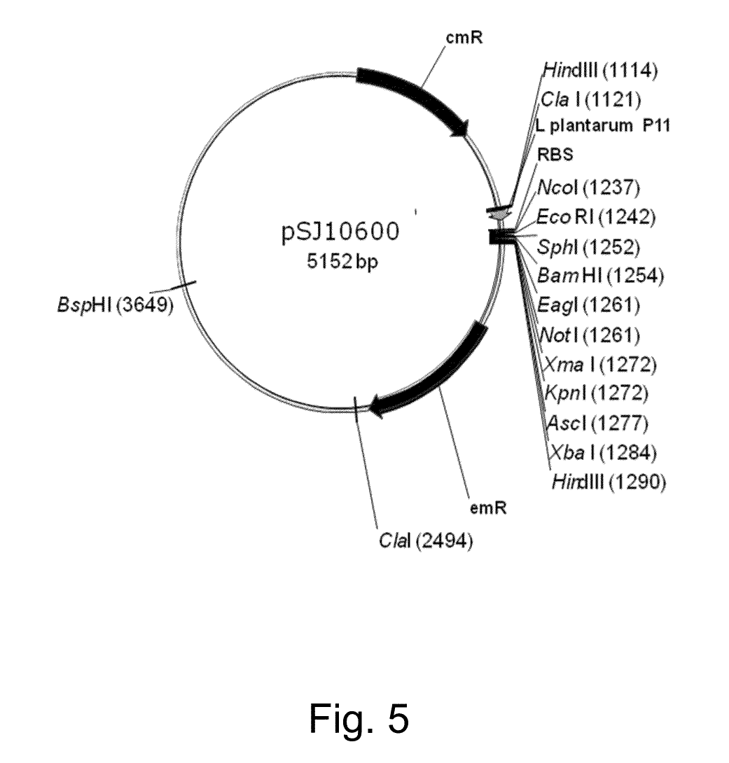

To insert P11 into pVS2, the P11-containing 176 bp HindIII fragment was excised and purified by agarose gel electrophoresis from pSJ10585, and ligated to HindIII-digested pVS2, which had been prepared from SJ10583. The ligation mixture was transformed by electroporation into E. coli MG1655, selecting erythromycin resistance (200 microgram/ml) on LB agar plates, and two transformants, which both harbor plasmids with the promoter insert in one particular of the two possible orientations, were kept as SJ10600 and SJ10601, containing pSJ10600 (FIG. 5) and pSJ10601.

Another transformant, having the promoter insert in the other of the two possible orientations, was kept as SJ10602, containing pSJ10602. The plasmid preparation from SJ10602 appeared to contain less DNA than the comparable preparations from SJ10600 and SJ10601, and, upon further work, pSJ10602 appeared to be rather unstable, with deletion derivatives dominating in the plasmid population.

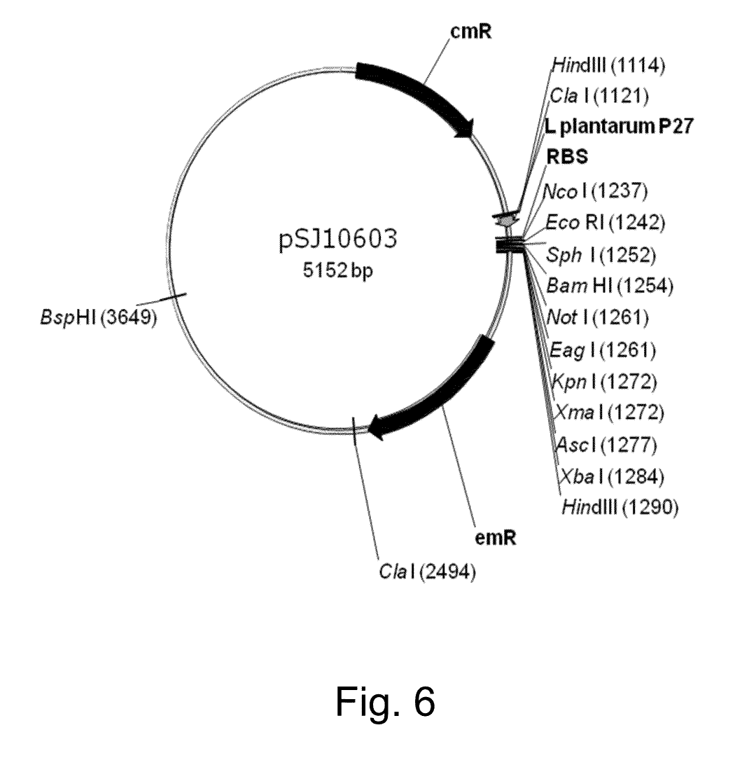

To insert P27 into pVS2, the P27-containing 176 bp HindIII fragment was excised and purified by agarose gel electrophoresis from pSJ10588, and ligated to HindIII-digested pVS2, which had been prepared from SJ10583. The ligation mixture was transformed by electroporation into E. coli MG1655, selecting erythromycin resistance (200 microgram/ml) on LB agar plates, and two transformants, which both harbor plasmids with the promoter insert in one particular of the two possible orientations, were kept as SJ10603 and SJ10604, containing pSJ10603 (FIG. 6) and pSJ10604.

Another transformant, having the promoter insert in the other of the two possible orientations, was kept as SJ10605, containing pSJ10605. The promoter orientation in this plasmid is the same as in pSJ10602, described above. The plasmid preparation from SJ10605 appeared to contain less DNA than the comparable preparations from SJ10603 and SJ10604, and, upon further work, pSJ10605 appeared to be rather unstable, with deletion derivatives dominating in the plasmid population.

### Example 7

### Fermentation Product Analysis

Acetone, 1-propanol and isopropanol in fermentation broths described herein were detectable by GC-FID. Samples were diluted 1+1 with 0.05% tetrahydrofuran in methanol and analyzed. GC parameters are listed in Table 1.

TABLE 1

| | | Approx. Retention |
|---|---|---|
| Parameter | | time (min) |
| GC column | DB-WAX 30 m-0.25 mm | |
| | i.d-0.50 μm film part-no | |
| | 122-7033 from J&W Scientific | |
| Carrier gas | Hydrogen | |
| Temp. gradient | 0-4.5 min: 50° C. | |
| | 4.5-9.93 min: 50-240° C. linear | |
| | gradient | |
| Detection | FID | |
| Internal | Tetrahydrofuran | 2.4 |
| standard | | |
| External | Acetone (Analytical grade) | 2.0 |
| standards | 1-propanol (Analytical grade) | 5.4 |
| | isopropanol (HPLC grade) | 3.3 |

### Example 8

### Cloning of Isopropanol Pathway Genes

Cloning of a Clostridium acetobutylicum Thiolase Gene and Construction of Vector pSJ10705.

The 1176 bp coding sequence (without stop codon) of a thiolase gene identified in Clostridium acetobutylicum was designed for optimized expression in the three organisms Escherichia coli, Lactobacillus plantarum, and Lactobacillus reuteri and synthetically constructed into pSJ10705. The DNA fragment containing the codon optimized coding sequence was designed with the sequence 5′-AAGCTTTC-3′ immediately prior to the start codon (to add a HindIII site and convert the start region to a NcoI-compatible BspHI site), and the sequence 5′-TAGTCTAGACTCGAGGAATTCGGTACC-3′ immediately downstream (to add a stop codon, and restriction sites XbaI-XhoI-EcoRI-KpnI).

The resulting sequence was then submitted to and synthesized by Geneart AG (Regenburg, Germany) and delivered in the pMA backbone vector containing the β-lactamase encoding gene blaTEM-1. The DNA preparation delivered from Geneart was transformed into E. coli SJ2 by electroporation, selecting ampicillin resistance (200 microgram/ml) and two transformants kept, as SJ10705 (SJ2/pSJ10705) and SJ10706 (SJ2/pSJ10706).

The wild-type nucleotide sequence (WT), codon-optimized nucleotide sequence (CO), and deduced amino acid sequence of the C. acetobutylicum thiolase gene are SEQ ID NOs: 1, 2, and 3, respectively. The coding sequence is 1179 bp including the stop codon and the encoded predicted protein is 392 amino acids. Using the SignalP program (Nielsen et al., 1997, Protein Engineering 10:1-6), no signal peptide in the sequence was predicted. Based on this program, the predicted mature protein contains 392 amino acids with a predicted molecular mass of 41.4 kDa and an isoelectric pH of 7.08.

Cloning of a Lactobacillus reuteri Thiolase Gene and Construction of Vector DSJ10694.

The 1176 bp thiolase coding sequence (withough stop codon) from Lactobacillus reuteri was amplified from chromosomal DNA of SJ10468 (supra) using primers 671826 and 671827 shown below.

Primer 671826:
(SEQ ID NO: 87)
5′-AGTCAAGCTTCCATGGAGAAGGTTTACATTGTTGC-3′
Primer 671827:
(SEQ ID NO: 88)
5′-ATGCGGTACCGAATTCCTCGAGTCTAGACTAAATTTTCTTAAGCAG
AACCG-3′

The PCR reaction was programmed for 94° C. for 2 minutes; and then 19 cycles each at 95° C. for 30 seconds, 59° C. for 1 minute, and 72° C. for 2 minute; then one cycle at 72° C. for 5 minutes. A PCR amplified fragment of approximately 1.2 kb was digested with NcoI+EcoRI, purified by agarose gel electrophoresis, and then ligated to the agarose gel electrophoresis purified EcoRI-NcoI vector fragment of plasmid pSIP409. The ligation mixture was transformed into E. coli SJ2, selecting ampicillin resistance (200 microgram/ml), and a transformant, deemed correct by restriction digest and DNA sequencing, was kept as SJ10694 (SJ2/pSJ10694).

The codon-optimized nucleotide sequence (CO), and deduced amino acid sequence of the L. reuteri thiolase gene are SEQ ID NOs: 34 and 35, respectively. The coding sequence is 1179 bp including the stop codon and the encoded predicted protein is 392 amino acids. Using the SignalP program (Nielsen et al., 1997, Protein Engineering 10:1-6), no signal peptide in the sequence was predicted. Based on this program, the predicted mature protein contains 392 amino acids with a predicted molecular mass of 41.0 kDa and an isoelectric pH of 5.4.

Cloning of a Propionibacterium freudenreichii Thiolase Gene and Construction of Vector pSJ10676.

The 1152 bp coding sequence (without stop codon) of a thiolase gene identified in Propionibacterium freudenreichii was optimized for expression in the three organisms Escherichia coli, Lactobacillus plantarum, and Lactobacillus reuteri and synthetically constructed into pSJ10676. The DNA fragment containing the codon optimized CDS was designed with the sequence 5′-AAGCTTTC-3′ immediately prior to the start codon (to add a HindIII site and convert the start region to a NcoI-compatible BspHI site), and the sequence 5′-TAGTCTAGACTCGAGGAATTCGGTACC-3′ (SEQ ID NO: 112) immediately downstream (to add a stop codon, and restriction sites XbaI-XhoI-EcoRI-KpnI).

The resulting sequence was then submitted to and synthesized by Geneart AG (Regenburg, Germany) and delivered in the pMA backbone vector containing the β-lactamase encoding gene blaTEM-1. The DNA preparation delivered from Geneart was transformed into E. coli SJ2 by electroporation, selecting ampicillin resistance (200 microgram/ml) and two transformants kept, as SJ10676 (SJ2/pSJ10676) and SJ10677 (SJ2/pSJ10677).

The codon-optimized nucleotide sequence (CO), and deduced amino acid sequence of the P. freudenreichii thiolase gene are SEQ ID NOs: 113 and 114, respectively. The coding sequence is 1155 bp including the stop codon and the encoded predicted protein is 384 amino acids. Using the SignalP program (Nielsen et al., 1997, Protein Engineering 10:1-6), no signal peptide in the sequence was predicted. Based on this program, the predicted mature protein contains 384 amino acids with a predicted molecular mass of 39.8 kDa and an isoelectric pH of 6.1.

Cloning of a Lactobacillus brevis Thiolase Gene and Construction of Vector pSJ10699.

The 1167 bp coding sequence (without stop codon) of a thiolase gene identified in Lactobacillus brevis was optimized for expression in the three organisms Escherichia coli, Lactobacillus plantarum, and Lactobacillus reuteri and synthetically constructed into pSJ10699. The DNA fragment containing the codon optimized CDS was designed with the sequence 5′-AAGCTTCC-3′ immediately prior to the start codon (to add a HindIII site and convert the start region to a NcoI site), and the sequence 5′-TAGTCTAGACTCGAGGAATTCGGTACC-3′ (SEQ ID NO: 112) immediately downstream (to add a stop codon, and restriction sites XbaI-XhoI-EcoRI-KpnI).

The resulting sequence was then submitted to and synthesized by Geneart AG (Regenburg, Germany) and delivered in the pMA backbone vector containing the β-lactamase encoding gene blaTEM-1. The DNA preparation delivered from Geneart was transformed into E. coli SJ2 by electroporation, selecting ampicillin resistance (200 microgram/ml) and two transformants kept, as SJ10699 (SJ2/pSJ10699) and SJ10700 (SJ2/pSJ10700).

The codon-optimized nucleotide sequence (CO), and deduced amino acid sequence of the L. brevis thiolase gene are SEQ ID NOs: 115 and 116, respectively. The coding sequence is 1170 bp including the stop codon and the encoded predicted protein is 389 amino acids. Using the SignalP program (Nielsen et al., 1997, Protein Engineering 10:1-6), no signal peptide in the sequence was predicted. Based on this program, the predicted mature protein contains 389 amino acids with a predicted molecular mass of 40.4 kDa and an isoelectric pH of 6.5.

Cloning of B. subtilis Succinyl-CoA:Acetoacetate Transferase Genes and Construction of Vectors pSJ10695 and pSJ10697.

The 699 bp coding sequence (without stop codon) of the scoA subunit of the B. subtilis succinyl-CoA:acetoacetate transferase and the 648 bp coding sequence of the scoB subunit of the B. subtilis succinyl-CoA:acetoacetate transferase were designed for optimized expression in the three organisms Escherichia coli, Lactobacillus plantarum, and Lactobacillus reuteri and synthetically constructed into pSJ10695 and pSJ10697, respectively.

The DNA fragment containing the codon-optimized scoA coding sequence was designed with the sequence 5′-AAGCT TCTCG AGACT ATTAC AAGGA GATTT TAGCC-3′ (SEQ ID NO: 89) immediately prior to the start codon (to add a HindIII site, a Lactobacillus RBS, and to have the start codon within a NcoI site), and an EcoRI restriction site immediately downstream. The designed construct was obtained from Geneart AG and transformed as described above, resulting in SJ10695 (SJ2/pSJ10695) and SJ10696 (SJ2/pSJ10696).

The wild-type nucleotide sequence (WT), codon-optimized nucleotide sequence (CO), and deduced amino acid sequence of the B. subtilis scoA subunit of the succinyl-CoA:acetoacetate transferase are SEQ ID NOs: 4, 5, and 6, respectively. The coding sequence is 702 bp including the stop codon and the encoded predicted protein is 233 amino acids. Using the SignalP program (Nielsen et al., supra), no signal peptide in the sequence was predicted. Based on this program, the predicted mature protein contains 233 amino acids with a predicted molecular mass of 25.1 kDa and an isoelectric pH of 6.50.

The DNA fragment containing the codon optimized scoB coding sequence was designed with the sequence 5′-GAATT CACTA TTACA AGGAG ATTTT AGTC-3′ (SEQ ID NO: 90) immediately prior to the start codon (to add a EcoRI site, a Lactobacillus RBS, and to have the start codon within a NcoI-compatible BspHI site), and EagI and KpnI restriction sites immediately downstream. The designed construct was obtained from Geneart AG and transformed as described above, resulting in SJ10697 (SJ2/pSJ10697) and SJ10698 (SJ2/pSJ10698).

The wild-type nucleotide sequence (WT), codon-optimized nucleotide sequence (CO), and deduced amino acid sequence of the B. subtilis scoB subunit of the succinyl-CoA:acetoacetate transferase are SEQ ID NOs: 7, 8, and 9, respectively. The coding sequence is 651 bp including the stop codon and the encoded predicted protein is 216 amino acids. Using the SignalP program (Nielsen et al., supra), no signal peptide in the sequence was predicted. Based on this program, the predicted mature protein contains 216 amino acids with a predicted molecular mass of 23.4 kDa and an isoelectric pH of 5.07.

Cloning of B. mojavensis Succinyl-CoA:Acetoacetate Transferase Genes and Construction of Vectors pSJ10721 and pSJ10723.

The 711 bp coding sequence (without stop codon) of the scoA subunit of the B. mojavensis succinyl-CoA:acetoacetate transferase and the 654 bp coding sequence (without stop codon) of the scoB subunit of the B. mojavensis succinyl-CoA:acetoacetate transferase were designed for optimized expression in the three organisms Escherichia coli, Lactobacillus plantarum, and Lactobacillus reuteri and synthetically constructed into pSJ10721 and pSJ10723, respectively.

The DNA fragment containing the codon-optimized scoA coding sequence was designed with the sequence 5′-AAGCT TCTCG AGACT ATTAC AAGGA GATTT TAGCC-3′ (SEQ ID NO: 89) immediately prior to the start codon (to add a HindIII site, a Lactobacillus RBS, and to have the start codon within a NcoI site), and an EcoRI restriction site immediately downstream. The designed construct was obtained from Geneart AG and transformed as described above, resulting in SJ10721 (SJ2/pSJ10721) and SJ10722 (SJ2/pSJ10722).

The wild-type nucleotide sequence (WT), codon-optimized nucleotide sequence (CO), and deduced amino acid sequence of the B. mojavensis scoA subunit of the succinyl-CoA:acetoacetate transferase are SEQ ID NOs: 10, 11, and 12, respectively. The coding sequence is 714 bp including the stop codon and the encoded predicted protein is 237 amino acids. Using the SignalP program (Nielsen et al., supra), no signal peptide in the sequence was predicted. Based on this program, the predicted mature protein contains 237 amino acids with a predicted molecular mass of 25.5 kDa and an isoelectric pH of 5.82.

The DNA fragment containing the codon optimized scoB nucleotide coding sequence was designed with the sequence 5′-GAATT CACTA TTACA AGGAG ATTTT AGTC-3′ (SEQ ID NO: 90) immediately prior to the start codon (to add a EcoRI site, a Lactobacillus RBS, and to have the start codon within a NcoI-compatible BspHI site), and EagI and KpnI restriction sites immediately downstream. The designed construct was obtained from Geneart AG and transformed as described above, resulting in SJ10723 (SJ2/pSJ10723) and SJ10724 (SJ2/pSJ10724).

The wild-type nucleotide sequence (WT), codon-optimized nucleotide sequence (CO), and deduced amino acid sequence of the B. mojavensis scoB subunit of the succinyl-CoA:acetoacetate transferase are SEQ ID NOs: 13, 14, and 15, respectively. The coding sequence is 657 bp including the stop codon and the encoded predicted protein is 218 amino acids. Using the SignalP program (Nielsen et al., supra), no signal peptide in the sequence was predicted. Based on this program, the predicted mature protein contains 218 amino acids with a predicted molecular mass of 23.7 kDa and an isoelectric pH of 5.40.

Cloning of E. coli Acetoacetyl-CoA Transferase Genes and Construction of Vectors pSJ10715 and pSJ10717.

The 648 bp coding sequence (without stop codon) of the atoA subunit (uniprot:P76459) of the E. coli acetyl-CoA transferase and the 660 bp coding sequence (without stop codon) of the atoD subunit (uniprot:P76458) of the E. coli acetyl-CoA transferase were optimized for expression in the three organisms Escherichia coli, Lactobacillus plantarum, and Lactobacillus reuteri and synthetically constructed into pSJ10715 and pSJ10717, respectively.

The DNA fragment containing the codon-optimized atoA subunit nucleotide coding sequence was designed with the sequence 5′-AAGCT TCTCG AGACT ATTAC AAGGA GATTT TAGCC-3′ (SEQ ID NO: 89) immediately prior to the start codon (to add HindIII and XhoI sites, a Lactobacillus RBS, and to have the start codon within a NcoI site), and an EcoRI restriction site immediately downstream. The designed construct was obtained from Geneart AG and transformed as described above, resulting in SJ10715 (SJ2/pSJ10715) and SJ10716 (SJ2/pSJ10716).

The codon-optimized nucleotide sequence (CO) and deduced amino acid sequence of the E. coli atoA subunit of the acetoacetyl-CoA transferase are SEQ ID NOs: 36 and 37, respectively. The coding sequence is 651 bp including the stop codon and the encoded predicted protein is 216 amino acids. Using the SignalP program (Nielsen et al., supra), no signal peptide in the sequence was predicted. Based on this program, the predicted mature protein contains 216 amino acids with a predicted molecular mass of 23.0 kDa and an isoelectric pH of 5.9.

The DNA fragment containing the codon optimized atoD nucleotide coding sequence was designed with the sequence 5′-GAATT CACTA TTACA AGGAG ATTTT AGTC-3′ (SEQ ID NO: 90) immediately prior to the start codon (to add a EcoRI site, a Lactobacillus RBS, and to have the start codon within a NcoI-compatible BspHI site), and EagI and KpnI restriction sites immediately downstream. The designed construct was obtained from Geneart AG and transformed as described above, resulting in SJ10717 (SJ2/pSJ10717) and SJ10718 (SJ2/pSJ10718).

The codon-optimized nucleotide sequence (CO) and deduced amino acid sequence of the E. coli atoD subunit of the acetoacetyl-CoA transferase are SEQ ID NOs: 38 and 39, respectively. The coding sequence is 663 bp including the stop codon and the encoded predicted protein is 220 amino acids. Using the SignalP program (Nielsen et al., supra), no signal peptide in the sequence was predicted. Based on this program, the predicted mature protein contains 220 amino acids with a predicted molecular mass of 23.5 kDa and an isoelectric pH of 4.9.

Cloning of Clostridium acetobutylicum Acetoacetyl-CoA Transferase Genes and Construction of Vectors pSJ10727 and pSJ10731.

The 654 bp coding sequence (without stop codon) of the ctfA subunit (uniprot:P33752) of the C. acetobutylicum acetyl-CoA transferase and the 663 bp coding sequence (without stop codon) of the ctfB subunit (uniprot:P23673) of the C. acetobutylicum acetyl-CoA transferase were optimized for expression in the three organisms Escherichia coli, Lactobacillus plantarum, and Lactobacillus reuteri and synthetically constructed into pSJ10727 and pSJ10731, respectively.

The DNA fragment containing the codon optimized ctfA subunit coding sequence was designed with the sequence 5′-AAGCT TCTCG AGACT ATTAC AAGGA GATTT TAGTC-3′ (SEQ ID NO: 91) immediately prior to the start codon (to add HindIII and XhoI sites, a Lactobacillus RBS, and to have the start codon within a NcoI-compatible BspHI site), and an EcoRI restriction site immediately downstream. The designed construct was obtained from Geneart AG and transformed as described above, resulting in SJ10727 (SJ2/pSJ10727) and SJ10728 (SJ2/pSJ10728).

The codon-optimized nucleotide sequence (CO) and deduced amino acid sequence of the C. acetobutylicum ctfA subunit of the acetoacetyl-CoA transferase are SEQ ID NOs: 40 and 41, respectively. The coding sequence is 657 bp including the stop codon and the encoded predicted protein is 218 amino acids. Using the SignalP program (Nielsen et al., supra), no signal peptide in the sequence was predicted. Based on this program, the predicted mature protein contains 218 amino acids with a predicted molecular mass of 23.6 kDa and an isoelectric pH of 9.3.

The DNA fragment containing the codon optimized ctfB subunit coding sequence was designed with the sequence 5′-GAATT CACTA TTACA AGGAG ATTTT AGTC-3′ (SEQ ID NO: 90) immediately prior to the start codon (to add a EcoRI site, a Lactobacillus RBS, and to have the start codon within a NcoI-compatible BspHI site), and EagI and KpnI restriction sites immediately downstream. The designed construct was obtained from Geneart AG and transformed as described above, resulting in SJ10731 (SJ2/pSJ10731) and SJ10732 (SJ2/pSJ10732).

The codon-optimized nucleotide sequence (CO) and deduced amino acid sequence of the C. acetobutylicum ctfB subunit of the acetoacetyl-CoA transferase are SEQ ID NOs: 42 and 43, respectively. The coding sequence is 666 bp including the stop codon and the encoded predicted protein is 221 amino acids. Using the SignalP program (Nielsen et al., supra), no signal peptide in the sequence was predicted. Based on this program, the predicted mature protein contains 221 amino acids with a predicted molecular mass of 23.6 kDa and an isoelectric pH of 8.5.

Cloning of a Clostridium acetobutylicum Acetoacetate Decarboxylase Gene and Construction of Vector pSJ10711.

The 777 bp coding sequence (without stop codon) of the acetoacetate decarboxylase (uniprot:P23670) from C. acetobutylicum was optimized for expression in the three organisms Escherichia coli, Lactobacillus plantarum, and Lactobacillus reuteri and synthetically constructed into pSJ10711.

The DNA fragment containing the codon-optimized acetoacetate decarboxylase coding sequence (adc) was designed with the sequence 5′-AAGCT TCGGC CGACT ATTAC AAGGA GATTT TAGCC-3′ (SEQ ID NO: 92) immediately prior to the start codon (to add HindIII and EagI sites and a Lactobacillus RBS), and a KpnI restriction site immediately downstream. The designed construct was obtained from Geneart AG and transformed as described above, resulting in SJ10711 (SJ2/pSJ10711) and SJ10712 (SJ2/pSJ10712).

The codon-optimized nucleotide sequence (CO) and deduced amino acid sequence of the C. acetobutylicum acetoacetate decarboxylase gene are SEQ ID NOs: 44 and 45, respectively. The coding sequence is 780 bp including the stop codon and the encoded predicted protein is 259 amino acids. Using the SignalP program (Nielsen et al., supra), no signal peptide in the sequence was predicted. Based on this program, the predicted mature protein contains 259 amino acids with a predicted molecular mass of 27.5 kDa and an isoelectric pH of 6.2.

Cloning of a Clostridium beijerinckii Acetoacetate Decarboxylase Gene and Construction of Vector pSJ10713.

The 738 bp coding sequence (without stop codon) of the acetoacetate decarboxylase (uniprot:Q716S5) from C. beijerinckii was optimized for expression in the three organisms Escherichia coli, Lactobacillus plantarum, and Lactobacillus reuteri and synthetically constructed into pSJ10713.

The DNA fragment containing the codon optimized acetoacetate decarboxylase coding sequence (adc Cb) was designed with the sequence 5′-AAGCT TCGGC CGACT ATTAC AAGGA GATTT TAGCC-3′ (SEQ ID NO: 92) immediately prior to the start codon (to add HindIII and EagI sites and a Lactobacillus RBS), and a KpnI restriction site immediately downstream. The desigined construct was obtained from Geneart AG and transformed as described above, resulting in SJ10713 (SJ2/pSJ10713) and SJ10714 (SJ2/pSJ10714).

The wild-type nucleotide sequence (WT), codon-optimized nucleotide sequence (CO), and deduced amino acid sequence of the C. beijerinckii acetoacetate decarboxylase gene is SEQ ID NO: 16, 17, and 18, respectively. The coding sequence is 741 bp including the stop codon and the encoded predicted protein is 246 amino acids. Using the SignalP program (Nielsen et al., supra), no signal peptide in the sequence was predicted. Based on this program, the predicted mature protein contains 246 amino acids with a predicted molecular mass of 27.5 kDa and an isoelectric pH of 6.18.

Cloning of a Lactobacillus salvarius Acetoacetate Decarboxylase Gene and Construction of Vector pSJ10707.

The 831 bp CDS (without stop codon) of the acetoacetate decarboxylase (SWISSPROT:Q1WVG5) from L. salvarius was optimized for expression in the three organisms Escherichia coli, Lactobacillus plantarum, and Lactobacillus reuteri and synthetically constructed into pSJ10707.

The DNA fragment containing the codon optimized acetoacetate decarboxylase CDS (adc Ls) was designed with the sequence 5′-AAGCT TCGGC CGACT ATTAC AAGGA GATTT TAGAC-3′ (SEQ ID NO: 92) immediately prior to the start codon (to add HindIII and EagI sites and a Lactobacillus RBS), and a KpnI restriction site immediately downstream. The constructs were obtained from Geneart AG and transformed as previously described, resulting in SJ10707 (SJ2/pSJ10707) and SJ10708 (SJ2/pSJ10708).

The codon-optimized nucleotide sequence (CO) and deduced amino acid sequence of the L. salvarius acetoacetate decarboxylase gene is SEQ ID NO: 117 and 118, respectively. The coding sequence is 834 bp including the stop codon and the encoded predicted protein is 277 amino acids. Using the SignalP program (Nielsen et al., supra), no signal peptide in the sequence was predicted. Based on this program, the predicted mature protein contains 277 amino acids with a predicted molecular mass of 30.9 kDa and an isoelectric pH of 4.6.

Cloning of a Lactobacillus plantarum Acetoacetate Decarboxylase Gene and Construction of Vector pSJ10701.

The 843 bp CDS (without stop codon) of the acetoacetate decarboxylase (SWISSPROT:Q890G0) from L. plantarum was optimized for expression in the three organisms Escherichia coli, Lactobacillus plantarum, and Lactobacillus reuteri and synthetically constructed into pSJ10701.

The DNA fragment containing the codon optimized acetoacetate decarboxylase CDS (adc Lp) was designed with the sequence 5′-AAGCT TCGGC CGACT ATTAC AAGGA GATTT TAGCC-3′ (SEQ ID NO: 92) immediately prior to the start codon (to add HindIII and EagI sites and a Lactobacillus RBS), and a KpnI restriction site immediately downstream. The constructs were obtained from Geneart AG and transformed as previously described, resulting in SJ10701 (SJ2/pSJ10701) and SJ10702 (SJ2/pSJ10702).

The codon-optimized nucleotide sequence (CO) and deduced amino acid sequence of the L. plantarum acetoacetate decarboxylase gene is SEQ ID NO: 119 and 120, respectively. The coding sequence is 846 bp including the stop codon and the encoded predicted protein is 281 amino acids. Using the SignalP program (Nielsen et al., supra), no signal peptide in the sequence was predicted. Based on this program, the predicted mature protein contains 281 amino acids with a predicted molecular mass of 30.8 kDa and an isoelectric pH of 4.7.

Cloning of a Thermoanaerobacter ethanolicus Isopropanol Dehydrogenase Gene and Construction of Vector pSJ10719.

The 1056 bp coding sequence (without stop codon) of the isopropanol dehydrogenase (uniprot:Q2MJT8) from T. ethanolicus was optimized for expression in the three organisms Escherichia coli, Lactobacillus plantarum, and Lactobacillus reuteri and synthetically constructed into pSJ10719.

The DNA fragment containing the codon optimized isopropanol dehydrogenase coding sequence (adh Te) was designed with the sequence 5′-GGTAC CACTA TTACA AGGAG ATTTT AGTC-3′ (SEQ ID NO: 95) immediately prior to the start codon (to add a KpnI site, a Lactobacillus RBS, and to have the start codon within a NcoI-compatible BspHI site), and XmaI and HindIII restriction sites immediately downstream. The desigined construct was obtained from Geneart AG and transformed as described above, resulting in SJ10719 (SJ2/pSJ10719) and SJ10720 (SJ2/pSJ10720).

The wild-type nucleotide sequence (WT), codon-optimized nucleotide sequence (CO), and deduced amino acid sequence of the T. ethanolicus isopropanol dehydrogenase gene is SEQ ID NO: 22, 23, and 24, respectively. The coding sequence is 1059 bp including the stop codon and the encoded predicted protein is 352 amino acids. Using the SignalP program (Nielsen et al., supra), no signal peptide in the sequence was predicted. Based on this program, the predicted mature protein contains 352 amino acids with a predicted molecular mass of 37.7 kDa and an isoelectric pH of 6.23.

Cloning of a Clostridium beijerinckii Isopropanol Dehydrogenase Gene and Construction of Vector pSJ10725.

The 1053 bp coding sequence (without stop codon) of the isopropanol dehydrogenase (uniprot:P25984) from C. beijerinckii was optimized for expression in the three organisms Escherichia coli, Lactobacillus plantarum, and Lactobacillus reuteri and synthetically constructed into pSJ10725.

The DNA fragment containing the codon optimized isopropanol dehydrogenase coding sequence (adh Cb) was designed with the sequence 5′-GGTAC CACTA TTACA AGGAG ATTTT AGTC-3′ (SEQ ID NO: 95) immediately prior to the start codon (to add a KpnI site, a Lactobacillus RBS, and to have the start codon within a NcoI-compatible BspHI site), and XmaI and HindIII restriction sites immediately downstream. The desigined construct was obtained from Geneart AG and transformed as described above, resulting in SJ10725 (SJ2/pSJ10725) and SJ10726 (SJ2/pSJ10726).

The wild-type nucleotide sequence (WT), codon-optimized nucleotide sequence (CO), and deduced amino acid sequence of the C. beijerinckii isopropanol dehydrogenase gene is SEQ ID NO: 19, 20, and 21, respectively. The coding sequence is 1056 bp including the stop codon and the encoded predicted protein is 351 amino acids. Using the SignalP program (Nielsen et al., supra), no signal peptide in the sequence was predicted. Based on this program, the predicted mature protein contains 351 amino acids with a predicted molecular mass of 37.8 kDa and an isoelectric pH of 6.64.

Cloning of a Lactobacillus antri Isopropanol Dehydrogenase Gene and Construction of Vector pSJ10709.

The 1068 bp coding sequence (without stop codon) of the isopropanol dehydrogenase (SWISSPROT:C8P9V7) from L. antri was optimized for expression in the three organisms Escherichia coli, Lactobacillus plantarum, and Lactobacillus reuteri and synthetically constructed into pSJ10709.

The DNA fragment containing the codon-optimized isopropanol dehydrogenase coding sequence (sadh La) was designed with the sequence 5′-GGTAC CACTA TTACA AGGAG ATTTT AGTC-3′ (SEQ ID NO: 95) immediately prior to the start codon (to add a KpnI site and a Lactobacillus RBS), and XmaI and HindIII restriction sites immediately downstream. The desigined construct was obtained from Geneart AG and transformed as described above, resulting in SJ10709 (SJ2/pSJ10709) and SJ10710 (SJ2/pSJ10710).

The codon-optimized nucleotide sequence (CO) and deduced amino acid sequence of the L. antri isopropanol dehydrogenase gene is SEQ ID NO: 46 and 47, respectively. The coding sequence is 1071 bp including the stop codon and the encoded predicted protein is 356 amino acids. Using the SignalP program (Nielsen et al., supra), no signal peptide in the sequence was predicted. Based on this program, the predicted mature protein contains 356 amino acids with a predicted molecular mass of 38.0 kDa and an isoelectric pH of 4.9.

Cloning of a Lactobacillus fermentum Isopropanol Dehydrogenase Gene and Construction of Vector pSJ10703.

The 1068 bp CDS (without stop codon) of the isopropanol dehydrogenase (SWISSPROT:B2GDH6) from L. fermentum was optimized for expression in the three organisms Escherichia coli, Lactobacillus plantarum, and Lactobacillus reuteri and synthetically constructed into pSJ10703.

The DNA fragment containing the codon optimized isopropanol dehydrogenase CDS (sadh Lf) was designed with the sequence 5′-GGTAC CACTA TTACA AGGAG ATTTT AGTC-3′ (SEQ ID NO: 95) immediately prior to the start codon (to add a KpnI site and a Lactobacillus RBS), and XmaI and HindIII restriction sites immediately downstream. The constructs were obtained from Geneart AG and transformed as previously described, resulting in SJ10703 (SJ2/pSJ10703) and SJ10704 (SJ2/pSJ10704).

The codon-optimized nucleotide sequence (CO) and deduced amino acid sequence of the L. fermentum isopropanol dehydrogenase gene is SEQ ID NO: 121 and 122, respectively. The coding sequence is 1071 bp including the stop codon and the encoded predicted protein is 356 amino acids. Using the SignalP program (Nielsen et al., supra), no signal peptide in the sequence was predicted. Based on this program, the predicted mature protein contains 356 amino acids with a predicted molecular mass of 37.9 kDa and an isoelectric pH of 5.2.

### Example 9

### Construction and Transformation of Pathway Constructs for Isopropanol Production in E. coli

Construction of pSJ10843 Containing a C. beijerinckii Acetoacetate Decarboxylase Gene and a C. beijerinckii Alcohol Dehydrogenase Gene.

Plasmids pSJ10725 and pSJ10713 were digested individually with KpnI+AlwNI. Plasmid pSJ10725 was further digested with PvuI to reduce the size of unwanted fragments. The resulting 1689 bp fragment of pSJ10725 and the 2557 bp fragment of pSJ10713 were each purified using gel electrophoresis and subsequently ligated as outlined herein. An aliquot of the ligation mixture was used for transformation of E. coli SJ2 chemically competent cells, and transformants selected on LB plates with 200 microgram/ml ampicillin. Four colonies, picked among more than 100 transformants, were all deemed to contain the desired recombinant plasmid by restriction analysis using HindIII, and two of these were kept, resulting in SJ10843 (SJ2/pSJ10843) and SJ10844 (SJ2/pSJ10844).

Construction of pSJ10841 Containing a C. acetobutylicum Acetoacetate Decarboxylase Gene and a C. beijerinckii Alcohol Dehydrogenase Gene.

Plasmids pSJ10725 and pSJ10711 were digested individually with KpnI+AlwNI; in addition, pSJ10725 was digested with PvuI to reduce the size of unwanted fragments. The resulting 1689 bp fragment of pSJ10725 and the 2596 bp fragment of pSJ10711 were each purified using gel electrophoresis and subsequently ligated as outlined herein. An aliquot of the ligation mixture was used for transformation of E. coli SJ2 chemically competent cells, and transformants selected on LB plates with 200 microgram/ml ampicillin. 4 colonies, picked among more than 100 transformants, were all deemed to contain the desired recombinant plasmid by restriction analysis using BsgI, and two of these were kept, resulting in SJ10841 (SJ2/pSJ10841) and SJ10842 (SJ2/pSJ10842).

Construction of pSJ10748 Containing a B. subtilis Succinyl-CoA:Acetoacetate Transferase Genes.

Plasmids pSJ10697 and pSJ10695 were each digested with EcoRI and KpnI. The resulting 690 bp fragment of pSJ10697 and the 3106 bp fragment of pSJ10695 were each purified using gel electrophoresis and subsequently ligated as outlined herein.

An aliquot of the ligation mixture was used for transformation of E. coli SJ2 by electroporation, and transformants selected on LB plates with 200 microgram/ml ampicillin. 3 colonies, picked among more than 50 transformants, were all deemed to contain the desired recombinant plasmid by restriction analysis using PvuI, and two of these were kept, resulting in SJ10748 (SJ2/pSJ10748) and SJ10749 (SJ2/pSJ10749).

Construction of pSJ10777 Containing a B. mojavensis Succinyl-CoA:Acetoacetate Transferase Genes.

Plasmids pSJ10723 and pSJ10721 were each digested with EcoRI+KpnI. The resulting 696 bp fragment of pSJ10723 and the 3118 bp fragment of pSJ10721 were each purified using gel electrophoresis and subsequently ligated as outlined herein.

An aliquot of the ligation mixture was used for transformation of E. coli SJ2 chemically competent cells, and transformants selected on LB plates with 200 microgram/ml ampicillin. 4 colonies, picked among more than 500 transformants, were analyzed and one, deemed to contain the desired recombinant plasmid by restriction analysis using PvuI, was kept, resulting in SJ10777 (SJ2/pSJ10777).

Construction of pSJ10750 Containing a E. coli Acetoacetyl-CoA Transferase Genes.

Plasmids pSJ10717 and pSJ10715 were each digested with EcoRI+KpnI. The resulting 702 bp fragment of pSJ10717 and the 3051 bp fragment of pSJ10715 were each purified using gel electrophoresis and subsequently ligated as outlined herein.

An aliquot of the ligation mixture was used for transformation of E. coli SJ2 by electroporation, and transformants selected on LB plates with 200 microgram/ml ampicillin. 3 colonies, picked among more than 50 transformants, were all deemed to contain the desired recombinant plasmid by restriction analysis using ApaLI, and two of these were kept, resulting in SJ10750 (SJ2/pSJ10750) and SJ10751 (SJ2/pSJ10751).

Construction of pSJ10752 Containing a Clostridium acetobutylicum Acetoacetyl-CoA Transferase Genes.

Plasmids pSJ10731 and pSJ10727 were each digested with EcoRI+KpnI. The resulting 705 bp fragment of pSJ10731 and the 3061 bp fragment of pSJ10727 were each purified using gel electrophoresis and subsequently ligated as outlined herein.

An aliquot of the ligation mixture was used for transformation of E. coli SJ2 by electroporation, and transformants selected on LB plates with 200 microgram/ml ampicillin. 3 colonies, picked among more than 50 transformants, were all deemed to contain the desired recombinant plasmid by restriction analysis using PvuI, and two of these were kept, resulting in SJ10752 (SJ2/pSJ10752) and SJ10753 (SJ2/pSJ10753).

Construction of Expression Vector pSJ10798 Containing a Clostridium acetobutylicum Thiolase Gene.

Plasmid pSJ10705 was digested with BspHI and EcoRI, whereas pSJ10600 was digested with NcoI and EcoRI. The resulting 1193 bp fragment of pSJ10705 and the 5147 bp fragment of pSJ10600 were each purified using gel electrophoresis and subsequently ligated as outlined herein.

An aliquot of the ligation mixture was used for transformation of E. coli TG1 by electroporation, and transformants selected on LB plates with 200 microgram/ml erythromycin. 3 of 4 colonies analyzed were deemed to contain the desired recombinant plasmid by restriction analysis using NsiI as well as DNA sequencing, and two of these were kept, resulting in SJ10798 (TG1/pSJ10798) and SJ10799 (TG1/pSJ10799).

Construction of Expression Vector pSJ10796 Containing a L. reuteri Thiolase Gene.

Plasmid pSJ10694 was digested with NcoI and EcoRI, and the resulting 1.19 kb fragment purified using gel electrophoresis. Plasmid pSJ10600 was digested with NcoI and EcoRI, and the 5.2 kb fragment purified using gel electrophoresis. The purified fragments were mixed, ligated, and the ligation mixture transformed into TG1 electrocompetent cells, selecting erythromycin resistance (200 microgram/ml) on LB plates at 37° C. Four of the resulting colonies were analyzed and deemed to contain the desired recombinant plasmid by restriction analysis using NsiI, and two of these, further verified by DNA sequencing, were kept, resulting in SJ10796 (TG1/pSJ10796) and SJ10797 (TG1/pSJ10797).

Construction of Expression Vector pSJ10795 Containing a Propionibacterium Freudenreichii Thiolase Gene.

Plasmid pSJ10676 was digested with BspHI and EcoRI, and the resulting 1.17 kb fragment purified using gel electrophoresis. Plasmid pSJ10600 was digested with NcoI and EcoRI, and the 5.2 kb fragment purified using gel electrophoresis. The purified fragments were mixed, ligated, and the ligation mixture transformed into TG1 electrocompetent cells, selecting erythromycin resistance (200 microgram/ml) on LB plates at 37° C. Four of the resulting colonies were analyzed and deemed to contain the desired recombinant plasmid by restriction analysis using NsiI, and one of these, further verified by DNA sequencing, was kept, resulting in SJ10795 (TG1/pSJ10795).

Construction of Expression Vector pSJ10743 Containing a Lactobacillus brevis Thiolase Gene.

Plasmid pSJ10699 was digested with NcoI and EcoRI, and the resulting 1.18 kb fragment purified using gel electrophoresis. Plasmid pSJ10600 was digested with NcoI and EcoRI, and the 5.2 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. 16 of the resulting colonies were analyzed and two, deemed to contain the desired recombinant plasmid by restriction analysis using ClaI and further verified by DNA sequencing, were kept, resulting in SJ10743 (TG1/pSJ10743) and SJ10757 (TG1/pSJ10757).

Construction of Expression Vector pSJ10886 Containing a Bacillus subtilis Succinyl-CoA:Acetoacetate Transferase Genes.

Plasmid pSJ10748 was digested with NcoI and KpnI, and the resulting 1.4 kb fragment purified using gel electrophoresis. Plasmid pSJ10600 was digested with NcoI and KpnI, and the resulting 5.1 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using HindIII, and two of these, further verified by DNA sequencing, were kept, resulting in SJ10886 (TG1/pSJ10886) and SJ10887 (TG1/pSJ10887).

Construction of Expression Vector pSJ10888 Containing E. coli Acetoacetyl-CoA Transferase Genes.

Plasmid pSJ10750 was digested with NcoI and KpnI, and the resulting 1.35 kb fragment purified using gel electrophoresis. Plasmid pSJ10600 was digested with NcoI and KpnI, and the resulting 5.1 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using HindIII, and two of these, further verified by DNA sequencing, were kept, resulting in SJ10888 (TG1/pSJ10888) and SJ10889 (TG1/pSJ10889).

Construction of Expression Vector pSJ10756 Containing a C. beijerinckii Acetoacetate Decarboxylase Gene.

Plasmid pSJ10713 was digested with EagI and KpnI, and the resulting 0.77 kb fragment purified using gel electrophoresis. Plasmid pSJ10600 was digested with EagI and KpnI, and the resulting 5.1 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Four of the resulting colonies were analyzed and one, deemed to contain the desired recombinant plasmid by restriction analysis using ClaI and verified by DNA sequencing, was kept as SJ10756 (TG1/pSJ10756).

Construction of Expression Vector pSJ10754 Containing a C. acetobutylicum Acetoacetate Decarboxylase Gene.

Plasmid pSJ10711 was digested with EagI and KpnI, and the resulting 0.81 kb fragment purified using gel electrophoresis. Plasmid pSJ10600 was digested with EagI and KpnI, and the resulting 5.1 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. Four of the resulting colonies were analyzed, three deemed to contain the desired recombinant plasmid by restriction analysis using ClaI and two, verified by DNA sequencing, were kept as SJ10754 (MG1655/pSJ10754) and SJ10755 (MG1655/pSJ10755).

Construction of Expression Vector pSJ10780 Containing a L. salvarius Acetoacetate Decarboxylase Gene.

Plasmid pSJ10707 was digested with Pcil and KpnI, and the resulting 0.84 kb fragment purified using gel electrophoresis. Plasmid pSJ10600 was digested with NcoI and KpnI, and the resulting 5.1 kb fragment purified using gel electrophoresis. The purified fragments were mixed, ligated, and the ligation mixture transformed into MG1655 chemically competent cells, selecting erythromycin resistance (200 microgram/ml) on LB plates at 37° C. Four of the resulting colonies were analyzed, all deemed to contain the desired recombinant plasmid by restriction analysis using ClaI and two, verified by DNA sequencing, were kept as SJ10780 (MG1655/pSJ10780) and SJ10781 (MG1655/pSJ10781).

Construction of Expression Vector pSJ10778 Containing a L. plantarum Acetoacetate Decarboxylase Gene.

Plasmid pSJ10701 was digested with NcoI and KpnI, and the resulting 0.85 kb fragment purified using gel electrophoresis. Plasmid pSJ10600 was digested with NcoI and KpnI, and the resulting 5.1 kb fragment purified using gel electrophoresis. The purified fragments were mixed, ligated, and the ligation mixture transformed into MG1655 chemically competent cells, selecting erythromycin resistance (200 microgram/ml) on LB plates at 37° C. Four of the resulting colonies were analyzed, all deemed to contain the desired recombinant plasmid by restriction analysis using ClaI and two, verified by DNA sequencing, were kept as SJ10778 (MG1655/pSJ10778) and SJ10779 (MG1655/pSJ10779).

Construction of Expression Vector pSJ10768 Containing a Lactobacillus antri Isopropanol Dehydrogenase Gene.

Plasmid pSJ10709 was digested with KpnI and XmaI, and the resulting 1.1 kb fragment purified using gel electrophoresis. Plasmid pSJ10600 was digested with XmaI and KpnI, and the resulting 5.1 kb fragment purified using gel electrophoresis. The purified fragments were mixed, ligated, and the ligation mixture transformed into TG1 electrocompetent cells, selecting erythromycin resistance (200 microgram/ml) on LB plates at 37° C. Four of the resulting colonies were analyzed and two deemed to contain the desired recombinant plasmid by restriction analysis using ClaI and verified by DNA sequencing, were kept as SJ10768 (TG1/pSJ10768) and SJ10769 (TG1/pSJ10769).

Construction of Expression Vectors pSJ10745, pSJ10763, pSJ10764, and pSJ10767, Containing a Thermoanaerobacter ethanolicus Isopropanol Dehydrogenase Gene.

Plasmid pSJ10719 was digested with BspHI and XmaI, and the resulting 1.06 kb fragment purified using gel electrophoresis. Plasmid pSJ10600 was digested with NcoI and XmaI, and the resulting 5.1 kb fragment purified using gel electrophoresis. The purified fragments were mixed and ligated. The ligation mixture was transformed into MG1655 electrocompetent cells, and one of the resulting colonies, deemed to contain the desired recombinant plasmid by restriction analysis using ClaI and verified by DNA sequencing, was kept as SJ10745 (MG1655/pSJ10745). The ligation mixture was also tranformed into electrocompetent E. coli JM103, where two of four colonies were deemed to contain the desired plasmid by restriction analysis using ClaI, and these kept as SJ10763 (JM103/pSJ10763) and SJ10764 (JM103/pSJ10764).

Finally, the ligation mixture was transformed into electrocompetent TG1, where three of four colonies were deemed to contain the desired plasmid by restriction analysis using ClaI, and one, SJ10767 (JM103/pSJ10767), was verified by DNA sequencing.

Construction of Expression Vector pSJ10782 Containing a Clostridium beijerinckii Isopropanol Dehydrogenase Gene.

Plasmid pSJ10725 was digested with BspHI and XmaI, and the resulting 1.06 kb fragment purified using gel electrophoresis. Plasmid pSJ10600 was digested with NcoI and XmaI, and the resulting 5.1 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. Four of the resulting colonies were analyzed and two, deemed to contain the desired recombinant plasmid by restriction analysis using ClaI and verified by DNA sequencing, were kept as SJ10782 (TG1/pSJ10782) and SJ10783 (TG1/pSJ10783).

Construction of Expression Vector pSJ10762 Containing a Lactobacillus fermentum Isopropanol Dehydrogenase Gene.

Plasmid pSJ10703 was digested with BspHI and XmaI, and the resulting 1.1 kb fragment purified using gel electrophoresis. Plasmid pSJ10600 was digested with XmaI and NcoI, and the resulting 5.1 kb fragment purified using gel electrophoresis. The purified fragments were mixed, ligated, and the ligation mixture transformed into JM103 as well as TG1 electrocompetent cells, selecting erythromycin resistance (200 microgram/ml) on LB plates at 37° C. Transformants were analyzed and two (one from each host strain), deemed to contain the desired recombinant plasmid by restriction analysis using ClaI and verified by DNA sequencing, were kept as SJ10762 (JM103/pSJ10762) and SJ10765 (TG1/pSJ10765). Transformant SJ10766 (JM103/pSJ10766) was also verified to contain the Lactobacillus fermentum isopropanol dehydrogenase gene.

Construction of Expression Vector pSJ10954 Containing a C. acetobutylicum Thiolase Gene, B. mojavensis Succinyl-CoA:Acetoacetate Transferase Genes (Both Subunits), a C. Beijerinckii Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10777 was digested with XhoI and EagI, and the resulting 1.43 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10954 (TG1/pSJ10954) and SJ10955 (TG1/pSJ10955).

Construction of Expression Vector pSJ10956 Containing a C. acetobutylicum Thiolase Gene, B. mojavensis Succinyl-CoA:Acetoacetate Transferase Genes (Both Subunits), a C. acetobutylicum Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10777 was digested with XhoI and EagI, and the resulting 1.43 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10956 (TG1/pSJ10956) and SJ10957 (TG1/pSJ10957).

From an independent construction process (digestion, fragment purification, ligation, transformation by electroporation) one transformant, deemed to contain the desired recombinant plasmid by restriction analysis using XbaI, was kept as SJ10926 (TG1 pSJ10926).

Construction of Expression Vector pSJ10942 Containing a C. acetobutylicum Thiolase Gene, B. subtilis Succinyl-CoA:Acetoacetate Transferase Genes (Both Subunits), a C. beijerinckii Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10748 was digested with XhoI and EagI, and the resulting 1.43 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10942 (TG1/pSJ10942) and SJ10943 (TG1/pSJ10943).

Construction of Expression Vector pSJ10944 Containing a C. acetobutylicum Thiolase Gene, B. subtilis Succinyl-CoA:Acetoacetate Transferase Genes (Both Subunits), a C. acetobutylicum Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10748 was digested with XhoI and EagI, and the resulting 1.43 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10944 (TG1/pSJ10944) and SJ10945 (TG1/pSJ10945).

Construction of Expression Vector pSJ10946 Containing a C. acetobutylicum Thiolase Gene, an E. coli Acetoacetyl-CoA Transferase Genes (Both Subunits), a C. beijerinckii Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10750 was digested with XhoI and EagI, and the resulting 1.37 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10946 (TG1/pSJ10946) and SJ10947 (TG1/pSJ10947).

Construction of Expression Vector pSJ10948 Containing a C. acetobutylicum Thiolase Gene, E. coli Acetoacetyl-CoA Transferase Genes (Both Subunits), a C. acetobutylicum Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10750 was digested with XhoI and EagI, and the resulting 1.37 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10948 (TG1/pSJ10948) and SJ10949 (TG1/pSJ10949).

Construction of Expression Vector pSJ10950 Containing a C. acetobutylicum Thiolase Gene, C. acetobutylicum Acetoacetyl-CoA Transferase Genes (Both Subunits), a C. beijerinckii Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10752 was digested with XhoI and EagI, and the resulting 1.38 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10950 (TG1/pSJ10950) and SJ10951 (TG1/pSJ10951).

Construction of Expression Vector pSJ10952 Containing a C. acetobutylicum Thiolase Gene, C. acetobutylicum Acetoacetyl-CoA Transferase Genes (Both Subunits), a C. acetobutylicum Acetoacetate Decarboxylase Gene, and a C. beijerinckii Alcohol Dehydrogenase Gene.

Plasmid pSJ10798 was digested with XhoI and XmaI, and the resulting 6.3 kb fragment purified using gel electrophoresis. Plasmid pSJ10752 was digested with XhoI and EagI, and the resulting 1.38 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ10952 (TG1/pSJ10952) and SJ10953 (TG1/pSJ10953).

Construction of Expression Vector pSJ10790 Containing a C. acetobutylicum Thiolase Gene, B. mojavensis Succinyl-CoA:Acetoacetate Transferase Genes (Both Subunits), a C. beijerinckii Acetoacetate Decarboxylase Gene, and a C. beijerinckii Alcohol Dehydrogenase Gene Under Control of the P11 Promoter.

Plasmid pTRGU00178 (see U.S. Provisional Patent Application No. 61/408,138, filed Oct. 29, 2010) was digested with NcoI and BamHI, and the resulting 1.2 kb fragment purified using gel electrophoresis. pTRGU00178 was also digested with BamHI and SalI, and the resulting 2.1 kb fragment purified using gel electrophoresis. pSIP409 was digested with NcoI and XhoI, and the resulting 5.7 kb fragment purified using gel electrophoresis. The three purified fragments were mixed, ligated, and the ligation mixture transformed into SJ2 electrocompetent cells, selecting erythromycin resistance (200 microgram/ml) on LB plates at 37° C. Two transformants, deemed to contain the desired recombinant plasmid by restriction analysis using EcoRI, BglII, and HindIII, were kept as SJ10562 (SJ2/pSJ10562) and SJ10563 (SJ2/pSJ10563).

Plasmid pSJ10562 was digested with XbaI and NotI, and the resulting 7.57 kb fragment purified using gel electrophoresis. Plasmid pTRGU00200 (supra) was digested with XbaI and NotI, and the resulting 2.52 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. Two transformants, deemed to contain the desired recombinant plasmid by restriction analysis using NotI+XbaI, were kept as SJ10593 (MG1655/pSJ10593) and SJ10594 (MG1655/pSJ10594).

Plasmid pTRGU00200 was digested with EcoRI and BamHI, and the resulting 1.2 kb fragment purified using gel electrophoresis. pSJ10600 was digested with EcoRI and BamHI, and the resulting 5.2 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. Two transformants, deemed to contain the desired recombinant plasmid by restriction analysis using EcoRI+BamHI, were kept as SJ10690 (MG1655/pSJ10690) and SJ10691 (MG1655/pSJ10691).

Plasmid pSJ10593 was digested with BamHI and XbaI, and the resulting 3.25 kb fragment purified using gel electrophoresis. pSJ10690 was digested with BamHI and XbaI, and the resulting 6.3 kb fragment purified using gel electrophoresis. The purified fragments were mixed, ligated, and the ligation mixture transformed into TG1 electrocompetent cells, selecting erythromycin resistance (200 microgram/ml) on LB plates at 37° C. Two transformants, deemed to contain the desired recombinant plasmid by restriction analysis using NsiI, were kept as SJ10790 (TG1/pSJ10790) and SJ10791 (TG1/pSJ10791).

Construction of pSJ10792 Containing a C. acetobutylicum Thiolase Gene, B. moiavensis Succinyl-CoA:Acetoacetate Transferase Genes (Both Subunits), a C. beiierinckii Acetoacetate Decarboxylase Gene, and a C. beiierinckii Alcohol Dehydrogenase Gene Under Control of the P27 Promoter.

Plasmid pTRGU00200 was digested with EcoRI and BamHI, and the resulting 1.2 kb fragment purified using gel electrophoresis. pSJ10603 was digested with EcoRI and BamHI, and the resulting 5.2 kb fragment purified using gel electrophoresis. The purified fragments were mixed, ligated, and the ligation mixture transformed into MG1655 electrocompetent cells, selecting erythromycin resistance (200 microgram/ml) on LB plates at 37° C. Two transformants, deemed to contain the desired recombinant plasmid by restriction analysis using EcoRI+BamHI, were kept as SJ10692 (MG1655/pSJ10692) and SJ10693 (MG1655/pSJ10693).

Plasmid pSJ10593 was digested with BamHI and XbaI, and the resulting 3.25 kb fragment purified using gel electrophoresis. pSJ10692 was digested with BamHI and XbaI, and the resulting 6.3 kb fragment purified using gel electrophoresis. The purified fragments were mixed, ligated, and the ligation mixture transformed into TG1 electrocompetent cells, selecting erythromycin resistance (200 microgram/ml) on LB plates at 37° C. Two transformants, deemed to contain the desired recombinant plasmid by restriction analysis using NsiI, were kept as SJ10792 (TG1/pSJ10792) and SJ10793 (TG1/pSJ10793).

Construction of Expression Vector pSJ11208 Containing a L. reuteri Thiolase Gene, B. mojavensis Succinyl-CoA:Acetoacetate Transferase Genes (Both Subunits), a C. beijerinckii Acetoacetate Decarboxylase Gene, and a C. beijerinckii Alcohol Dehydrogenase Gene.

Plasmid pSJ10796 (described below) was digested with XhoI and XmaI, and the resulting 6.3 kb fragment purified using gel electrophoresis. Plasmid pSJ10954 was digested with XhoI and XmaI, and the resulting 3.28 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Three of the resulting colonies were analyzed and deemed to contain the desired recombinant plasmid by restriction analysis using XbaI, and two of these were kept, resulting in SJ11208 (TG1/pSJ11208) and SJ11209 (TG1/pSJ11209).

Construction of Expression Vector pSJ11204 Containing a L. reuteri Thiolase Gene, B. subtilis Succinyl-CoA:Acetoacetate Transferase Genes (Both Subunits), a C. beijerinckii Acetoacetate Decarboxylase Gene, and a C. beijerinckii Alcohol Dehydrogenase Gene.

Plasmid pSJ10796 (described below) was digested with XhoI and XmaI, and the resulting 6.3 kb fragment purified using gel electrophoresis. Plasmid pSJ10942 was digested with XhoI and XmaI, and the resulting 3.26 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Four of the resulting colonies were analyzed and deemed to contain the desired recombinant plasmid by restriction analysis using XbaI, and two of these were kept, resulting in SJ11204 (TG1/pSJ11204) and SJ11205 (TG1/pSJ11205).

Construction of Expression Vector rSJ11230 Containing a L. reuteri Thiolase Gene, E. coli Acetoacetyl-CoA Transferase Genes (Both Subunits), a C. beijerinckii Acetoacetate Decarboxylase Gene, and a C. beijerinckii Alcohol Dehydrogenase Gene.

Plasmid pSJ10796 (described below) was digested with XhoI and XmaI, and the resulting 6.3 kb fragment purified using gel electrophoresis. Plasmid pSJ10946 was digested with XhoI and XmaI, and the resulting 3.23 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Seven of the resulting colonies were analyzed and 5 deemed to contain the desired recombinant plasmid by restriction analysis using XbaI, and two of these were kept, resulting in SJ11230 (TG1/pSJ11230) and SJ11231 (TG1/pSJ11231).

Construction of Expression Vector pSJ11206 Containing a L. reuteri Thiolase Gene, C. acetobutylicum Acetoacetyl-CoA Transferase Genes (Both Subunits), a C. beijerinckii Acetoacetate Decarboxylase Gene, and a C. beijerinckii Alcohol Dehydrogenase Gene.

Plasmid pSJ10796 (described below) was digested with XhoI and XmaI, and the resulting 6.3 kb fragment purified using gel electrophoresis. Plasmid pSJ10951 was digested with XhoI and XmaI, and the resulting 3.23 kb fragment purified using gel electrophoresis. The purified fragments were mixed, ligated, and the ligation mixture transformed into TG1 chemically competent cells, selecting erythromycin resistance (200 microgram/ml) on LB plates at 37° C. Four of the resulting colonies were analyzed and two, deemed to contain the desired recombinant plasmid by restriction analysis using XbaI, were kept as SJ11206 (TG1/pSJ11206) and SJ11207 (TG1/pSJ11207).

### Example 10

### Production of Acetone and Isopropanol During Small Scale Batch Propagation of E. coli

E. coli strains described in Example 9 were inoculated directly from the −80° C. stock cultures, and grown overnight in LB medium supplemented with 1% glucose and 100 microgram/ml erythromycin, with shaking at 300 rpm at 37° C.

A 1.5 mL sample from each medium was withdrawn after 24 hours. Each sample was centrifuged at 15000×g using a table centrifuge and the supernatant was analyzed using gas chromatography. Acetone and isopropanol in fermentation broths were detected by GC-FID as described above. Results are shown in Table 2, wherein the gene constructs are represented with the following abbreviations:

thl_Ca: C. acetobutylicum thiolase gene
adh_Cb: C. beijerinckii alcohol dehydrogenase
scoAB_Bm: B. mojavensis succinyl-CoA:acetoacetate transferase genes (both subunits)
scoAB_Bs: B. subtilis succinyl-CoA:acetoacetate transferase genes (both subunits)
atoAD_Ec: E. coli acetoacetyl-CoA transferase genes (both subunits)
ctfAB_Ca: C. acetobutylicum acetoacetyl-CoA transferase genes (both subunits)
adc_Cb: C. beijerinckii acetoacetate decarboxylase gene
adc_Ca: C. acetobutylicum acetoacetate decarboxylase gene

As control strains, E. coli SJ10766 (containing the same expression vector backbone, but harbouring only an isopropanol dehydrogenase gene L. fermentum (sadh_Lf) of SEQ ID NO: 121, and E. coli SJ10799 (containing the same expression vector, but harbouring only the C. acetobutylicum thiolase gene of SEQ ID NO: 2) were inoculated in the same manner.

TABLE 2

| | | | Ace- | iso- |
|---|---|---|---|---|
| | | | tone | propanol |
| Strain | Construct | SEQ ID Nos | (%) | (%) |
| SJ10942 | thl_Ca, scoAB_Bs, | 2, 5, 8, 17, 20 | 0.073 | 0.122 |
| SJ10943 | adc_Cb, adh_Cb | | 0.020 | 0.104 |
| SJ10944 | thl_Ca, scoAB_Bs, | 2, 5, 8, 20, 44 | 0.012 | 0.088 |
| SJ10945 | adc_Ca, adh_Cb | | 0.013 | 0.103 |
| SJ10946 | thl_Ca, atoAD_Ec, | 2, 17, 20, 36, 38 | 0.028 | 0.142 |
| SJ10947 | adc_Cb, adh_Cb | | 0.018 | 0.078 |
| SJ10948 | thl_Ca, atoAD_Ec, | 2, 20, 36, 38, 44 | 0.011 | 0.091 |
| SJ10949 | adc_Ca, adh_Cb | | 0.011 | 0.071 |
| SJ10950 | thl_Ca, ctfAB_Ca, | 2, 17, 20, 40, 42 | 0.022 | 0.116 |
| SJ10951 | adc_Cb, adh_Cb | | 0.009 | 0.074 |
| SJ10952 | thl_Ca, ctfAB_Ca, | 2, 20, 40, 42, 44 | 0.011 | 0.108 |
| SJ10953 | adc_Ca, adh_Cb | | 0.010 | 0.093 |
| SJ10926 | thl_Ca, scoAB_Bm, | 2, 11, 14, 20, 44 | 0.014 | 0.050 |
| SJ10956 | adc_Ca, adh_Cb | | 0.007 | 0.039 |
| SJ10957 | | | 0.007 | 0.042 |
| SJ10954 | thl_Ca, scoAB_Bm, | 2, 11, 14, 17, 20 | 0.010 | 0.060 |
| SJ10955 | adc_Cb, adh_Cb | | 0.007 | 0.032 |
| SJ10790 | | | 0.006 | 0.058 |
| SJ10791 | | | 0.005 | 0.057 |
| SJ10792 | | | 0.008 | 0.099 |
| SJ10793 | | | 0.007 | 0.079 |
| SJ10766 | (Control) sadh_Lf | 121 | 0.004 | nd |
| SJ10799 | (Control) thl_Ca | 2 | 0.003 | nd |

*nd means not detected.

Similar cultures were incubated without shaking; in all of these, 2-propanol levels were between 0.001% and 0.009%, except for the two control strains SJ10766 and SJ10799, where isopropanol was not detected.

### Example 11

### Production of Acetone and Isopropanol During Small Scale Batch Propagation of E. coli Under Varying Glucose Concentrations

Fermentation media (LB with 100 microgram/ml erythromycin, and either 1, 2, 5 or 10% glucose to a total volumer of 10 ml) was inoculated with strains directly from the frozen stock cultures, and incubated at 37° C. with shaking. Supernatant samples were taken after 1, 2, and 3 days, and analyzed for acetone and isopropanol content as described above. Strain SJ10766 (containing the same expression vector backbone, but harbouring only an alcohol dehydrogenase gene sadh_Lf) was included as a negative control.

Results are shown in Table 3, wherein the gene constructs are represented with the abbreviations shown in Example 3. All isopropanol operon strains are able to produce more than 1 g/l of isopropanol, with the highest yielding strain in this experiment, SJ10946, producing 0.208% isopropanol.

TABLE 3

| | | | Glucose | | Acetone | 2-propanol |
|---|---|---|---|---|---|---|
| Strain | Construct | SEQ ID Nos | (%) | Day | (%) | (%) |
| SJ10926 | thl_Ca, | 2, 11, 14, 20, | 1 | 1 | 0.008 | 0.055 |
| | scoAB_Bm, | 44 | | 2 | 0.013 | 0.085 |
| | adc_Ca, adh_Cb | | | 3 | 0.028 | 0.021 |
| | | | 2 | 1 | 0.012 | 0.07 |
| | | | | 2 | 0.019 | 0.146 |
| | | | | 3 | 0.01 | 0.156 |
| | | | 5 | 1 | 0.014 | 0.059 |
| | | | | 2 | 0.018 | 0.144 |
| | | | | 3 | 0.014 | 0.119 |
| | | | 10 | 1 | 0.01 | 0.021 |
| | | | | 2 | 0.021 | 0.157 |
| | | | | 3 | 0.013 | 0.132 |
| SJ10942 | thl_Ca, scoAB_Bs, | 2, 5, 8, 17, 20 | 1 | 1 | 0.009 | 0.079 |
| | adc_Cb, adh_Cb | | | 2 | 0.012 | 0.077 |
| | | | | 3 | 0.052 | 0.034 |
| | | | 2 | 1 | 0.011 | 0.085 |
| | | | | 2 | 0.009 | 0.143 |
| | | | | 3 | 0.012 | 0.19 |
| | | | 5 | 1 | 0.011 | 0.054 |
| | | | | 2 | 0.021 | 0.191 |
| | | | | 3 | 0.014 | 0.153 |
| | | | 10 | 1 | 0.008 | 0.003 |
| | | | | 2 | 0.008 | 0.02 |
| | | | | 3 | 0.014 | 0.022 |
| SJ10946 | thl_Ca, atoAD_Ec, | 2, 17, 20, 36, | 1 | 1 | 0.024 | 0.079 |
| | adc_Cb, adh_Cb | 38 | | 2 | 0.042 | 0.101 |
| | | | | 3 | 0.041 | 0.053 |
| | | | 2 | 1 | 0.026 | 0.082 |
| | | | | 2 | 0.06 | 0.192 |
| | | | | 3 | 0.054 | 0.208 |
| | | | 5 | 1 | 0.018 | 0.056 |
| | | | | 2 | 0.046 | 0.161 |
| | | | | 3 | 0.039 | 0.181 |
| | | | 10 | 1 | 0.007 | nd |
| | | | | 2 | 0.01 | 0.001 |
| | | | | 3 | 0.012 | 0.001 |
| SJ10950 | thl_Ca, ctfAB_Ca, | 2, 17, 20, 40, 4 | 1 | 1 | 0.035 | 0.107 |
| | adc_Cb, adh_Cb | | | 2 | 0.063 | 0.076 |
| | | | | 3 | 0.037 | 0.036 |
| | | | 2 | 1 | 0.029 | 0.117 |
| | | | | 2 | 0.031 | 0.191 |
| | | | | 3 | 0.067 | 0.074 |
| | | | 5 | 1 | 0.026 | 0.005 |
| | | | | 2 | 0.027 | 0.011 |
| | | | | 3 | 0.018 | 0.014 |
| | | | 10 | 1 | 0.009 | nd |
| | | | | 2 | 0.014 | 0.002 |
| | | | | 3 | 0.011 | 0.001 |
| SJ10766 | (Control) sadh_Lf | 121 | 1 | 1 | 0.002 | nd |
| | | | | 2 | 0.001 | nd |
| | | | | 3 | 0.001 | nd |
| | | | 2 | 1 | 0.003 | nd |
| | | | | 2 | 0.002 | nd |
| | | | | 3 | 0.004 | nd |
| | | | 5 | 1 | 0.002 | nd |
| | | | | 2 | 0.002 | nd |
| | | | | 3 | 0.008 | nd |
| | | | 10 | 1 | 0.008 | nd |
| | | | | 2 | 0.006 | nd |
| | | | | 3 | 0.009 | nd |

*nd means not detected.

### Example 12

### Production of Acetone and Isopropanol During Small Scale Batch Propagation of E. coli.

Selected E. coli strains described above were inoculated in duplicate directly from the −80° C. stock cultures, and grown overnight in LB medium supplemented with 1% glucose and 100 microgram/ml erythromycin, in 10 ml tubes with shaking at 300 rpm at 37° C. A 1.5 mL sample from each medium was withdrawn after 24 hours. Each sample was centrifuged at 15000×g and the supernatant was for acetone and isopropanol content as described above.

Results are shown in Table 4, wherein gene constructs are represented with the abbreviations shown in Example 3, and thl_Lr represents the L. reuteri thiolase gene construct.

TABLE 4

| | | | Iso- | Ace- |
|---|---|---|---|---|
| | | | propanol | tone |
| Strain | Construct | SEQ ID Nos | (%) | (%) |
| SJ11204 | thl_Lr, scoAB_Bs, | 5, 8, 17, 20, 34 | 0.027 | 0.005 |
| SJ11204 | adc_Cb, adh_Cb | | 0.036 | 0.007 |
| SJ11205 | | | 0.030 | 0.005 |
| SJ11205 | | | 0.032 | 0.005 |
| SJ11206 | thl_Lr, ctfAB_Ca, | 17, 20, 34, 40, 42 | 0.041 | 0.007 |
| SJ11206 | adc_Cb, adh_Cb | | 0.039 | 0.007 |
| SJ11207 | | | 0.036 | 0.006 |
| SJ11207 | | | 0.039 | 0.007 |
| SJ11208 | thl_Lr, scoAB_Bm, | 11, 14, 17, 20, 34 | 0.031 | 0.005 |
| SJ11208 | adc_Cb, adh_Cb | | 0.035 | 0.005 |
| SJ11209 | | | 0.033 | 0.006 |
| SJ11209 | | | 0.036 | 0.006 |
| SJ11230 | thl_Lr, atoAD_Ec, | 17, 20, 34, 36, 38 | 0.042 | 0.007 |
| SJ11230 | adc_Cb, adh_Cb | | 0.042 | 0.007 |
| SJ11231 | | | 0.040 | 0.007 |
| SJ11231 | | | 0.047 | 0.008 |

This expriment demonstrates that E. coli TG1 harbouring expression vectors based on pSJ10600 comprising the L. reuteri thiolase gene are capable of producing a significant amount of isopropanol.

### Example 13

### Construction and Transformation of Peptide-Inducible Pathway Constructs for Isopropanol Production in L. Plantarum

Construction of Expression Vector pSJ10776 Containing a Clostridium acetobutylicum Thiolase Gene.

Plasmid pSJ10705 was digested with BspHI and EcoRI, and pSIP409 was digested with NcoI and EcoRI. The resulting 1.19 kb fragment of pSJ10705 and the 5.6 kb fragment of pSIP409 were each purified using gel electrophoresis and subsequently ligated as outlined herein.

An aliquot of the ligation mixture was used for transformation of E. coli MG1655 chemically competent cells as described herein, and transformants selected on LB plates with 200 microgram/ml erythromycin, at 37° C. One transformant, deemed to contain the desired recombinant plasmid by restriction analysis using PstI+NsiI, as well as DNA sequencing, was kept as SJ10776 (MG1655/pSJ10776).

Construction of Expression Vector pSJ10903 Containing a C. acetobutylicum Thiolase Gene, a B. subtilis Succinyl-CoA:Acetoacetate Transferase Gene(s), a C. beijerinckii Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10748 was digested with XhoI and EagI, and the resulting 1.43 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and 2 strains, deemed to contain the desired recombinant plasmid by restriction analysis using BspHI were kept, resulting in SJ10903 (TG1/pSJ10903) and SJ10904 (TG1/pSJ10904).

Construction of Expression Vector pSJ10905 Containing a C. acetobutylicum Thiolase Gene, a B. subtilis Succinyl-CoA:Acetoacetate Transferase Gene(s), a C. acetobutylicum Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10748 was digested with XhoI and EagI, and the resulting 1.43 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed, three deemed to contain the desired recombinant plasmid by restriction analysis using BspHI, and two of these were kept, resulting in SJ10905 (TG1/pSJ10905) and SJ10906 (TG1/pSJ10906).

Construction of Expression Vector pSJ10907 Containing a C. acetobutylicum Thiolase Gene, an E. coli Acetoacetyl-CoA Transferase Gene(s), a C. beijerinckii Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10750 was digested with XhoI and EagI, and the resulting 1.37 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed, three deemed to contain the desired recombinant plasmid by restriction analysis using BspHI, and two of these were kept, resulting in SJ10907 (TG1/pSJ10907) and SJ10908 (TG1/pSJ10908).

Construction of Expression Vector pSJ10909 Containing a C. acetobutylicum Thiolase Gene, an E. coli Acetoacetyl-CoA Transferase Gene(s), a C. acetobutylicum Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10750 was digested with XhoI and EagI, and the resulting 1.37 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed, three deemed to contain the desired recombinant plasmid by restriction analysis using BspHI, and two of these were kept, resulting in SJ10909 (TG1/pSJ10909) and SJ10910 (TG1/pSJ10910).

Construction of Expression Vector pSJ10911 Containing a C. acetobutylicum Thiolase Gene, a B. moiavensis Succinyl-CoA:Acetoacetate Transferase Gene(s), a C. beijerinckii Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10777 was digested with XhoI and EagI, and the resulting 1.43 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Four of the resulting colonies were analyzed and two, deemed to contain the desired recombinant plasmid by restriction analysis using BspHI, were kept, resulting in SJ10911 (TG1/pSJ10911) and SJ10912 (TG1/pSJ10912).

Construction of Expression Vector pSJ10940 Containing a C. acetobutylicum Thiolase Gene, a B. mojavensis Succinyl-CoA:Acetoacetate Transferase Gene(s), a C. acetobutylicum Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10777 was digested with XhoI and EagI, and the resulting 1.43 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Several resulting colonies were analyzed and two, deemed to contain the desired recombinant plasmid by restriction analysis using BspHI, were kept, resulting in SJ10940 (TG1/pSJ10940) and SJ10941 (TG1/pSJ10941).

Construction of Expression Vector pSJ10973 Containing a C. acetobutylicum Thiolase Gene, a C. acetobutylicum Acetoacetyl-CoA Transferase Gene(s), a C. beijerinckii Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10752 was digested with XhoI and EagI, and the resulting 1.38 kb fragment purified using gel electrophoresis. Plasmid pSJ10843 was digested with EagI and XmaI, and the resulting 1.85 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Several resulting colonies were analyzed and two, deemed to contain the desired recombinant plasmid by restriction analysis using PstI as well as ApaLI, were kept as SJ10973 (TG1/pSJ10973) and SJ10974 (TG1/pSJ10974).

Construction of Expression Vector pSJ10975 Containing a C. acetobutylicum Thiolase Gene, a C. acetobutylicum Acetoacetyl-CoA Transferase Gene(s), a C. acetobutylicum Acetoacetate Decarboxylase Gene, and a C. beijerinckii Alcohol Dehydrogenase Gene.

Plasmid pSJ10776 was digested with XhoI and XmaI, and the resulting 6.8 kb fragment purified using gel electrophoresis. Plasmid pSJ10752 was digested with XhoI and EagI, and the resulting 1.38 kb fragment purified using gel electrophoresis. Plasmid pSJ10841 was digested with EagI and XmaI, and the resulting 1.89 kb fragment purified using gel electrophoresis. The three purified fragments were mixed, ligated, and the ligation mixture transformed into TG1 chemically competent cells, selecting erythromycin resistance (200 microgram/ml) on LB plates at 37° C. Several resulting colonies were analyzed and three deemed to contain the desired recombinant plasmid by restriction analysis using PstI as well as ApaLI. Two of these were kept as SJ10975 (TG1/pSJ10975) and SJ10976 (TG1/pSJ10976).

Transformation of L. plantarum SJ10656 with Expression Vectors Containing Peptide-Inducible Isopropanol Operon Constructs.

L. plantarum SJ10656 was transformed with plasmids by electroporation as described herein, and transformants with each of the plasmids were obtained and saved (see Table 5). Constructs are represented with the abbreviations shown in the Examples above.

TABLE 5

| | L. plantarum | | |
|---|---|---|---|
| Plasmid | transformant | Construct | SEQ ID Nos |
| pSJ10903 | SJ10930 | Thl_Ca, scoAB_Bs, | 2, 5, 8, 17, 20 |
| pSJ10904 | SJ10931 | adc_Cb, adh_Cb | |
| pSJ10905 | SJ10932 | Thl_Ca, scoAB_Bs, | 2, 5, 8, 20, 44 |
| pSJ10906 | SJ10933 | adc_Ca, adh_Cb | |
| pSJ10907 | SJ10962 | Thl_Ca, atoAD_Ec, | 2, 17, 20, 36, 38 |
| pSJ10908 | SJ10934 | adc_Cb, adh_Cb | |
| pSJ10909 | SJ10935 | Thl_Ca, atoAD_Ec, | 2, 20, 36, 38, 44 |
| pSJ10910 | SJ10936 | adc_Ca, adh_Cb | |
| pSJ10911 | SJ10937 | Thl_Ca, scoAB_Bm, | 2, 11, 14, 17, 20 |
| pSJ10912 | SJ10938 | adc_Cb, adh_Cb | |
| pSJ10940 | SJ11017 | Thl_Ca, scoAB_Bm, | 2, 11, 14, 20, 44 |
| pSJ10941 | SJ11018 | adc_Ca, adh_Cb | |
| pSJ10973 | SJ11019 | Thl_Ca, ctfAB_Ca, | 2, 17, 20, 40, 42 |
| pSJ10974 | SJ11020 | adc_Cb, adh_Cb | |
| pSJ10975 | SJ11021 | Thl_Ca, ctfAB_Ca, | 2, 20, 40, 42, 44 |
| pSJ10976 | SJ11022 | adc_Ca, adh_Cb | |
| pSJ11204 | SJ11262 | Thl_Lr, scoAB_Bs, | 5, 8, 17, 20, 34 |
| pSJ11205 | SJ11263 | adc_Cb, adh_Cb | |
| pSJ11206 | SJ11264 | Thl_Lr, ctfAB_Ca, | 17, 20, 34, 40, 42 |
| pSJ11207 | SJ11265 | adc_Cb, adh_Cb | |
| pSJ11208 | SJ11266 | Thl_Lr, scoAB_Bm, | 11, 14, 17, 20, 34 |
| pSJ11209 | SJ11267 | adc_Cb, adh_Cb | |
| pSJ11230 | SJ11268 | Thl_Lr, atoAD_Ec, | 17, 20, 34, 36, 38 |
| pSJ11231 | SJ11269 | adc_Cb, adh_Cb | |

### Example 14

### Isopropanol and Acetone Production in L. plantarum with a Subset of the Transformed Strains

MRS medium (2 ml total volume with 10 μg/ml erythromycin) was inoculated with recombinant L. plantarum strains from the stock vials kept at −80° C. into 2 ml eppendorf tubes and incubated overnight at 37° C. without shaking. The following day, a 50 microliter volume of broth from these cultures were used, for each strain, to inoculate each of two 10 ml vials with MRS+10 microgram/ml erythromycin, one containing the inducing peptide (M-19-R) for the pSIP vector system at a concentration approximately 50 ng/ml. Vials were closed and incubated without shaking at 37° C. Supernatant samples were harvested after 1 and 2 days incubation, and analyzed for acetone and isopropanol content as described herein. Results are shown in Table 6. Constructs are represented with the abbreviations shown in the Examples above.

TABLE 6

| | | | | | Acetone | Isopropanol |
|---|---|---|---|---|---|---|
| Strain | Construct | SEQ ID Nos | Induction | Day | (%) | (%) |
| SJ10930 | Thl_Ca, | 2, 5, 8, 17, 20 | − | 1 | 0.001 | nd |
| | scoAB_Bs, | | − | 2 | 0.002 | nd |
| | adc_Cb, | | + | 1 | 0.001 | 0.001 |
| | adh_Cb | | + | 2 | 0.002 | 0.001 |
| SJ10931 | | | − | 1 | 0.001 | nd |
| | | | − | 2 | 0.002 | nd |
| | | | + | 1 | 0.001 | 0.001 |
| | | | + | 2 | 0.002 | 0.001 |
| SJ10932 | Thl_Ca, | 2, 5, 8, 20, 44 | − | 1 | 0.002 | 0.000 |
| | scoAB_Bs, | | − | 2 | 0.002 | nd |
| | adc_Ca, | | + | 1 | 0.002 | 0.001 |
| | adh_Cb | | + | 2 | 0.002 | 0.001 |
| SJ10933 | | | − | 1 | 0.001 | nd |
| | | | − | 2 | 0.002 | nd |
| | | | + | 1 | 0.001 | 0.001 |
| | | | + | 2 | 0.002 | 0.001 |
| SJ10934 | Thl_Ca, | 2, 17, 20, 36, 38 | − | 1 | 0.002 | nd |
| | atoAD_Ec, | | − | 2 | 0.003 | 0.000 |
| | adc_Cb, | | + | 1 | 0.002 | 0.003 |
| | adh_Cb | | + | 2 | 0.003 | 0.003 |
| SJ10962 | | | − | 1 | 0.002 | nd |
| | | | − | 2 | 0.002 | nd |
| | | | + | 1 | 0.002 | 0.003 |
| | | | + | 2 | 0.002 | 0.003 |
| SJ10935 | Thl_Ca, | 2, 20, 36, 38, 44 | − | 1 | 0.001 | nd |
| | atoAD_Ec, | | − | 2 | 0.002 | nd |
| | adc_Ca, | | + | 1 | 0.003 | 0.002 |
| | adh_Cb | | + | 2 | 0.003 | 0.002 |
| SJ10936 | | | − | 1 | 0.001 | nd |
| | | | − | 2 | 0.002 | nd |
| | | | + | 1 | 0.003 | 0.002 |
| | | | + | 2 | 0.003 | 0.002 |
| SJ10937 | Thl_Ca, | 2, 11, 14, 17, 20 | − | 1 | 0.002 | nd |
| | scoAB_Bm, | | − | 2 | 0.002 | nd |
| | adc_Cb, | | + | 1 | 0.003 | 0.001 |
| | adh_Cb | | + | 2 | 0.003 | 0.002 |
| SJ10938 | | | − | 1 | 0.002 | nd |
| | | | − | 2 | 0.002 | nd |
| | | | + | 1 | 0.002 | 0.001 |
| | | | + | 2 | 0.002 | 0.002 |

“nd” means not detected;
“0.000” means that the compound was detected.

### Example 14

### Isopropanol and Acetone Production in L. plantarum and Effects of Acetone Addition

Recombinant L. plantarum strains were grown in stationary MRS medium with 10 microgram/ml erythromycin at 37° C. for 3 days. Cultures contained the inducing M-19-R polypeptide (50 ng/ml) and/or acetone (5 ml/l), as indicated in the Table 7. The supernatants were analyzed for acetone and isopropanol as described herein. Control strain SJ10678 contains the “empty” pSJ10600 expression vector. Results are shown in Table 7. Constructs are represented with the abbreviations shown in the Examples above.

TABLE 7

| | | | | | Acetone | Isopropanol |
|---|---|---|---|---|---|---|
| Strain | Construct | SEQ ID Nos | Acetone | Induction | (%) | (%) |
| SJ10930 | Thl_Ca, | 2, 5, 8, 17, 20 | − | − | 0.003 | nd |
| | scoAB_Bs, | | − | + | 0.002 | 0.001 |
| | adc_Cb, | | + | − | 0.287 | 0.001 |
| | adh_Cb | | + | + | 0.258 | 0.009 |
| SJ10931 | | | − | − | 0.003 | nd |
| | | | − | + | 0.002 | 0.001 |
| | | | + | − | 0.275 | 0.001 |
| | | | + | + | 0.252 | 0.009 |
| SJ10932 | Thl_Ca, | 2, 5, 8, 20, 44 | − | − | 0.003 | nd |
| | scoAB_Bs, | | − | + | 0.003 | 0.001 |
| | adc_Ca, | | + | − | 0.284 | 0.001 |
| | adh_Cb | | + | + | 0.256 | 0.006 |
| SJ10933 | | | − | − | 0.003 | nd |
| | | | − | + | 0.002 | 0.001 |
| | | | + | − | 0.291 | 0.001 |
| | | | + | + | 0.263 | 0.008 |
| SJ10962 | Thl_Ca, | 2, 17, 20, 36, | − | − | 0.003 | nd |
| | atoAD_Ec, | 38 | − | + | 0.003 | 0.003 |
| | adc_Cb, | | + | − | 0.291 | 0.003 |
| | adh_Cb | | + | + | 0.257 | 0.009 |
| SJ10934 | | | − | − | 0.003 | nd |
| | | | − | + | 0.003 | 0.002 |
| | | | + | − | 0.284 | 0.001 |
| | | | + | + | 0.258 | 0.009 |
| SJ10935 | Thl_Ca, | 2, 20, 36, 38, | − | − | 0.003 | nd |
| | atoAD_Ec, | 44 | − | + | 0.003 | 0.001 |
| | adc_Ca, | | + | − | 0.316 | 0.001 |
| | adh_Cb | | + | + | 0.26 | 0.006 |
| SJ10936 | | | − | − | 0.003 | nd |
| | | | − | + | 0.003 | 0.001 |
| | | | + | − | 0.293 | 0.001 |
| | | | + | + | 0.259 | 0.006 |
| SJ10937 | Thl_Ca, | 2, 11, 14, 17, | − | − | 0.003 | nd |
| | scoAB_Bm, | 20 | − | + | 0.003 | 0.002 |
| | adc_Cb, | | + | − | 0.299 | 0.002 |
| | adh_Cb | | + | + | 0.275 | 0.02 |
| SJ10938 | | | − | − | 0.003 | nd |
| | | | − | + | 0.003 | 0.002 |
| | | | + | − | 0.285 | 0.002 |
| | | | + | + | 0.257 | 0.012 |
| SJ11017 | Thl_Ca, | 2, 11, 14, 20, | − | − | 0.003 | nd |
| | scoAB_Bm, | 44 | − | + | 0.003 | 0.001 |
| | adc_Ca, | | + | − | 0.286 | 0.003 |
| | adh_Cb | | + | + | 0.258 | 0.008 |
| SJ11018 | | | − | − | 0.003 | nd |
| | | | − | + | 0.003 | 0.001 |
| | | | + | − | 0.284 | 0.001 |
| | | | + | + | 0.262 | 0.009 |
| SJ11019 | Thl_Ca, | 2, 17, 20, 40, | − | − | 0.002 | nd |
| | ctfAB_Ca, | 42 | − | + | 0.003 | 0.002 |
| | adc_Cb, | | + | − | 0.287 | 0.002 |
| | adh_Cb | | + | + | 0.264 | 0.008 |
| SJ11020 | | | − | − | 0.003 | nd |
| | | | − | + | 0.003 | 0.002 |
| | | | + | − | 0.287 | 0.002 |
| | | | + | + | 0.259 | 0.01 |
| SJ11021 | Thl_Ca, | 2, 20, 40, 42, | − | − | 0.003 | nd |
| | ctfAB_Ca, | 44 | − | + | 0.003 | 0.002 |
| | adc_Ca, | | + | − | 0.284 | 0.002 |
| | adh_Cb | | + | + | 0.26 | 0.008 |
| SJ11022 | | | − | − | 0.003 | nd |
| | | | − | + | 0.003 | 0.002 |
| | | | + | − | 0.291 | 0.001 |
| | | | + | + | 0.261 | 0.008 |
| SJ10678 | pSJ10600, | N/A | − | − | 0.003 | nd |
| | “empty” | | − | + | 0.003 | nd |
| | control. | | + | − | 0.282 | nd |
| | | | + | + | 0.266 | nd |
| No | N/A | N/A | − | − | 0.004 | nd |
| inoculation | | | − | + | 0.006 | nd |
| | | | + | − | 0.294 | nd |
| | | | + | + | 0.275 | nd |

“nd” means not detected;
“0.000” means that the compound was detected.

As shown in Table 7, isopropanol is detected in all isopropanol-operon containing strains upon induction. Unsupplemented and uninduced cultures, produced no detectable isopropanol. With addition of acetone, isopropanol is detected in a small amount for the uninduced isopropanol operon cultures (but not in the controls), and is significantly increased upon induction with the inducing peptide.

### Example 15

### Isopropanol and Acetone Production in L. plantarum with Expression Vectors Containing Constructs Having a L. reuteri Thiolase

Selected recombinant L. plantarum strains above (as well as additional transformant colonies from preparation, indicted as -B, -C, -D, etc.) were inoculated into 2 ml eppendorf tubes containing MRS medium (containing 10 microgram/ml erythromycin), and stored at 37° C. overnight without shaking. A 0.5 ml supernatant sample for each innoculation was analyzed for acetone and isopropanol content as described herein. Results are shown in Table 8. Constructs are represented with the abbreviations shown in the Examples above.

TABLE 8

| | | | Ace- | Iso- |
|---|---|---|---|---|
| | | | tone | propanol |
| Construct | SEQ ID Nos | Strain | (%) | (%) |
| Thl_Lr, scoAB_Bs, | 5, 8, 17, 20, 34 | SJ11262 | 0.003 | 0.003 |
| adc_Cb, adh_Cb | | SJ11262-B | 0.003 | 0.003 |
| | | SJ11262-C | 0.002 | 0.003 |
| | | SJ11262-D | 0.001 | 0.003 |
| | | SJ11262-E | 0.002 | 0.003 |
| | | SJ11262-F | 0.002 | 0.003 |
| | | SJ11262-G | 0.001 | 0.003 |
| | | SJ11263 | 0.003 | 0.003 |
| | | SJ11263-B | 0.003 | 0.002 |
| | | SJ11263-C | 0.002 | 0.003 |
| | | SJ11263-D | 0.002 | 0.003 |
| | | SJ11263-E | 0.001 | 0.003 |
| | | SJ11263-F | 0.002 | 0.003 |
| | | SJ11263-G | 0.002 | 0.003 |
| Thl_Lr, ctfAB_Ca, | 17, 20, 34, 40, | SJ11264 | 0.005 | 0.005 |
| adc_Cb, adh_Cb | 42 | SJ11264-B | 0.005 | 0.005 |
| | | SJ11264-C | 0.002 | Nd |
| | | SJ11264-D | 0.003 | 0.004 |
| | | SJ11264-E | 0.004 | 0.005 |
| | | SJ11264-F | 0.006 | 0.006 |
| | | SJ11264-G | 0.004 | 0.006 |
| | | SJ11265 | 0.005 | 0.005 |
| | | SJ11265-B | 0.004 | 0.005 |
| | | SJ11265-C | 0.002 | 0.004 |
| | | SJ11265-D | 0.003 | 0.006 |
| | | SJ11265-E | 0.005 | 0.007 |
| | | SJ11265-F | 0.003 | 0.006 |
| | | SJ11265-G | 0.003 | 0.005 |
| Thl_Lr, | 11, 14, 17, 20, | SJ11266 | 0.003 | 0.003 |
| scoAB_Bm, | 34 | SJ11266-B | 0.002 | 0.003 |
| adc_Cb, adh_Cb | | SJ11266-C | 0.001 | 0.003 |
| | | SJ11266-D | 0.001 | 0.003 |
| | | SJ11266-E | 0.001 | 0.003 |
| | | SJ11266-F | 0.002 | 0.003 |
| | | SJ11266-G | 0.002 | 0.003 |
| | | SJ11267 | 0.001 | 0.003 |
| | | SJ11267-B | 0.002 | 0.003 |
| | | SJ11267-C | 0.001 | 0.003 |
| | | SJ11267-D | 0.002 | 0.003 |
| Thl_Lr, atoAD_Ec, | 17, 20, 34, 36, | SJ11268 | 0.004 | 0.006 |
| adc_Cb, adh_Cb | 38 | SJ11268-B | 0.005 | 0.005 |
| | | SJ11268-C | 0.002 | Nd |
| | | SJ11268-D | 0.001 | 0.003 |
| | | SJ11269 | 0.005 | 0.006 |
| | | SJ11269-B | 0.002 | 0.003 |
| | | SJ11269-C | 0.002 | 0.003 |
| | | SJ11269-D | 0.004 | 0.006 |
| | | SJ11269-E | 0.005 | 0.005 |
| | | SJ11269-F | 0.001 | 0.003 |
| | | SJ11269-G | 0.001 | 0.003 |

“nd” means not detected;
“0.000” means that the compound was detected.

### Example 16

### Isopropanol Pathway Enzyme Expression

Thiolase Expression and Activity in L. plantarum.

Plasmids pSJ10796 and pSJ10798 were introduced into L. plantarum SJ10656 by electroporation as previously described, selecting erythromycin resistance (10 microgram/ml) on MRS agar plates. After 3 days incubation at 30° C., two colonies from each tranformation were inoculated into MRS medium with erythromycin (10 microgram/ml), and a cell aliquot harvested by centrifugation after overnight incubation at 37° C.

DNA was extracted with the “Extract-Amp™ Plant Kit” (Sigma) and a PCR amplification with primers 663783 and 663784 (below) was used to verify the presence of the erythromycin resistance gene carried on the plasmid.

Primer 663783:
(SEQ ID NO: 123)
5′-CTGATAAGTGAGCTATTC-3′
Primer 663784:
(SEQ ID NO: 124)
5′-CAGCACAGTTCATTATC-3′

A transformants with pSJ10796 or pSJ10798, where kept as SJ10858 and SJ10859, respectively.

The following four strains of L. plantarum were used to verify thiolase expression:

SJ10857: Containing a gene encoding a Propionibacterium freudenreichii thiolase of SEQ ID NO: 114, with an unwanted deletion.
SJ10858: Containing pSJ10796, encoding a Lactobacillus reuteri thiolase of SEQ ID NO: 35.
SJ10859: Containing pSJ10798, encoding a Clostridium acetobutylicum thiolase of SEQ ID NO: 3.
SJ10870: Containing a gene encoding a Lactobacillus brevis thiolase of SEQ ID NO: 116.

The strains were propagated in MRS medium with 10 microgram/ml erythromycin, in stationary cultures at 37° C. for 1 day. The cultures were then pooled and the cells harvested by centrifugation.

The cell pellet was mechanically disrupted by treatment with glass balls, in 500 microliters of buffer (0.1 M Tris pH 7.5, 2 mM DTT) in 1.5 ml eppendorf tubes, for 3 cycles at 40 seconds in a “Bead Beater” (FastPrep FP120, BIO101 Savant) with cooling on ice in between cycles. Cell debris was removed by centrifugation, and the supernatant used for analysis.

Thiolase enzyme activity in the mixed sample was confirmed as described below:

Thiolase activity was measured by mixing 50 μl 200 μM acetoacetyl-CoA (Sigma A1625), 50 μl 200 μM Coenzyme A (Sigma C3144), 50 μl buffer (100 mM Tris, 60 mM MgCl₂, pH 8.0) and 50 μl supernatant from cell lysis (diluted 20-80× with MilliQ water) in the well of a microtiter plate. Kinetics of the disappearance of acetoacetyl-CoA complexes with magnesium due to thiolase catalyzed formation of acetyl-CoA were subsequently measured spectrophotometrically at 310 nm (measured every 20 seconds for 20 min) in a plate reader (Molecular Devices, SpectraMax Plus). Blank samples without Coenzyme A added were included and subtracted. Thiolase activity was calculated from the initial absorbance slope using the equation: Activity=−(Slope sample−Slope blank)*Dilution factor. Activity in the mixed cell lysate was found to be 400±70 mOD/min.

The mixed sample was subjected to protein analysis by Mass Spectrometry as described in the Examples below comparing peptide spectra to a database consisting of Lactobacillus plantarum WCFS1 proteins deduced from the genome sequence, with addition of the four thiolase protein sequences deduced from the recombinant plasmids introduced.

Among 279 proteins identified, the Clostridium acetobutylicum thiolase was identified with an emPAI value of 4.02, and the Lactobacillus reuteri thiolase identified with an emPAI value of 1.53.

In a separate experiment, strains SJ10857, SJ10858, SJ10859, SJ10870, and SJ10927 (containing a correct Propionibacterium freudenreichii thiolase gene) were propagated in MRS medium with 10 microgram/ml erythromycin, in stationary cultures at 37° C. for 1 day, and the cells from a 1 ml culture volume harvested by centrifugation.

The individual cell pellets were mechanically disrupted by treatment with glass balls, in 50 microliters of buffer (0.1 M Tris pH 7.5, 2 mM DTT) in 1.5 ml eppendorf tubes, for 4 cycles at 40 seconds in a “Bead Beater” (FastPrep FP120, BIO101 Savant) with cooling on ice in between cycles. 450 microliter of the buffer was added, cell debris was removed by centrifugation, and the supernatant used for analysis.

Significant thiolase enzyme activity was detected in the lysates from SJ10858 and SJ10859 (i.e. the strains containing constructs with the Lactobacillus reuteri and the Clostridium acetobutylicum thiolases) using the assay described above. Activities of 220 mOD/min and 19 mOD/min were found in SJ10858 and SJ10859, respectively.

Thiolase Expression and Activity in L. reuteri.

Electrocompetent cells of L. reuteri SJ10655 were prepared and transformed as previously described with plasmids comprising polynucleotides encoding selected thiolases. The following plasmids resulted in the indicated transformants, selected on MRS agar plates with 10 microgram/ml erythromycin, incubated at 37° C. in an anaerobic chamber.

pSJ10795 (containing thl_Pf; SEQ ID NO: 113): SJ11175
pSJ10798 (containing thl_Ca; SEQ ID NO: 2): SJ11177
pSJ10743 (containing thl_Lb; SEQ ID NO: 115): SJ11179
pSJ10796 (containing thl_Lr; SEQ ID NO: 34): SJ11181

These strains were propagated in MRS medium with 10 microgram/ml erythromycin, in stationary cultures at 37° C. for 1 day, and the cells from a 4 ml culture volume harvested by centrifugation.

Cells were washed once in 500 microliters of buffer (0.1 M Tris pH 7.5, 2 mM DTT), resuspended in 50 microliters of the buffer and mechanically disrupted by treatment with glass balls in 1.5 ml eppendorf tubes, for 4 cycles at 40 seconds at setting 4.0 m/s in a “Bead Beater” (FastPrep FP120, BIO101 Savant) with cooling on ice in between cycles. 450 microliter buffer was added, and cell debris was removed by centrifugation, and the supernatant used for enzyme activity analysis.

Similarly, 1 ml of each of the cultures were mixed, the mixed sample washed and disrupted as above, and this mixed sample was used for protein analysis by Mass Spectrometry, as elsewhere described.

The Clostridium acetobutylicum thiolase was detected with a relative emPAI value of 8.16, the Lactobacillus reuteri thiolase detected with a relative emPAI value of 1.2, and the Lactobacillus brevis thiolase detected with a relative emPAI value of 0.46.

The individual samples were used for enzyme activity analysis, where the following relative activity levels were obtained:

A. pSJ10795 (containing thl_Pf; SEQ ID NO: 113): SJ11175=36

B. pSJ10798 (containing thl_Ca; SEQ ID NO: 2): SJ11177=22000

C. pSJ10743 (containing thl_Lb; SEQ ID NO: 115): SJ11179=2100

D. pSJ10796 (containing thl_Lr; SEQ ID NO: 34): SJ11181=3000

CoA Transferase Expression and Activity in L. plantarum

Plasmids pSJ10886 and pSJ10887 were introduced into L. plantarum SJ10656 by electroporation as previously described, and the presence of the erythromycin resistance gene of the vector was confirmed by PCR amplification with primers 663783 and 663784 (supra).

A transformant with pSJ10886 was kept as SJ10922, and a transformant with pSJ10887 kept as SJ10923.

Likewise, pSJ10888 was introduced into SJ10656 resulting in SJ10988, and pSJ10889 was introduced into SJ10656 resulting in SJ10929.

Strains SJ10922 and SJ10923 (containing the B. subtilis scoAB gene pair) and strains SJ10929 and SJ10988 (containing the E. coli atoAD gene pair) were propagated in MRS medium with 10 microgram/ml erythromycin, in stationary 2 ml cultures at 37° C. for 1 day, and the cells harvested by centrifugation.

The individual cell pellets were mechanically disrupted by treatment with glass balls, in 50 microliters of buffer (0.1 M Tris pH 7.5, 2 mM DTT) in 1.5 ml eppendorf tubes, for 5 cycles at 40 seconds in a “Bead Beater” (FastPrep FP120, BIO101 Savant) with cooling on ice in between cycles. 450 microliter of the buffer was added, cell debris was removed by centrifugation, and the supernatant used for analysis.

The lysates from SJ10929 and SJ10923 were pooled and analyzed by Mass Spectrometry. Among 461 proteins identified, the AtoD subunit was identified with an emPAI value of 3.9, and the ScoB subunit identified with an emPAI value of 0.14.

Likewise, the lysate from SJ10922 was pooled with a similarly obtained lysate, from a L. plantarum strain containing an expression plasmid harbouring the scoAB genes from B. mojavensis, and analyzed. Among 472 proteins identified, the B. subtilis ScoA subunit was identified with an emPAI value of 0.65, and the B. subtilis ScoB subunit was identified with an emPAI value of 0.14.

Succinyl-CoA acetoacetate transferase activity was measured in the cell lysates using the following protocol. In the well of a microtiter plate 50 μl 80 mM Li-acetoacetate (Sigma A8509), 50 μl 400 μM succinyl-CoA (Sigma S1129), 50 μl buffer (200 mM Tris, 60 mM MgCl₂, pH 9.1) and 50 μl cell lysate (diluted 5-20× with MilliQ water) was mixed. The acetoacetyl-CoA formed in the enzymatic reaction complexes with magnesium and was detected spectrophotometrically in a plate reader (Molecular Devices, SpectraMax Plus) by measuring absorbance at 310 nm every 20 seconds for 20 min. Blank samples without cell lysates were included. Transferase activity was calculated from the initial slope of the increase in absorbance using the equation: Activity=(Slope sample−Slope Blank)*Dilution factor. In the cell lysate from SJ10922 an activity of 5.6±0.5 mOD/min was found.

CoA Transferase Expression and Activity in L. reuteri.

Electrocompetent cells of L. reuteri SJ10655 were prepared and transformed as previously described with plasmids comprising polynucleotides encoding selected CoA transferases. The following plasmids resulted in the indicated transformants, selected on MRS agar plates with 10 microgram/ml erythromycin, incubated at 37° C. in an anaerobic chamber.

pSJ10887 (containing scoAB_Bs; SEQ ID Nos: 5+8): SJ11197
pSJ10888 (containing atoAD_Ec; SEQ ID Nos: 36+38): SJ11199
pSJ10990 (containing ctfAB_Ca; SEQ ID Nos: 40+42): SJ11221

These strains were propagated in MRS medium with 10 microgram/ml erythromycin, in stationary cultures at 37° C. for 1 day, and the cells from a 4 ml culture volume harvested by centrifugation.

Cells were washed once in 500 microliters of buffer (0.1 M Tris pH 7.5, 2 mM DTT), resuspended in 50 microliters of the buffer and mechanically disrupted by treatment with glass balls in 1.5 ml eppendorf tubes, for 4 cycles at 40 seconds at setting 4.0 m/s in a “Bead Beater” (FastPrep FP120, BIO101 Savant) with cooling on ice in between cycles. 450 microliter buffer was added, and cell debris was removed by centrifugation, and the supernatant used for enzyme activity analysis.

Similarly, 1 ml of each of the cultures were mixed, the mixed sample washed and disrupted as above, and this mixed sample was used for protein analysis by Mass Spectrometry, as elsewhere described.

The ScoA subunit from Bacillus subtilis was detected with a relative emPAI value of 0.33, the ScoB subunit from Bacillus subtilis was detected with a relative emPAI value of 0.08, and the AtoA subunit from Escherichia coli was detected with a relative emPAI value of 0.06.

The individual samples were used for enzyme activity analysis, where the following relative activity levels were obtained:

A. pSJ10887 (containing scoAB_Bs; SEQ ID Nos: 5+8): SJ11197
AtoAD activity=80±30; ScoAB activity=320±40

B. pSJ10888 (containing atoAD_Ec; SEQ ID Nos: 36+38): SJ11199
AtoAD activity=6±4; ScoAB activity=1±2

C. pSJ10990 (containing ctfAB_Ca; SEQ ID Nos: 40+42): SJ11221
AtoAD activity=1±1; ScoAB activity=1±3
   Acetoacetate Decarboxylase Expression and Activity in L. plantarum.

Plasmid pSJ10756 was introduced into L. plantarum SJ10511 (identical to SJ10656) by electroporation as previously described, and the presence of the erythromycin resistance gene of the vector was confirmed by PCR amplification with primers 663783 and 663784 (supra). Two transformants were kept, as SJ10788 and SJ10789.

Similarly, plasmids pSJ10754 and pSJ10755 were transformed into SJ10511, resulting in SJ10786 and SJ10787, plasmids pSJ10778 and pSJ10779 were tranformed into SJ10656 resulting in SJ10849 and SJ10850, and plasmids pSJ10780 and pSJ10781 were transformed into SJ10656 resulting in SJ10851 and SJ10852.

The following 8 strains were used to verify acetoacetate decarboxylase expression:

SJ10786 and SJ10787, both containing a gene encoding the Clostridium acetobutylicum acetoacetate decarboxylase of SEQ ID NO: 45.
SJ10788 and SJ10789, both containing pSJ10756 encoding a Clostridium beijerinckii acetoacetate decarboxylase of SEQ ID NO: 18.
SJ10851 and SJ10852, both containing a gene encoding a Lactobacillus salvarius acetoacetate decarboxylase of SEQ ID NO: 118.
SJ10849 and SJ10850, both containing a gene encoding a Lactobacillus plantarum acetoacetate decarboxylase of SEQ ID NO: 120.

The strains were propagated in MRS medium with 10 microgram/ml erythromycin, in stationary cultures at 37° C. for 1 day, and the cultures pooled and the cells harvested by centrifugation. Cells were suspended in ⅓ the original volume of buffer (0.1 M Tris pH 7.5, 2 mM DTT), and mechanically disrupted by treatment with glass balls, in 500 microliters aliquots in 1.5 ml eppendorf tubes, for 5 cycles at 40 seconds at setting 4.0 m/s in a “Bead Beater” (FastPrep FP120, BIO101 Savant) with cooling on ice in between cycles. Cell debris was removed by centrifugation, and the supernatant used for analysis.

This pooled sample was used for protein analysis by Mass Spectrometry, as previously described, and among 245 proteins identified, the Clostridium beijerinckii acetoacetate decarboxylase was identified with an emPAI value of 0.26.

Acetoacetate Decarboxylase Expression and Activity in L. reuteri.

Electrocompetent cells of L. reuteri SJ10655 were prepared and transformed as previously described with plasmids comprising polynucleotides encoding selected acetoacetate decarboxylases. The following plasmids resulted in the indicated transformants, selected on MRS agar plates with 10 microgram/ml erythromycin, incubated at 37° C. in an anaerobic chamber.

pSJ10754 (containing adc_Ca; SEQ ID No: 44): SJ11183
pSJ10756 (containing adc_Cb; SEQ ID No: 17): SJ11185
pSJ10780 (containing adc_Ls; SEQ ID No: 117): SJ11187
pSJ10778 (containing adc_Lp; SEQ ID No: 119): SJ11189

These strains were propagated in MRS medium with 10 microgram/ml erythromycin, in stationary cultures at 37° C. for 1 day, and the cells from a 4 ml culture volume harvested by centrifugation.

Cells were washed once in 500 microliters of buffer (0.1 M Tris pH 7.5, 2 mM DTT), resuspended in 50 microliters of the buffer and mechanically disrupted by treatment with glass balls in 1.5 ml eppendorf tubes, for 4 cycles at 40 seconds at setting 4.0 m/s in a “Bead Beater” (FastPrep FP120, BIO101 Savant) with cooling on ice in between cycles. 450 microliter buffer was added, and cell debris was removed by centrifugation, and the supernatant used for enzyme activity analysis.

Similarly, 1 ml of each of the cultures were mixed, the mixed sample washed and disrupted as above, and this mixed sample was used for protein analysis by Mass Spectrometry, as elsewhere described.

The acetoacetate decarboxylase from Lactobacillus plantarum was detected with a relative emPAI value of 0.08, and the acetoacetate decarboxylase from Clostridium acetobutylicum was detected with a relative emPAI value of 0.08.

The individual samples were used for enzyme activity analysis, where the following relative activity levels were obtained:

A. pSJ10754 (containing adc_Ca; SEQ ID No: 44): SJ11183=6±13

B. pSJ10756 (containing adc_Cb; SEQ ID No: 17): SJ11185=-1±16

C. pSJ10780 (containing adc_Ls; SEQ ID No: 117): SJ11187=7±12

D. pSJ10778 (containing adc_Lp; SEQ ID No: 119): SJ11189=5±9

Alcohol Dehydrogenase Expression and Activity in L. plantarum.

Plasmid pSJ10745 was introduced into L. plantarum SJ10511 (identical to SJ10656) by electroporation as previously described, and the presence of the erythromycin resistance gene of the vector was confirmed by PCR amplification with primers 663783 and 663784 (supra). Two transformants were kept, as SJ10784 and SJ10785.

Likewise, plasmids pSJ10768 and pSJ10769 were introduced into SJ10656 resulting in SJ10883 and SJ10898, respectively, plasmids pSJ10782 and pSJ10783 were introduced into SJ10656 resulting in SJ10884 and SJ10885, respectively, and plasmids pSJ10762 and pSJ10765 were introduced into SJ10656 resulting in SJ10896 and SJ10897, respectively. In all cases, the presence of the erythromycin resistance gene was confirmed by PCR amplification.

The following 8 strains were used to verify alcohol dehydrogenase expression:

SJ10883 and SJ10898, both containing a gene encoding a Lactobacillus antri alcohol dehydrogenase of SEQ ID NO: 47.
SJ10896 and SJ10897, both containing pSJ10756 encoding a Lactobacillus fermentum alcohol dehydrogenase of SEQ ID NO: 122.
SJ10784 and SJ10785, both containing a gene encoding a Thermoanaerobacter ethanolicus alcohol dehydrogenase of SEQ ID NO: 24.
SJ10884 and SJ10885, both containing a gene encoding a Clostridium beijerinckii alcohol dehydrogenase of SEQ ID NO: 21.

The strains were propagated in MRS medium with 10 microgram/ml erythromycin, in stationary cultures at 37° C. for 1 day (1.5 ml culture volume in 2 ml eppendorf tubes), and the cultures pooled and the cells harvested by centrifugation. Cells were suspended in ⅓ the original volume of buffer (0.1 M Tris pH 7.5, 2 mM DTT), and mechanically disrupted by treatment with glass balls, in 500 microliters aliquots in 1.5 ml eppendorf tubes, for 5 cycles at 40 seconds at setting 4.0 m/s in a “Bead Beater” (FastPrep FP120, BIO101 Savant) with cooling on ice in between cycles. Cell debris was removed by centrifugation, and the supernatant used for analysis.

This pooled sample was used for protein analysis by Mass Spectrometry, as previously described, and among 160 proteins identified, the Clostridium beijerinckii alcohol dehydrogenase was identified with an emPAI value of 0.09.

The same pooled sample was analyzed for isopropanol dehydrogenase activity as described below:

Isopropanol dehydrogenase activity was measured by mixing 50 μl 1 200 mM acetone, 50 μl 400 μM NADPH (Sigma N1630), 50 μl buffer (100 mM potassium phosphate, pH 7.2) and 50 μl pooled cell lysate (diluted 1-20× with MilliQ water) in the well of a microtiter plate. The disappearance of NADPH was monitored by measuring absorbance at 340 nm every 20 seconds for 20 min in a plate reader (Molecular Devices, SpectraMax Plus). Isopropanol dehydrogenase activity was calculated from initial slope using the equation: Activity=Slope sample*Dilution factor. An activity of 10.4±0.8 mOD/min was found in the sample.

Alcohol Dehydrogenase Expression and Activity in L. reuteri.

Electrocompetent cells of L. reuteri SJ10655 were prepared and transformed as previously described with plasmids comprising polynucleotides encoding selected alcohol dehydrogenases. The following plasmids resulted in the indicated transformants, selected on MRS agar plates with 10 microgram/ml erythromycin, incubated at 37° C. in an anaerobic chamber.

pSJ10768 (containing sadh_La; SEQ ID No: 46): SJ11191
pSJ10762 (containing sadh_Lf): SEQ ID No: 121: SJ11201
pSJ10766 (containing sadh_Lf; SEQ ID No: 121): SJ11193
pSJ10782 (containing adh_Cb; SEQ ID No: 20): SJ11195

These strains were propagated in MRS medium with 10 microgram/ml erythromycin, in stationary cultures at 37° C. for 1 day, and the cells from a 4 ml culture volume harvested by centrifugation.

Cells were washed once in 500 microliters of buffer (0.1 M Tris pH 7.5, 2 mM DTT), resuspended in 50 microliters of the buffer and mechanically disrupted by treatment with glass balls in 1.5 ml eppendorf tubes, for 4 cycles at 40 seconds at setting 4.0 m/s in a “Bead Beater” (FastPrep FP120, BI0101 Savant) with cooling on ice in between cycles. 450 microliter buffer was added, and cell debris was removed by centrifugation, and the supernatant used for enzyme activity analysis.

Similarly, 1 ml of each of the cultures were mixed, the mixed sample washed and disrupted as above, and this mixed sample was used for protein analysis by Mass Spectrometry, as elsewhere described.

The alcohol dehydrogenase from Clostridium beijerinckii was detected with a relative emPAI value of 0.12, the alcohol dehydrogenase from Lactobacillus fermentum was detected with a relative emPAI value of 0.04, and the alcohol dehydrogenase from Lactobacillus antri was detected with a relative emPAI value of 0.04.

The individual samples were used for enzyme activity analysis, where the following relative activity levels were obtained:

A. pSJ10768 (containing sadh_La; SEQ ID No: 46): SJ11191=5±2

B. pSJ10762 (containing sadh_Lf): SEQ ID No: 121: SJ11201=1±1

C. pSJ10766 (containing sadh_Lf; SEQ ID No: 121): SJ11193=1900

D. pSJ10782 (containing adh_Cb; SEQ ID No: 20): SJ11195=3±4

### Example 17

### Isopropanol Production from Acetone with L. plantarum Alcohol Dehydrogenase Expression Strains

Strains carrying expression vectors containing alcohol dehydrogenase genes, as well as a strain (SJ10678) carrying the “empty” expression vector, were propagated in MRS medium with 10 microgram/ml erythromycin, in stationary cultures at 37° C. (1.5 ml culture volume in 2 ml eppendorf tubes), in duplicate, wherein the medium in one set of cultures had been supplemented with acetone (approximately 100 microliters of acetone/liter). After 1 day of incubation, 100 microliters of supernatant was harvested by centrifugation. After a total of 4 days incubation, another 100 microliter supernatant was harvested, and the samples analyzed for acetone and isopropanol content as described herein. Results are shown in Table 9. Constructs are represented with the abbreviations shown in the Examples above.

TABLE 9

| | Alcohol | | Acetone | Ace- | iso- |
|---|---|---|---|---|---|
| | dehydrogenase | | addition | tone | propanol |
| Strain | gene | Day | (100 μl/l) | (%) | (%) |
| SJ10883 | Lactobacillus antri | 1 | − | 0.003 | 0.001 |
| | (SEQ ID NO: 46) | | + | 0.002 | 0.003 |
| | | 4 | − | 0.001 | 0.001 |
| | | | + | 0.001 | 0.004 |
| SJ10898 | | 1 | − | 0.002 | 0.002 |
| | | | + | 0.002 | 0.003 |
| | | 4 | − | 0.001 | 0.001 |
| | | | + | 0.001 | 0.004 |
| SJ10896 | Lactobacillus | 1 | − | 0.004 | nd |
| | fermentum | | + | 0.005 | 0.000 |
| | (SEQ ID NO: 121) | 4 | − | 0.002 | nd |
| | | | + | 0.004 | 0.000 |
| SJ10897 | | 1 | − | 0.003 | nd |
| | | | + | 0.005 | nd |
| | | 4 | − | 0.002 | nd |
| | | | + | 0.004 | 0.000 |
| SJ10784 | Thermoanaerobacter | 1 | − | 0.002 | 0.001 |
| | ethanolicus | | + | 0.002 | 0.003 |
| | (SEQ ID NO: 23) | 4 | − | 0.001 | 0.001 |
| | | | + | 0.001 | 0.003 |
| SJ10785 | | 1 | − | 0.002 | 0.001 |
| | | | + | 0.002 | 0.003 |
| | | 4 | − | 0.001 | 0.002 |
| | | | + | 0.001 | 0.004 |
| SJ10884 | Clostridium | 1 | − | 0.002 | 0.001 |
| | beijerinckii | | + | 0.003 | 0.003 |
| | (SEQ ID NO: 20) | 4 | − | 0.001 | 0.001 |
| | | | + | 0.001 | 0.003 |
| SJ10885 | | 1 | − | 0.003 | 0.001 |
| | | | + | 0.002 | 0.003 |
| | | 4 | − | 0.001 | 0.002 |
| | | | + | 0.001 | 0.004 |
| SJ10678 | none | 1 | − | 0.002 | nd |
| | | | + | 0.004 | nd |
| | | 4 | − | 0.002 | nd |
| | | | + | 0.004 | nd |

“nd” means not detected;
“0.000” means that the compound was detected.

Acetone addition increases the isopropanol concentration measured for strains expressing the alcohol dehydrogenases from Lactobacillus antri, from Thermoanaerobacter ethanolicus, and from Clostridium beijerinckii. In all fermentations a small amount of acetone is detected. The control strain SJ10678, as well as the strains containing the Lactobacillus fermentum construct, did not produce isopropanol under these test conditions.

### Example 18

### Effect of Varying Acetone Concentration on Isopropanol Production in L. plantarum Alcohol Dehydrogenase Expression Strains

Strains SJ10898, SJ10785, and SJ10885 were fermented along with control strain SJ10678 in media with different levels of supplemental acetone. Strains were inoculated from the frozen strain collection vials into 1.8 ml MRS containing 10 microgram/ml erythromycin, in 2 ml eppendorf tubes which were incubated overnight at 37° C. without shaking. 50 microliters from these cultures were used to inoculate 1.8 ml MRS medium containing 10 microgram/ml erythromycin and the indicated acetone levels. 100 microliter supernatants were harvested for analysis of acetone and 2-propanol content after 1 and 4 days fermentation as described above. Results are shown in Table 10. Constructs are represented with the abbreviations shown in the Examples above.

TABLE 10

| | Alcohol | Acetone | | Ace- | |
|---|---|---|---|---|---|
| | dehydrogenase | addition | | tone | Isopropanol |
| Strain | gene | (ml/l) | Day | (%) | (%) |
| SJ10898 | Lactobacillus antri | 0 | 1 | 0.002 | 0.001 |
| | (SEQ ID NO: 46) | | 4 | 0.002 | 0.002 |
| | | 0.1 | 1 | 0.001 | 0.003 |
| | | | 4 | 0.002 | 0.004 |
| | | 0.5 | 1 | 0.002 | 0.021 |
| | | | 4 | 0.002 | 0.022 |
| | | 1 | 1 | 0.003 | 0.059 |
| | | | 4 | 0.002 | 0.044 |
| | | 5 | 1 | 0.141 | 0.104 |
| | | | 4 | 0.138 | 0.114 |
| | | 10 | 1 | 0.369 | 0.095 |
| | | | 4 | 0.375 | 0.111 |
| SJ10785 | Thermoanaerobacter | 0 | 1 | 0.001 | 0.001 |
| | ethanolicus | | 4 | 0.002 | 0.003 |
| | (SEQ ID NO: 23) | 0.1 | 1 | 0.002 | 0.005 |
| | | | 4 | 0.005 | 0.007 |
| | | 0.5 | 1 | 0.002 | 0.023 |
| | | | 4 | 0.002 | 0.019 |
| | | 1 | 1 | 0.003 | 0.039 |
| | | | 4 | 0.002 | 0.036 |
| | | 5 | 1 | 0.194 | 0.070 |
| | | | 4 | 0.190 | 0.070 |
| | | 10 | 1 | 0.456 | 0.116 |
| | | | 4 | 0.419 | 0.071 |
| SJ10885 | Clostridium | 0 | 1 | 0.002 | 0.002 |
| | beijerinckii | | 4 | 0.001 | 0.002 |
| | (SEQ ID NO: 20) | 0.1 | 1 | 0.002 | 0.004 |
| | | | 4 | 0.002 | 0.004 |
| | | 0.5 | 1 | 0.002 | 0.022 |
| | | | 4 | 0.003 | 0.024 |
| | | 1 | 1 | 0.001 | 0.042 |
| | | | 4 | 0.024 | 0.253 |
| | | 5 | 1 | 0.046 | 0.230 |
| | | | 4 | 0.058 | 0.198 |
| | | 10 | 1 | 0.298 | 0.271 |
| | | | 4 | 0.316 | 0.339 |
| SJ10678 | none | 0 | 1 | 0.003 | nd |
| | | | 4 | 0.004 | nd |
| | | 0.1 | 1 | 0.004 | nd |
| | | | 4 | 0.004 | nd |
| | | 0.5 | 1 | 0.019 | nd |
| | | | 4 | 0.020 | nd |
| | | 1 | 1 | 0.036 | nd |
| | | | 4 | 0.037 | nd |
| | | 5 | 1 | 0.228 | nd |
| | | | 4 | 0.001 | nd |
| | | 10 | 1 | 0.466 | nd |
| | | | 4 | 0.480 | nd |

“nd” means not detected.

Significant conversion of acetone into isopropanol is observed for the three alcohol dehydrogenase expressing strains, whereas no isopropanol is detected with the control strain SJ10678.

### Example 19

### Isopropanol Production in E. coli from Lactobacillus Inducible Isopropanol-Operon Constructs

Isopropanol operons controlled by a peptide-inducible Lactobacillus promoter system were described above, wherein the plasmids were constructed in E. coli. These E. coil strains were tested for isopropanol production by fermentation in LB+100 microgram/ml erythromycin+1% glucose, with or without inducing peptide added, 37° C., 1 day, shaking 300 rpm as described above. The strains were inoculated directly from the frozen stock culture into fermentation medium (10 ml in test tubes). Results are shown in Table 11A. Constructs are represented with the abbreviations shown in the Examples above.

TABLE 11A

| | | | | Ace- | Iso- |
|---|---|---|---|---|---|
| | | | Inducing | tone | propanol |
| Strain | Construct | SEQ ID Nos | peptide | (%) | (%) |
| SJ10903 | Thl_Ca, | 2, 5, 8, 17, 20 | − | 0.008 | 0.061 |
| | scoAB_Bs, | | + | 0.007 | 0.062 |
| SJ10904 | adc_Cb, | | − | 0.007 | 0.056 |
| | adh_Cb | | + | 0.007 | 0.057 |
| SJ10905 | Thl_Ca, | 2, 5, 8, 20, 44 | − | 0.005 | 0.057 |
| | scoAB_Bs, | | + | 0.006 | 0.061 |
| SJ10906 | adc_Ca, | | − | 0.006 | 0.063 |
| | adh_Cb | | + | 0.006 | 0.061 |
| SJ10907 | Thl_Ca, | 2, 17, 20, 36, | − | 0.009 | 0.1 |
| | atoAD_Ec, | 38 | + | 0.009 | 0.095 |
| SJ10908 | adc_Cb, | | − | 0.009 | 0.104 |
| | adh_Cb | | + | 0.01 | 0.106 |
| SJ10909 | Thl_Ca, | 2, 20, 36, 38, | − | 0.011 | 0.093 |
| | atoAD_Ec, | 44 | + | 0.008 | 0.074 |
| SJ10910 | adc_Ca, | | − | 0.008 | 0.076 |
| | adh_Cb | | + | 0.009 | 0.076 |
| SJ10911 | Thl_Ca, | 2, 11, 14, 17, | − | 0.003 | 0.035 |
| | scoAB_Bm, | 20 | + | 0.003 | 0.033 |
| SJ10912 | adc_Cb, | | − | 0.003 | 0.022 |
| | adh_Cb | | + | 0.003 | 0.02 |
| SJ10940 | Thl_Ca, | 2, 11, 14, 20, | − | 0.006 | 0.048 |
| | scoAB_Bm, | 44 | + | 0.006 | 0.05 |
| SJ10941 | adc_Ca, | | − | 0.007 | 0.054 |
| | adh_Cb | | + | 0.007 | 0.051 |
| SJ10973 | Thl_Ca, | 2, 17, 20, 40, | − | 0.005 | 0.07 |
| | ctfAB_Ca, | 42 | + | 0.005 | 0.071 |
| SJ10974 | adc_Cb, | | − | 0.005 | 0.063 |
| | adh_Cb | | + | 0.005 | 0.065 |
| SJ10975 | Thl_Ca, | 2, 20, 40, 42, | − | 0.006 | 0.068 |
| | ctfAB_Ca, | 44 | + | 0.006 | 0.07 |
| SJ10976 | adc_Ca, | | − | 0.008 | 0.079 |
| | adh_Cb | | + | 0.007 | 0.074 |
| SJ10766 | (Control) | 121 | − | 0.003 | nd |
| | sadh_Lf | | + | 0.002 | nd |

“nd” means not detected.

A significant isopropanol production is observed in E. coli from all the isopropanol operon constructs tested.

### Example 20

### Isopropanol Production from L. reuteri Alcohol Dehydrogenase Expression Strains Supplemented with Acetone and/or 1,2-Propanediol

Electrocompetent cells of L. reuteri SJ10655 were prepared and transformed as previously described with plasmids comprising polynucleotides encoding selected alcohol dehydrogenases. The following plasmids resulted in the indicated transformants which were verified by restriction analysis of extracted plasmids.

pSJ10600: SJ11011 and SJ11012
pSJ10765: SJ11013 and SJ11014
pSJ10769: SJ11015 and SJ11016
pSJ10783: SJ11024
pSJ10745: SJ11053 and SJ11054

Transformants were selected on LCM agar plates with 10 microgram/ml erythromycin, incubated at 37° C. in an anaerobic chamber.

In another experiment, electrocompetent cells of L. reuteri SJ10655 were prepared, and transformed as previously described. The following plasmids resulted in the indicated transformants which were verified by restriction analysis of extracted plasmids.

The following strains were kept:
pSJ10768: SJ11191 and SJ11192:
pSJ10766: SJ11193 and SJ11194
pSJ10782: SJ11195 and SJ11196
pSJ10762: SJ11201 and SJ11202

Selected L. reuteri transformants were inoculated directly from the frozen stock culture into 2 ml MRS medium cultures supplemented with erythromycin (10 microgram/ml) and acetone (5 ml/l), and tubes incubated without shaking at 37° C. for 3 days. Supernatants were harvested and analyzed for 1-propanol, 2-propanol and acetone content as described above. Results are shown in Table 11B.

TABLE 11B

| | | SEQ ID | | n-propanol | Isopropanol | Acetone |
|---|---|---|---|---|---|---|
| Plasmid | Construct | NO | Strain | (%) | (%) | (%) |
| none | none | N/A | none | 0.009 | nd | 0.377 |
| pSJ10600 | Empty vector | N/A | SJ11011 | 0.011 | 0.021 | 0.364 |
| | | | SJ11012 | 0.011 | 0.022 | 0.361 |
| pSJ10765 | sadh_Lf | 121 | SJ11013 | 0.011 | 0.025 | 0.362 |
| | | | SJ11014 | 0.011 | 0.018 | 0.358 |
| pSJ10766 | | | SJ11193 | 0.011 | 0.025 | 0.363 |
| | | | SJ11194 | 0.011 | 0.035 | 0.351 |
| pSJ10762 | | | SJ11201 | 0.011 | 0.023 | 0.355 |
| | | | SJ11202 | 0.011 | 0.033 | 0.332 |
| pSJ10769 | sadh_La | 46 | SJ11015 | 0.011 | 0.123 | 0.227 |
| | | | SJ11016 | 0.011 | 0.034 | 0.353 |
| pSJ10768 | | | SJ11191 | 0.011 | 0.141 | 0.212 |
| | | | SJ11192 | 0.011 | 0.164 | 0.193 |
| pSJ10783 | adh_Cb | 20 | SJ11024 | 0.011 | 0.208 | 0.137 |
| pSJ10782 | | | SJ11195 | 0.010 | 0.325 | 0.015 |
| | | | SJ11196 | 0.011 | 0.317 | 0.011 |

In an additional experiment, strains SJ11024, SJ11053 and SJ11054 were inoculated in MRS containing 10 microgram/ml erythromycin and incubated at 37° C. overnight. These cultures were used to inoculate 2 ml eppendorf tubes containing MRS medium containing 10 microgram/ml erythromycin, supplemented with acetone and/or 1,2-propanediol as indicated in the tables below and incubated at 37° C. for two days without shaking. A 1 ml supernatant sample then was analyzed as described herein, with results shown in Tables 11C, 11D, 11E, and 11F, for n-propanol, isopropanol, acetone, and 1,2-propanediol content, respectively.

TABLE 11C

| | Resulting n-propanol content (%) | | | | | | |
|---|---|---|---|---|---|---|---|
| | Supplemental Acetone | | | Supplemental Acetone + 1,2propandiol | | | |
| | 1 mL/L | 5 mL/L | 10 mL/L | 1 + 1 mL/L | 5 + 5 mL/L | 10 + 10 mL/L | Control |
| MRS- | nd | nd | nd | nd | nd | nd | nd |
| 10erm | | | | | | | |
| SJ11024 | 0.002 | 0.002 | 0.002 | 0.071 | 0.243 | 0.243 | 0.002 |
| SJ11053 | 0.002 | 0.002 | 0.002 | 0.070 | 0.198 | 0.255 | 0.002 |
| SJ11054 | 0.002 | 0.002 | 0.002 | 0.070 | 0.216 | 0.265 | 0.002 |

“nd” means not detected.

TABLE 11D

| | Resulting isopropanol content (%) | | | | | | |
|---|---|---|---|---|---|---|---|
| | Supplemental Acetone | | | Supplemental Acetone + 1,2propandiol | | | |
| | 1 mL/L | 5 mL/L | 10 mL/L | 1 + 1 mL/L | 5 + 5 mL/L | 10 + 10 mL/L | Control |
| MRS- | nd | nd | nd | nd | nd | nd | nd |
| 10erm | | | | | | | |
| SJ11024 | 0.064 | 0.292 | 0.314 | 0.067 | 0.213 | 0.227 | 0.002 |
| SJ11053 | 0.012 | 0.019 | 0.033 | 0.012 | 0.014 | 0.017 | 0.002 |
| SJ11054 | 0.013 | 0.021 | 0.035 | 0.013 | 0.013 | 0.017 | 0.001 |

“nd” means not detected.

TABLE 11E

| | Resulting acetone content (%) | | | | | | |
|---|---|---|---|---|---|---|---|
| | Supplemental Acetone | | | Supplemental Acetone + 1,2propandiol | | | |
| | 1 mL/L | 5 mL/L | 10 mL/L | 1 + 1 mL/L | 5 + 5 mL/L | 10 + 10 mL/L | Control |
| MRS- | 0.068 | 0.315 | 0.630 | 0.072 | 0.317 | 0.658 | 0.003 |
| 10erm | | | | | | | |
| SJ11024 | 0.004 | 0.029 | 0.304 | 0.002 | 0.101 | 0.424 | 0.002 |
| SJ11053 | 0.054 | 0.284 | 0.566 | 0.057 | 0.295 | 0.627 | 0.003 |
| SJ11054 | 0.053 | 0.282 | 0.569 | 0.053 | 0.291 | 0.627 | 0.002 |

TABLE 11F

| | Resulting 1,2-propandiol content (%) | | | | | | |
|---|---|---|---|---|---|---|---|
| | Supplemental Acetone | | | Supplemental Acetone + 1,2propandiol | | | |
| | 1 mL/L | 5 mL/L | 10 mL/L | 1 + 1 mL/L | 5 + 5 mL/L | 10 + 10 mL/L | Control |
| MRS- | nd | nd | nd | 0.118 | 0.536 | 1.125 | nd |
| 10erm | | | | | | | |
| SJ11024 | nd | nd | nd | nd | 0.143 | 0.711 | nd |
| SJ11053 | nd | nd | nd | nd | 0.209 | 0.681 | nd |
| SJ11054 | nd | nd | nd | nd | 0.183 | 0.663 | nd |

“nd” means not detected.

### Example 21

### Isopropanol Production with L. reuteri Expression Strains

L. reuteri SJ11044 was transformed with selected recombinant plasmids by electroporation using the protocol previously described. Selected transformed strains (as well as additional transformant colonies from preparation, indicted as -B, -C, -D, etc.) were inoculated (from colonies on plates) into 2 ml eppendorf tubes containing MRS medium containing 10 microgram/ml erythromycin, and incubated at 37° C. overnight without shaking. A 0.5 ml supernatant sample then was analyzed for acetone and isopropanol content as described herein. Results are shown in Table 12A. Constructs are represented with the abbreviations shown in the Examples above.

TABLE 12A

| | | | | Acetone | Isopropanol |
|---|---|---|---|---|---|
| Plasmid | Construct | SEQ ID Nos | Strain | (%) | (%) |
| pSJ11204 | Thl_Lr, scoAB_Bs, | 5, 8, 17, 20, 34 | SJ11270 | 0.002 | 0.004 |
| | adc_Cb, adh_Cb | | SJ11270-B | 0.002 | 0.004 |
| | | | SJ11270-C | 0.002 | nd |
| | | | SJ11270-D | 0.001 | 0.003 |
| | | | SJ11270-E | 0.001 | 0.003 |
| | | | SJ11270-F | 0.001 | 0.003 |
| | | | SJ11270-G | 0.002 | 0.003 |
| pSJ11205 | | | SJ11271 | 0.002 | 0.004 |
| | | | SJ11271-B | 0.002 | 0.004 |
| | | | SJ11271-C | 0.001 | 0.004 |
| | | | SJ11271-D | 0.002 | 0.004 |
| | | | SJ11271-E | 0.002 | 0.004 |
| | | | SJ11271-F | 0.002 | 0.004 |
| | | | SJ11271-G | 0.002 | 0.004 |
| pSJ11206 | Thl_Lr, ctfAB_Ca, | 17, 20, 34, 40, | SJ11272 | 0.002 | 0.006 |
| | adc_Cb, adh_Cb | 42 | SJ11272-B | 0.002 | 0.006 |
| | | | SJ11272-C | 0.002 | 0.003 |
| | | | SJ11272-D | 0.002 | 0.003 |
| | | | SJ11272-E | 0.002 | 0.002 |
| | | | SJ11272-F | 0.002 | 0.005 |
| | | | SJ11272-G | 0.002 | Nd |
| pSJ11207 | | | SJ11273 | 0.003 | 0.006 |
| | | | SJ11273-B | 0.002 | Nd |
| | | | SJ11273-C | 0.002 | 0.005 |
| | | | SJ11273-D | 0.002 | 0.002 |
| | | | SJ11273-E | 0.002 | 0.003 |
| | | | SJ11273-F | 0.002 | 0.005 |
| | | | SJ11273-G | 0.002 | 0.004 |
| pSJ11208 | Thl_Lr, scoAB_Bm, | 11, 14, 17, 20, | SJ11274 | 0.002 | 0.005 |
| | adc_Cb, adh_Cb | 34 | SJ11274-B | 0.001 | 0.003 |
| | | | SJ11274-C | 0.001 | 0.003 |
| | | | SJ11274-D | 0.002 | 0.003 |
| | | | SJ11274-E | 0.002 | 0.003 |
| | | | SJ11274-F | 0.002 | 0.003 |
| | | | SJ11274-G | 0.002 | 0.003 |
| pSJ11209 | | | SJ11275 | 0.002 | 0.005 |
| | | | SJ11275-B | 0.002 | 0.005 |
| | | | SJ11275-C | 0.002 | 0.005 |
| | | | SJ11275-D | 0.002 | 0.005 |
| | | | SJ11275-E | 0.002 | 0.005 |
| | | | SJ11275-F | 0.002 | 0.005 |
| | | | SJ11275-G | 0.002 | 0.005 |
| pSJ11230 | Thl_Lr, atoAD_Ec, | 17, 20, 34, 36, | SJ11276 | 0.002 | 0.010 |
| | adc_Cb, adh_Cb | 38 | SJ11276-B | 0.002 | 0.010 |
| | | | SJ11276-C | 0.001 | 0.003 |
| | | | SJ11276-D | 0.002 | 0.009 |
| | | | SJ11276-E | 0.002 | 0.003 |
| | | | SJ11276-F | 0.002 | nd |
| | | | SJ11276-G | 0.002 | nd |
| pSJ11231 | | | SJ11277 | 0.003 | 0.011 |
| | | | SJ11278 | 0.002 | 0.010 |
| | | | SJ11278-B | 0.002 | 0.010 |
| | | | SJ11278-C | 0.003 | 0.011 |
| | | | SJ11278-D | 0.003 | 0.011 |
| | | | SJ11278-E | 0.003 | 0.010 |
| | | | SJ11278-F | 0.003 | 0.011 |

“nd” means not detected;
“0.000” means that the compound was detected.

Four different Lactobacillus reuteri strains, as well as a non-inoculated media control sample, were incubated for 2 days at 37° C., in 2 ml stationary cultures, in a number of different media. Samples were then was analyzed for acetone, n-propanol, and isopropanol content as described herein. Results are shown in Table 12B. Constructs are represented with the abbreviations shown in the Examples above.

TABLE 12B

| | | | | n- | | |
|---|---|---|---|---|---|---|
| | | | SEQ ID | propanol | Isopropanol | Acetone |
| Medium | Strain | Construct | NOs | (%) | (%) | (%) |
| MRS-G + 2% | SJ11272 | Thl_Lr, ctfAB_Ca, | 17, 20, 34, | 0.002 | 0.006 | 0.004 |
| sucrose + 10 erm | | adc_Cb, adh_Cb | 40, 42 | | | |
| | SJ11275 | Thl_Lr, | 11, 14, 17, | 0.002 | 0.005 | 0.003 |
| | | scoAB_Bm, | 20, 34 | | | |
| | | adc_Cb, adh_Cb | | | | |
| | SJ11278 | Thl_Lr, | 17, 20, 34, | 0.002 | 0.007 | 0.003 |
| | | atoAD_Ec, | 36, 38 | | | |
| | | adc_Cb, adh_Cb | | | | |
| | SJ11011 | N/A | N/A | 0.002 | 0.001 | 0.003 |
| | None | N/A | N/A | nd | nd | 0.004 |
| MRS-G + 5% | SJ11272 | Thl_Lr, ctfAB_Ca, | 17, 20, 34, | 0.002 | 0.004 | 0.006 |
| sucrose +10 erm | | adc_Cb, adh_Cb | 40, 42 | | | |
| | SJ11275 | Thl_Lr, | 11, 14, 17, | 0.001 | 0.004 | 0.006 |
| | | scoAB_Bm, | 20, 34 | | | |
| | | adc_Cb, adh_Cb | | | | |
| | SJ11278 | Thl_Lr, | 17, 20, 34, | 0.002 | 0.006 | 0.005 |
| | | atoAD_Ec, | 36, 38 | | | |
| | | adc_Cb, adh_Cb | | | | |
| | SJ11011 | N/A | N/A | 0.001 | 0.001 | 0.005 |
| | None | N/A | N/A | nd | nd | 0.004 |
| LCM + 10% | SJ11272 | Thl_Lr, ctfAB_Ca, | 17, 20, 34, | 0.001 | 0.006 | 0.004 |
| sucrose + 10 erm | | adc_Cb, adh_Cb | 40, 42 | | | |
| | SJ11275 | Thl_Lr, | 11, 14, 17, | 0.001 | 0.006 | 0.005 |
| | | scoAB_Bm, | 20, 34 | | | |
| | | adc_Cb, adh_Cb | | | | |
| | SJ11278 | Thl_Lr, | 17, 20, 34, | 0.002 | 0.010 | 0.006 |
| | | atoAD_Ec, | 36, 38 | | | |
| | | adc_Cb, adh_Cb | | | | |
| | SJ11011 | N/A | N/A | 0.001 | 0.001 | 0.004 |
| | None | N/A | N/A | nd | nd | 0.003 |
| LCM + 10% | SJ11272 | Thl_Lr, ctfAB_Ca, | 17, 20, 34, | 0.001 | 0.007 | 0.014 |
| glucose + 10 erm | | adc_Cb, adh_Cb | 40, 42 | | | |
| | SJ11275 | Thl_Lr, | 11, 14, 17, | 0.001 | 0.009 | 0.015 |
| | | scoAB_Bm, | 20, 34 | | | |
| | | adc_Cb, adh_Cb | | | | |
| | SJ11278 | Thl_Lr, | 17, 20, 34, | 0.001 | 0.008 | 0.015 |
| | | atoAD_Ec, | 36, 38 | | | |
| | | adc_Cb, adh_Cb | | | | |
| | SJ11011 | N/A | N/A | 0.001 | 0.003 | 0.014 |
| | None | N/A | N/A | nd | nd | 0.012 |
| MRS-G + 2% | SJ11272 | Thl_Lr, ctfAB_Ca, | 17, 20, 34, | 0.002 | 0.006 | 0.004 |
| ribose + 10 erm | | adc_Cb, adh_Cb | 40, 42 | | | |
| | SJ11275 | Thl_Lr, | 11, 14, 17, | 0.002 | 0.006 | 0.004 |
| | | scoAB_Bm, | 20, 34 | | | |
| | | adc_Cb, adh_Cb | | | | |
| | SJ11278 | Thl_Lr, | 17, 20, 34, | 0.002 | 0.008 | 0.004 |
| | | atoAD_Ec, | 36, 38 | | | |
| | | adc_Cb, adh_Cb | | | | |
| | SJ11011 | N/A | N/A | 0.002 | 0.002 | 0.004 |
| | None | N/A | N/A | nd | nd | 0.004 |
| MRS-G + 2% | SJ11272 | Thl_Lr, ctfAB_Ca, | 17, 20, 34, | 0.002 | 0.004 | 0.002 |
| sucrose + 10erm + | | adc_Cb, adh_Cb | 40, 42 | | | |
| D-pantothenic | SJ11275 | Thl_Lr, | 11, 14, 17, | 0.002 | 0.005 | 0.002 |
| acid | | scoAB_Bm, | 20, 34 | | | |
| | | adc_Cb, adh_Cb | | | | |
| | SJ11278 | Thl_Lr, | 17, 20, 34, | 0.002 | 0.007 | 0.002 |
| | | atoAD_Ec, | 36, 38 | | | |
| | | adc_Cb, adh_Cb | | | | |
| | SJ11011 | N/A | N/A | 0.002 | 0.001 | 0.003 |
| | None | N/A | N/A | nd | nd | 0.003 |
| MRS-G + 5% | SJ11272 | Thl_Lr, ctfAB_Ca, | 17, 20, 34, | 0.001 | 0.004 | 0.004 |
| sucrose + 10erm + | | adc_Cb, adh_Cb | 40, 42 | | | |
| D-pantothenic | SJ11275 | Thl_Lr, | 11, 14, 17, | 0.001 | 0.004 | 0.004 |
| acid | | scoAB_Bm, | 20, 34 | | | |
| | | adc_Cb, adh_Cb | | | | |
| | SJ11278 | Thl_Lr, | 17, 20, 34, | 0.001 | 0.006 | 0.004 |
| | | atoAD_Ec, | 36, 38 | | | |
| | | adc_Cb, adh_Cb | | | | |
| | SJ11011 | N/A | N/A | 0.001 | 0.001 | 0.004 |
| | None | N/A | N/A | nd | nd | 0.003 |
| LCM + 10% | SJ11272 | Thl_Lr, ctfAB_Ca, | 17, 20, 34, | 0.001 | 0.006 | 0.004 |
| sucrose + 10erm + | | adc_Cb, adh_Cb | 40, 42 | | | |
| D-pantothenic | SJ11275 | Thl_Lr, | 11, 14, 17, | 0.001 | 0.006 | 0.004 |
| acid | | scoAB_Bm, | 20, 34 | | | |
| | | adc_Cb, adh_Cb | | | | |
| | SJ11278 | Thl_Lr, | 17, 20, 34, | 0.001 | 0.009 | 0.006 |
| | | atoAD_Ec, | 36, 38 | | | |
| | | adc_Cb, adh_Cb | | | | |
| | SJ11011 | N/A | N/A | 0.001 | 0.001 | 0.004 |
| | None | N/A | N/A | nd | nd | 0.003 |
| LCM + 10% | SJ11272 | Thl_Lr, ctfAB_Ca, | 17, 20, 34, | 0.001 | 0.008 | 0.010 |
| glucose + 10erm + | | adc_Cb, adh_Cb | 40, 42 | | | |
| D-pantothenic | SJ11275 | Thl_Lr, | 11, 14, 17, | 0.001 | 0.009 | 0.010 |
| acid | | scoAB_Bm, | 20, 34 | | | |
| | | adc_Cb, adh_Cb | | | | |
| | SJ11278 | Thl_Lr, | 17, 20, 34, | 0.001 | 0.008 | 0.011 |
| | | atoAD_Ec, | 36, 38 | | | |
| | | adc_Cb, adh_Cb | | | | |
| | SJ11011 | N/A | N/A | 0.001 | 0.003 | 0.011 |
| | None | N/A | N/A | nd | nd | 0.009 |
| MRS-G + 2% | SJ11272 | Thl_Lr, ctfAB_Ca, | 17, 20, 34, | 0.002 | 0.007 | 0.004 |
| ribose + 10erm + | | adc_Cb, adh_Cb | 40, 42 | | | |
| D-pantothenic | SJ11275 | Thl_Lr, | 11, 14, 17, | 0.002 | 0.005 | 0.003 |
| acid | | scoAB_Bm, | 20, 34 | | | |
| | | adc_Cb, adh_Cb | | | | |
| | SJ11278 | Thl_Lr, | 17, 20, 34, | 0.002 | 0.007 | 0.003 |
| | | atoAD_Ec, | 36, 38 | | | |
| | | adc_Cb, adh_Cb | | | | |
| | SJ11011 | N/A | N/A | 0.002 | 0.002 | 0.003 |
| | None | N/A | N/A | nd | nd | 0.004 |

### Example 22

### Isopropanol Production with L. reuteri Expression Strains in Sugar Cane Juice

Strain SJ11278 was propagated in sugar cane juice medium (BRIX=5) containing yeast extract (10 g/l), Tween 80 (1 g/l), MnSO₄.H2O (50 mg/l) and erythromycin. The culture was incubated for one day at 37° C.

50 mL of the above culture was used to inoculate a fermentor containing 1950 mL medium with the following composition: Sugar cane juice (adjusted to BRIX 10); Pluronic/Dowfax 63N, 1 mL/L; Bacto yeast extract, 10 g/L; Tween 80, 1 g/L; MnSO₄, H₂O, 25 mg/L; Phytic acid, 650 mg/L; erythromycin, 4 mL of a 5 mg/mL solution in ethanol.

The fermentation was sparged with nitrogen (0.1 L/min) and agitated at a rate of 400 RPM. Temperature and pH was held constant at 37 degrees Celsius and pH 6.5, respectively.

After three days of fermentation, the isopropanol concentration was found to be 0.3 mL/L. No isopropanol could be detected in a control experiment (fermentation ID: GPP099) with the untransformed host strain grown under identical conditions (but without additions of erythromycin). The n-propanol concentration at the same time point was measured to 0.07 mL/L and 0.08 mL/L for the fermentations with SJ11278 and the control strain, respectively.

The SJ11278 culture obtained after 3 days of fermentation was analyzed for contamination, and it was found that the fermentation with SJ11278 was contaminated with Lactobacillus plantarum. The inoculum was subsequently re-tested and found to be Lactobacillus reuteri, strain SJ11278. Had the culture been uncontaminated, it is conceivable that a greater titer of isopropanol would have been obtained.

### Example 23

### n-Propanol Tolerance in Lactobacillus reuteri

Lactobacillus reuteri was shown to be resistant to n-propanol under the conditions described below.

To prepare the inoculum for the tank fermentation, a preculture of a strain of Lactobacillus reuteri was performed as described above. 50 mL of this culture was used to inoculate a fermentor containing 1950 mL of a medium prepared as described in the following:

Medium composition: Concentrated sugar cane juice (BRIX 53) adjusted to final BRIX of 5 with tap water was used as the base component. To this diluted sugar cane juice, yeast extract (Bacto) was added in the amount of 10 g/L and antifoam (Pluronic/Dowfax 63N) was added in the amount of 1 mL/L. This mixture was transferred to a labscale fermentor (3 liter vessel) and autoclaved for 30 minutes at 121-123° C. After autoclavation, temperature was adjusted to 37° C. and 80 mL (corresponding to 40 ml/L) of n-propanol was added to the tank by sterile filtration.

Following inoculation, the temperature was held at about 37° C. and the pH maintained at either pH 6.5 or pH 3.8 (e.g., by the addition of 10% (w/w) NH₄OH). A small inflow (0.1 liter per minute) of N₂ ensured that the culture was anaerobic during agitation at 400 rpm. OD₆₅₀ measurements were taken throughout the fermentation to monitor cell growth.

Lactobacillus reuteri was capable of growth at both pH 6.5 and pH 3.8 in 4% n-propanol. At pH 3.8, the growth rate was somewhat delayed, but achieved the same maximum OD after about 40 hours of fermentation. A gas chromatography-mass spectrometry (GCMS) based analysis of a fermentation sample taken after 112 hours of fermentation showed that of the initial amount of n-propanol, the pH 6.5 and pH 3.8 contained 79.8% and 93.1%, respectively. It was determined that the n-propanol used for the experiment initially contained approximately 4% isopropanol in addition to 96% n-propanol.

### Example 24

### n-Propanol Produced in wt Lactobacillus reuteri

Wild-type Lactobacillus reuteri O4ZXV was shown to produce n-propanol under the conditions described below.

A preculture of wt Lactobacillus reuteri O4ZXV for the tank fermentations was grown for two days at 37° C. in MRS-medium without aeration or shaking. A 50 mL sample of this culture was used to inoculate a fermentor containing 1950 mL of the following medium:

Medium composition: Concentrated sugar cane juice (BRIX 53) adjusted to final BRIX of 5 with tap water was used as the base component. To this diluted sugar cane juice, yeast extract (Bacto) was added in the amount of 10 g/L and antifoam (Pluronic/Dowfax 63N) was added in the amount of 1 mL/L. This mixture was transferred to a labscale fermentor (3 liter vessel) and autoclaved for 30 minutes at 121-123° C.

Following inoculation, the pH was kept constant at 6.5 by the addition of 10% (w/w) NH₄OH, and the temperature was kept at 37° C. The culture was kept anaerobic by a small flow of pumped N₂ (0.1 liter per minute) and the agitation rate was 400 rpm.

A gas chromatography-mass spectrometry (GCMS) based analysis of a fermentation sample taken after 48 hours of fermentation indicated that the culture contained approximately 40 μL/L n-propanol.

In another experiment performed with the same strain as above and under the same conditions but with pH being kept constant at pH 3.8 instead of pH 6.5, a sample taken after 48 hours of fermentation showed that the culture contained approximately 40 μL/L n-propanol.

### Example 25

### Cloning of n-Propanol Aldehyde Dehydrogenase Genes

Cloning of a P. freudenreichii aldehyde dehydrogenase gene (pduP_P_syn2) and construction of vector pTRGU30.

The 1500 bp coding sequence of an aldehyde dehydrogenase gene identified in P. freudenreichii was optimized for expression in E. coli and synthetically constructed into pTRGU30. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was NotI-BamHI-RBS-CDS-XbaI-HindIII, resulting in pTRGU30.

The wild-type nucleotide sequence (WT), codon-optimized nucleotide sequence (CO), and deduced amino acid sequence of the P. freudenreichii aldehyde dehydrogenase gene are listed as SEQ ID NO: 25, 26, and 27, respectively. The coding sequence is 1503 bp including the stop codon and the encoded predicted protein is 500 amino acids. Using the SignalP program (Nielsen et al., 1997, Protein Engineering 10:1-6), no signal peptide in the sequence was predicted. Based on this program, the predicted mature protein contains 500 amino acids with a predicted molecular mass of 53.7 kDa and an isoelectric pH of 6.39.

Cloning of a L. coffinoides Aldehyde Dehydrogenase Gene (pduP_Lc) and Construction of Vector pTRGU31.

The 1443 bp coding sequence of an aldehyde dehydrogenase gene identified in L. coffinoides was optimized for expression in E. coli and synthetically constructed into pTRGU31. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was PacI-NotI-RBS-CDS-HindIII-AscI, resulting in pTRGU31.

The wild-type nucleotide sequence (WT), codon-optimized nucleotide sequence (CO), and deduced amino acid sequence of the L. collinoides aldehyde dehydrogenase gene are listed as SEQ ID NO: 28, 29, and 30, respectively. The coding sequence is 1446 bp including the stop codon and the encoded predicted protein is 481 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 481 amino acids with a predicted molecular mass of 51.2 kDa and an isoelectric pH of 5.24.

Cloning of a C. beijerinckii Aldehyde Dehydrogenase Gene (DduP_Cb) and Construction of Vector pTRG U85.

The 1404 bp coding sequence of an aldehyde dehydrogenase gene identified in C. beijerinckii was optimized for expression in E. coli and synthetically constructed into pTRGU85.

The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was PacI-NotI-RBS-CDS-HindIII-AscI, resulting in pTRGU85.

The wild-type nucleotide sequence (WT), codon-optimized nucleotide sequence (CO), and deduced amino acid sequence of the P. freudenreichii aldehyde dehydrogenase gene are listed in SEQ ID NO: 31, 32, and 33, respectively. The coding sequence is 1407 bp including the stop codon and the encoded predicted protein is 468 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 468 amino acids with a predicted molecular mass of 51.3 kDa and an isoelectric pH of 5.88.

Cloning of a P. freudenreichii Aldehyde Dehydrogenase Gene (pduP_Pf_Syn2a) and Construction of Vector pTRGU300.

Two potential start codons were detected in pduP_Pf_syn2: one applied in the terminus of the pduP_Pf_syn2 nucleotide sequences, and a second located 93 bp downstream of the initial start codon. Applying the second start codon yields a 1407 bp coding sequence of the aldehyde dehydrogenase gene identified in P. freudenreichii. This sequence was identical to the sequence applied above except for the initial 93 bp and thus was optimized for expression in E. coli. The sequence was synthetically constructed into pTRGU300. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was PacI-NotI-BamHI-RBS-CDS-XbaI-HindIII-AscI, resulting in pTRGU300.

The wild-type nucleotide sequence (WT), codon-optimized nucleotide sequence (CO), and deduced amino acid sequence of the P. freudenreichii aldehyde dehydrogenase gene are listed as SEQ ID NO: 48, 49, and 51, respectively. The coding sequence is 1410 bp including the stop codon and the encoded predicted protein is 469 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 469 amino acids with a predicted molecular mass of 50.1 kDa and an isoelectric pH of 5.69.

Cloning of a P. freudenreichii Aldehyde Dehydrogenase Gene (pduP_Pf_Syn2b) and Construction of Vector pTRGU399.

Cloning of pduP_Pf_syn2a described above indicated that the gene potentially possessed secondary structures which could lower in vivo transcription efficiency. Hence, the 1407 bp coding sequence of the same aldehyde dehydrogenase gene identified in P. freudenreichii was re-optimized for expression in E. coli in order to alter the DNA sequence and maintaining the amino acid sequence of the protein. The re-optimized sequence was synthetically constructed into pTRGU399. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was PacI-NotI-BamHI-RBS-CDS-XbaI-HindIII-AscI, resulting in pTRGU399.

This second codon-optimized nucleotide sequence (CO) of the P. freudenreichii aldehyde dehydrogenase gene is listed as SEQ ID NO: 50. The coding sequence is 1410 bp including the stop codon and the encoded predicted protein is identical to the sequence above (SEQ ID NO: 51).

Cloning of a R. palustris Aldehyde Dehydrogenase Gene (DduP_Rp) and Construction of Vector pTRGU344.

The 1392 bp coding sequence of an aldehyde dehydrogenase gene identified in R. palustris was optimized for expression in E. coli and synthetically constructed into pTRGU344. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was EcoRI-PacI-RBS-CDS-SbfI-HindIII-XbaI, resulting in pTRGU85.

The wild-type nucleotide sequence (WT), codon-optimized nucleotide sequence (CO), and deduced amino acid sequence of the R. palustris aldehyde dehydrogenase gene are listed in SEQ ID NO: 52, 53, and 54, respectively. The coding sequence is 1395 bp including the stop codon and the encoded predicted protein is 464 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 464 amino acids with a predicted molecular mass of 49.3 kDa and an isoelectric pH of 5.98.

Cloning of a R. capsulatus Aldehyde Dehydrogenase Gene (pduP_Rc) and Construction of Vector pTRGU346.

The 1599 bp coding sequence of an aldehyde dehydrogenase gene identified in R. capsulatus was optimized for expression in E. coli and synthetically constructed into pTRGU346. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was EcoRI-PacI-RBS-CDS-SbfI-HindIII-XbaI, resulting in pTRGU346.

The wild-type nucleotide sequence (WT), codon-optimized nucleotide sequence (CO), and deduced amino acid sequence of the R. capsulatus aldehyde dehydrogenase gene are listed in SEQ ID NO: 55, 56, and 57, respectively. The coding sequence is 1602 bp including the stop codon and the encoded predicted protein is 533 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 533 amino acids with a predicted molecular mass of 55.9 kDa and an isoelectric pH of 6.32.

Cloning of a R. rubrum Aldehyde Dehydrogenase Gene (pduP_Rr) and Construction of Vector pTRGU348.

The 1590 bp coding sequence of an aldehyde dehydrogenase gene identified in R. rubrum was optimized for expression in E. coli and synthetically constructed into pTRGU348. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was EcoRI-PacI-RBS-CDS-SbfI-HindIII-XbaI, resulting in pTRGU348.

The wild-type nucleotide sequence (WT), codon-optimized nucleotide sequence (CO), and deduced amino acid sequence of the R. rubrum aldehyde dehydrogenase gene are listed in SEQ ID NO: 58, 59, and 60, respectively. The coding sequence is 1593 bp including the stop codon and the encoded predicted protein is 530 amino acids. Using the SignalP program (Nielsen et al., 1997, Protein Engineering 10:1-6), a signal peptide in the sequence was predicted. Based on Vector NTI (Invitrogen, Paisley, UK) analyses, the predicted mature protein contains 498 amino acids with a predicted molecular mass of 52.3 kDa and an isoelectric pH of 6.06.

Cloning of an E. hallii Aldehyde Dehydrogenase Gene (pduP_Eh) and Construction of Vector pTRGU361.

The 1404 bp coding sequence of an aldehyde dehydrogenase gene identified in E. hallii was optimized for expression in E. coli and synthetically constructed into pTRGU360. The DNA fragment containing the codon-optimized coding sequence was designed with a ribosomal binding site (RBS, sequence 5′-GAAGGAGATATACC-3′) immediately prior to the start codon.

The resulting sequence was then submitted to and synthesized by Geneart AG (Regenburg, Germany) and delivered in the pMA backbone vector containing the β-lactamase encoding gene blaTEM-1. When synthesized, the coding sequence and RBS fragment was flanked by restriction sites to facilitate subsequent cloning steps. The entire synthetic fragment cloned into the pMA vector was EcoRI-PacI-RBS-CDS-SbfI-HindIII-XbaI, resulting in pTRGU346.

The wild-type nucleotide sequence (WT), codon-optimized nucleotide sequence (CO), and deduced amino acid sequence of the E. hallii aldehyde dehydrogenase gene are listed in SEQ ID NO: 61, 62, and 63, respectively. The coding sequence is 1407 bp including the stop codon and the encoded predicted protein is 468 amino acids. Using the SignalP program (Nielsen et al., 1997, Protein Engineering 10:1-6), no signal peptide in the sequence was predicted. Based on Vector NTI (Invitrogen, Paisley, UK) analyses, the predicted mature protein contains 533 amino acids with a predicted molecular mass of 50.9 kDa and an isoelectric pH of 5.79.

### Example 26

### Construction and Transformation of Pathway Constructs Containing Aldehyde Dehydrogenase for n-Propanol Production

Construction and Transformation of pTRGU44 Expressing P. freudenreichii Aldehyde Dehydrogenase Gene (DduP Pf_syn2).

A 1536 bp fragment containing the aldehyde dehydrogenase gene was amplified from pTRGU30 (Example 15) using primers P0017 and P0021 shown below.

Primer P0017:
(SEQ ID NO: 96)
5′-ATCCTCTAGAGAAGGAGATATACCATGCGT-3′
Primer P0021:
(SEQ ID NO: 97)
5′-TGCAAGCTTTTAGCGGATATTCAGGCCAC-3′

For the PCR reaction was used Phusion® Hot Start DNA polymerase (Finnzymes, Finland) and the amplification reaction was programmed for 29 cycles at 95° C. for 2 minutes; 95° C. for 30 seconds, 55° C. for 1 minute, 72° C. for 1 minute; then one cycle at 72° C. for 5 minutes. The resulting PCR product was purified with a PCR Purification Kit (Qiagen) according to manufacturer's instructions. Subsequently, both the PCR product and pTrc99A (E. Amann and J. Brosius, 1985, Gene 40(2-3), 183-190) were digested overnight at 37° C. with XbaI (New England Biolabs (NEB), Ipswich, Mass., USA) and HindIII (NEB) (restriction sites are underlined in the above primers). The enzymes were heat inactivated at 65° C. for 20 minutes and the pTrc99A reaction mixture was dephosphorylated with 1 U Calf intestine phosphatase (CIP) (NEB) for 30 minutes at 37° C. The digested pTrc99A and PCR products were run on a 1% agarose gel, and then purified using a QIAquick Gel Extraction Kit (Qiagen, Hilden, Germany) according to manufacturer's instructions.

The digested PCR product was ligated to the 4152 bp fragment of pTrc99A overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation. Transformants were plated onto LB plates containing 200 μg/mL ampicillin and incubated at 37° C. overnight. Selected colonies were then streaked on LB plates with 200 μg/mL ampicillin. One colony, E. coli TRGU44, was inoculated in liquid TY bouillon medium with 200 μg/mL ampicillin and incubated over night at 37° C. The corresponding plasmid pTRGU44 was isolated using a Qiaprep® Spin Miniprep Kit (Qiagen) and subjected to DNA sequencing to confirm that the aldehyde dehydrogenase gene was integrated into the vector. E. coli TRGU44 from the liquid overnight culture containing pTRGU44 was stored in 30% glycerol at −80° C.

Construction and Transformation of pTRGU42 Expressing L. collinoides Aldehyde Dehydrogenase Gene (pduP_Lc).

A 1479 bp fragment containing the aldehyde dehydrogenase gene was amplified from pTRGU31 using primers P0013 and P0019 shown below.

Primer P0013:
(SEQ ID NO: 98)
5′-ATCCTCTAGAGAAGGAGATATACCATGGCC-3′
Primer P0019:
(SEQ ID NO: 99)
5′-TGCAAGCTTTTAGACCTCCCAGGAACGCA-3′

For the PCR reaction was used Phusion® Hot Start DNA polymerase (Finnzymes, Finland) and the amplification reaction was programmed for 29 cycles at 95° C. for 2 minutes; 95° C. for 30 seconds, 55° C. for 1 minute, 72° C. for 1 minute; then one cycle at 72° C. for 5 minutes. The resulting PCR product was purified with a PCR Purification Kit (Qiagen, Hilden, Germany) according to manufacturer's instructions. Subsequently, both the PCR product and pTrc99A (E. Amann and J. Brosius, 1985, Gene 40(2-3), 183-190) were digested overnight at 37° C. with XbaI (New England Biolabs (NEB), Ipswich, Mass., USA) and HindIII (NEB) (restriction sites are underlined in the above primers). The enzymes were heat inactivated at 65° C. for 20 minutes and the pTrc99A reaction mixture was dephosphorylated with 1 U Calf intestine phosphatase (CIP) (NEB) for 30 minutes at 37° C. The digested pTrc99A and PCR products were run on a 1% agarose gel, and then purified using a QIAquick Gel Extraction Kit (Qiagen, Hilden, Germany) according to manufacturer's instructions.

The digested PCR product was ligated to the 4152 bp fragment of pTrc99A overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation. Transformants were plated onto LB plates containing 200 μg/mL ampicillin and incubated at 37° C. overnight. Selected colonies were then streaked on LB plates with 200 μg/mL ampicillin. One colony, E. coli TRGU42, was inoculated in liquid TY bouillon medium with 200 μg/mL ampicillin and incubated overnight at 37° C. The corresponding plasmid pTRGU42 was isolated using a Qiaprep® Spin Miniprep Kit (Qiagen) and subjected to DNA sequencing to confirm that the aldehyde dehydrogenase gene was integrated into the vector. E. coli TRGU42 from the liquid overnight culture containing pTRGU42 was stored in 30% glycerol at −80° C.

Construction and Transformation of pTRGU91 Expressing C. beijerinckii Aldehyde Dehydrogenase Gene (nduP_Cb).

A 1440 bp fragment containing the aldehyde dehydrogenase gene was amplified from pTRGU85 using primers P0015 and P0020 shown below.

Primer P0015:
(SEQ ID NO: 100)
5′-ATCCTCTAGAGAAGGAGATATACCATGAAT-3′
Primer P0020:
(SEQ ID NO: 101)
5′-TGCAAGCTTTTAGCCCGCCAGCACGCAAC-3′

For the PCR reaction was used Phusion® Hot Start DNA polymerase (Finnzymes, Finland) and the amplification reaction was programmed for 29 cycles at 95° C. for 2 minutes; 95° C. for 30 seconds, 55° C. for 1 minute, 72° C. for 1 minute; then one cycle at 72° C. for 5 minutes. The resulting PCR product was purified with a PCR Purification Kit (Qiagen, Hilden, Germany) according to manufacturer's instructions. Subsequently, both the PCR product and pTrc99A (E. Amann and J. Brosius, 1985, Gene 40(2-3), 183-190) were digested overnight at 37° C. with XbaI (New England Biolabs (NEB), Ipswich, Mass., USA) and HindIII (NEB) (restriction sites are underlined in the above primers). The enzymes were heat inactivated at 65° C. for 20 minutes and the pTrc99A reaction mixture was dephosphorylated with 1 U Calf intestine phosphatase (CIP) (NEB) for 30 minutes at 37° C. The digested pTrc99A and PCR products were run on a 1% agarose gel, and then purified using a QIAquick Gel Extraction Kit (Qiagen, Hilden, Germany) according to manufacturer's instructions.

The digested PCR product was ligated to the 4152 bp fragment of pTrc99A overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation. Transformants were plated onto LB plates containing 200 μg/mL ampicillin and incubated at 37° C. overnight. Selected colonies were then streaked on LB plates with 200 μg/mL ampicillin. One colony, E. coli TRGU91, was inoculated in liquid TY bouillon medium with 200 μg/mL ampicillin and incubated overnight at 37° C. The corresponding plasmid pTRGU91 was isolated using a Qiaprep® Spin Miniprep Kit (Qiagen) and subjected to DNA sequencing to confirm that the aldehyde dehydrogenase gene was integrated into the vector. E. coli TRGU91 from the liquid overnight culture containing pTRGU91 was stored in 30% glycerol at −80° C.

Construction and Transformation of pTRGU531 Expressing P. freudenreichii Aldehyde Dehydrogenase Gene (pduP_Pf_Syn2a).

The gene pduP_Pf_syn2a was cloned into vector pTRGU88 using the flanking sites BamHI and XbaI in pTRGU300. Both pTRGU88 and pTRGU300 were digested using 20 ul vector, 5 μl NEB 2 buffer, 2 μl XbaI, 2 μl BamHI, 0.5 μl BSA and 20 μl H₂O. Both pTRGU88 and pTRGU300 were digested overnight at 37° C. The enzymes were heat inactivated at 65° C. for 20 minutes and the pTRGU88 reaction mixture was dephosphorylated with 1 U Calf intestine phosphatase (CIP) (NEB) for 30 minutes at 37° C. The digested pTRGU88 and pTRGU300 were run on a 1% agarose gel, and bands of the expected sizes (pTRGU88: 4518 bp; pTRGU300: 1430 bp) were then purified using a QIAquick Gel Extraction Kit (Qiagen, Hilden, Germany) according to the manufacturer's instructions.

The isolated DNA fragments were ligated overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation.

Transformants were plated onto LB plates containing 20 μg/mL kanamycin and incubated at 37° C. overnight. Selected colonies were then streaked on LB plates with 20 μg/mL kanamycin. One colony, E. coli TRGU304, was inoculated in liquid TY bouillon medium with 10 μg/mL kanamycin and incubated overnight at 37° C. The corresponding plasmid pTRGU304 was isolated using a Qiaprep® Spin Miniprep Kit (Qiagen) and subjected to restriction analysis with BamHI and XbaI, which resulted in the bands BamHI-XbaI: 1430 bp and XbaI-BamHI: 4518 bp which confirmed correct insertion of the gene in pTRGU88. E. coli TRGU304 from the liquid overnight culture containing pTRGU304 was stored in 30% glycerol at −80° C.

Plasmid pTRGU304 was transformed using standard electroporation techniques into E. coli MG1655. Transformants were plated onto LB plates containing 20 μg/mL kanamycin and incubated at 37° C. overnight. Selected colonies were then streaked on LB plates with 20 μg/mL kanamycin. One colony, E. coli TRGU531, was inoculated in liquid TY bouillon medium with 10 μg/mL kanamycin and incubated overnight at 37° C. The corresponding plasmid pTRGU531 was isolated using a Qiaprep® Spin Miniprep Kit (Qiagen) and subjected to restriction analysis with BamHI and XbaI, which resulted in the bands BamHI-XbaI: 1430 bp and XbaI-BamHI: 4518 bp which confirmed correct insertion of the gene in pTRGU88. E. coli TRGU304 from the liquid overnight culture containing pTRGU304 was stored in 30% glycerol at −80° C.

Construction and Transformation of pTRGU551 Expressing P. freudenreichii Aldehyde Dehydrogenase Gene (pduP_Pf_Syn2b).

The gene pduP_Pf_syn2b was cloned into vector pTRGU88 using the flanking sites EcoRI and XbaI in pTRGU399. Both pTRGU88 and pTRGU399 were digested using 20 ul vector, 5 μl NEB 2 buffer, 2 μl XbaI, 2 μl EcoRI, 0.5 μl BSA and 20 μl H₂O. Both pTRGU88 and pTRGU399 were digested overnight at 37° C. The enzymes were heat inactivated at 65° C. for 20 minutes and the pTRGU88 reaction mixture was dephosphorylated with 1 U Calf intestine phosphatase (CIP) (NEB) for 30 minutes at 37° C. The digested pTRGU88 and pTRGU399 were run on a 1% agarose gel, and bands of the expected sizes (pTRGU88: 4497 bp; pTRGU399: 1452 bp) were then purified using a QIAquick Gel Extraction Kit (Qiagen, Hilden, Germany) according to the manufacturer's instructions.

The isolated DNA fragments were ligated overnight at 16° C. using T4 DNA ligase in T4 DNA ligase buffer containing 10 mM ATP (F. Hoffmann-La Roche Ltd, Basel Switzerland). A 1 μL aliquot of the ligation mix was transformed into E. coli TOP10 via electroporation. Transformants were plated onto LB plates containing 20 μg/mL kanamycin and incubated at 37° C. overnight. Selected colonies were then streaked on LB plates with 20 μg/mL kanamcyin. One colony, E. coli TRGU541, was inoculated in liquid TY bouillon medium with 20 μg/mL ampicillin and incubated overnight at 37° C. The corresponding plasmid pTRGU541 was isolated using a Qiaprep® Spin Miniprep Kit (Qiagen) and subjected to restriction analysis with EcoRI and XbaI, which resulted in the bands EcoRI-XbaI: 1452 bp and XbaI-EcoRI: 4497 bp which confirmed correct insertion of the gene in pTRGU88. E. coli TRGU541 from the liquid overnight culture containing pTRGU541 was stored in 30% glycerol at −80° C.

Plasmid pTRGU541 was transformed using standard electroporation techniques into E. coli MG1655. Transformants were plated onto LB plates containing 20 μg/mL kanamycin and incubated at 37° C. overnight. Selected colonies were then streaked on LB plates with 20 μg/mL kanamycin. One colony, E. coli TRGU551, was inoculated in liquid TY bouillon medium with 10 μg/mL kanamcyin and incubated overnight at 37° C. The corresponding plasmid pTRGU551 was isolated using a Qiaprep® Spin Miniprep Kit (Qiagen) and subjected to restriction analysis with EcoRI and XbaI, which resulted in the bands EcoRI-XbaI: 1452 bp and XbaI-EcoRI: 4497 bp which confirms correct insertion of the gene in pTRGU88. E. coli TRGU551 from the liquid overnight culture containing pTRGU551 was stored in 30% glycerol at −80° C.

Construction and Transformation of pTRGU543 Expressing R. palustris Aldehyde Dehydrogenase Gene (pduP_Rp).

The gene pduP_Rp was cloned into pTRGU88 essentially as described above. The EcoRI-XbaI fragment containing the gene was excised from vector pTRGU344 and purified from an agarose gel by isolating the 1437 bp DNA band. E. coli TOP10 was successfully transformed with the ligation mix of pTRGU88 and pduP_Rp and one colony, TRGU533, contained the gene correctly inserted into pTRGU88. The corresponding plasmid, pTRGU533, was isolated and transformed into E. coli MG1655. One transformant, TRGU543, contained the correct plasmid as verified by restriction analyses and was stored in 30% glycerol at −80° C.

Construction and Transformation of pTRGU545 Expressing R. capsulatus Aldehyde Dehydrogenase Gene (pduP_Rc).

The gene pduP_Rc was cloned into pTRGU88 essentially as described above. The EcoRI-XbaI fragment containing the gene was excised from vector pTRGU346 and purified from an agarose gel by isolating the 1644 bp DNA band. E. coli TOP10 was successfully transformed with the ligation mix of pTRGU88 and pduP_Rc and one colony, TRGU535, contained the gene correctly inserted into pTRGU88. The corresponding plasmid, pTRGU535, was isolated and transformed into E. coli MG1655. One transformant, TRGU545, contained the correct plasmid as verified by restriction analyses and was stored in 30% glycerol at −80° C.

Construction and Transformation of pTRGU547 Expressing R. rubrum Aldehyde Dehydrogenase Gene (pduP_Rr).

The gene pduP_Rr was cloned into pTRGU88 essentially as described above. The EcoRI-XbaI fragment containing the gene was excised from vector pTRGU348 and purified from an agarose gel by isolating the 1635 bp DNA band. E. coli TOP10 was successfully transformed with the ligation mix of pTRGU88 and pduP_Rr and one colony, TRGU537, contained the gene correctly inserted into pTRGU88. The corresponding plasmid, pTRGU537, was isolated and transformed into E. coli MG1655. One transformant, TRGU547, contained the correct plasmid as verified by restriction analyses and was stored in 30% glycerol at −80° C.

Construction and Transformation of pTRGU549 Expressing E. hallii Aldehyde Dehydrogenase Gene (pduP_Eh).

The gene pduP_Eh was cloned into pTRGU88 essentially as described above. The EcoRI-XbaI fragment containing the gene was excised from vector pTRGU361 and purified from an agarose gel by isolating the 1449 bp DNA band. E. coli TOP10 was successfully transformed with the ligation mix of pTRGU88 and pduP_Eh and one colony, TRGU539, contained the gene correctly inserted into pTRGU88. The corresponding plasmid, pTRGU539, was isolated and transformed into E. coli MG1655. One transformant, TRGU549, contained the correct plasmid as verified by restriction analyses and was stored in 30% glycerol at −80° C.

### Example 27

### Production of n-Propanol in Recombinant E. coli TOP10 Containing Heterologous Aldehyde Dehydrogenase

E. coli strains Trc99A (negative control) and TRGU44, TRGU42, and TRGU91 were grown overnight with shaking (250 rpm) in 10 mL LB medium containing 100 μg/mL ampicillin and 1 mM isopropyl-beta-thio galactopyranoside (IPTG). A 0.5 mL sample of each strain was withdrawn after overnight incubation. Each sample was centrifuged at 15000×g for 1 minute using a table centrifuge and the supernatant discarded. The cells of E. coli Trc99A and E. coli TRGU44, TRGU42, and TRGU91 were resuspended in 0.5 mL minimal medium (MM) supplemented with leucine (1 mM) which was used to inoculate one new 10 mL culture for each sample. The cultures were incubated for 72 hours at 37° C. with shaking (250 rpm). A 2 mL sample was withdrawn at the end of incubation and subsequently analyzed by gas chromatography with standards for acetone, n-propanol and isopropanol as described herein.

As indicated in Table 13, n-propanol was produced in significant amount by E. coli TRGU44 but not by Trc99A (negative control), TRGU42, and TRGU91.

TABLE 13

| | | | Propion- |
|---|---|---|---|
| | | n-propanol | aldehyde |
| | | detected | detected |
| Medium/Strain | SEQ ID No | (mg/L) | (mg/L) |
| MM + leucine | N/A | 0 | nd |
| MM + leucine + Trc99A | N/A | Trace/not | nd |
| | | quantifiable | |
| MM + leucine + TRGU44 | 26 | 50 | nd |
| MM + leucine + TRGU42 | 29 | nd | nd |
| MM + leucine + TRGU91 | 32 | nd | nd |

“nd” means not detected

### Example 28

### Production of n-Propanol in Recombinant E. coli MG1655 Containing Heterologous Aldehyde Dehydrogenase

E. coli strains TRGU269 (negative control), TRGU531, TRGU551, TRGU543, TRGU545, TRGU547, TRGU549 were grown overnight with shaking (250 rpm) in 10 mL LB medium containing 100 μg/mL ampicillin and 1 mM isopropyl-beta-thio galactopyranoside (IPTG). OD was measured and a volume corresponding to 2 ml of OD(600 nm)=1 was withdrawn after overnight incubation (all between 1.39 ml and 2.95 ml). Each sample was centrifuged at 5000×g for 5 minutes using a table centrifuge and the supernatant discarded. The cells of TRGU269 (negative control), TRGU531, TRGU551, TRGU543, TRGU545, TRGU547, TRGU549 were resuspended in 0.5 mL minimal medium (MM) supplemented with 1 μM adenosyl cobalamine (vitamin B12), which was used to inoculate one new 10 mL culture for each sample. The cultures were incubated for 116 hours at 37° C. with shaking (250 rpm). A 2 mL sample was withdrawn after 20 hours, 44 hours, and 116 hours of incubation and subsequently analyzed by gas chromatography with standards for n-propanol and propionaldehyde. Acetone, 1-propanol and isopropanol in fermentation broths were detectable by GC-FID. Samples were diluted 1+1 with 0.05% tetrahydrofuran in methanol and analyzed as described above.

As indicated in Table 14, n-propanol was produced in significant amount by E. coli TRGU551, TRGU543, TRGU545, TRGU547, and TRGU549 but not by TRGU269 (negative control) nor by E. coli TRGU531. As pduP_Pf_syn2a and pduP_Pf_syn2b encodes identical enzymes but differ in nucleotide sequences, the difference detected here with respect to n-propanol production is likely to be caused by differences in transcription profiles of the two genes.

TABLE 14

| Strain/ | Gene | | Propanol (mg/L) | | |
|---|---|---|---|---|---|
| sample | expressed | SEQ ID NO | 20 h | 44 h | 116 h |
| Minimal | N/A | N/A | nd | — | — |
| Medium | | | | | |
| (control) | | | | | |
| TRGU269 | N/A | N/A | nd | 0.000 | 0.000 |
| (control) | | | | | |
| TRGU531 | pduP_Pf_syn2a | 49 | nd | 0.000 | 0.000 |
| TRGU551 | pduP_Pf_syn2b | 50 | 10 | 10 | 0.000 |
| TRGU543 | pduP_Rp | 53 | nd | 0.000 | 20 |
| TRGU545 | pduP_Rc | 56 | 30 | 20 | 0.000 |
| TRGU547 | pduP_Rr | 59 | 10 | 20 | 0.000 |
| TRGU549 | pduP_Eh | 62 | 10 | 10 | 0.000 |

“nd” means not detected.
“0.000” means that the compound was detected.

### Example 29

### Cloning of P. freudenreichii Methylmalonyl-CoA Mutase Small Subunit Gene (mutA) and Large Subunit Gene (mutB), Kinase ArgK Gene (argK), and Methylmalonyl-CoA Epimerase Gene (mme) and Construction of Vectors pTRGU320 (mutA), pTRGU322 (mutB), pTRGU324 (argK), and pTRGU350 (mme)

The coding sequences of the wild type sequences of mutA, mutB, argK, and mme were optimized for expression in E. coli and synthetically constructed into pTRGU320 (mutA), pTRGU322 (mutB), pTRGU324 (argK), and pTRGU350 (mme). The DNA fragments containing the codon-optimized coding sequences were designed with ribosomal binding sites (RBS, sequence 5′-GAAGGAGATATACC-3′) immediately prior to the start codon.

The resulting sequences were then submitted to and synthesized by Geneart AG (Regenburg, Germany) and delivered in the pMA backbone vector containing the β-lactamase encoding gene blaTEM-1. When synthesized, each coding sequence and RBS fragment was flanked by restriction sites to facilitate subsequent cloning steps. The entire synthetic fragment cloned into the pMA vector was EcoRI-RBS-CDS1-BamHI-HindIII-XbaI for mutA as listed in Table 15, resulting in pTRGU320. Similarly, mutB was flanked by EcoRI, BamHI and NotI, HindIII, XbaI, which enabled successive cloning of mutA and mutB into one operon, where the coding sequences were separated by a BamHI restriction site and the RBS. The SEQ ID numbers of wild-type nucleotide sequences (WT), codon-optimized nucleotide sequences (CO), and deduced amino acid sequences of all remaining synthetically optimized genes are also listed in Table 15.

TABLE 15

| | | | Gene | |
|---|---|---|---|---|
| | | Gene | SEQ ID | SEQ ID |
| CDS | | SEQ ID | (codon- | (expressed |
| (gene) | Restriction site pattern | (wild-type) | optimized) | enzyme) |
| mutA | EcoRI-RBS-CDS- | 64 | 65 | 66 |
| | BamHI-HindIII- | | | |
| | XbaI | | | |
| mutB | EcoRI-BamHI-RBS- | 67 | 68 | 69 |
| | CDS-NotI-HindIII- | | | |
| | XbaI | | | |
| argK | EcoRI-NotI-RBS-CDS- | 70 | 71 | 72 |
| | AscI-HindIII-XbaI | | | |
| mme | EcoRI-AscI-RBS-CDS- | 73 | 74 | 75 |
| | FseI-HindIII-XbaI | | | |

### Example 30

### Cloning of E. coli Methylmalonyl-CoA Mutase Gene (Sbm), E. coli Protein Kinase Gene (ygfD), E. coli Methylmalonyl-CoA Decarboxylase Gene (ydgG), and Construction of Various n-Propanol Pathway Gene Combinations

The E. coli methylmalonyl-CoA mutase (sbm) gene, E. coli protein kinase gene (ygfD), and E. coli methylmalonyl-CoA decarboxylase gene (ydgG) were amplified from the E. coli genome with PCR incorporating restriction sites, RBS and stop codon as listed in Table 16. Additionally, the gene pduP_Pf_syn2a in pTRGU300 (supra) was synthesized without the necessary restriction sites and thus was amplified from pTRGU300 with the correct restriction sites as described below.

TABLE 17

| | | | SEQ ID |
|---|---|---|---|
| CDS | | Gene | (expressed |
| (gene) | Restriction site pattern | SEQ ID | enzyme) |
| sbm | EcoRI-RBS-CDS-BamHI-HindIII-XbaI | 79 | 93 |
| ygfD | EcoRI-NotI-RBS-CDS-AscI-HindIII-XbaI | 81 | 94 |
| ygfG | EcoRI-FseI-RBS-CDS-PacI-HindIII-XbaI | 102 | 103 |
| pduP | EcoRI-PacI-RBS-CDS-SbfI-HindIII-XbaI | 49 | 51 |

The following primers were used for the PCR reactions:

### Cloning of sbm

### EcoRI-RBS-sbm-BamHI-NotI-HindIII-XbaI

Primer P217 (SEQ ID NO: 104):
5′-CACCGAATTCAAGAAGGAGATATACCATGTCTAACGTGCAGGAGTGGCAAC-3′
Primer P218 (SEQ ID NO: 105):
5′-CTAGTCTAGAAAGCTTGCGGCCGCGGATCCTTAATCATGATGCTGGCTTATCAGA-3′

Cloning of ygfD

### EcoRI-NotI-RBS-CDS3-AscI-FseI-HindIII-XbaI

Primer P219 (SEQ ID NO: 106):
5′-CACCG AATTC GCGGC CGCAA GAAGG AGATA TACCA TGATT AATGA AGCCA CGCTG
GCAG-3′
Primer P220 (SEQ ID NO: 107):
5′-CTAGTCTAGAAAGCTTGGCCGGCCGGCGCGCCTTAATCAAAATATTGCGTCTGGATA-3′

Cloning of ygfG

### EcoRI-AscI-FseI-RBS-CDS5-PacI-HindIII-XbaI

AscI and XbaI when cloning into a pathway without mme; FseI and XbaI when cloning into a pathway with mme.

Primer P229 (SEQ ID NO: 108)
5′-CACCG AATTC GGCGC GCCGG CCGGC CAAGA AGGAG ATATA CCATG TCTTA
TCAGT ATGTT AACGT TG-3′
Primer P222 (SEQ ID NO: 109):
5′-CTAGTCTAGAAAGCTTTTAATTAACTAATGACCAACGAAATTAGGTTTA-3′

Cloning of pduP_syn2a

### EcoRI-Pad-RBS-CDS6-SbfI-HindIII-XbaI

Primer P223 (SEQ ID NO: 110):
5′-CACCGAATTCTTAATTAAAAGGAGATATACCATGACCATCA-3′
Primer P224 (SEQ ID NO: 111):
5′-CTAGTCTAGAAAGCTTCCTGCAGGTTAGCGGATATTCAGGCCACTC
TTT-3′

The PCR reactions were carried out using Phusion® Hot Start DNA polymerase (Finnzymes, Finland). The resulting PCR products were purified with a PCR Purification Kit (Qiagen, Hilden, Germany) according to manufacturer's instructions. Subsequently, each PCR product and the cloning vectors were digested overnight at 37° C. with the restriction enzymes listed in Table 18. The enzymes were heat inactivated at 65° C. for 20 minutes and the cloning vector reaction mixtures were dephosphorylated with 1 U Calf intestine phosphatase (CIP) (NEB) for 30 minutes at 37° C. The digested vectors and PCR products were run on a 1% agarose gel, and then purified using a QIAquick Gel Extraction Kit (Qiagen, Hilden, Germany) according to manufacturer's instructions.

Insertion of Sbm and ygfD or arqK into pTRGU187 Via a 3 Fragment Ligation.

Sbm was amplified for 30 cycles at 96° C. for 2 minutes; 96° C. for 30 seconds, 58° C. for 30 seconds, 72° C. for 1 minute 10 seconds; then one cycle at 72° C. for 5 minutes. ygfD was amplified for 30 cycles at 96° C. for 2 minutes; 96° C. for 30 seconds, 55° C. for 30 seconds, 72° C. for 40 seconds; then one cycle at 72° C. for 5 minutes. The PCR purification, digest with EcoRI and NotI for sbm and NotI and XbaI for ygfD was carried out essentially as described herein. The 3 fragment ligation of pTRGU187 digested with EcoRI and XbaI, sbm digested with EcoRI and NotI, and ygfD digested with NotI and XbaI was carried out essentially as described herein, with one additional DNA fragment in the reaction. A 1 μL aliquot of the ligation mix was transformed into E. coli TOP10 via chemical transformation. Transformants were plated onto LB plates containing 20 μg/mL kanamycin and incubated at 37° C. overnight. Selected colonies were then streaked on LB plates with 20 μg/mL kanamycin. One colony, E. coli TRGU367, was inoculated in liquid LB bouillon medium with 10 μg/mL kanamycin and incubated overnight at 37° C. The corresponding plasmid pTRGU367 was isolated using a Qiaprep® Spin Miniprep Kit (Qiagen) and subjected to DNA sequencing to confirm that the sbm and ygfD genes were integrated correctly into the vector. E. coli TRGU367 from the liquid overnight culture containing pTRGU367 was stored in 30% glycerol at −80° C. Cloning of all the n-propanol biosynthesis pathway genes followed essentially the same procedure. Applied restriction enzymes and DNA fragments for the cloning of the entire n-propanol biosynthesis gene pathways are outlined below in Table 18.

TABLE 18

| Gene(s) | SEQ ID | | Restriction | Fragments | Inserted into vector/ |
|---|---|---|---|---|---|
| cloned | NOs | Cloned from | enzymes | ligated | Restriction sites |
| sbmI | 79 | PCR: P217, P218 | EcoRI, NotI | 3 | pTRGU187/ |
| ygfD | 81 | PCR: P219, P220 | NotI, XbaI | | EcoRI, XbaI |
| sbmI | 79 | PCR: P217, P218 | EcoRI, NotI | 3 | pTRGU187/ |
| argK | 71 | pTRGU324 | NotI, XbaI | | EcoRI, XbaI |
| mutA | 65 | pTRGU320 | EcoRI, BamHI | 3 | pTRGU187/ |
| mutB | 67 | pTRGU322 | BamHI, XbaI | | EcoRI, XbaI |
| argK | 71 | pTRGU324 | NotI, XbaI | 2 | pTRGU187[mutAB] |
| | | | | | NotI, XbaI |
| ygfD | 81 | PCR: P219, P220 | NotI/XbaI | 2 | pTRGU187[mutAB] |
| | | | | | NotI, XbaI |
| ygfG | 102 | PCR: P222, P229 | AscI, XbaI | 2 | pTRGU187[sbm ygfD] |
| | | | | | or |
| | | | | | pTRGU187[sbm argK] |
| | | | | | AscI, XbaI |
| ygfG | 102 | PCR: P222, P229 | AscI, XbaI | 2 | pTRGU187[mutAB ygfD] |
| | | | | | or |
| | | | | | pTRGU187[mutAB |
| | | | | | argK] |
| | | | | | AscI, XbaI |
| mmeI | 74 | pTRGU350 | AscI, FseI | 3 | pTRGU187[sbm ygfD] |
| ygfG | 102 | PCR: P222, P229 | FseI, XbaI | | or |
| | | | | | pTRGU187[sbm argK] |
| | | | | | AscI, XbaI |
| mmeI | 74 | pTRGU350 | AscI, FseI | 3 | pTRGU187[mutAB ygfD] |
| ygfG | 102 | PCR: P222, P229 | FseI, XbaI | | or |
| | | | | | pTRGU187[mutAB |
| | | | | | argK] |
| | | | | | AscI, XbaI |
| pduP | 49, | PCR: P223, | PacI, XbaI | 2 | pTRGU187[sbm ygfD |
| | 50, | P224 or | | | ygfG] or |
| | 62, | pTRGU399, | | | pTRGU187[sbm argK |
| | 53, | pTRGU360, | | | ygfG] |
| | 59, or | pTRGU344, | | | PacI, XbaI |
| | 56 | pTRGU348, or | | | |
| | | pTRGU346 | | | |
| pduP | 49, | PCR: P223, | PacI, XbaI | 2 | pTRGU187[mutAB ygfD |
| | 50, | P224 or | | | ygfG] or |
| | 62, | pTRGU399, | | | pTRGU187[mutAB argK |
| | 53, | pTRGU360, | | | ygfG] |
| | 59, or | pTRGU344, | | | PacI, XbaI |
| | 56 | pTRGU348, or | | | |
| | | pTRGU346 | | | |
| pduP | 49, | PCR: P223, | PacI, XbaI | 2 | pTRGU187[sbm ygfD |
| | 50, | P224 or | | | mme ygfG] or |
| | 62, | pTRGU399, | | | pTRGU187[sbm argK |
| | 53, | pTRGU360, | | | mme ygfG] |
| | 59, or | pTRGU344, | | | PacI, XbaI |
| | 56 | pTRGU348, or | | | |
| | | pTRGU346 | | | |
| pduP | 49, | PCR: P223, | PacI, XbaI | 2 | pTRGU187[mutAB ygfD |
| | 50, | P224 or | | | mme ygfG] or |
| | 62, | pTRGU399, | | | pTRGU187[mutAB argK |
| | 53, | pTRGU360, | | | mme ygfG] |
| | 59, or | pTRGU344, | | | PacI, XbaI |
| | 56 | pTRGU348, or | | | |
| | | pTRGU346 | | | |

Transformants of the n-propanol biosynthesis genes, TRGU362-517, are listed in Table 19. The corresponding plasmids pTRGU362-517 were isolated using a Qiaprep®Spin Miniprep Kit (Qiagen) and subjected to DNA sequencing in cases where PCR products had been cloned in order to confirm that the cloned genes were integrated without errors into the vector. E. coli TRGU362-517 from the liquid overnight culture containing pTRGU362-517 were stored in 30% glycerol at −80° C.

TABLE 19

| | | | | | Aldehyde |
|---|---|---|---|---|---|
| | Mutase | Chaperone | Epimerase | Decarboxylase | dehydrogenase |
| Strain | (SEQ ID NO) | (SEQ ID NO) | (SEQ ID NO) | (SEQ ID NO) | (SEQ ID NO) |
| TRGU187 | — | — | — | — | — |
| pTRGU362 | mutAB | — | — | — | — |
| | (65/68) | | | | |
| pTRGU364 | mutAB | argK | — | — | — |
| | (65/68) | (71) | | | |
| pTRGU366 | mutAB | ygfD | — | — | — |
| | (65/68) | (81) | | | |
| pTRGU367 | sbm | ygfD | — | — | — |
| | (79) | (81) | | | |
| pTRGU369 | sbm | argK | — | — | — |
| | (79) | (71) | | | |
| TRGU409 | sbm | ygfD | mme | ygfG | — |
| | (79) | (81) | (74) | (102) | |
| TRGU410 | sbm | argK | mme | ygfG | — |
| | (79) | (71) | (74) | (102) | |
| TRGU412 | sbm | ygfD | — | ygfG | — |
| | (79) | (81) | | (102) | |
| TRGU414 | sbm | argK | — | ygfG | — |
| | (79) | (71) | | (102) | |
| TRGU416 | mutAB | ygfD | mme | ygfG | — |
| | (65/68) | (81) | (74) | (102) | |
| TRGU418 | mutAB | argK | mme | ygfG | — |
| | (65/68) | (71) | (74) | (102) | |
| TRGU420 | mutAB | ygfD | — | ygfG | — |
| | (65/68) | (81) | | (102) | |
| TRGU422 | mutAB | argK | — | ygfG | — |
| | (65/68) | (71) | | (102) | |
| TRGU424 | sbm | ygfD | mme | ygfG | pduP_Rp |
| | (79) | (81) | (74) | (102) | (53) |
| TRGU426 | sbm | ygfD | mme | ygfG | pduP_Rc |
| | (79) | (81) | (74) | (102) | (56) |
| TRGU428 | sbm | ygfD | mme | ygfG | pduP_Rr |
| | (79) | (81) | (74) | (102) | (59) |
| TRGU430 | sbm | ygfD | mme | ygfG | pduP_Eh |
| | (79) | (81) | (74) | (102) | (62) |
| TRGU432 | sbm | argK | mme | ygfG | pduP_Rp |
| | (79) | (71) | (74) | (102) | (53) |
| TRGU433 | sbm | argK | mme | ygfG | pduP_Rr |
| | (79) | (71) | (74) | (102) | (59) |
| TRGU434 | sbm | ygfD | — | ygfG | pduP_Rp |
| | (79) | (81) | | (102) | (53) |
| TRGU436 | sbm | ygfD | — | ygfG | pduP_Rr |
| | (79) | (81) | | (102) | (59) |
| TRGU438 | sbm | ygfD | — | ygfG | pduP_Eh |
| | (79) | (81) | | (102) | (62) |
| TRGU440 | sbm | argK | — | ygfG | pduP_Rp |
| | (79) | (71) | | (102) | (53) |
| TRGU442 | sbm | argK | — | ygfG | pduP_Rc |
| | (79) | (71) | | (102) | (56) |
| TRGU444 | sbm | argK | — | ygfG | pduP_Rr |
| | (79) | (71) | | (102) | (59) |
| TRGU446 | sbm | argK | — | ygfG | pduP_Eh |
| | (79) | (71) | | (102) | (62) |
| TRGU448 | mutAB | ygfD | mme | ygfG | pduP_Rp |
| | (65/68) | (81) | (74) | (102) | (53) |
| TRGU449 | mutAB | ygfD | mme | ygfG | pduP_Rr |
| | (65/68) | (81) | (74) | (102) | (59) |
| TRGU451 | mutAB | ygfD | mme | ygfG | pduP_Eh |
| | (65/68) | (81) | (74) | (102) | (62) |
| TRGU452 | mutAB | argK | mme | ygfG | pduP_Rp |
| | (65/68) | (71) | (74) | (102) | (53) |
| TRGU454 | mutAB | argK | mme | ygfG | pduP_Rr |
| | (65/68) | (71) | (74) | (102) | (59) |
| TRGU456 | mutAB | argK | mme | ygfG | pduP_Eh |
| | (65/68) | (71) | (74) | (102) | (62) |
| TRGU458 | mutAB | ygfD | — | ygfG | pduP_Rp |
| | (65/68) | (81) | | (102) | (53) |
| TRGU459 | mutAB | ygfD | — | ygfG | pduP_Rc |
| | (65/68) | (81) | | (102) | (56) |
| TRGU460 | mutAB | ygfD | — | ygfG | pduP_Rr |
| | (65/68) | (81) | | (102) | (59) |
| TRGU462 | mutAB | ygfD | — | ygfG | pduP_Eh |
| | (65/68) | (81) | | (102) | (62) |
| TRGU464 | mutAB | argK | — | ygfG | pduP_Rp |
| | (65/68) | (71) | | (102) | (53) |
| TRGU466 | mutAB | argK | — | ygfG | pduP_Rr |
| | (65/68) | (71) | | (102) | (59) |
| TRGU468 | mutAB | argK | — | ygfG | pduP_Eh |
| | (65/68) | (71) | | (102) | (62) |
| TRGU484 | sbm | argK | mme | ygfG | pduP_Eh |
| | (79) | (71) | (74) | (102) | (62) |
| TRGU489 | sbm | argK | mme | ygfG | pduP_Rc |
| | (79) | (71) | (74) | (102) | (56) |
| TRGU491 | sbm | ygfD | — | ygfG | pduP_Rc |
| | (79) | (81) | | (102) | (56) |
| TRGU493 | mutAB | ygfD | mme | ygfG | pduP_Rc |
| | (65/68) | (81) | (74) | (102) | (56) |
| TRGU495 | mutAB | argK | mme | ygfG | pduP_Rc |
| | (65/68) | (71) | (74) | (102) | (56) |
| TRGU497 | mutAB | argK | — | ygfG | pduP_Rc |
| | (65/68) | (71) | | (102) | (56) |
| TRGU503 | sbm | ygfD | mme | ygfG | pduP_Pf_syn2a |
| | (79) | (81) | (74) | (102) | (49) |
| TRGU505 | sbm | argK | mme | ygfG | pduP_Pf_syn2a |
| | (79) | (71) | (74) | (102) | (49) |
| TRGU507 | sbm | ygfD | — | ygfG | pduP_Pf_syn2a |
| | (79) | (81) | | (102) | (49) |
| TRGU509 | sbm | argK | — | ygfG | pduP_Pf_syn2a |
| | (79) | (71) | | (102) | (49) |
| TRGU511 | mutAB | ygfD | mme | ygfG | pduP_Pf_syn2a |
| | (65/68) | (81) | (74) | (102) | (49) |
| TRGU513 | mutAB | argK | mme | ygfG | pduP_Pf_syn2a |
| | (65/68) | (71) | (74) | (102) | (49) |
| TRGU515 | sbm | ygfD | mme | ygfG | pduP_Pf_syn2b |
| | (79) | (81) | (74) | (102) | (50) |
| TRGU517 | sbm | argK | — | ygfG | pduP_Pf_syn2b |
| | (79) | (71) | | (102) | (50) |

### Example 31

### Production of n-Propanol in Recombinant E. coli TOP10 Containing Various Heterologous n-Propanol Pathway Gene Combinations

E. coli TOP10 strains harboring the plasmids listed in Table 19 (supra) were grown individually overnight with shaking (250 rpm) at 37° C. in 10 ml MM containing 10 μg/ml kanamycin and 1 mM isopropyl-beta-thio galactopyranoside. Initially, TRGU409-TRGU468 from Table 19 were cultivated and subsequently the remaining strains were cultivated in a separate experiment under essentially identical conditions.

In the primary cultivation experiment, a 2 ml sample from each medium was withdrawn after 17 hours, and 41 hours and selected strains were also sampled after 65 hours. Each sample was analyzed using gas chromatography as above. The propanol titers produced by each strain are listed in Table 20.

TABLE 20

| Sample | Strain | Genotype | 17 hours | 41 hours | 65 hours |
|---|---|---|---|---|---|
| Medium | — | — | 0 | — | — |
| 1 | TRGU187 | pTRGU187 | 0 | 0 | 0 |
| 2 | TRGU409 | pTRGU187[sbm-ygfD-mme-ygfG] | 10 | 10 | 10 |
| 3 | TRGU410 | pTRGU187[sbm-argK-mme-ygfG] | 10 | 10 | 0 |
| 4 | TRGU412 | pTRGU187[sbm-ygfD-ygfG] | 10 | 10 | 10 |
| 5 | TRGU414 | pTRGU187[sbm-argK-ygfG] | 10 | 10 | 0 |
| 6 | TRGU416 | pTRGU187[mutAB-ygfD-mme- | 10 | 10 | 0 |
| | | ygfG] | | | |
| 7 | TRGU418 | pTRGU187[mutAB-argK-mme- | 10 | 0 | 0 |
| | | ygfG] | | | |
| 8 | TRGU420 | pTRGU187[mutAB-ygfD-ygfG] | 10 | 10 | 10 |
| 9 | TRGU422 | pTRGU187[mutAB-argK-ygfG] | 10 | 10 | 0 |
| 10 | TRGU424 | pTRGU187[sbm-ygfD-mme-ygfG- | 40 | 30 | 30 |
| | | pduP_Rp] | | | |
| 11 | TRGU426 | pTRGU187[sbm-ygfD-mme-ygfG- | 30 | 30 | 20 |
| | | pduP_Rc] | | | |
| 12 | TRGU428 | pTRGU187[sbm-ygfD-mme-ygfG- | 20 | 20 | 10 |
| | | pduP_Rr] | | | |
| 13 | TRGU430 | pTRGU187[sbm-ygfD-mme-ygfG- | 50 | 30 | 30 |
| | | pduP_Eh] | | | |
| 14 | TRGU432 | pTRGU187[sbm-argK-mme-ygfG- | 30 | 20 | 20 |
| | | pduP_Rp] | | | |
| 15 | TRGU433 | pTRGU187[sbm-argK-mme-ygfG- | 10 | 10 | 10 |
| | | pduP_Rr] | | | |
| 16 | TRGU434 | pTRGU187[sbm-ygfD-ygfG- | 40 | 30 | 30 |
| | | pduP_Rp] | | | |
| 17 | TRGU436 | pTRGU187[sbm-ygfD-ygfG- | 20 | 20 | 20 |
| | | pduP_Rr] | | | |
| 18 | TRGU438 | pTRGU187[sbm-ygfD-ygfG- | 30 | 40 | 30 |
| | | pduP_Eh] | | | |
| 19 | TRGU440 | pTRGU187[sbm-argK-ygfG- | 30 | 20 | 20 |
| | | pduP_Rp] | | | |
| 20 | TRGU442 | pTRGU187[sbm-argK-ygfG- | 30 | 20 | 20 |
| | | pduP_Rc] | | | |
| 21 | TRGU444 | pTRGU187[sbm-argK-ygfG- | 30 | 20 | 20 |
| | | pduP_Rr] | | | |
| 22 | TRGU446 | pTRGU187[sbm-argK-ygfG- | 40 | 40 | 20 |
| | | pduP_Eh] | | | |
| 23 | TRGU448 | pTRGU187[mutAB-ygfD-mme- | 10 | 20 | 10 |
| | | ygfG-pduP_Rp] | | | |
| 24 | TRGU449 | pTRGU187[mutAB-ygfD-mme- | 10 | 10 | 10 |
| | | ygfG-pduP_Rr] | | | |
| 25 | TRGU451 | pTRGU187[mutAB-ygfD-mme- | 10 | 20 | 30 |
| | | ygfG-pduP_Eh] | | | |
| 26 | TRGU452 | pTRGU187[mutAB-argK-mme- | 10 | 20 | 20 |
| | | ygfG-pduP_Rp] | | | |
| 27 | TRGU454 | pTRGU187[mutAB-argK-mme- | 10 | 10 | 10 |
| | | ygfG-pduP_Rr] | | | |
| 28 | TRGU456 | pTRGU187[mutAB-argK-mme- | 10 | 10 | 20 |
| | | ygfG-pduP_Eh] | | | |
| 29 | TRGU458 | pTRGU187[mutAB-ygfD-ygfG- | 10 | 10 | 20 |
| | | pduP_Rp] | | | |
| 30 | TRGU459 | pTRGU187[mutAB-ygfD-ygfG- | 20 | 20 | 20 |
| | | pduP_Rc] | | | |
| 31 | TRGU460 | pTRGU187[mutAB-ygfD-ygfG- | 10 | 10 | 10 |
| | | pduP_Rr] | | | |
| 32 | TRGU462 | pTRGU187[mutAB-ygfD-ygfG- | 20 | 20 | 30 |
| | | pduP_Eh] | | | |
| 33 | TRGU464 | pTRGU187[mutAB-argK-ygfG- | 10 | 10 | 20 |
| | | pduP_Rp] | | | |
| 34 | TRGU466 | pTRGU187[mutAB-argK-ygfG- | 10 | 10 | 10 |
| | | pduP_Rr] | | | |
| 35 | TRGU468 | pTRGU187[mutAB-argK-ygfG- | 10 | 20 | 20 |
| | | pduP_Eh] | | | |

The results obtained in Table 20 show as expected that TRGU187 harboring the empty vector produces no propanol. Furthermore, when the first 3 genes of the n-propanol biosynthesis pathway are expressed in E. coli, small amounts of 1-propanol are detected. This suggests that E. coli is able to slowly reduce propionyl-CoA to n-propanol with a native aldehyde dehydrogenase and alcohol dehydrogenase. However, expression of the aldehyde dehydrogenases pduP_Rp, pduP_Rc, pduP_Rr and pduP_Eh increases the propanol production several fold.

Cultivation of the remaining strains from Table 19 (supra) was carried out as described above, and the results are listed in Table 21.

TABLE 21

| | | | 16 hours | 45 hours |
|---|---|---|---|---|
| Sample | Strain | Genotype | [mg/L] | [mg/L] |
| 1 | Trc99A | pTrc99A | 10 | 10 |
| 2 | TRGU284 | pTrc99A | 0 | 0 |
| | | pTRGU88 | | |
| 3 | TRGU187 | pTRGU187 | 0 | 0 |
| 4 | TRGU44 | pTrc99A[pduP_Pf_syn2] | 40 | 60 |
| 5 | TRGU302 | pTrc99A[pduP_Pf_syn2a] | 100 | 60 |
| 6 | TRGU304 | pTRGU88[pduP_Pf_syn2a] | 20 | 30 |
| 7 | TRGU409 | pTRGU187[sbm-ygfD-mme-ygfG] | 10 | 10 |
| 8 | TRGU410 | pTRGU187[sbm-argK-mme-ygfG] | 10 | 10 |
| 9 | TRGU412 | pTRGU187[sbm-ygfD-ygfG] | 10 | 10 |
| 10 | TRGU414 | pTRGU187[sbm-argK-ygfG] | 10 | 10 |
| 11 | TRGU416 | pTRGU187[mutAB-ygfD-mme- | 10 | 0 |
| | | ygfG] | | |
| 12 | TRGU418 | pTRGU187[mutAB-argK-mme- | 10 | 10 |
| | | ygfG] | | |
| 13 | TRGU420 | pTRGU187[mutAB-ygfD-ygfG] | 10 | 10 |
| 14 | TRGU422 | pTRGU187[mutAB-argK-ygfG] | 10 | 10 |
| 15 | TRGU424 | pTRGU187[sbm-ygfD-mme-ygfG- | 50 | 170 |
| | | pduP_Rp] | | |
| 16 | TRGU430 | pTRGU187[sbm-ygfD-mme-ygfG- | 50 | 90 |
| | | pduP_Eh] | | |
| 17 | TRGU434 | pTRGU187[sbm-ygfD-ygfG- | 50 | 130 |
| | | pduP_Rp] | | |
| 18 | TRGU446 | pTRGU187[sbm-argK-ygfG- | 30 | 50 |
| | | pduP_Eh] | | |
| 19 | TRGU484 | pTRGU187[sbm-argK-mme-ygfG- | 10 | 20 |
| | | pduP_Eh] | | |
| 20 | TRGU489 | pTRGU187[sbm-argK-mme-ygfG- | 40 | 110 |
| | | pduP_Rc] | | |
| 21 | TRGU491 | pTRGU187[sbm-ygfD-ygfG- | 60 | 130 |
| | | pduP_Rc] | | |
| 22 | TRGU493 | pTRGU187[mutAB-ygfD-mme- | 30 | 50 |
| | | ygfG-pduP_Rc] | | |
| 23 | TRGU495 | pTRGU187[mutAB-argK-mme- | 30 | 120 |
| | | ygfG-pduP_Rc] | | |
| 24 | TRGU497 | pTRGU187[mutAB-argK-ygfG- | 50 | 40 |
| | | pduP_Rc] | | |
| 25 | TRGU503 | pTRGU187[sbm-ygfD-mme-ygfG- | 50 | 140 |
| | | pduP_Pf_syn2a] | | |
| 26 | TRGU505 | pTRGU187[sbm-argK-mme-ygfG- | 30 | 70 |
| | | pduP_Pf_syn2a] | | |
| 27 | TRGU507 | pTRGU187[sbm-ygfD-ygfG- | 60 | 160 |
| | | pduP_Pf_syn2a] | | |
| 28 | TRGU509 | pTRGU187[sbm-argK-ygfG- | 40 | 50 |
| | | pduP_Pf_syn2a] | | |
| 29 | TRGU511 | pTRGU187[mutAB-ygfD-mme- | 30 | 100 |
| | | ygfG-pduP_Pf_syn2a] | | |
| 30 | TRGU513 | pTRGU187[mutAB-argK-mme- | 20 | 40 |
| | | ygfG-pduP_Pf_syn2a] | | |
| 31 | TRGU515 | pTRGU187[sbm-ygfD-mme-ygfG- | 50 | 140 |
| | | pduP_Pf_syn2b] | | |
| 32 | TRGU517 | pTRGU187[sbm-argK-ygfG- | 30 | 110 |
| | | pduP_Pf_syn2b] | | |

All constructs with expressed n-propanol biosynthesis genes in E. coli TOP10 (Table 21) resulted in production of n-propanol.

### Example 32

### Semi-Quantitative Analysis Using In-Gel Digest and LC-MS/MS Analysis of Strains Isolated from Example 31

Cultures from Example 31 were sampled at each indicated time point. The samples were centrifuged at 5000×g for 5 min, the supernatant discarded, and cells frozen at −20° C. Selected samples were analyzed using mass spectrometry and the relative levels of identified proteins determined according to the following procedures:

### A. Reduction and Alkylation

Each 50 μL sample was mixed with 20 μl NuPage LDS sample buffer (Prod no. NP0007), 4 μl 1 M DTT and incubated for 10 min at 95° C. The samples were subsequently allowed to cool before 6 μl 1 M iodoacetamide in 0.5M Tris-HCl pH 9.2 was added. The samples then were incubated in the dark for 20 min at room temperature.

### B. Electrophoresis

SDS-PAGE was performed for each sample in NuPAGE 4-12% Bis-Tris gels (Prod no. NP0321) using NuPAGE MES SDS Running buffer (Prod no. NP0002) according to the recommendations of the manufacturer. The gels were stained using expedeon InstantBlue™ (Prod no. ISB01L) according to the recommendation of the manufacturer.

### C. InGel Digest and Peptide Extraction

6-8 bands from each lane were cut out and each slice was transferred to a different position in a 96 well plate. The gel slices were washed ×2 with 150 μl 50% ethanol/50 mM NH₄HCO₃ for 30 min and subsequently shrunk by adding 100 μl acetonitrile. The solvent was removed after 15 min and the gel slices were dried in a SpeedVac for 10 min. The gel slices were re-swelled in 15 μl 25 mM NH₄HCO₃ containing 25 mg trypsin (Roche, prod. no. 11418475001) pr ml. 25 μl 25 mM NH₄HCO₃ was added to each well after 10-15 min. The 96 well plate then was incubated over night at 37° C. The tryptic peptides were extracted by adding 50 μl 70% acetonitrile/0.1% TFA and incubating the samples for 15 min at room temperature. The supernatants were transferred to HPLC vials and the extraction was repeated. The combined extracts were dried in a SpeedVac and reconstituted in 50 μl 5% formic acid.

### D. Mass Spectrometry

The released tryptic peptides were analyzed using an Orbitrap Velos instrument (Thermo Scientific) equipped with a Nano LC chromatographic system (Easy nLC II, Thermo Scientific). The chromatographic system was mounted with a 2 cm, ID 100 μm, 5 μm 018-A1 guard column (Proxeon, prod. no. SC001) and a 10 cm, ID 75 μm, 3 μm C18-A2 separation column (Proxeon, prod. no. SC200) and operated using the conditions shown in Table 22. 1 μL of each sample was injected for analysis.

TABLE 22

| | | | % 0.1 Formic | % 0.1 Formic acid in |
|---|---|---|---|---|
| Time | Duration | Flow | acid in water | Acetonitrile |
| (min) | (min) | nl/min | — | — |
| 0.00 | — | 300 | 95 | 5 |
| 10.00 | 10.00 | 300 | 65 | 35 |
| 12.00 | 2.00 | 300 | 0 | 100 |
| 20.00 | 8.00 | 300 | 0 | 100 |

The MS experiment was performed as an n^{th} order double play with MS/MS analysis of the top 10 peaks using HCD activation. The MS scan was performed in the Orbitrap using a resolution of 7500 and a scan range, 350-1750 m/z. The MS/MS scans were performed in the Orbitrap using the settings shown in Table 23 (only enabled settings are listed).

TABLE 23

| Parameter | Setting | |
|---|---|---|
| Activation type | HCD | |
| Minimum signal required | 5000 | |
| Isolation width | 4.00 | m/z |
| Normalized collision energy | 40 | |
| Default charge stage | 2 | |
| Activation time | 0.100 | msec. |
| FT first mass value | 100 | m/z |
| Lock mass | 445.120025 | m/z |
| FT master scan preview | Enabled | |
| Charge state screening | Enabled | |
| Monoisotopic precursor selection | Enabled | |
| Charge state rejection | Enabled | |
| Unassigned charge state | Rejected | |
| Charge state 1 | Rejected | |
| Predict ion injection time | Enabled | |
| Dynamic exclusion | Enabled | |
| Repeat count | 1 | |
| Repeat duration | 30 | sec. |
| Exclusion list size | 500 | |
| Exclusion duration | 90 | sec. |
| Exclusion mass width relative to low | 10 | ppm |
| Exclusion mass width relative to high | 10 | ppm |

| | |
|---|---|
| Expiration count | 2 |
| Expiration S/N Threshold | 2.0 |

### E. Data Base Searches

Raw files were submitted to sequence searches using Mascot and Mascot deamon ver. 2.3.0 and in-house genome databases which included sequences for the relevant heterologous proteins. Raw files from each lane were merged. The emPAI values were extracted from the Mascot search results and the mol % was calculated according to Ishihama, Y et al (Yasushi Ishihama et al. (2005) Molecular & Cellular Proteomics, 4, 1265-1272). The settings shown in Table 24 were used for the Mascot search.

TABLE 24

| Parameter | Setting |
|---|---|
| Enzyme | Trypsin |
| Max. missed cleavage | 3 |
| Peptide tolerance | 10 ppm |
| MS/MS tolerance | 0.02 Da |
| Fixed modifications | Carbamidomethyl (C) |
| Variable modifications | Oxidation (M) |
| Significance threshold | p < 0.05 |

### MS Results of Selected Samples

Selected samples were analyzed twice to confirm the results, such as MS 1, and MS 7. The obtained results are listed in Table 25.

TABLE 25

| | | | | emPAI content |
|---|---|---|---|---|
| Sample | Strain | Sample from | Proteins | (mol %) |
| MS 1 | TRGU187 | Table 21 | empty vector | 1: ArgK: 0.09 |
| | | | | 2: None Observed |
| MS 2 | TRGU302 | Table 21 | PduP_Pf_syn2a | 0.20 |
| MS 3 | TRGU304 | Table 21 | PduP_Pf_syn2a | 0.06 |
| MS 4 | TRGU412 | Table 21 | Sbm | 1.74 |
| | | | YgfD | 0.77 |
| | | | YgfG | 2.57 |
| MS 5 | TRGU507 | Table 21 | Sbm | 1.06 |
| | | | YgfD | 0.84 |
| | | | YgfG | 1.03 |
| | | | PduP_Pf_syn2a | 0.61 |
| MS 6 | TRGU509 | Table 21 | Sbm | 1.66 |
| | | | ArgK | 0.92 |
| | | | YgfG | 1.99 |
| | | | PduP_Pf_syn2a | 1.17 |
| MS 7 | TRGU434 | Table 21 | Sbm | 1.20/0.48 |
| | | | YgfD | 0.65/0.29 |
| | | | YgfG | 1.48/2.73 |
| | | | PduP_Rp | 0.15/0.14 |
| MS 8 | TRGU462 | Table 20 | MutA | Not detected |
| | | | MutB | Not detected |
| | | | YgfD | 0.25 |
| | | | YgfG | 1.33 |
| | | | PduP_Eh | 1.00 |
| MS 9 | TRGU422 | Table 21 | MutA | Not detected |
| | | | MutB | Not detected |
| | | | ArgK | 0.21 |
| | | | YgfG | Not detected |

The results in Table 25 indicate that expression of pduP_Pf_syn2a is higher in E. coli TOP10 from the high copy number plasmid pTrc99A in TRGU302 than from the low copy number plasmid pTRGU304 (Table 21). In all cases except TRGU462 and TRGU422, the produced proteins were detected by MS.

### Example 33

### Production of n-Propanol in Recombinant E. coli MG1655 Containing Various Heterologous n-Propanol Pathway Gene Combinations

E. coli MG1655 harboring plasmids from pTRGU409 to pTRGU517 listed in Table 19 were grown individually overnight with shaking (250 rpm) at 37° C. in 10 ml MM containing 10 μg/ml kanamycin and 1 mM isopropyl-beta-thio galactopyranoside. A 2 ml sample from each medium was withdrawn after 17, 44, and 116 hours. Each sample was analyzed using gas chromatography as outlined herein. The results of the cultivation experiment are listed in Table 26.

TABLE 26

| Sample | Strain | Genotype | E. coli | 17 hours | 44 hours | 116 hours |
|---|---|---|---|---|---|---|
| Medium | — | — | — | 0 | — | — |
| 1 | TRGU267 | pTrc99A[NO GENES] | MG1655 | 0 | 0 | 0 |
| 2 | TRGU269 | pTRGU88[NO GENES] | MG1655 | 0 | 0 | 0 |
| 4 | TRGU531 | pTRGU88[pduP_Pf_syn2a] | MG1655 | 0 | 0 | 0 |
| 5 | TRGU551 | pTRGU88[pduP_Pf_syn2b] | MG1655 | 10 | 10 | 0 |
| 6 | TRGU543 | pTRGU88[pduP_Rp] | MG1655 | 0 | 0 | 20 |
| 7 | TRGU545 | pTRGU88[pduP_Rc] | MG1655 | 30 | 20 | 0 |
| 8 | TRGU547 | pTRGU88[pduP_Rr] | MG1655 | 10 | 20 | 0 |
| 9 | TRGU549 | pTRGU88[pduP_Eh] | MG1655 | 10 | 10 | 0 |
| 11 | TRGU553 | pTRGU187[sbm-ygfD-mme-ygfG- | MG1655 | 90 | 110 | 70 |
| | | pduP_Rp] | | | | |
| 12 | TRGU555 | pTRGU187[sbm-ygfD-mme-ygfG- | MG1655 | 50 | 70 | 50 |
| | | pduP_Rc] | | | | |
| 13 | TRGU557 | pTRGU187[sbm-ygfD-mme-ygfG- | MG1655 | 40 | 40 | 20 |
| | | pduP_Rr] | | | | |
| 14 | TRGU559 | pTRGU187[sbm-ygfD-mme-ygfG- | MG1655 | 20 | 20 | 20 |
| | | pduP_Eh] | | | | |
| 15 | TRGU604 | pTRGU187[sbm-argK-mme-ygfG- | MG1655 | 30 | 30 | 20 |
| | | pduP_Rp] | | | | |
| 16 | TRGU561 | pTRGU187[sbm-argK-mme-ygfG- | MG1655 | 10 | 0 | 0 |
| | | pduP_Rr] | | | | |
| 17 | TRGU521 | pTRGU187[sbm-ygfD-ygfG- | MG1655 | 80 | 30 | 70 |
| | | pduP_Rp] | | | | |
| 18 | TRGU563 | pTRGU187[sbm-ygfD-ygfG- | MG1655 | 40 | 90 | 10 |
| | | pduP_Rr] | | | | |
| 19 | TRGU565 | pTRGU187[sbm-ygfD-ygfG- | MG1655 | 70 | 50 | 40 |
| | | pduP_Eh] | | | | |
| 20 | TRGU567 | pTRGU187[sbm-argK-ygfG- | MG1655 | 30 | 40 | 30 |
| | | pduP_Rp] | | | | |
| 21 | TRGU569 | pTRGU187[sbm-argK-ygfG- | MG1655 | 10 | 10 | 10 |
| | | pduP_Rc] | | | | |
| 22 | TRGU570 | pTRGU187[sbm-argK-ygfG- | MG1655 | 50 | 60 | 20 |
| | | pduP_Rr] | | | | |
| 23 | TRGU523 | pTRGU187[sbm-argK-ygfG- | MG1655 | 10 | 10 | 0 |
| | | pduP_Eh] | | | | |
| 24 | TRGU571 | pTRGU187[mutAB-ygfD-mme- | MG1655 | 10 | 0 | 0 |
| | | ygfG-pduP_Rp] | | | | |
| 25 | TRGU573 | pTRGU187[mutAB-ygfD-mme- | MG1655 | 10 | 10 | 0 |
| | | ygfG-pduP_Rr] | | | | |
| 26 | TRGU575 | pTRGU187[mutAB-ygfD-mme- | MG1655 | 10 | 10 | 0 |
| | | ygfG-pduP_Eh] | | | | |
| 27 | TRGU605 | pTRGU187[mutAB-argK-mme- | MG1655 | No | 0 | 0 |
| | | ygfG-pduP_Rp] | | growth | | |
| 28 | TRGU606 | pTRGU187[mutAB-argK-mme- | MG1655 | No | 0 | 0 |
| | | ygfG-pduP_Rr] | | growth | | |
| 29 | TRGU607 | pTRGU187[mutAB-argK-mme- | MG1655 | No | 0 | 0 |
| | | ygfG-pduP_Eh] | | growth | | |
| 30 | TRGU577 | pTRGU187[mutAB-ygfD-ygfG- | MG1655 | 10 | 0 | 0 |
| | | pduP_Rp] | | | | |
| 31 | TRGU579 | pTRGU187[mutAB-ygfD-ygfG- | MG1655 | 10 | 10 | 0 |
| | | pduP_Rc] | | | | |
| 32 | TRGU581 | pTRGU187[mutAB-ygfD-ygfG- | MG1655 | 10 | 10 | 0 |
| | | pduP_Rr] | | | | |
| 33 | TRGU583 | pTRGU187[mutAB-ygfD-ygfG- | MG1655 | 10 | 10 | 0 |
| | | pduP_Eh] | | | | |
| 34 | TRGU585 | pTRGU187[mutAB-argK-ygfG- | MG1655 | 10 | 0 | 0 |
| | | pduP_Rp] | | | | |
| 35 | TRGU587 | pTRGU187[mutAB-argK-ygfG- | MG1655 | 10 | 10 | 0 |
| | | pduP_Rr] | | | | |
| 36 | TRGU589 | pTRGU187[mutAB-argK-ygfG- | MG1655 | 10 | 0 | 0 |
| | | pduP_Eh] | | | | |
| 37 | TRGU591 | pTRGU187[sbm-argK-mme-ygfG- | MG1655 | 20 | 10 | 0 |
| | | pduP_Eh] | | | | |
| 38 | TRGU608 | pTRGU187[sbm-argK-mme-ygfG- | MG1655 | No | 0 | 0 |
| | | pduP_Rc] | | growth | | |
| 39 | TRGU592 | pTRGU187[sbm-ygfD-ygfG- | MG1655 | 80 | 110 | 40 |
| | | pduP_Rc] | | | | |
| 40 | TRGU594 | pTRGU187[mutAB-ygfD-mme- | MG1655 | 10 | 10 | 0 |
| | | ygfG-pduP_Rc] | | | | |
| 41 | TRGU609 | pTRGU187[mutAB-argK-mme- | MG1655 | No | 0 | 0 |
| | | ygfG-pduP_Rc] | | growth | | |
| 42 | TRGU610 | pTRGU187[mutAB-argK-ygfG- | MG1655 | 10 | 10 | 0 |
| | | pduP_Rc] | | | | |
| 43 | TRGU596 | pTRGU187[sbm-ygfD-mme-ygfG- | MG1655 | 50 | 60 | 30 |
| | | pduP_Pf_syn2a] | | | | |
| 44 | TRGU611 | pTRGU187[sbm-argK-mme-ygfG- | MG1655 | No | 0 | 0 |
| | | pduP_Pf_syn2a] | | growth | | |
| 45 | TRGU598 | pTRGU187[sbm-ygfD-ygfG- | MG1655 | 50 | 70 | 40 |
| | | pduP_Pf_syn2a] | | | | |
| 46 | TRGU600 | pTRGU187[mutAB-ygfD-mme- | MG1655 | 10 | 0 | 50 |
| | | ygfG-pduP_Pf_syn2a] | | | | |
| 47 | TRGU602 | pTRGU187[sbm-ygfD-mme-ygfG- | MG1655 | 70 | 80 | 0 |
| | | pduP_Pf_syn2b] | | | | |
| 48 | TRGU612 | pTRGU187[sbm-argK-ygfG- | MG1655 | No | 0 | 0 |
| | | pduP_Pf_syn2b] | | growth | | |

The results in Table 26 show that E. coli MG1655 is able to produce small amounts of n-propanol when transformed with the pTRGU88 expression vector containing either of the tested PduP genes. Inserting the remaining genes of the supposed n-propanol biosynthesis pathways in most cases increase the amounts of propanol produced. Also shown are several examples of gene combinations in which the propanol production is increased, compared to single pduP gene expression, such as no. 11-15, 17-20, 22, 37, 39, 43, 45, and 47. Among these, most gene combinations contain the Sbm gene as compared to the MutAB genes, although no. 46 with MutAB in combination with YgfD, Mme, YgfG, and PduP_syn2a did result in increased propanol concentration after 116 hours compared to expression of pduP_Pf_syn2a alone.

### Example 34

### Production of Isopropanol and n-Propanol in Recombinant E. coli TOP10

The expression vectors pTrc99A and pTRGU88 were simultaneously transformed into E. coli TOP10 via electroporation as described above. Transformants were selected on LB agar plates containing 200 μg/mL ampicillin and 20 μg/mL kanamycin. Selected colonies were then streaked on LB medium agar plates containing 200 μg/mL ampicillin and 20 μg/mL kanamycin and incubated at 37° C. overnight. Two colonies were picked and used for inoculating tubes of 10 mL TY bouillon medium containing 100 μg/mL ampicillin and 20 μg/mL kanamycin, and then incubated overnight at 37° C. with shaking (250 rpm). The cultures were then harvested by centrifugation and the plasmids isolated using a Qiaprep® Spin Miniprep Kit (Qiagen). The plasmids were digested with XbaI and the presence of two plasmids in each transformant was confirmed by the presence of two bands at 4176 bp and 4524 bp when analyzed with gel electrophoresis as described above. The constructed E. coli strain TRGU284 was stored in 30% glycerol at −80° C.

The expression vectors pTRGU44 (supra) and pTRGU196 (expressing a C. acetobuylicum thiolase gene, a B. subtilis succinyl-CoA:acetoacetate transferase gene, a C. beijerinckii acetoacetate decarboxylase gene, and a C. beijerinckii isopropanol dehydrogenase gene; see U.S. Provisional Patent Application No. 61/408,138, filed Oct. 29, 2010) were simultaneously transformed into E. coli TOP10 via electroporation as described above. Transformants were selected on LB agar plates containing 200 μg/mL ampicillin and 20 μg/mL kanamycin. Selected colonies were then streaked on LB medium agar plates containing 200 μg/mL ampicillin and 20 μg/mL kanamycin, and then incubated at 37° C. overnight. Two colonies were picked and used for inoculating tubes of 10 mL TY bouillon medium containing 100 μg/mL ampicillin and 20 μg/mL kanamycin, and then incubated at 37° C. with shaking (250 rpm). The cultures were then harvested by centrifugation and plasmids isolated using a Qiaprep® Spin Miniprep Kit (Qiagen). The plasmids were digested with XbaI and the presence of two plasmids in each transformant was confirmed by detection of two bands at 5676 bp and 8930 bp for pTRGU44 and pTRGU196, when analyzed with gel electrophoresis. The constructed E. coli strain TRGU261 was stored in 30% glycerol at −80° C.

E. coli strains Trc99A, TRGU44, TRGU196, and TRGU261 were incubated overnight at 37° C. with shaking (250 rpm) in 10 mL LB medium containing 100 μg/mL ampicillin and 1 mM isopropyl-beta-thio galactopyranoside (IPTG). A 0.5 mL sample of each strain was withdrawn and centrifuged at 15000×g for 1 minute using a table centrifuge and the supernatant discarded. Each strain was then resuspended in 0.5 mL minimal medium (MM) without any supplements. The samples were subsequently used to inoculate a new 10 mL culture for each strain. The cultures were incubated for 119 hours at 37° C. with shaking (250 rpm). A 2 mL sample was withdrawn at the end of the cultivations, centrifuged and the supernatant of each sample was analyzed by gas chromatography. Acetone, 1-propanol and isopropanol in fermentation broths were detectable by GC-FID using the procedures described herein. Samples were diluted 1+1 with 0.05% tetrahydrofuran in methanol and analyzed.

TABLE 27

| | | Leucine | Vitamin B12 | IPTG | |
|---|---|---|---|---|---|
| No. | Strain | (1 mM) | (5 μM) | (1 mM) | Antibiotic(s) |
| 1 | Trc99A | + | + | + | Ampicillin |
| 2 | TRGU284 | + | + | + | Ampicillin/ |
| | | | | | Kanamycin |
| 3 | TRGU44 | + | + | + | Ampicillin |
| 4 | TRGU196 | + | + | + | Kanamycin |
| 5 | TRGU261 | + | + | + | Ampicillin/ |
| | | | | | Kanamycin |

As indicated in Table 28, n-propanol was produced at 20 mg/L by E. coli TRGU44 and only trace amounts could be detected in the negative control strain E. coli Trc99A. Isopropanol was produced at 10 mg/L by E. coli TRGU196. Surprisingly, co-expression of heterologous pduP and the heterologous isopropanol pathway genes in E. coli TOP10 resulted in n-propanol produced at 20 mg/L and a 27-fold upregulation of isopropanol.

TABLE 28

| | | Production (mg/L) | |
|---|---|---|---|
| ID/Strain | Description/Constructs | n-propanol | isopropanol |
| MM + supplements | MM + supplements | 0 | 0 |
| Trc99A | pTrc99A (empty vector) | Trace amounts | 0 |
| TRGU284 | pTrc99A/pTRGU88 | 0 | 0 |
| | (empty vectors) | | |
| TRGU44 | Heterologous pduP | 20 | 0 |
| TRGU196 | Heterologous isopropanol pathway | Trace amounts | 10 |
| | genes | | |
| TRGU261 | Heterologous pduP + heterologous | 20 | 270 |
| | isopropanol pathway genes | | |

### Example 35

### Production of Isopropanol and 1-Propanol in Recombinant E. coli

Plasmids pSJ10942 and pTRGu668 were simultaneously transformed into E. coli TG1 chemically competent cells, selecting erythromycin (100 microgram/ml) and kanamycin (50 microgram/ml) resistance on LB agar plates, and further propagation in TY medium with erythromycin (100 microgram/ml) and kanamycin (20 microgram/ml) and a strain judged by restriction analysis using HindIII was deemed to contain the two plasmids was kept as SJ11046.

Plasmids pSJ10942 and pTRGu671 were simultaneously transformed into E. coli TG1 as above and two strains judged by restriction analysis using HindIII was deemed to contain the two plasmids were kept as SJ11047 and SJ11048.

Strain SJ10942 was propagated with 100 microgram/ml erythromycin and prepared for electroporation as previously described. This strain was transformed with plasmid pTRGu507 selecting erythromycin (200 microgram/ml) and kanamycin (30 microgram/ml) on LB agar plates. Two transformants deemed to contain the desired plasmids as judged by restriction analysis using HindIII, were kept as SJ11051 and SJ11052.

Strains constructed as described herein, as well as SJ10942 containing an isopropanol operon only, were inoculated directly from the frozen vials in the strain collection into 10 ml tubes with LB medium supplemented with glucose (1%) and B12 vitamin (10 microliters of a 5 mM (7.9 mg/ml) stock solution). The B12 vitamin addition was repeated after 2 days fermentation. Antibiotics were added to 100 microgram/ml for erythromycin (all strains), and 20 microgram/ml for kanamycin (strains SJ11046, -47, -48, -51 and -52).

Cultures were shaken at either 26° C., 30° C., or 37° C., as indicated in the Tables 29, 30, and 31, respectively. 1-propanol, 2-propanol, and acetone levels were measured after 1, 2 and 4 days fermentation, as previously described.

TABLE 29

| | | | | | n- | |
|---|---|---|---|---|---|---|
| | | | | isopropanol | propanol | Acetone |
| Strain | Construct(s) | SEQ ID NOs | Day | (%) | (%) | (%) |
| SJ10942 | pSJ10942: thl_Ca, | 2, 5, 8, 17, 20 | 1 | nd | nd | 0.008 |
| | scoAB_Bs, | | 2 | nd | 0.093 | 0.009 |
| | adc_Cb, adh_Cb | | 4 | nd | 0.055 | 0.051 |
| | No 1-propanol | | | | | |
| | pathway | | | | | |
| SJ11046 | pSJ10942: thl_Ca, | 2, 5, 8, 17, 20 | 1 | nd | nd | 0.008 |
| | scoAB_Bs, | 80, 82, 129, 127 | 2 | 0.000 | 0.056 | 0.006 |
| | adc_Cb, adh_Cb | | 4 | 0.000 | 0.056 | 0.025 |
| | pTRGu668: | | | | | |
| | Sbm_Ec - | | | | | |
| | YgfD_Ec - | | | | | |
| | YgfG_Ec - | | | | | |
| | PduP_Rp | | | | | |
| SJ11047 | pSJ10942: thl_Ca, | 2, 5, 8, 17, 20 | 1 | Nd | nd | 0.006 |
| | scoAB_Bs, | 80, 82, 129, 128 | 2 | 0.000 | 0.069 | 0.008 |
| | adc_Cb, adh_Cb | | 4 | 0.000 | 0.058 | 0.050 |
| SJ11048 | pTRGu671: | | 1 | Nd | nd | 0.006 |
| | Sbm_Ec - | | 2 | Nd | 0.068 | 0.007 |
| | YgfD_Ec - | | 4 | 0.000 | 0.053 | 0.030 |
| | YgfG_Ec - | | | | | |
| | PduP_Pf_syn2a | | | | | |
| SJ11051 | pSJ10942: thl_Ca, | 2, 5, 8, 17, 20 | 1 | nd | nd | 0.005 |
| | scoAB_Bs, | 79, 81, 102, 49 | 2 | 0.004 | 0.053 | 0.006 |
| | adc_Cb, adh_Cb | | 4 | 0.004 | 0.032 | 0.038 |
| SJ11052 | pTRGu507: | | 1 | nd | nd | 0.005 |
| | Sbm_Ec - | | 2 | 0.005 | 0.051 | 0.006 |
| | YgfD_Ec - | | 4 | 0.003 | 0.023 | 0.035 |
| | YgfG_Ec - | | | | | |
| | PduP_Pf_syn2a | | | | | |

“nd” means not detected; “0.000” means that the compound was detected.

TABLE 30

| | | | | | n- | |
|---|---|---|---|---|---|---|
| | | | | isopropanol | propanol | Acetone |
| Strain | Construct(s) | SEQ ID NOs | Day | (%) | (%) | (%) |
| SJ10942 | pSJ10942: thl_Ca, | 2, 5, 8, 17, 20 | 1 | nd | 0.098 | 0.010 |
| | scoAB_Bs, | | 2 | nd | 0.043 | 0.078 |
| | adc_Cb, adh_Cb | | 4 | | | |
| | No 1-propanol | | | | | |
| | pathway | | | nd | nd | 0.030 |
| SJ11046 | pSJ10942: thl_Ca, | 2, 5, 8, 17, 20 | 1 | 0.000 | 0.086 | 0.009 |
| | scoAB_Bs, | 80, 82, 129, 127 | 2 | 0.000 | 0.058 | 0.050 |
| | adc_Cb, adh_Cb | | 4 | 0.000 | 0.000 | 0.023 |
| | pTRGu668: | | | | | |
| | Sbm_Ec - | | | | | |
| | YgfD_Ec - | | | | | |
| | YgfG_Ec - | | | | | |
| | PduP_Rp | | | | | |
| SJ11047 | pSJ10942: thl_Ca, | 2, 5, 8, 17, 20 | 1 | 0.000 | 0.085 | 0.009 |
| | scoAB_Bs, | 80, 82, 129, 128 | 2 | 0.000 | 0.057 | 0.058 |
| | adc_Cb, adh_Cb | | 4 | 0.000 | 0.000 | 0.029 |
| SJ11048 | pTRGu671: | | 1 | 0.000 | 0.081 | 0.009 |
| | Sbm_Ec - | | 2 | 0.000 | 0.051 | 0.055 |
| | YgfD_Ec - | | 4 | 0.000 | nd | 0.024 |
| | YgfG_Ec - | | | | | |
| | PduP_Pf_syn2a | | | | | |
| SJ11051 | pSJ10942: thl_Ca, | 2, 5, 8, 17, 20 | 1 | 0.005 | 0.045 | 0.006 |
| | scoAB_Bs, | 79, 81, 102, 49 | 2 | 0.009 | 0.052 | 0.042 |
| | adc_Cb, adh_Cb | | 4 | 0.001 | nd | 0.017 |
| SJ11052 | pTRGu507: | | 1 | 0.004 | 0.032 | 0.005 |
| | Sbm_Ec - | | 2 | 0.008 | 0.043 | 0.038 |
| | YgfD_Ec - | | 4 | 0.000 | nd | 0.008 |
| | YgfG_Ec - | | | | | |
| | PduP_Pf_syn2a | | | | | |

“nd” means not detected; “0.000” means that the compound was detected.

TABLE 31

| | | | | | n- | |
|---|---|---|---|---|---|---|
| | | | | isopropanol | propanol | Acetone |
| Strain | Construct(s) | SEQ ID NOs | Day | (%) | (%) | (%) |
| SJ10942 | pSJ10942: thl_Ca, | 2, 5, 8, 17, 20 | 1 | nd | 0.151 | 0.040 |
| | scoAB_Bs, | | 2 | nd | 0.003 | 0.063 |
| | adc_Cb, adh_Cb | | 4 | nd | nd | 0.005 |
| | No 1-propanol | | | | | |
| | pathway | | | | | |
| SJ11046 | pSJ10942: thl_Ca, | 2, 5, 8, 17, 20 | 1 | 0.003 | 0.163 | 0.029 |
| | scoAB_Bs, | 80, 82, 129, 127 | 2 | 0.002 | 0.005 | 0.093 |
| | adc_Cb, adh_Cb | | 4 | 0.001 | nd | 0.011 |
| | pTRGu668: | | | | | |
| | Sbm_Ec - | | | | | |
| | YgfD_Ec - | | | | | |
| | YgfG_Ec - | | | | | |
| | PduP_Rp | | | | | |
| SJ11047 | pSJ10942: thl_Ca, | 2, 5, 8, 17, 20 | 1 | 0.003 | 0.163 | 0.023 |
| | scoAB_Bs, | 80, 82, 129, 128 | 2 | 0.002 | 0.006 | 0.091 |
| | adc_Cb, adh_Cb | | 4 | 0.001 | nd | 0.014 |
| SJ11048 | pTRGu671: | | 1 | 0.002 | 0.151 | 0.031 |
| | Sbm_Ec - | | 2 | 0.002 | 0.005 | 0.079 |
| | YgfD_Ec - | | 4 | 0.000 | nd | 0.010 |
| | YgfG_Ec - | | | | | |
| | PduP_Pf_syn2a | | | | | |
| SJ11051 | pSJ10942: thl_Ca, | 2, 5, 8, 17, 20 | 1 | 0.004 | 0.041 | 0.003 |
| | scoAB_Bs, | 79, 81, 102, 49 | 2 | 0.003 | 0.058 | 0.007 |
| | adc_Cb, adh_Cb | | 4 | 0.002 | 0.030 | 0.002 |
| SJ11052 | pTRGu507: | | 1 | 0.003 | 0.029 | 0.003 |
| | Sbm_Ec - | | 2 | 0.003 | 0.040 | 0.005 |
| | YgfD_Ec - | | 4 | 0.002 | 0.025 | 0.002 |
| | YgfG_Ec - | | | | | |
| | PduP_Pf_syn2a | | | | | |

“nd” means not detected; “0.000” means that the compound was detected.

Both isopropanol and n-propanol are produced from the strains harbouring both pathways, whereas no n-propanol is produced by the strain harbouring only the isopropanol pathway.

### Example 36

### Production of Isopropanol and n-Propanol in Recombinant L. reuteri from Metabolic Intermediates

Strains SJ11011, SJ11012, SJ11015, SJ11016, and SJ11024 (supra) were inoculated into 2 ml MRS with 10 microgram/ml erythromycin, in eppendorf tubes, and incubated without shaking at 37° C. overnight. 50 microliter aliquots were then used to inoculate new 2 ml MRS tubes with 10 microgram/ml erythromycin, supplemented with acetone and 1,2-propanediol at varying concentrations as indicated in the tables below. Cultures were incubated at 37° C. for two days, and supernatant samples analyzed for n-propanol, isopropanol, acetone, and 1,2-propanediol, as described above. Resulting n-propanol, isopropanol, acetone, and 1,2-propanediol levels are shown in Tables 32, 33, 34, and 35, respectively. MRS-10erm indicates culture medium that was not inoculated with any strain, but just carried through the incubation and analysis.

### Example 36

### Production of Isopropanol and n-Propanol in Recombinant L. reuteri from Metabolic Intermediates

Strains SJ11011, SJ11012, SJ11015, SJ11016, and SJ11024 (supra) were inoculated into 2 ml MRS with 10 microgram/ml erythromycin, in eppendorf tubes, and incubated without shaking at 37° C. overnight. 50 microliter aliquots were then used to inoculate new 2 ml MRS tubes with 10 microgram/ml erythromycin, supplemented with acetone and 1,2-propanediol at varying concentrations as indicated in the tables below. Cultures were incubated at 37° C. for two days, and supernatant samples analyzed for n-propanol, isopropanol, acetone, and 1,2-propanediol, as described above. Resulting n-propanol, isopropanol, acetone, and 1,2-propanediol levels are shown in Tables 32, 33, 34, and 35, respectively. MRS-10erm indicates culture medium that was not inoculated with any strain, but just carried through the incubation and analysis.

TABLE 32

| | n-propanol (%) | | | | | | | |
|---|---|---|---|---|---|---|---|---|
| | Acetone addition | | | Acetone + 1,2-propandiol addition | | | No | |
| Strain | 1 mL/L | 5 mL/L | 10 mL/L | 1 + 1 mL/L | 5 + 5 mL/L | 10 + 10 mL/L | addition | SEQ ID NOs |
| MRS- | nd | nd | nd | nd | nd | nd | nd | N/A |
| 10erm | | | | | | | | |
| SJ11011 | 0.003 | 0.003 | 0.002 | 0.083 | 0.360 | 0.710 | 0.002 | N/A |
| SJ11012 | 0.003 | 0.003 | 0.002 | 0.084 | 0.371 | 0.714 | 0.002 | |
| SJ11015 | 0.003 | 0.003 | 0.002 | 0.083 | 0.361 | 0.626 | 0.003 | 46 |
| SJ11016 | 0.003 | 0.003 | 0.002 | 0.083 | 0.365 | 0.735 | 0.002 | |
| SJ11024 | 0.003 | 0.003 | 0.003 | 0.083 | 0.297 | 0.356 | 0.003 | 20 |

“nd” means not detected.

TABLE 33

| | isopropanol (%) | | | | | | | |
|---|---|---|---|---|---|---|---|---|
| | Acetone addition | | | Acetone + 1,2-propandiol addition | | | No | |
| Strain | 1 mL/L | 5 mL/L | 10 mL/L | 1 + 1 mL/L | 5 + 5 mL/L | 10 + 10 mL/L | addition | SEQ ID NOs |
| MRS- | nd | nd | nd | nd | 0.001 | nd | nd | N/A |
| 10erm | | | | | | | | |
| SJ11011 | 0.012 | 0.024 | 0.025 | 0.012 | 0.027 | 0.015 | 0.001 | N/A |
| SJ11012 | 0.010 | 0.022 | 0.027 | 0.010 | 0.023 | 0.009 | 0.001 | |
| SJ11015 | 0.082 | 0.184 | 0.220 | 0.081 | 0.150 | 0.146 | 0.001 | 46 |
| SJ11016 | 0.083 | 0.179 | 0.261 | 0.080 | 0.149 | 0.122 | 0.001 | |
| SJ11024 | 0.081 | 0.376 | 0.690 | 0.079 | 0.377 | 0.633 | 0.001 | 20 |

“nd” means not detected.

TABLE 34

| | Acetone (%) | | | | | | | |
|---|---|---|---|---|---|---|---|---|
| | Acetone addition | | | Acetone + 1,2-propandiol addition | | | No | |
| Strain | 1 mL/L | 5 mL/L | 10 mL/L | 1 + 1 mL/L | 5 + 5 mL/L | 10 + 10 mL/L | addition | SEQ ID NOs |
| MRS- | 0.086 | 0.382 | 0.754 | 0.084 | 0.382 | 0.760 | 0.002 | N/A |
| 10erm | | | | | | | | |
| SJ11011 | 0.066 | 0.346 | 0.716 | 0.067 | 0.342 | 0.724 | 0.001 | N/A |
| SJ11012 | 0.067 | 0.341 | 0.722 | 0.069 | 0.353 | 0.733 | 0.001 | |
| SJ11015 | 0.002 | 0.188 | 0.526 | 0.002 | 0.227 | 0.606 | 0.001 | 46 |
| SJ11016 | 0.002 | 0.191 | 0.499 | 0.002 | 0.226 | 0.625 | 0.001 | |
| SJ11024 | 0.002 | 0.005 | 0.084 | 0.002 | 0.014 | 0.143 | 0.001 | 20 |

“nd” means not detected.

TABLE 35

| | 1,2-propanediol (%) | | | | | | | |
|---|---|---|---|---|---|---|---|---|
| | Acetone addition | | | Acetone + 1,2-propandiol addition | | | No | |
| Strain | 1 mL/L | 5 mL/L | 10 mL/L | 1 + 1 mL/L | 5 + 5 mL/L | 10 + 10 mL/L | addition | SEQ ID NOs |
| MRS- | nd | nd | nd | 0.121 | 0.535 | 1.167 | nd | N/A |
| 10erm | | | | | | | | |
| SJ11011 | nd | nd | nd | nd | nd | 0.020 | nd | N/A |
| SJ11012 | nd | nd | nd | nd | nd | 0.025 | nd | |
| SJ11015 | nd | nd | nd | nd | nd | 0.091 | nd | 46 |
| SJ11016 | nd | nd | nd | nd | nd | 0.047 | nd | |
| SJ11024 | nd | nd | nd | nd | 0.091 | 0.524 | nd | 20 |

“nd” means not detected.

The example demonstrates that recombinant L. reuteri is able to produce both isopropanol and 1-propanol from metabolic intermediates at titers exceeding 1 g/l in small scale batch cultures.

### Example 37

### Production of Isopropanol in Recombinant B. subtilis

The genes encoding C. acetobuylicum thiolase (SEQ ID NO: 3), B. subtilis succinyl-CoA:acetoacetate transferase (SEQ ID Nos: 6 and 9), C. beijerinckii acetoacetate decarboxylase (SEQ ID NO: 18), and C. beijerinckii alcohol dehydrogenase (SEQ ID NO: 21), were amplified by PCR from plasmid pTRGU196. The primers (see below) incorporated the amyL ribosomal binding site immidiately prior to the thiolase gene. Underlined sequences were complementary to the coding sequences of the thiolase (P265) and the alcohol dehydrogenase (P266) genes.

(SEQ ID NO: 125)
Primer P265: 5′-CCACA TTGAA AGGGG AGGAG AATCA TGAAG GAAGT TGTGA TTGCT
TCT-3′
(SEQ ID NO: 126)
Primer P266: 5′-AGTCG ACGCG GCCGC TAGCA CGCGT TATAA GATGA CAACG GCTTT
GAT-3′

The resulting fragment was modified to include suitable promoters and transformed into naturally competent B. subtilis JA1343 cells targeting the pel locus using standard procedures. Transformants were selected on LB medium plates supplemented with 0.01M KH2PO4/K2HPO4 (pH 7), 0.4% glucose, and 180 μg/ml spectinomycin. Of the resulting transformants, five were tested for isopropanol production. Of these, three transformants resulted in detectable isopropanol productions using the procedures described above, of which one resulted in 20 mg/l isopropanol.

Using a similar approach to above, the genes encoding C. acetobuylicum thiolase (SEQ ID NO: 3), B. mojavensis succinyl-CoA:acetoacetate transferase (SEQ ID Nos: 12 and 15), C. beijerinckii acetoacetate decarboxylase (SEQ ID NO: 18), and C. beijerinckii alcohol dehydrogenase (SEQ ID NO: 21), were amplified by PCR from plasmid pTRGU200 using the primers shown in SEQ ID NOs: 125 and 126.

The resulting fragment was modified and transformed into naturally competent B. subtilis JA1343 cells targeting the pel locus using standard procedures as described above. Three transformants were tested for isopropanol production and all resulted in production of 10 mg/l isopropanol. A negative control was tested and confirmed that no isopropanol was produced without the recombinant gene sequences under these conditions.

### Deposit of Biological Material

The following biological material has been deposited under the terms of the Budapest Treaty with the Deutsche Sammlung von Mikroorganismen and Zellkulturen GmbH (DSMZ), Mascheroder Weg 1 B, D-38124 Braunschweig, Germany, and given the following accession number:

| Deposit | Accession Number | Date of Deposit |
|---|---|---|
| Escherichia coli NN059298 | DSM 24122 | Oct. 26, 2010 |
| Escherichia coli NN059299 | DSM 24123 | Oct. 26, 2010 |

The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The present invention may be further described by the following numbered paragraphs:

[A1] A recombinant host cell comprising a heterologous polynucleotide encoding an aldehyde dehydrogenase, wherein the recombinant host cell is capable of producing n-propanol.
[A2] The recombinant host cell of paragraph A1, wherein the host cell is prokaryotic.
[A3] The recombinant host cell paragraph A2, wherein the host cell is a member of a genus selected from the group consisting of Bacillus, Clostridium, Enterococcus, Geobacillus, Lactobacillus, Lactococcus, Oceanobacillus, Propionibacterium, Staphylococcus, Streptococcus, Streptomyces, Campylobacter, Escherichia, Flavobacterium, Fusobacterium, Helicobacter, Ilyobacter, Neisseria, Pseudomonas, Salmonella, and Ureaplasma.
[A4] The recombinant host cell of paragraph A3, wherein the host cell is a member of the Lactobacillus genus (e.g., Lactobacillus plantarum, Lactobacillus fructivorans, or Lactobacillus reuteri), or Propionibacterium genus (e.g., Propionibacterium freudenreichii).
[A5] The recombinant host cell of any of paragraphs A1-A4, wherein the aldehyde dehydrogenase is selected from:

(a) an aldehyde dehydrogenase having at least 60% sequence identity to the mature polypeptide of SEQ ID NO: 27, 30, 33, 51, 54, 57, 60, or 63;

(b) an aldehyde dehydrogenase encoded by a polynucleotide that hybridizes under at least low stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof; and

(c) an aldehyde dehydrogenase encoded by a polynucleotide having at least 60% sequence identity to the mature polypeptide coding sequence of SEQ ID NO: 25, 26, 28, 29, 31, 32, 48, 49, 50, 52, 53, 55, 56, 58, 59, 61, or 62.

[A6] The recombinant host cell any of paragraphs A1-A5, wherein the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 27, 30, 33, 51, 54, 57, 60, or 63.
[A7] The recombinant host cell any of paragraphs A1-A6, wherein the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof.
[A8] The recombinant host cell any of paragraphs A1-A7, wherein the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 25, 26, 28, 29, 31, 32, 48, 49, 50, 52, 53, 55, 56, 58, 59, 61, or 62.
[A9] The recombinant host cell any of paragraphs A1-A8, wherein the aldehyde dehydrogenase comprises or consists of the amino acid sequence of SEQ ID NO: 27, 30, 33, 51, 54, 57, 60, or 63.
[A10] The recombinant host cell any of paragraphs A1-A9, wherein the aldehyde dehydrogenase comprises or consists of the amino acid sequence of mature polypeptide of SEQ ID NO: 27, 30, 33, 51, 54, 57, 60, or 63.
[A11] The recombinant host cell any of paragraphs A1-A10, wherein the heterologous polynucleotide encoding the aldehyde dehydrogenase is operably linked to a promoter foreign to the polynucleotide.
[A12] The recombinant host cell any of paragraphs A1-A11, wherein the cell further comprises one or more (several) heterologous polynucleotides encoding a methylmalonyl-CoA mutase; a heterologous polynucleotide encoding a methylmalonyl-CoA decarboxylase; a heterologous polynucleotide encoding a methylmalonyl-CoA epimerase; or a heterologous polynucleotide encoding an n-propanol dehydrogenase.
[A13] The recombinant host cell of paragraph A12, wherein the methylmalonyl-CoA mutase selected from:

(a) a methylmalonyl-CoA mutase having at least 60% sequence identity to the mature polypeptide of SEQ ID NO: 93;

(b) a methylmalonyl-CoA mutase encoded by a polynucleotide that hybridizes under low stringency conditions with mature polypeptide coding sequence of SEQ ID NO: 79 or 80, or the full-length complementary strand thereof; and

(c) a methylmalonyl-CoA mutase encoded by a polynucleotide having at least 60% sequence identity to mature polypeptide coding sequence of SEQ ID NO: 79 or 80.

[A14] The recombinant host cell of paragraph A13, wherein the methylmalonyl-CoA mutase is a protein complex, and wherein the one or more heterologous polynucleotides encoding the methylmalonyl-CoA mutase comprises a heterologous polynucleotide encoding a first polypeptide subunit and a heterologous polynucleotide encoding a second polypeptide subunit.
[A15] The recombinant host cell of paragraph A14, wherein the first polypeptide subunit is selected from: (a) a polypeptide having at least 60% sequence identity to the mature polypeptide SEQ ID NO: 66; (b) a polypeptide encoded by a polynucleotide that hybridizes under at least low stringency conditions with the mature polypeptide coding sequence of SEQ ID NO: 64 or 65, or the full-length complementary strand thereof; and (c) a polypeptide encoded by a polynucleotide having at least 60% sequence identity to the mature polypeptide coding sequence of SEQ ID NO: 64 or 65;

and the second polypeptide subunit is selected from: (a) a polypeptide having at least 60% sequence identity to the mature polypeptide of SEQ ID NO: 69; (b) a polypeptide encoded by a polynucleotide that hybridizes under at least low stringency conditions with the mature polypeptide coding sequence of SEQ ID NO: 67 or 68, or the full-length complementary strand thereof; and (c) a polypeptide encoded by a polynucleotide having at least 60% sequence identity the mature polypeptide coding sequence of SEQ ID NO: 67 or 68.

[A16] The recombinant host cell of any of paragraphs A12-A15, wherein the heterologous polynucleotide encoding a methylmalonyl-CoA mutase or a subunit thereof is operably linked to a foreign promoter.
[A17] The recombinant host cell of any one of paragraphs A12-A16, wherein the cell further comprises a heterologous polynucleotide encoding polypeptide that associates or complexes with the methylmalonyl-CoA mutase.
[A18] The recombinant host cell of paragraph A17, wherein, the polypeptide that associates or complexes with the methylmalonyl-CoA mutase is selected from:

(a) a polypeptide having at least 60% sequence identity to the mature polypeptide of SEQ ID NO: 72 or 94;

(b) a polypeptide encoded by a polynucleotide that hybridizes under low stringency conditions with mature polypeptide coding sequence of SEQ ID NO: 70, 71, 81, or 82, or the full-length complementary strand thereof; and

(c) a polypeptide encoded by a polynucleotide having at least 60% sequence identity to mature polypeptide coding sequence of SEQ ID NO: 70, 71, 81, or 82.

[A19] The recombinant host cell of paragraph A17 or A18, wherein the heterologous polynucleotide encoding the polypeptide that associates or complexes with the methylmalonyl-CoA mutase is operably linked to a promoter foreign to the polynucleotide.
[A20] The recombinant host cell of paragraph A12, wherein the methylmalonyl-CoA decarboxylase is selected from:

(a) a methylmalonyl-CoA decarboxylase having at least 60% sequence identity to the mature polypeptide of SEQ ID NO: 103;

(b) a methylmalonyl-CoA decarboxylase encoded by a polynucleotide that hybridizes under low stringency conditions with the mature polypeptide coding sequence of SEQ ID NO: 102, or the full-length complementary strand thereof; and

(c) a methylmalonyl-CoA decarboxylase encoded by a polynucleotide having at least 60% sequence identity to the mature polypeptide coding sequence of SEQ ID NO: 102.

[A21] The recombinant host cell of paragraph A20, wherein the heterologous polynucleotide encoding the methylmalonyl-CoA decarboxylase is operably linked to a promoter foreign to the polynucleotide.
[A22] The recombinant host cell of paragraph A12, wherein the methylmalonyl-CoA epimerase is selected from:

(a) a methylmalonyl-CoA epimerase having at least 60% sequence identity to the mature polypeptide of SEQ ID NO: 75;

(b) a methylmalonyl-CoA epimerase encoded by a polynucleotide that hybridizes under low stringency conditions with the mature polypeptide coding sequence of SEQ ID NO: 73 or 74, or the full-length complementary strand thereof; and

(c) a methylmalonyl-CoA epimerase encoded by a polynucleotide having at least 60% sequence identity to the mature polypeptide coding sequence of SEQ ID NO: 73 or 74.

[A23] The recombinant host cell of paragraph A22, wherein the heterologous polynucleotide encoding the methylmalonyl-CoA epimerase is operably linked to a promoter foreign to the polynucleotide.
[A24] The recombinant host cell of paragraph A22, wherein the heterologous polynucleotide encoding the n-propanol dehydrogenase is operably linked to a promoter foreign to the polynucleotide.
[A25] The recombinant host cell of any of paragraphs A1-A24, wherein the cell comprises a heterologous polynucleotide encoding a methylmalonyl-CoA mutase and a heterologous polynucleotide encoding a methylmalonyl-CoA decarboxylase.
[A26] The recombinant host cell of paragraph A25, wherein the cell comprises and a heterologous polynucleotide encoding an n-propanol dehydrogenase.
[A27] The recombinant host cell of paragraph A25 or A26, wherein the cell comprises a heterologous polynucleotide encoding a methylmalonyl-CoA epimerase.
[A28] A composition comprising the recombinant host cell of any of paragraphs A1-A27.
[A29] The composition of paragraph A28, wherein the medium comprises a fermentable substrate.
[A30] The composition of paragraph A29, wherein the fermentable substrate is sugarcane juice (e.g., non-sterilized sugarcane juice).
[A31] The composition of any of paragraphs A28-A30, further comprising n-propanol.
[A32] The composition of paragraph A31, wherein the n-propanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[A33] A method of producing n-propanol, comprising:

(a) cultivating the recombinant host cell of paragraphs A1-A33 in a medium under suitable conditions to produce n-propanol; and

(b) recovering the n-propanol.

[A34] The method of paragraph A33, wherein the medium is a fermentable medium.
[A35] The method of paragraph A34, wherein the fermentable medium comprises sugarcane juice (e.g., non-sterilized sugarcane juice).
[A36] The method of any of paragraphs A33-A35, wherein the produced n-propanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[A37] The method of any of paragraphs A33-A36, further comprising purifying the recovered n-propanol by distillation.
[A38] The method of any of paragraph A33-A37, further comprising purifying the recovered n-propanol by converting propionaldehyde contaminant to n-propanol in the presence of a reducing agent.
[A39] The method of any of paragraph A33-A37, wherein the resulting n-propanol is substantially pure.
[A40] A method of producing propylene, comprising:

(a) cultivating the recombinant host cell of any of paragraphs A1-A27 in a medium under suitable conditions to produce n-propanol;

(b) recovering the n-propanol;

(c) dehydrating the n-propanol under suitable conditions to produce propylene; and

(d) recovering the propylene.

[A41] The method of paragraph A40, wherein the medium is a fermentable medium.
[A42] The method of paragraph A41, wherein the fermentable medium comprises sugarcane juice (e.g., non-sterilized sugarcane juice).
[A43] The method of any of paragraphs A40-A42, wherein the produced n-propanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[A44] The method of any one of paragraphs A40-A43, wherein dehydrating the n-propanol comprises treating the n-propanol with an acid catalyst.
[B1] A recombinant host cell comprising:

thiolase activity;

succinyl-CoA:acetoacetate transferase activity;

acetoacetate decarboxylase activity; and

isopropanol dehydrogenase activity;

wherein the recombinant host cell is capable of producing isopropanol.

[B2] A recombinant host cell comprising a heterologous polynucleotide encoding a thiolase; one or more (several) heterologous polynucleotides encoding a CoA-transferase; a heterologous polynucleotide encoding an acetoacetate decarboxylase; and/or a heterologous polynucleotide encoding an isopropanol dehydrogenase, wherein the recombinant host cell is capable of producing isopropanol.
[B3] The recombinant host cell of paragraph B1 or B2, wherein the host cell is prokaryotic.
[B4] The recombinant host cell paragraph B3, wherein the host cell is a member of a genus selected from the group consisting of Bacillus, Clostridium, Enterococcus, Geobacillus, Lactobacillus, Lactococcus, Oceanobacillus, Propionibacterium, Staphylococcus, Streptococcus, Streptomyces, Campylobacter, Escherichia, Flavobacterium, Fusobacterium, Helicobacter, Ilyobacter, Neisseria, Pseudomonas, Salmonella, and Ureaplasma.
[B5] The recombinant host cell of paragraph B4, wherein the host cell is a member of the Lactobacillus genus (e.g., Lactobacillus plantarum, Lactobacillus fructivorans, or Lactobacillus reuteri), or Propionibacterium genus (e.g., Propionibacterium freudenreichii).
[B6] The recombinant host cell of any of paragraphs B1-B5, wherein the cell comprises a heterologous polynucleotide encoding a thiolase.
[B7] The recombinant host cell of any of paragraphs B1-B6, wherein the cell comprises one or more (several) heterologous polynucleotides encoding a CoA-transferase.
[B8] The recombinant host cell of any of paragraphs B1-B7, wherein the cell comprises a heterologous polynucleotide encoding an acetoacetate decarboxylase.
[B9] The recombinant host cell of any of paragraphs B1-B8, wherein the cell comprises a heterologous polynucleotide encoding an isopropanol dehydrogenase.
[B10] The recombinant host cell of any of paragraphs B1-B9, wherein the cell comprises a heterologous polynucleotide encoding a thiolase; one or more (several) polynucleotides encoding a CoA-transferase; a heterologous polynucleotide encoding an acetoacetate decarboxylase; and a heterologous polynucleotide encoding an isopropanol dehydrogenase.
[B11] The recombinant host cell of any of paragraphs B7-B10, wherein the thiolase is selected from:

(a) a thiolase having at least 60% sequence identity to the mature polypeptide of SEQ ID NO: 3, 35, 114, or 116;

(b) a thiolase encoded by a polynucleotide that hybridizes under at least low stringency conditions with the mature polypeptide coding sequence of SEQ ID NO: 1, 2, 34, 113, or 115, or the full-length complementary strand thereof; and

(c) a thiolase encoded by a polynucleotide having at least 60% sequence identity to the mature polypeptide coding sequence of SEQ ID NO: 1, 2, 34, 113, or 115.

[B12] The recombinant host cell of any of paragraphs B7-B10, wherein the heterologous polynucleotide encoding the thiolase is operably linked to a promoter foreign to the polynucleotide.
[B13] The recombinant host cell of any of paragraphs B7-B12, wherein the CoA-transferase is a succinyl-CoA:acetoacetate transferase.
[B14] The recombinant host cell of any of paragraphs B7-B12, wherein the CoA-transferase is an acetoacetyl-CoA transferase.
[B15] The recombinant host cell of any of paragraphs B7-B14, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 6; (b) a polypeptide encoded by a polynucleotide that hybridizes under at least low stringency conditions with the mature polypeptide coding sequence of SEQ ID NO: 4 or 5, or the full-length complementary strand thereof; and (c) a polypeptide encoded by a polynucleotide having at least 60% sequence identity to the mature polypeptide coding sequence of SEQ ID NO: 4 or 5;

and the second polypeptide subunit is selected from: (a) a polypeptide having at least 60% sequence identity to the mature polypeptide of SEQ ID NO: 9; (b) a polypeptide encoded by a polynucleotide that hybridizes under at least low stringency conditions with the mature polypeptide coding sequence of SEQ ID NO: 7 or 8, or the full-length complementary strand thereof; and (c) a polypeptide encoded by a polynucleotide having at least 60% sequence identity to the mature polypeptide coding sequence of SEQ ID NO: 7 or 8.

[B16] The recombinant host cell of any of paragraphs B7-B14, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 12; (b) a polypeptide encoded by a polynucleotide that hybridizes under at least low stringency conditions with the mature polypeptide coding sequence of SEQ ID NO: 10 or 11, or the full-length complementary strand thereof; and (c) a polypeptide encoded by a polynucleotide having at least 60% sequence identity to the mature polypeptide coding sequence of SEQ ID NO: 10 or 11;

and the second polypeptide subunit is selected from: (a) a polypeptide having at least 60% sequence identity to the mature polypeptide of SEQ ID NO: 15; (b) a polypeptide encoded by a polynucleotide that hybridizes under at least low stringency conditions with the mature polypeptide coding sequence of SEQ ID NO: 13 or 14, or the full-length complementary strand thereof; and (c) a polypeptide encoded by a polynucleotide having at least 60% sequence identity to the mature polypeptide coding sequence of SEQ ID NO: 13 or 14.

[B17] The recombinant host cell of any of paragraphs B7-B14, wherein the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 37; (b) a polypeptide encoded by a polynucleotide that hybridizes under at least low stringency conditions with the mature polypeptide coding sequence of SEQ ID NO: 36, or the full-length complementary strand thereof; and (c) a polypeptide encoded by a polynucleotide having at least 60% sequence identity to the mature polypeptide coding sequence of SEQ ID NO: 36;

and the second polypeptide subunit is selected from: (a) a polypeptide having at least 60% sequence identity to the mature polypeptide of SEQ ID NO: 39; (b) a polypeptide encoded by a polynucleotide that hybridizes under at least low stringency conditions with the mature polypeptide coding sequence of SEQ ID NO: 38, or the full-length complementary strand thereof; and (c) a polypeptide encoded by a polynucleotide having at least 60% sequence identity to the mature polypeptide coding sequence of SEQ ID NO: 38.

[B18] The recombinant host cell of any of paragraphs B7-B14, wherein

the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 41; (b) a polypeptide encoded by a polynucleotide that hybridizes under at least low stringency conditions with the mature polypeptide coding sequence of SEQ ID NO: 40, or the full-length complementary strand thereof; and (c) a polypeptide encoded by a polynucleotide having at least 60% sequence identity to the mature polypeptide coding sequence of SEQ ID NO: 40;

and the second polypeptide subunit is selected from: (a) a polypeptide having at least 60% sequence identity to the mature polypeptide of SEQ ID NO: 43; (b) a polypeptide encoded by a polynucleotide that hybridizes under at least low stringency conditions with the mature polypeptide coding sequence of SEQ ID NO: 42, or the full-length complementary strand thereof; and (c) a polypeptide encoded by a polynucleotide having at least 60% sequence identity to the mature polypeptide coding sequence of SEQ ID NO: 42.

[B18] The recombinant host cell of any of paragraphs B7-B14, wherein the one or more (several) heterologous polynucleotides encoding a CoA-transferase are operably linked to a foreign promoter.
[B19] The recombinant host cell of any of paragraphs B8-B18, wherein the acetoacetate decarboxylase is selected from:

(a) an acetoacetate decarboxylase having at least 60% sequence identity to the mature polypeptide of SEQ ID NO: 18, 45, 118, or 120;

(b) an acetoacetate decarboxylase encoded by a polynucleotide that hybridizes under at least low stringency conditions with the mature polypeptide coding sequence of SEQ ID NO: 16, 17, 44, 117, or 119, or the full-length complementary strand thereof; and

(c) an acetoacetate decarboxylase encoded by a polynucleotide having at least 60% sequence identity to the mature polypeptide coding sequence of SEQ ID NO: 16, 17, 44, 117, or 119.

[B20] The recombinant host cell of any of paragraphs B8-B19, wherein the heterologous polynucleotide encoding the acetoacetate decarboxylase is operably linked to a promoter foreign to the polynucleotide.
[B21] The recombinant host cell of any of paragraphs B9-B20, wherein the isopropanol dehydrogenase is selected from the group consisting of:

(a) an isopropanol dehydrogenase having at least 60% sequence identity to the mature polypeptide of SEQ ID NO: 21, 24 47, or 122;

(b) an isopropanol dehydrogenase encoded by a polynucleotide that hybridizes under at least low stringency conditions with the mature polypeptide coding sequence of SEQ ID NO: 19, 20, 22, 23, 46, or 121, or the full-length complementary strand thereof; and

(c) an isopropanol dehydrogenase encoded by a polynucleotide having at least 60% sequence identity to the mature polypeptide coding sequence of SEQ ID NO: 19, 20, 22, 23, 46, or 121.

[B22] The recombinant host cell of any of paragraphs B9-B21, wherein the heterologous polynucleotide encoding the isopropanol dehydrogenase is operably linked to a promoter foreign to the polynucleotide.
[B23] A composition comprising the recombinant host cell of any of paragraphs B1-B22.
[B24] The composition of paragraph B23, wherein the medium comprises a fermentable substrate.
[B25] The composition of paragraph B24, wherein the fermentable substrate is sugarcane juice (e.g., non-sterilized sugarcane juice).
[B26] The composition of any of paragraphs B23-B25, further comprising isopropanol.
[B27] The composition of paragraph B26, wherein the isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[B28] A method of producing isopropanol, comprising:

(a) cultivating the recombinant host cell of paragraphs B1-B22 in a medium under suitable conditions to produce isopropanol; and

(b) recovering the isopropanol.

[B29] The method of paragraph B28, wherein the medium is a fermentable medium.
[B30] The method of paragraph B29, wherein the fermentable medium comprises sugarcane juice (e.g., non-sterilized sugarcane juice).
[B31] The method of any of paragraphs B28-B30, wherein the produced isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[B32] The method of any of paragraphs B28-B31, further comprising purifying the recovered isopropanol by distillation.
[B33] The method of any of paragraph B28-B32, further comprising purifying the recovered isopropanol by converting acetone contaminant to isopropanol in the presence of a reducing agent.
[B34] The method of any of paragraph B28-B33, wherein the resulting isopropanol is substantially pure.
[B35] A method of producing propylene, comprising:

(a) cultivating the recombinant host cell of any of paragraphs B1-B22 in a medium under suitable conditions to produce isopropanol;

(b) recovering the isopropanol;

(c) dehydrating the isopropanol under suitable conditions to produce propylene; and

(d) recovering the propylene.

[B36] The method of paragraph B35, wherein the medium is a fermentable medium.
[B37] The method of paragraph B36, wherein the fermentable medium comprises sugarcane juice (e.g., non-sterilized sugarcane juice).
[B38] The method of any of paragraphs B35-B37, wherein the produced isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[B39] The method of any one of paragraphs B35-B38, wherein dehydrating the n-propanol comprises treating the n-propanol with an acid catalyst.
[C1] A recombinant host cell capable of producing n-propanol and isopropanol.
[C2] The recombinant host cell of paragraph C1, comprising:

thiolase activity;

CoA-transferase activity;

acetoacetate decarboxylase activity;

isopropanol dehydrogenase activity; and

aldehyde dehydrogenase activity;

wherein the host cell is capable of producing n-propanol and isopropanol.
[C3] The recombinant host cell of paragraph C1 or C2, comprising:

a heterologous polynucleotide encoding a thiolase;

one or more (several) heterologous polynucleotides encoding a CoA-transferase;

a heterologous polynucleotide encoding an acetoacetate decarboxylase;

a heterologous polynucleotide encoding an isopropanol dehydrogenase; and

a heterologous polynucleotide encoding an aldehyde dehydrogenase;

wherein the host cell is capable of producing n-propanol and isopropanol.

[C4] The recombinant host cell of paragraph C3, further comprising:

one or more (several) heterologous polynucleotides encoding a methylmalonyl-CoA mutase;

a heterologous polynucleotide encoding a methylmalonyl-CoA decarboxylase;

a heterologous polynucleotide encoding a methylmalonyl-CoA epimerase; and/or

a heterologous polynucleotide encoding an n-propanol dehydrogenase.

[C5] The recombinant host cell of any of paragraphs C1-C4, wherein the host cell is prokaryotic.
[C6] The recombinant host cell paragraph C5, wherein the host cell is a member of a genus selected from the group consisting of Bacillus, Clostridium, Enterococcus, Geobacillus, Lactobacillus, Lactococcus, Oceanobacillus, Propionibacterium, Staphylococcus, Streptococcus, Streptomyces, Campylobacter, Escherichia, Flavobacterium, Fusobacterium, Helicobacter, Ilyobacter, Neisseria, Pseudomonas, Salmonella, and Ureaplasma.
[C7] The recombinant host cell of paragraph C6, wherein the host cell is a member of the Lactobacillus genus (e.g., Lactobacillus plantarum, Lactobacillus fructivorans, or Lactobacillus reuteri), or Propionibacterium genus (e.g., Propionibacterium freudenreichii).
[C8] The recombinant host cell of any of paragraphs C3-C7, wherein the aldehyde dehydrogenase is selected from:

(a) an aldehyde dehydrogenase having at least 60% sequence identity to the mature polypeptide of SEQ ID NO: 27, 30, 33, 51, 54, 57, 60, or 63;

(b) an aldehyde dehydrogenase encoded by a polynucleotide that hybridizes under at least low stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof; and

(c) an aldehyde dehydrogenase encoded by a polynucleotide having at least 60% sequence identity to the mature polypeptide coding sequence of SEQ ID NO: 25, 26, 28, 29, 31, 32, 48, 49, 50, 52, 53, 55, 56, 58, 59, 61, or 62.

[C9] The recombinant host cell any of paragraphs C3-C8, wherein the aldehyde dehydrogenase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 27, 30, 33, 51, 54, 57, 60, or 63.
[C10] The recombinant host cell any of paragraphs C3-C9, wherein the aldehyde dehydrogenase is encoded by a polynucleotide that hybridizes under at least low stringency conditions, e.g., medium stringency conditions, medium-high stringency conditions, high stringency conditions, or very high stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof.
[C11] The recombinant host cell any of paragraphs C3-C10, wherein the aldehyde dehydrogenase is encoded by a polynucleotide having at least 60%, e.g., at least 65%, at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 25, 26, 28, 29, 31, 32, 48, 49, 50, 52, 53, 55, 56, 58, 59, 61, or 62.
[C12] The recombinant host cell any of paragraphs C3-C11, wherein the aldehyde dehydrogenase comprises or consists of the amino acid sequence of SEQ ID NO: 27, 30, 33, 51, 54, 57, 60, or 63.
[C13] The recombinant host cell any of paragraphs C3-C12, wherein the aldehyde dehydrogenase comprises or consists of the amino acid sequence of mature polypeptide of SEQ ID NO: 27, 30, 33, 51, 54, 57, 60, or 63.
[C14] The recombinant host cell any of paragraphs C3-C13, wherein the heterologous polynucleotide encoding the aldehyde dehydrogenase is operably linked to a promoter foreign to the polynucleotide.
[C15] The recombinant host cell of any of paragraphs C3-C14, wherein the thiolase is selected from:

(a) a thiolase having at least 60% sequence identity to the mature polypeptide of SEQ ID NO: 3, 35, 114, or 116;

(b) a thiolase encoded by a polynucleotide that hybridizes under at least low stringency conditions with the mature polypeptide coding sequence of SEQ ID NO: 1, 2, 34, 113, or 115, or the full-length complementary strand thereof; and

(c) a thiolase encoded by a polynucleotide having at least 60% sequence identity to the mature polypeptide coding sequence of SEQ ID NO: 1, 2, 34, 113, or 115.

[C16] The recombinant host cell of any of paragraphs C3-C15, wherein the heterologous polynucleotide encoding the thiolase is operably linked to a promoter foreign to the polynucleotide.
[C17] The recombinant host cell of any of paragraphs C3-C16, wherein the CoA-transferase is a succinyl-CoA:acetoacetate transferase.
[C18] The recombinant host cell of any of paragraphs C3-C16, wherein the CoA-transferase is an acetoacetyl-CoA transferase.
[C19] The recombinant host cell of any of paragraphs C3-C18, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 6; (b) a polypeptide encoded by a polynucleotide that hybridizes under at least low stringency conditions, or the full-length complementary strand thereof; and (c) a polypeptide encoded by a polynucleotide having at least 60% sequence identity to the mature polypeptide coding sequence of SEQ ID NO: 4 or 5;

and the second polypeptide subunit is selected from: (a) a polypeptide having at least 60% sequence identity to the mature polypeptide of SEQ ID NO: 9; (b) a polypeptide encoded by a polynucleotide that hybridizes under at least low stringency conditions with the mature polypeptide coding sequence of SEQ ID NO: 7 or 8, or the full-length complementary strand thereof; and (c) a polypeptide encoded by a polynucleotide having at least 60% sequence identity to the mature polypeptide coding sequence of SEQ ID NO: 7 or 8.

[C20] The recombinant host cell of any of paragraphs C3-C18, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 12; (b) a polypeptide encoded by a polynucleotide that hybridizes under at least low stringency conditions with the mature polypeptide coding sequence of SEQ ID NO: 10 or 11, or the full-length complementary strand thereof; and (c) a polypeptide encoded by a polynucleotide having at least 60% sequence identity to the mature polypeptide coding sequence of SEQ ID NO: 10 or 11;

and the second polypeptide subunit is selected from: (a) a polypeptide having at least 60% sequence identity to the mature polypeptide of SEQ ID NO: 15; (b) a polypeptide encoded by a polynucleotide that hybridizes under at least low stringency conditions with the mature polypeptide coding sequence of SEQ ID NO: 13 or 14, or the full-length complementary strand thereof; and (c) a polypeptide encoded by a polynucleotide having at least 60% sequence identity to the mature polypeptide coding sequence of SEQ ID NO: 13 or 14.

[C21] The recombinant host cell of any of paragraphs C3-C18, wherein the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 37; (b) a polypeptide encoded by a polynucleotide that hybridizes under at least low stringency conditions with the mature polypeptide coding sequence of SEQ ID NO: 36, or the full-length complementary strand thereof; and (c) a polypeptide encoded by a polynucleotide having at least 60% sequence identity to the mature polypeptide coding sequence of SEQ ID NO: 36;

and the second polypeptide subunit is selected from: (a) a polypeptide having at least 60% sequence identity to the mature polypeptide of SEQ ID NO: 39; (b) a polypeptide encoded by a polynucleotide that hybridizes under at least low stringency conditions with the mature polypeptide coding sequence of SEQ ID NO: 38, or the full-length complementary strand thereof; and (c) a polypeptide encoded by a polynucleotide having at least 60% sequence identity to the mature polypeptide coding sequence of SEQ ID NO: 38.

[C22] The recombinant host cell of any of paragraphs C3-C18, wherein

the CoA-transferase is a protein complex having acetoacetyl-CoA transferase activity comprising a heterologous polynucleotide encoding a first polypeptide subunit, and the heterologous polynucleotide encoding a second polypeptide subunit,

wherein the first polypeptide subunit is selected from: (a) a polypeptide having at least 60% sequence identity to the mature polypeptide of SEQ ID NO: 41; (b) a polypeptide encoded by a polynucleotide that hybridizes under at least low stringency conditions with the mature polypeptide coding sequence of SEQ ID NO: 40, or the full-length complementary strand thereof; and (c) a polypeptide encoded by a polynucleotide having at least 60% sequence identity to the mature polypeptide coding sequence of SEQ ID NO: 40;

and the second polypeptide subunit is selected from: (a) a polypeptide having at least 60% sequence identity to the mature polypeptide of SEQ ID NO: 43; (b) a polypeptide encoded by a polynucleotide that hybridizes under at least low stringency conditions with the mature polypeptide coding sequence of SEQ ID NO: 42, or the full-length complementary strand thereof; and (c) a polypeptide encoded by a polynucleotide having at least 60% sequence identity to the mature polypeptide coding sequence of SEQ ID NO: 42.

[C23] The recombinant host cell of any of paragraphs C3-C22, wherein the one or more (several) heterologous polynucleotides encoding a CoA-transferase are operably linked to a foreign promoter.
[C24] The recombinant host cell of any of paragraphs C3-C23, wherein the acetoacetate decarboxylase is selected from:

(a) an acetoacetate decarboxylase having at least 60% sequence identity to the mature polypeptide of SEQ ID NO: 18, 45, 118, or 120;

(b) an acetoacetate decarboxylase encoded by a polynucleotide that hybridizes under at least low stringency conditions with the mature polypeptide coding sequence of SEQ ID NO: 16, 17, 44, 117, or 119, or the full-length complementary strand thereof; and

(c) an acetoacetate decarboxylase encoded by a polynucleotide having at least 60% sequence identity to the mature polypeptide coding sequence of SEQ ID NO: 16, 17, 44, 117, or 119.

[C25] The recombinant host cell of any of paragraphs C3-C24, wherein the heterologous polynucleotide encoding the acetoacetate decarboxylase is operably linked to a promoter foreign to the polynucleotide.
[C26] The recombinant host cell of any of paragraphs C3-C25, wherein the isopropanol dehydrogenase is selected from the group consisting of:

(a) an isopropanol dehydrogenase having at least 60% sequence identity to the mature polypeptide of SEQ ID NO: 21, 24, 47, or 122;

(b) an isopropanol dehydrogenase encoded by a polynucleotide that hybridizes under at least low stringency conditions with the mature polypeptide coding sequence of SEQ ID NO: 19, 20, 22, 23, 46, or 121, or the full-length complementary strand thereof; and

(c) an isopropanol dehydrogenase encoded by a polynucleotide having at least 60% sequence identity to the mature polypeptide coding sequence of SEQ ID NO: 19, 20, 22, 23, 46, or 121.

[C27] The recombinant host cell of any of paragraphs C3-C26, wherein the heterologous polynucleotide encoding the isopropanol dehydrogenase is operably linked to a promoter foreign to the polynucleotide.
[C28] The recombinant host cell of any of paragraphs C1-C27, wherein the host cell is capable of isopropanol and/or n-propanol volumetric productivity greater than about 0.1 g/L per hour, e.g., greater than about 0.2 g/L per hour, 0.5 g/L per hour, 0.75 g/L per hour, 1.0 g/L per hour, 1.25 g/L per hour, 1.5 g/L per hour, 1.75 g/L per hour, 2.0 g/L per hour, 2.25 g/L per hour, 2.5 g/L per hour, or 3.0 g/L per hour.
[C29] A composition comprising the recombinant host cell of any of paragraphs C1-C28.
[C30] The composition of paragraph C29, wherein the medium comprises a fermentable substrate.
[C31] The composition of paragraph C30, wherein the fermentable substrate is sugarcane juice (e.g., non-sterilized sugarcane juice).
[C32] The composition of any of paragraphs C29-C31, further comprising isopropanol and/or n-propanol.
[C33] The composition of paragraph C32, wherein the isopropanol and/or n-propanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[C34] A method of producing n-propanol and isopropanol, comprising:

(a) cultivating the recombinant host cell of paragraphs C1-C28 in a medium under suitable conditions to produce n-propanol and isopropanol; and

(b) recovering the n-propanol and isopropanol.

[C35] The method of paragraph C34, wherein the medium is a fermentable medium.
[C36] The method of paragraph C35, wherein the fermentable medium comprises sugarcane juice (e.g., non-sterilized sugarcane juice).
[C37] The method of any of paragraphs C34-C36, wherein the produced n-propanol and/or isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[C38] The method of any of paragraphs C34-C37, further comprising purifying the recovered n-propanol and isopropanol by distillation.
[C39] The method of any of paragraph C34-C38, further comprising purifying the recovered n-propanol and isopropanol by converting propionaldehyde contaminant to n-propanol and/or converting acetone contaminant to isopropanol in the presence of a reducing agent.
[C40] The method of any of paragraph C34-C39, wherein the resulting n-propanol and isopropanol is substantially pure.
[C41] A method of producing propylene, comprising:

(a) cultivating the recombinant host cell of any of paragraphs C1-C28 in a medium under suitable conditions to produce n-propanol and isopropanol;

(b) recovering the n-propanol and isopropanol;

(c) dehydrating the n-propanol and isopropanol under suitable conditions to produce propylene; and

(d) recovering the propylene.

[C42] The method of paragraph C41, wherein the medium is a fermentable medium.
[C43] The method of paragraph C42, wherein the fermentable medium comprises sugarcane juice (e.g., non-sterilized sugarcane juice).
[C44] The method of any of paragraphs C41-C43, wherein the produced n-propanol and/or isopropanol is at a titer greater than about 0.01 g/L, e.g., greater than about 0.02 g/L, 0.05 g/L, 0.075 g/L, 0.1 g/L, 0.5 g/L, 1 g/L, 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
[C45] The method of any one of paragraphs C41-C43, wherein dehydrating the n-propanol and isopropanol comprises treating the n-propanol and isopropanol with an acid catalyst.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A recombinant Lactobacillus host cell comprising:
a heterologous polynucleotide encoding a thiolase;
one or more heterologous polynucleotides encoding a CoA-transferase;
a heterologous polynucleotide encoding an acetoacetate decarboxylase; and
a heterologous polynucleotide encoding an isopropanol dehydrogenase,
wherein the recombinant host cell is capable of producing isopropanol.

2. The recombinant host cell of claim 1, wherein the cell is a Lactobacillus plantarum, Lactobacillus fructivorans, or Lactobacillus reuteri cell.

3. The recombinant host cell of claim 1, wherein the thiolase is selected from:
(a) a thiolase having at least 80% sequence identity to the mature polypeptide of SEQ ID NO: 3, 35, 114, or 116;
(b) a thiolase encoded by a polynucleotide that hybridizes under at least medium-high stringency conditions with the mature polypeptide coding sequence of SEQ ID NO: 1, 2, 34, 113, or 115, or the full-length complementary strand thereof; and
(c) a thiolase encoded by a polynucleotide having at least 80% sequence identity to the mature polypeptide coding sequence of SEQ ID NO: 1, 2, 34, 113, or 115.

4. The recombinant host cell of claim 1, wherein the heterologous polynucleotide encoding the thiolase is operably linked to a promoter foreign to the polynucleotide.

5. The recombinant host cell of claim 1, wherein the CoA-transferase is a succinyl-CoA:acetoacetate transferase.

6. The recombinant host cell of claim 5, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,
wherein the first polypeptide subunit is selected from: (a) a polypeptide having at least 80% sequence identity to the mature polypeptide of SEQ ID NO: 6; (b) a polypeptide encoded by a polynucleotide that hybridizes under at least medium-high stringency conditions with the mature polypeptide coding sequence of SEQ ID NO: 4 or 5, or the full-length complementary strand thereof; and (c) a polypeptide encoded by a polynucleotide having at least 80% sequence identity to the mature polypeptide coding sequence of SEQ ID NO: 4 or 5; and
wherein the second polypeptide subunit is selected from: (a) a polypeptide having at least 80% sequence identity to the mature polypeptide of SEQ ID NO: 9; (b) a polypeptide encoded by a polynucleotide that hybridizes under at least medium-high stringency conditions with the mature polypeptide coding sequence of SEQ ID NO: 7 or 8, or the full-length complementary strand thereof; and (c) a polypeptide encoded by a polynucleotide having at least 80% sequence identity to the mature polypeptide coding sequence of SEQ ID NO: 7 or 8.

7. The recombinant host cell of claim 5, wherein the CoA-transferase is a protein complex having succinyl-CoA:acetoacetate transferase activity comprising a heterologous polynucleotide encoding a first polypeptide subunit, and the heterologous polynucleotide encoding a second polypeptide subunit,
wherein the first polypeptide subunit is selected from: (a) a polypeptide having at least 80% sequence identity to the mature polypeptide of SEQ ID NO: 12; (b) a polypeptide encoded by a polynucleotide that hybridizes under at least medium-high stringency conditions with the mature polypeptide coding sequence of SEQ ID NO: 10 or 11, or the full-length complementary strand thereof; and (c) a polypeptide encoded by a polynucleotide having at least 80% sequence identity to the mature polypeptide coding sequence of SEQ ID NO: 10 or 11;
and the second polypeptide subunit is selected from: (a) a polypeptide having at least 80% sequence identity to the mature polypeptide of SEQ ID NO: 15; (b) a polypeptide encoded by a polynucleotide that hybridizes under at least medium-high stringency conditions with the mature polypeptide coding sequence of SEQ ID NO: 13 or 14, or the full-length complementary strand thereof; and (c) a polypeptide encoded by a polynucleotide having at least 80% sequence identity to the mature polypeptide coding sequence of SEQ ID NO: 13 or 14.

8. The recombinant host cell of claim 1, wherein the CoA-transferase is an acetoacetyl-CoA transferase.

9. The recombinant host cell of claim 8, wherein the CoA-transferase is a protein complex having acetoacetyl-CoA transferase activity comprising a heterologous polynucleotide encoding a first polypeptide subunit, and the heterologous polynucleotide encoding a second polypeptide subunit,
wherein the first polypeptide subunit is selected from: (a) a polypeptide having at least 80% sequence identity to the mature polypeptide of SEQ ID NO: 37; (b) a polypeptide encoded by a polynucleotide that hybridizes under at least medium-high stringency conditions with the mature polypeptide coding sequence of SEQ ID NO: 36, or the full-length complementary strand thereof; and (c) a polypeptide encoded by a polynucleotide having at least 80% sequence identity to the mature polypeptide coding sequence of SEQ ID NO: 36;
and the second polypeptide subunit is selected from: (a) a polypeptide having at least 80% sequence identity to the mature polypeptide of SEQ ID NO: 39; (b) a polypeptide encoded by a polynucleotide that hybridizes under at least medium-high stringency conditions with the mature polypeptide coding sequence of SEQ ID NO: 38, or the full-length complementary strand thereof; and (c) a polypeptide encoded by a polynucleotide having at least 80% sequence identity to the mature polypeptide coding sequence of SEQ ID NO: 38.

10. The recombinant host cell of claim 8, wherein the CoA-transferase is a protein complex having acetoacetyl-CoA transferase activity comprising a heterologous polynucleotide encoding a first polypeptide subunit, and the heterologous polynucleotide encoding a second polypeptide subunit,
wherein the first polypeptide subunit is selected from: (a) a polypeptide having at least 80% sequence identity to the mature polypeptide of SEQ ID NO: 41; (b) a polypeptide encoded by a polynucleotide that hybridizes under at least medium-high stringency conditions with the mature polypeptide coding sequence of SEQ ID NO: 40, or the full-length complementary strand thereof; and (c) a polypeptide encoded by a polynucleotide having at least 80% sequence identity to the mature polypeptide coding sequence of SEQ ID NO: 40;
and the second polypeptide subunit is selected from: (a) a polypeptide having at least 80% sequence identity to the mature polypeptide of SEQ ID NO: 43; (b) a polypeptide encoded by a polynucleotide that hybridizes under at least medium-high stringency conditions with the mature polypeptide coding sequence of SEQ ID NO: 42, or the full-length complementary strand thereof; and (c) a polypeptide encoded by a polynucleotide having at least 80% sequence identity to the mature polypeptide coding sequence of SEQ ID NO: 42.

11. The recombinant host cell of claim 1, wherein the one or more heterologous polynucleotides encoding a CoA-transferase are operably linked to a foreign promoter.

12. The recombinant host cell of claim 1, wherein the acetoacetate decarboxylase is selected from:
(a) an acetoacetate decarboxylase having at least 80% sequence identity to the mature polypeptide of SEQ ID NO: 18, 45, 118, or 120;
(b) an acetoacetate decarboxylase encoded by a polynucleotide that hybridizes under at least medium-high stringency conditions with the mature polypeptide coding sequence of SEQ ID NO: 16, 17, 44, 117, or 119, or the full-length complementary strand thereof; and
(c) an acetoacetate decarboxylase encoded by a polynucleotide having at least 80% sequence identity to the mature polypeptide coding sequence of SEQ ID NO: 16, 17, 44, 117, or 119.

13. The recombinant host cell of claim 1, wherein the heterologous polynucleotide encoding the acetoacetate decarboxylase is operably linked to a promoter foreign to the polynucleotide.

14. The recombinant host cell of claim 1, wherein the isopropanol dehydrogenase is selected from the group consisting of:
(a) an isopropanol dehydrogenase having at least 80% sequence identity to the mature polypeptide of SEQ ID NO: 21, 24 47, or 122;
(b) an isopropanol dehydrogenase encoded by a polynucleotide that hybridizes under at least medium-high stringency conditions with the mature polypeptide coding sequence of SEQ ID NO: 19, 20, 22, 23, 46, or 121, or the full-length complementary strand thereof; and
(c) an isopropanol dehydrogenase encoded by a polynucleotide having at least 80% sequence identity to the mature polypeptide coding sequence of SEQ ID NO: 19, 20, 22, 23, 46, or 121.

15. The recombinant host cell of claim 1, wherein the heterologous polynucleotide encoding the isopropanol dehydrogenase is operably linked to a promoter foreign to the polynucleotide.

16. The recombinant host cell of claim 1, further comprising a heterologous polynucleotide encoding an aldehyde dehydrogenase, and wherein the host cell is capable of producing n-propanol.

17. The recombinant host cell of claim 16, wherein the aldehyde dehydrogenase is selected from:
(a) an aldehyde dehydrogenase having at least 80% sequence identity to the mature polypeptide of SEQ ID NO: 27, 30, 33, 51, 54, 57, 60, or 63;
(b) an aldehyde dehydrogenase encoded by a polynucleotide that hybridizes under at least medium-high stringency conditions with the mature polypeptide coding sequence of SEQ ID NO: 25, 26, 28, 29, 31, 32, 48, 49, 50, 52, 53, 55, 56, 58, 59, 61, or 62, or the full-length complementary strand thereof; and
(c) an aldehyde dehydrogenase encoded by a polynucleotide having at least 80% sequence identity to the mature polypeptide coding sequence of SEQ ID NO: 25, 26, 28, 29, 31, 32, 48, 49, 50, 52, 53, 55, 56, 58, 59, 61, or 62.

18. The recombinant host cell claim 16, wherein the heterologous polynucleotide encoding the aldehyde dehydrogenase is operably linked to a promoter foreign to the polynucleotide.

19. The recombinant host cell of claim 16, further comprising:
one or more (several) heterologous polynucleotides encoding a methylmalonyl-CoA mutase;
a heterologous polynucleotide encoding a methylmalonyl-CoA decarboxylase;
a heterologous polynucleotide encoding a methylmalonyl-CoA epimerase; and/or
a heterologous polynucleotide encoding an n-propanol dehydrogenase.

20. A method of producing isopropanol, comprising:
(a) cultivating the recombinant host cell of claim 1 in a medium under suitable conditions to produce isopropanol; and
(b) recovering the isopropanol.

21. A method of producing isopropanol and n-propanol, comprising:
(a) cultivating the recombinant host cell of claim 16 in a medium under suitable conditions to produce isopropanol and n-propanol; and
(b) recovering the isopropanol and n-propanol.

22. A method of producing propylene, comprising:
(a) cultivating the recombinant host cell of claim 1 in a medium under suitable conditions to produce isopropanol and/or n-propanol;
(b) recovering the isopropanol and/or n-propanol;
(c) dehydrating the isopropanol and/or n-propanol under suitable conditions to produce propylene; and
(d) recovering the propylene.

23. A method of claim 20, wherein the medium is a fermentable medium comprising sugarcane juice.


SEQUENCE LISTING (129 sequences)

SEQ ID NO: 1 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 2 - DNA, 1179 bp, Clostridium acetobutylicum
atgaaagaggtcgtcattgcatcggctgtgcgtactgctattggttcatacggtaagagtttgaaagacgtaccagcggtcgacttaggcgcaactgccatcaaagaagcagttaagaaagcagggattaagccggaagacgttaacgaagttattctggggaacgtcttgcaagccggtcttggtcaaaatccagcgcgtcaggcatctttcaaagcgggtcttccagtagaaattcctgccatgaccatcaacaaggtatgcggtagtggcctccgtaccgtctctctagctgctcagatcatcaaagcgggagacgccgatgttatcattgctggtggtatggaaaacatgagtcgggcaccctacctagcaaacaacgcacgttggggataccgtatgggtaacgccaaattcgtcgatgaaatgattactgacggcctctgggacgcattcaacgactaccacatgggaatcactgccgaaaacattgccgaacgttggaacattagtcgcgaagaacaagatgagttcgcgttggcgagccaaaagaaagctgaagaagcaatcaaatcaggtcaattcaaagatgaaattgttcctgtggttatcaaaggtcgcaaaggcgaaacggttgtcgacactgacgaacatccgcgtttcggaagtacgatcgaaggactcgcgaagttgaaacctgccttcaagaaggacggcaccgtgacggctggcaacgcaagcggcttaaacgactgcgcagccgtcctcgtcattatgagtgccgagaaagccaaagaactgggtgtgaaaccgctcgccaagattgttagctacggctctgctggtgttgacccagccattatgggttatggccctttctacgctactaaagccgctattgaaaaagccggatggaccgtagacgaacttgacctgattgaatcgaacgaggctttcgctgctcagtccttagccgtagcaaaagacttgaagttcgacatgaacaaagttaacgtaaacggtggtgccattgctttgggacacccgattggtgcctcgggtgctcggatcttagttacgctggttcacgcaatgcaaaagcgtgacgctaagaaagggttggcaacattatgcatcggtggtggacaaggcacagcgatccttcttgaaaagtgctag

SEQ ID NO: 3 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 4 - DNA, 702 bp, Bacillus subtilis
atgggaaaaggaaaagtgatggactcttatcaagatgctgctgcactgatcaaggacggagacaccctcatcgcaggaggcttcggcctgtgcggcattccggaacaattaattctcgcgatcagggacagcggcgtaaaaaacttgaccgttgtcagtaataactgcggtgttgatgattgggggttaggcctcttgcttgcaaaccggcagatcaaaaaaatggtcgcatcctatgtgggagaaaacaaaacctttgaacggcagtttttaagcggggagctggaagtcgaattggtgccccaaggaacgctcgcagaaagaattcgcgcgggaggggcgggcattccggcgttttacacacctgcgggcgtcggaacatcagtagccgagggaaaagaacataagacatttgacggacgcacctatcttctggagaaagggattacagggaacgccgccatcgtaaaggcctggaaagccgatcctctgggcaacctgatgttcagaaagacggcgagaaatttcaatccgctcgctgcaatggcaggcaaagtgacgattgcagaggctgaagaaatcgtcgatgccggagagcttgatccggatcaaattcatacgccgggtatttttgtgcagcacgtgctgctcggcgggattcatgaaaaacggattgaacgccgcaccgtccggcaagcatga

SEQ ID NO: 5 - DNA, 702 bp, Bacillus subtilis
atgggtaagggtaaagtaatggattcttatcaagatgcagcagcgttgatcaaagatggtgacaccttaatcgcaggcggttttggcctttgtggtattcctgagcagttaatccttgctatccgtgacagtggcgttaagaacttgaccgttgtgtccaacaattgtggggttgatgattgggggttgggtctgttgctcgccaatcggcagatcaagaagatggtcgcctcttatgtgggtgaaaacaaaacttttgaacgccagttcttaagcggtgaattagaagttgaacttgttcctcaaggaacgctggctgagcggattcgtgcaggtggtgcagggattcctgccttctatactcctgcgggtgtgggtacaagcgtcgcggaaggtaaggaacacaaaacctttgatggtcggacctatcttttggaaaaaggtatcaccggtaatgcagccattgtgaaagcctggaaagccgacccacttggtaatcttatgtttcggaaaactgcacgcaacttcaatccattagcagcaatggctggtaaagtgaccattgctgaagccgaagaaattgtcgatgctggtgaacttgatccagatcagattcacactccagggattttcgttcagcatgttttgcttggtggcatccatgagaaacgtattgaacgtcgtacggtccgtcaggcatag

SEQ ID NO: 6 - protein, 233 aa, Bacillus subtilis
MGKGKVMDSYQDAAALIKDGDTLIAGGFGLCGIPEQLILAIRDSGVKNLTVVSNNCGVDDWGLGLLLANRQIKKMVASYVGENKTFERQFLSGELEVELVPQGTLAERIRAGGAGIPAFYTPAGVGTSVAEGKEHKTFDGRTYLLEKGITGNAAIVKAWKADPLGNLMFRKTARNFNPLAAMAGKVTIAEAEEIVDAGELDPDQIHTPGIFVQHVLLGGIHEKRIERRTVRQA

SEQ ID NO: 7 - DNA, 651 bp, Bacillus subtilis
gtgaaggaagcgagaaaacgaatggtcaaacgggctgtacaagaaatcaaggacggcatgaatgtgaatctcgggattggaatgccgacgcttgtcgcaaatgagatacccgatggcgttcacgtcatgcttcagtcggaaaacggcttgctcggaattggcccctatcctctggaaggaacggaagacgcggatttgatcaatgcgggaaaggaaacgatcactgaagtgacaggcgcctcttattttgacagcgctgagtcattcgcgatgataagaggcgggcatatcgatttagctattctcggcggaatggaggtttcggagcagggggatttggccaattggatgatcccgggcaaaatggtaaaagggatgggcggcgccatggatctcgtcaacggggcgaaacgaatcgttgtcatcatggagcacgtcaataagcatggtgaatcaaaggtgaaaaaaacatgctcccttccgctgacaggccagaaagtcgtacacaggctgattacggatttggctgtatttgattttgtgaacggccgcatgacactgacggagcttcaggatggtgtcacaattgaagaggtttatgaaaaaacagaagctgatttcgctgtaagccagtctgtactcaattcttaa

SEQ ID NO: 8 - DNA, 651 bp, Bacillus subtilis
atgaaggaagctcgtaagcgtatggttaagcgtgcggttcaggaaatcaaagacggtatgaacgttaacttaggcatcgggatgccgacacttgttgcaaacgaaattccggacggtgtacacgttatgttacagagtgaaaacggattgttgggtattggtccctacccactcgaaggcaccgaagacgctgacttgatcaatgccggaaaggaaacgattacggaagtgactggcgcatcttacttcgacagtgcggagtcgttcgccatgattcgtggtggacacattgacctggctatcctcggtggaatggaggtgtctgaacagggggacttagcgaactggatgattccaggcaaaatggtcaagggtatgggtggtgcaatggacttggtcaatggcgctaaacggatcgtcgtgatcatggaacacgttaacaagcacggtgaaagtaaggttaagaagacatgctccttaccgttgacgggacagaaggtggttcaccgcttgattaccgacctcgcagttttcgacttcgttaacggacgtatgactcttacggaactacaggacggtgtcacgatcgaagaagtttacgaaaagactgaagcagacttcgctgtgtcccagagtgtgctcaacagttag

SEQ ID NO: 9 - protein, 216 aa, Bacillus subtilis
  misc_feature (1)..(1): Xaa can be any naturally occurring amino acid
XKEARKRMVKRAVQEIKDGMNVNLGIGMPTLVANEIPDGVHVMLQSENGLLGIGPYPLEGTEDADLINAGKETITEVTGASYFDSAESFAMIRGGHIDLAILGGMEVSEQGDLANWMIPGKMVKGMGGAMDLVNGAKRIVVIMEHVNKHGESKVKKTCSLPLTGQKVVHRLITDLAVFDFVNGRMTLTELQDGVTIEEVYEKTEADFAVSQSVLNS

SEQ ID NO: 10 - DNA, 714 bp, Bacillus mojavensis
atgggaaaagtgctgtcatcgagtaaggaagccgcggaactcattcgggagggggatacactgatcgcgggcggattcggcctgtgcggaattcccgagcagctcattctggcgataagggataaaggggtaaaagatttaaccgtcgtcagcaataattgcggagttgatgattgggggctcggtctgctgctggcaaacaagcaaatcaaaaaaatgatcgcttcctacgtcggagaaaacaaaatttttgaaaagcaatttttaagcggagaattggaagtggaattggttccccaagggaccctcgctgaaagaatccgagccggaggagcgggtataccgggattttacacagccacaggcgtcggaacatctatcgctgacgggaaagagcataaaacctttgacggacgcacttatgtgttagaaaaagggattactggggatgtcgccattgtaaaagcatggaaagcggacaccatggggaatttagtttttcggaaaacggcaagaaatttcaatccggttgccgccatggcgggaaagatcacaattgccgaggcagaagaaattgttgaggcgggagagctcgatcccgaccacatacacacgcctggtatttacgtacagcatgttgtgctcggcacacatgaaaagcggattgaaaaacgaactgttcagcaagcggagggaaaggaggcggcacaatga

SEQ ID NO: 11 - DNA, 714 bp, Bacillus mojavensis
atggggaaagtactcagttctagcaaagaagctgcggagttaatccgtgagggcgatacacttattgcgggtggtttcggcctctgcggtattccagaacagttgattttggccattcgggataaaggtgtaaaggatctgacagttgtcagcaataactgtggggttgatgactgggggttgggtctcttgttagcgaacaaacagatcaagaaaatgattgcttcgtatgtgggcgaaaacaagatctttgaaaagcagtttttgtcaggagagcttgaagtggaacttgtcccacaagggaccctagccgaacggattcgtgccggtggcgctggtattcctggcttctatacagctacaggagtaggaacatctatcgctgatggtaaagaacacaaaacgttcgatggtcgtacctatgtgctcgaaaagggtattactggggacgtggccattgtgaaagcgtggaaggctgatacgatgggtaatctagtgtttcggaagactgcccgtaacttcaatcctgttgctgcaatggcaggcaagattacgatcgctgaagcggaagaaatcgtagaagcgggtgaattagatcctgaccacattcatactccaggcatctacgtacagcatgtagtattgggtacacatgaaaaacgtatcgaaaaacgcacggtacaacaggccgaaggcaaagaagcagcgcaatag

SEQ ID NO: 12 - protein, 237 aa, Bacillus mojavensis
MGKVLSSSKEAAELIREGDTLIAGGFGLCGIPEQLILAIRDKGVKDLTVVSNNCGVDDWGLGLLLANKQIKKMIASYVGENKIFEKQFLSGELEVELVPQGTLAERIRAGGAGIPGFYTATGVGTSIADGKEHKTFDGRTYVLEKGITGDVAIVKAWKADTMGNLVFRKTARNFNPVAAMAGKITIAEAEEIVEAGELDPDHIHTPGIYVQHVVLGTHEKRIEKRTVQQAEGKEAAQ

SEQ ID NO: 13 - DNA, 657 bp, Bacillus mojavensis
atgaaggaagccagaaaacgaatggtcaaacgtgctgtaaaggaaataaaagacggtatgaacgtcaatcttgggatagggatgccgacacttgtggcaaatgaaataccggagggcgttcatgtgatgcttcaatcagaaaacggcttgcttgggatcggcccgtatccgctggacggaacggaagacccggatctgatcaatgcggggaaagaaacgatcaccgccgtaacaggcgcatcctattttgacagcgcagaatcctttgcgatgatacgaggcggtcatatcgacctggctatcctcgggggcatggaggtttctgagcaaggggatttggcgaactggatgatcccggggaaaatggtgaagggaatgggcggcgctatggatttggtcaacggggctaagcgaatcgttgtcatcatggagcacgtcaataaacatggggaatcgaaggtgaaaaaacaatgctccctcccgctgacaggacagaaagtcgttcatcggctgatcactgatttagctgtttttgattttgataacggccatatgacactgactgagctccaggacggcgtcacgctggaagaggtatatgagaaaactgaagctgacttcgccgtaagccagtcagtcatccggcaaaaatcttaa

SEQ ID NO: 14 - DNA, 657 bp, Bacillus mojavensis
atgaaagaagcccgtaagcgcatggttaagcgtgccgtaaaggaaatcaaggacgggatgaacgttaacttgggtattggtatgccgacacttgtagcaaacgaaattcctgaaggtgttcacgtcatgctccagagtgagaacggtcttttaggtattggtccctacccattagacggcacagaggacccagacttgattaacgcaggtaaagaaactatcaccgctgtaactggggcaagttacttcgactcagcagaatctttcgctatgattcgtggtgggcacatcgacttggcaattcttggtggtatggaagtttcagaacagggggacttggctaactggatgattccaggcaaaatggttaaggggatgggaggagctatggacttggtaaatggcgcaaagcggattgtggtcattatggaacacgttaacaagcacggtgaatcaaaggtcaagaaacagtgctcccttccattgactggtcagaaggttgttcaccgtttgatcaccgacctcgctgtattcgacttcgacaacggacacatgactttgaccgagttacaggacggtgtaacactagaagaggtttacgagaaaacagaagccgacttcgcagttagccagagtgttatccgtcagaaatcgtag

SEQ ID NO: 15 - protein, 218 aa, Bacillus mojavensis
MKEARKRMVKRAVKEIKDGMNVNLGIGMPTLVANEIPEGVHVMLQSENGLLGIGPYPLDGTEDPDLINAGKETITAVTGASYFDSAESFAMIRGGHIDLAILGGMEVSEQGDLANWMIPGKMVKGMGGAMDLVNGAKRIVVIMEHVNKHGESKVKKQCSLPLTGQKVVHRLITDLAVFDFDNGHMTLTELQDGVTLEEVYEKTEADFAVSQSVIRQKS

SEQ ID NO: 16 - DNA, 741 bp, Clostridium beijerinckii
atgttagaaagtgaagtatctaaacaaattacaactccacttgctgctccagcgtttcctagaggaccatataggtttcacaatagagaatatctaaacattatttatcgaactgatttagatgctcttcgaaaaatagtaccagagccacttgaattagatagagcatatgttagatttgaaatgatggctatgcctgatacaaccggactaggctcatatacagaatgtggtcaagctattccagtaaaatataatggtgttaagggtgactacttgcatatgatgtatctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 17 - DNA, 741 bp, Clostridium beijerinckii
atgctcgaaagtgaagtttctaaacagattactaccccattagcggcaccagcatttccccgtggtccataccgtttccacaaccgtgagtacttgaacatcatttaccgtacggacttagacgctttgcgcaagatcgttccagaaccgctcgaacttgaccgtgcttacgttcgtttcgaaatgatggcaatgccagacacgaccggtctcggctcatacacggaatgtgggcaagccattcccgtgaaatacaacggtgttaaaggagactacttacacatgatgtacttggataacgaacctgcaattgcagttggtcgcgaatcttccgcgtaccccaagaaactcggataccctaaacttttcgttgactcggacacgttggtaggtacgttgaaatacgggacactgccagtcgccacagccacaatgggttacaaacacgaacctcttgaccttaaggaagcatacgcccaaattgcgcgtcccaacttcatgttgaagatcattcaggggtacgacggcaaaccacggatttgcgaacttatctgcgcagaaaacaccgacattacaattcatggggcttggacaggtagtgctcgcctccagttattcagtcacgccttagccccactggcagacttacctgtacttgaaatcgtatcagcaagccacatccttaccgacttgactttgggtacaccgaaagttgtacacgactacctcagtgtcaagtag

SEQ ID NO: 18 - protein, 246 aa, Clostridium beijerinckii
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 19 - DNA, 1056 bp, Clostridium beijerinckii
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 20 - DNA, 1056 bp, Clostridium beijerinckii
atgaagggattcgccatgctagggattaacaagttaggttggattgagaaagaacgtccagtagcagggagctacgacgccatcgttcgtccccttgcggtttccccttgcacttcggacattcacaccgttttcgaaggcgctctgggcgaccgcaaaaacatgatcttagggcacgaagccgtaggtgaagtggtggaagtcgggagtgaggttaaagatttcaaacctggggatcgtgtcattgtaccgtgcacaactccggactggcgctcgttggaagtacaggctggattccaacagcactctaacgggatgttggctggctggaaattctcgaacttcaaagatggggtcttcggtgagtacttccacgtgaacgacgctgacatgaacttggcaattctgcctaaggacatgccactggaaaacgcagttatgattaccgacatgatgaccactgggttccacggtgcagaattagcagatatccagatgggaagttctgtggttgtaattggtattggagccgtcggcctcatggggatcgcaggtgccaaattgcgtggggcaggtcgcatcatcggagtggggtctcgtcccatttgcgtagaagcggctaagttctatggtgccacggacattctcaactacaagaacggccacatcgtcgaccaagttatgaagttgactaacggcaaaggtgttgaccgggtcattatggcaggtggtggtagtgagacattaagccaagctgttagtatggttaaaccgggtggcattatcagtaacatcaactaccacggaagcggtgatgctctgctgattccgcgtgtcgagtggggttgcggcatggcacacaagacaatcaaaggtggtctatgtcccggaggtcgccttcgtgcggagatgcttcgcgacatggttgtctacaaccgggtcgacttaagcaaattggtaacacacgtgtaccacggtttcgaccacattgaagaagcactgttgttaatgaaagacaagccaaaggacttgatcaaagccgttgtgatcttatag

SEQ ID NO: 21 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 22 - DNA, 1059 bp, Thermoanaerobacter ethanolicus
atgaaaggttttgcaatgctcagtatcggtaaagtcggttggattgaaaaagaaaagcctactcccggcccttttgacgctatcgtaagacctctagctgtggccccttgcacttcggacgttcataccgtttttgaaggtgctattggcgaaagacataacatgatactcggtcacgaagctgtaggtgaagtagttgaagtaggtagtgaggtaaaagattttaaacctggtgatcgcgttgttgtaccagctattacccctgattggcgaacctctgaagtacaaagaggatatcaccaacactctggtggaatgctggcaggctggaaattttcgaatataaaagatggtgtttttggtgaattttttcatgtgaacgatgctgatatgaatttagcacatctgcctaaggaaattccattggaagctgcagttatgattcccgatatgatgactactggctttcacggagccgaactggcagaaatagaattaggtgcaacggtagcggttttgggtattggtccagtaggtcttatggcagtcgctggtgccaaattgcggggtgctggaagaattattgcagtaggcagtagacctgtttgtgtagatgctgcaaaatactatggagctactgatattgtaaactataaaaatggtcctatcgaaagccagattatggatttaacggaaggcaaaggtgttgatgctgccatcatcgctggaggaaatgctgacattatggctacagcagttaagattgttaaaccaggcggcaccatcgctaatgtaaattattttggcgaaggagatgttctgcctgttcctcgtcttgaatggggttgcggcatggctcataaagctataaaaggcggtttatgccctggtggacgtctaagaatggaaagactgattgaccttgttttttataagcgtgtcgatccttccaaactcgtcactcatgtttttcaaggatttgataatattgaaaaagctctaatgctgatgaaagataaaccaaaagacctaatcaaacctgttgtaatattagcataa

SEQ ID NO: 23 - DNA, 1059 bp, Thermoanaerobacter ethanolicus
atgaaggggtttgcaatgctctcgattggtaaggtgggttggattgaaaaagaaaaaccaacaccaggtccttttgacgccattgtacgtccgttggccgttgctccgtgcacgtcagatgttcacacggttttcgaaggggcaattggtgaacgtcataacatgatcttaggccatgaggcagttggcgaagttgtagaggtgggtagtgaggttaaggacttcaaacccggtgatcgggtagtcgttccagcaattactccggattggcgtacaagtgaggtccaacgtggctaccatcagcactccggtggtatgctcgcaggttggaaattcagcaacatcaaagatggggtatttggcgagttcttccacgttaatgatgccgacatgaacttagctcatttacctaaagaaattccattagaagcggctgttatgattcccgatatgatgacgactggctttcatggtgcggaattggcagaaattgagctcggagcgaccgttgcagtcttgggcattggccctgtaggtttgatggcagttgcgggtgcgaaacttcgtggcgctggacggatcatcgctgtgggttcgcgtccagtctgcgtagatgcagccaagtactatggagctactgatattgtcaactacaagaatgggcctattgagtctcagattatggatcttaccgagggtaaaggtgtggatgcagcgatcattgcgggagggaatgcggacattatggctactgccgtgaagattgtaaaacctggtggcacaattgcaaacgttaactactttggtgaaggtgatgtcttgccggttccacgtcttgaatgggggtgtggtatggctcacaaagctatcaagggtgggttgtgcccaggtggtcggctgcgtatggaacgcctaattgatttagttttctacaaacgcgttgatccatccaagttagttactcatgtcttccaagggttcgacaacattgaaaaagcactcatgttaatgaaagataaaccgaaggacttgattaagccggttgtcatcttagcgtag

SEQ ID NO: 24 - protein, 352 aa, Thermoanaerobacter ethanolicus
MKGFAMLSIGKVGWIEKEKPTPGPFDAIVRPLAVAPCTSDVHTVFEGAIGERHNMILGHEAVGEVVEVGSEVKDFKPGDRVVVPAITPDWRTSEVQRGYHQHSGGMLAGWKFSNIKDGVFGEFFHVNDADMNLAHLPKEIPLEAAVMIPDMMTTGFHGAELAEIELGATVAVLGIGPVGLMAVAGAKLRGAGRIIAVGSRPVCVDAAKYYGATDIVNYKNGPIESQIMDLTEGKGVDAAIIAGGNADIMATAVKIVKPGGTIANVNYFGEGDVLPVPRLEWGCGMAHKAIKGGLCPGGRLRMERLIDLVFYKRVDPSKLVTHVFQGFDNIEKALMLMKDKPKDLIKPVVILA

SEQ ID NO: 25 - DNA, 1503 bp, Propionibacterium freudenreichii
gtgcgggggcattggtgtctccggcggcacggtcgaccaggacgtcaccattgccagttttgctatgtcgcgagccaaggaagcgagtcggtcatgaccatcagcccagaactaatccagcaggtcgttcgtgaaacggtgcgtgaggtcatctcacgccaggattccggcaccgatgcccccagcggcaccgacggcatcttcaccgacatgaacagtgcggtggacgccgccgatgtggcgtggcgccagtacatggactgctcgttgcgcgaccgcaaccggttcatccaggcgatccgcgacgtggccagcgagcccgacaacctggagtacatggccaccgccaccgtcgaggagacggggatgggcaatgtgccccacaagatcctcaagaatcgctacgccgccctgtacacaccgggcaccgaggacatcatcaccgaggcctggtcgggtgacgacggcctgaccaccgtcgagttctcgcccttcggcgtgatcggggcgatcacccccacgacgaatcccaccgagacggtgatcaacaacacgatcggcatgctcgcggccggcaacgcggtggtcttcagcccccatccgcgtgccaagaagatcaccctgtggctggtgcgcaagatcaaccgcgcgctcgccgctgcgggtgcgcccgccaacctggtggtgacggtcgaggagccctccatcgacaacaccaacgccatgatgtcgcacgagaaggtgcgcatgctcgtggcaaccggcggcccgggcatcgtcaaggccgtgctgtccagcggcaagaaggccatcggtgccggtgccggcaacccgccggccgtggtggatgacaccgccgacatcgccaaggcggcccgggacatcgtggacggggccagcttcgacaacaacctcccgtgcaccgccgagaaggaggtgctggcggttgattccattgccgacctgctcaagttcgagatgctcaagcacggctgcttcgagttgaaggatcgcgccgtgatggacaagctggccgccctggtgaccaagggccagcatgccaacgccgcctatgtgggcaagccggccgcccagctcgcctccgaggtgggcctgagcgcacccaaggacacgcgcctgctgatctgcgaggtgcccttcgaccatccgttcgtgcaggtggagctgatgatgccgatcctgccgatcgtgcggatgcccgatgtcgacaccgccatcgacaaggcggtggaggtggagcacggcaaccgtcacacggccgtgatgcactcgtcgaatgtcaacgcgctgaccaagatgggcaagctcatccagaccacgatcttcgtgaagaacggtccctcgtacaacggcatcggcatcgacggtgagggctttcccaccttcaccatcgccggcccgaccggtgagggcctgacctcggcccgctgcttcgcccgcaagcgtcgttgcgtgttgaagagcggtctcaacattcgctga

SEQ ID NO: 26 - DNA, 1503 bp, Propionibacterium freudenreichii
atgcgtgggcattggtgtctgcgtcgtcatgggcgtcctggtcgccatcattgtcagttctgttatgtggcgtcacagggttcggagagcgttatgaccatcagtccggagttaatccaacaagttgtccgcgaaactgtccgtgaagttatcagccgtcaagactctggcaccgatgcaccgtcaggtactgatggcatctttaccgatatgaatagcgctgttgatgcagcggatgttgcttggcgtcagtatatggattgtagcctgcgtgatcgcaaccgtttcattcaggctatccgcgatgtggccagtgagcctgataatctggagtatatggcgactgcgaccgttgaagaaaccgggatgggcaacgtgccacacaagattttgaagaatcgttatgcagcactgtatactccaggcaccgaagacattatcaccgaggcgtggagtggcgatgacggcttaacgaccgtagaattctcaccgtttggagtgattggggcaatcacgccaacgacaaatccgacagaaacggtgatcaacaatacgatcggcatgttagcagcaggcaacgccgtggtctttagccctcacccacgtgcgaagaagattaccctgtggctggttcgcaaaatcaaccgtgcgttggctgctgccggagcccctgcgaacctggtggtgactgttgaagaaccgtccattgacaacaccaacgccatgatgagccatgaaaaagtgcgcatgctggtggccaccggtggaccaggaattgtgaaagcagttctgagctccggcaagaaagccattggcgcaggcgcaggtaatccaccagcggtggtggacgatacggcagatatcgccaaagcagctcgcgatattgtagacggtgcgagcttcgataacaacctgccgtgtactgcggagaaagaagttctggctgttgactctattgccgatttgctgaaattcgagatgctgaaacatggctgttttgagctgaaagatcgcgcagttatggataaactggctgcgctggtgacgaaggggcagcacgcaaatgcagcgtacgtgggtaaaccggcagcccagctggcttctgaagtaggcttatctgcaccgaaggatacccgcttactgatttgcgaggttccgtttgatcatccgttcgtgcaggttgaactgatgatgccgatcttaccgattgtgcgcatgcctgacgttgacaccgcaatcgataaagcagttgaagtggaacacggcaaccgccacactgcggttatgcatagcagcaacgttaacgccctgaccaaaatggggaagctgattcagactaccattttcgtgaagaatggtccgagctacaatgggattgggatcgatggtgaaggattccctacattcacaattgccggtccaacaggtgagggtttaacctctgctcgttgctttgcccgtaaacgccgttgcgtgttaaagagtggcctgaatatccgctaa

SEQ ID NO: 27 - protein, 500 aa, Propionibacterium freudenreichii
  misc_feature (1)..(1): Xaa can be any naturally occurring amino acid
XRGHWCLRRHGRPGRHHCQFCYVASQGSESVMTISPELIQQVVRETVREVISRQDSGTDAPSGTDGIFTDMNSAVDAADVAWRQYMDCSLRDRNRFIQAIRDVASEPDNLEYMATATVEETGMGNVPHKILKNRYAALYTPGTEDIITEAWSGDDGLTTVEFSPFGVIGAITPTTNPTETVINNTIGMLAAGNAVVFSPHPRAKKITLWLVRKINRALAAAGAPANLVVTVEEPSIDNTNAMMSHEKVRMLVATGGPGIVKAVLSSGKKAIGAGAGNPPAVVDDTADIAKAARDIVDGASFDNNLPCTAEKEVLAVDSIADLLKFEMLKHGCFELKDRAVMDKLAALVTKGQHANAAYVGKPAAQLASEVGLSAPKDTRLLICEVPFDHPFVQVELMMPILPIVRMPDVDTAIDKAVEVEHGNRHTAVMHSSNVNALTKMGKLIQTTIFVKNGPSYNGIGIDGEGFPTFTIAGPTGEGLTSARCFARKRRCVLKSGLNIR

SEQ ID NO: 28 - DNA, 1446 bp, Lactobacillus collinoides
atggcagatcaaaatattgaagcagaaatcagacgaattttacaagaagaattaagcggtaacgcttcgtccagcgctgctggtacgactaccagtcaacctgatgggttaggcaacgggatcttcaccaacgtgaacgatgccattgctgctgctaagcaagctcaggcaatctaccaagataaaccacttgccttccgtaaaaaagtcgttcaagcaattaaagatggtttcggcccatacattgaatatatggcaaagcagacccgtgaagaaactggcatgggaactgccgaagctaagattgctaagttaaagaacgccctctacaacaccccaggcgttgaattactggacccagaagttgaaactggtgacggcgggatggtcatgtatgaatacacgccattcggtgttatcggtgccgttggaccaagtacaaacccttgtgaaacggttctgaacaactccatcatgatgatgtctgctgggaacgcagttgtctttggcgcccatcctggtgcaaagaacattactcgctgggcagttgaaaaattgaacgaattcgtttacaaggctactgggttgaagaacctcttagtttccttggacacaccatcaattgaatccgttcaagaaatgatgcaacatccagatgttgcaatgctggctgtaactggtggcccagctgttgtgcatcaagcattaacgagtggtaaaaaagccgttggtgccggtgctggtaacccgcctgcaatggttgatgcaactgctgatattgatttagcagctcataacctatttacttcagctaagtttgacaatgaaattctgtgtacttcagaaaaggaaatcattgctgaagattcaattaaggatgaacttcttcaaaagattgttgctaagggcgcttgcctagtaactgatcctaaagacatcaagcatttagctgacatgaccattggggacaacggtgcccctgaccggaaatatgttggtaaggatgccactgttatcttagatgccgctggtatttcatacaccggcgatcctaagttgatcatgatggatgttgataaagacaacccattggttaagacagaaatgttaatgccaatcttgcctatcgttgggtgcccagactttgacgccgttttggctacggctattgaagttgaaggtggcaatcaccatactgcttcaattcactcgaacaacatcctgcacatcaacaaggctgctcaccggatgaacacctcgatcttcgtcgcaaatggcccaacatttgccgcaactggtgtcggtgataacggttattacagtggtgctgctgcgctgacaattgctaccccaaccggtgaaggtactaccactactaagacctttacccgtcgtcgtcgtttcaactgtccacaagggttctcacttcgttcttgggaggtttaa

SEQ ID NO: 29 - DNA, 1443 bp, Lactobacillus collinoides
atggccgatcagaacatcgaagcggagattcgtcgcattctgcaggaagaactgagtgggaatgcgtcttcgtccgcagcgggaaccacgacctcgcagccggatggcttgggaaatcgcatcttcaccaacgttaatgacgccattgctgccgcaaaacaggctcaagcaatctaccaagataaaccactggcttttcgcaagaaagtggttcaagccatcaaagatggttttggtccgtacattgaatacatggccaaacaaactcgggaagagactggcatgggcaccgcagaagcgaagattgcgaaactgaaaaacgctctgtacaacactccgggtgtagaactgttggatccggaagtggaaaccggtgatggtggcatggtgatgtatgaatacaccccgtttggtgtcattggagcggttggtccgtcgactaacccatgcgaaacggttctgaataacagcatcatgatgatgagtgcagggaatgcactgttctttggcgctcatccgggtgcgaagaacattacccgttgggcagtggagaagttaaacgaattcgtgtacaaagcaactggcctgaaaaacctgctggtgtcgttagatacgccgtcaattgaatccgttcaggaaatgatgcagcacccggatgtcgccatgctggcggttacaggtggaccagcggtggtccaccaagctctgacctccggcaagaaagcggtgggagcaggtgcaggcaacccaccagcaatggtggatgcaactgccgatattgatttagcagcgcataacctgttcacctcagcgaagtttgataatgaaattctgtgcaccagtgaaaaagaaatcattgccgaagactctatcaaggatgagttactgcagaagattgtagctaaaggtgcctgcttagttacggatccgaaggatatcaaacatctggcggatatgacaattggagataatggtgccccagaccgcaaatacgtaggtaaagatgccaccgtgattctggatgctgcaggcatcagctacactggcgacccgaaactgattatgatggacgttgataaagataatccgctggtgaaaactgagatgctgatgccgatcctgcctatcgtgggttgcccagattttgatgcagtgctggctaccgctatcgaggtggaaggtgggaaccatcatactgctagtattcactctaacaatatcttgcatatcaacaaagctgcccatcgcatgaacaccagcatctttgtagcgaatggccctaccttcgcagcgacgggtgtaggggataacggctactacagtggtgccgctgcgctgaccattgccacgccaactggcgaaggtaccaccaccaccaaaacattcacgcgtcgtcgtcgcttcaactgtccgcaaggcttctcgctgcgttcctgggaggtc

SEQ ID NO: 30 - protein, 481 aa, Lactobacillus collinoides
MADQNIEAEIRRILQEELSGNASSSAAGTTTSQPDGLGNRIFTNVNDAIAAAKQAQAIYQDKPLAFRKKVVQAIKDGFGPYIEYMAKQTREETGMGTAEAKIAKLKNALYNTPGVELLDPEVETGDGGMVMYEYTPFGVIGAVGPSTNPCETVLNNSIMMMSAGNALFFGAHPGAKNITRWAVEKLNEFVYKATGLKNLLVSLDTPSIESVQEMMQHPDVAMLAVTGGPAVVHQALTSGKKAVGAGAGNPPAMVDATADIDLAAHNLFTSAKFDNEILCTSEKEIIAEDSIKDELLQKIVAKGACLVTDPKDIKHLADMTIGDNGAPDRKYVGKDATVILDAAGISYTGDPKLIMMDVDKDNPLVKTEMLMPILPIVGCPDFDAVLATAIEVEGGNHHTASIHSNNILHINKAAHRMNTSIFVANGPTFAATGVGDNGYYSGAAALTIATPTGEGTTTTKTFTRRRRFNCPQGFSLRSWEV

SEQ ID NO: 31 - DNA, 1407 bp, Clostridium beijerinckii
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 32 - DNA, 1404 bp, Clostridium beijerinckii
atgaataaagatacgctgattccgacgacgaaagatctgaaagtgaaaaccaatggcgagaacattaatctgaaaaactacaaagataattcttcctgctttggcgtgtttgagaacgtggaaaacgcgatttcaagcgcggtgcatgcgcagaaaattctgtcgctgcattatactaaagaacagcgtgagaaaattattacagagatccgtaaagcggcgctgcagaacaaagaggtgctggcgactatgattctggaggaaacccatatgggccgttacgaagacaaaatcctgaaacatgaactggtggcgaaatacaccccgggcaccgaagatctgactaccacggcgtggagtggcgataatggcctgaccgtggtggaaatgtctccgtatggcgtgattggcgcgatcaccccgtccacaaacccgaccgaaaccgtgatttgcaattcgatcggcatgatcgcggcgggcaacgcggtggtgttcaatggccacccgtgtgcgaaaaagtgtgtggcgtttgcggtggaaatgattaacaaagcgattatttcctgcggcggcccggagaatctggtgaccaccattaaaaatccaacaatggagtctctggatgcgattattaaacaccctagcattaaactgctgtgcggcacgggcggccctggcatggtgaagaccctgctgaactctggcaaaaaagcgattggcgcgggcgcgggcaatccgccggtgatcgtggacgatacggcggatatcgagaaagcgggccgtagtattattgagggctgcagttttgataacaacctgccgtgtatcgcggagaaagaagtgttcgtgtttgaaaacgtggcggatgacctgatttcgaacatgctgaaaaacaacgcggtgattattaatgaagatcaggtgtccaagctgattgatctggtgctgcagaaaaataatgaaacgcaggagtatttcattaacaagaaatgggtgggcaaagatgcgaaactgtttctggacgaaatcgatgtggaaagcccgagcaatgtgaagtgtattatttgcgaggtgaacgcgaaccatccatttgtgatgacggaactgatgatgcctatcctgccaatcgtacgtgtgaaagacattgatgaggcgattaaatatgcgaaaatcgcggaacagaaccgcaagcactcggcgtatatttatagcaaaaatattgataacctgaaccgcttcgaacgtgaaatcgacaccaccatttttgtgaagaatgcgaaaagtttcgcgggcgtgggctacgaagcggaaggctttaccacattcaccattgcgggctcgacgggcgaaggcattactagcgcgcgtaacttcacccgccagcgccgttgcgtgctggcgggc

SEQ ID NO: 33 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 34 - DNA, 1179 bp, Lactobacillus reuteri
atggagaaggtttacattgttgctgctcagcgaacaccaatcggtaagtttaacggtcagttagcttcaaagtctgcagttgaattaggagcgattgcaattaaagcggctgttgaaaaggcaaagctcagcgaaaacgatattgaccaagtgttaatggggaatgttattcaagcaggaacgggacaaaatccagcgcgtcaagcatcaatggccgctggattaggtgagcaggttccggcaataacgattaatgacgtctgtgcgtcgggaatgtccagtgttgacctggcagcaagtttgattcgcgcaggacaggctaatgtaattgttgccggggggatggaaagtatgtcccaagctccatatgttcttcctaaggcacggaatggctatcgatttgggaatggaaccttgcttgatgcgatgcaaagtgatgcattaaatgatgtttatggcggttatccgatgggaattactgctgaaaatatcaatgataagtatcagattactcgtcaccaacaagacgaatttgctttgatgagccaccaacgtgcggtaaaggctcaaaaagcaggctatttcgattcagaaattgtaccagttgaagttaagcagaaacgaacaacagtgactgtaactgttgacgaagctccacgaccagatacttcactagcggctttagcaaaattaaaaccagcttttaaatctgacggaagtgtgactgcagggaatgcctcgggaattaatgatggtggagctgccttggttcttgcttctgaaagtgcagtcaaccgattaggattaactccattggctgagtggcaaggatctgcagttgttggtcttgatccagcattgatgggtttgggaccatattatgcaatcaagaagctcttaaaaaatcagcagctgtcggcagatgacgtagatacttacgaaattaatgaggcgtttgcgacccaagcccttgtctgtcagaacttgcttcatcttgatcctagggctgtcaatccttggggtggggcaattgcattaggccatcctgttgggtgttcaggagcacgaattatcgtcacgatgattagtgaaatgcatagggataatcatgaactcggcattgcttcgctgtgtgtcgggggcggaatgggtgaagcggttctgcttaagaaaatttag

SEQ ID NO: 35 - protein, 392 aa, Lactobacillus reuteri
MEKVYIVAAQRTPIGKFNGQLASKSAVELGAIAIKAAVEKAKLSENDIDQVLMGNVIQAGTGQNPARQASMAAGLGEQVPAITINDVCASGMSSVDLAASLIRAGQANVIVAGGMESMSQAPYVLPKARNGYRFGNGTLLDAMQSDALNDVYGGYPMGITAENINDKYQITRHQQDEFALMSHQRAVKAQKAGYFDSEIVPVEVKQKRTTVTVTVDEAPRPDTSLAALAKLKPAFKSDGSVTAGNASGINDGGAALVLASESAVNRLGLTPLAEWQGSAVVGLDPALMGLGPYYAIKKLLKNQQLSADDVDTYEINEAFATQALVCQNLLHLDPRAVNPWGGAIALGHPVGCSGARIIVTMISEMHRDNHELGIASLCVGGGMGEAVLLKKI

SEQ ID NO: 36 - DNA, 651 bp, Escherichia coli
atggatgctaaacagcgcattgcacgtcgtgttgcacaggagttgcgtgatggcgatattgtcaatttggggattggtctgccaactatggtggccaattacctccctgagggcattcacattaccttacagtccgaaaatggctttttgggcttaggtccagttactactgcacatcccgacttagtgaatgcaggtgggcaaccctgtggggttcttccaggtgcggcaatgttcgattccgcaatgagctttgctttgattcggggaggtcacatcgacgcgtgcgttctgggtggtctccaagtcgatgaagaagccaatttggcaaattgggttgtacctggcaaaatggtgccaggcatgggaggggctatggatttagtcacgggttcacggaaagtcatcattgcaatggaacattgcgcaaaagatggcagtgccaagattcttcggcgttgcacaatgccgttaacagcgcaacacgcagttcatatgttagttactgaactggccgttttccgcttcattgatgggaaaatgtggttaactgaaattgcagatggttgcgatttggccacagttcgcgctaaaaccgaagcgcgttttgaggttgcagccgatctcaatacccagcgtggtgacttgtag

SEQ ID NO: 37 - protein, 216 aa, Escherichia coli
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 38 - DNA, 663 bp, Escherichia coli
atgaaaaccaaattgatgacactccaagatgcaacggggttctttcgtgatggcatgaccattatggttggtggctttatgggtattgggaccccatctcgcttggttgaagcattgctcgaatctggtgtgcgggatctgaccttgattgcaaacgatactgcatttgttgatacaggtattgggcctttgattgtcaatggtcgcgtccgtaaggtgatcgcatcccatattgggacaaatcctgaaactgggcgtcgtatgatttctggcgaaatggatgttgtcctcgtaccccagggtactctcatcgaacagattcgttgtggcggtgcaggtcttggtggcttcttgacccctacgggtgttgggactgtggtcgaagaaggaaaacaaacgctcacgctcgatggcaagacgtggttattggaacgtcccttacgtgcagatttagcattgattcgtgcccaccgttgcgacaccttaggtaatctcacatatcagttaagtgcccgtaactttaacccattgattgctcttgcagccgacatcaccttagtcgaacccgatgaattagttgaaactggtgagctgcaacctgatcatattgttacaccaggtgcggttattgaccacatcattgtgagccaagagtctaaatag

SEQ ID NO: 39 - protein, 220 aa, Escherichia coli
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 40 - DNA, 657 bp, Clostridium acetobutylicum
atgaacagtaagatcatccgtttcgagaacttacgtagcttcttcaaggacggtatgaccattatgattgggggtttcttaaactgcggtactcctaccaagttgatcgacttcttagtgaacttgaacatcaagaacttaaccatcatctcaaacgacacctgctaccctaacaccggaatcggtaagttaatctcaaacaaccaggttaagaagttaatcgcctcatacatcggcagcaaccctgacaccggcaagaagctcttcaacaacgagttagaggtagagttatcacctcaggggaccttagtggagcgcattcgggcagggggtagtgggctcggaggagttttgactaagacgggattaggcaccttgatcgagaagggcaagaagaagatcagtatcaacggcaccgagtacctgcttgagttacccttaaccgctgacgtagccttaatcaagggctcgatcgtcgacgaagcgggtaacaccttctacaagggtacaacgaagaacttcaacccgtacatggctatggcagcgaagaccgttatcgtagaggccgagaacttggtttcatgcgagaaacttgagaaggagaaggctatgacacctggtgttttgatcaactacatcgtgaaggagcctgcctag

SEQ ID NO: 41 - protein, 218 aa, Clostridium acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 42 - DNA, 666 bp, Clostridium acetobutylicum
atgatcaacgacaagaacttggctaaggagattatcgccaagcgcgttgcccgtgagttaaagaacggccagctcgttaaccttggtgtcggtctcccaacgatggttgccgactacatccccaagaacttcaagattacattccagtcggaaaacggtattgttggtatgggtgccagtccaaagatcaacgaggcagacaaggacgttgttaacgcaggtggggactacaccactgtgctgcccgacggaaccttcttcgacagttctgttagtttcagccttatccgtggtggacacgttgacgtcactgttttaggtgctttgcaggtcgacgaaaaggggaacatcgcaaactggatcgttcccggaaagatgttgtcaggaatgggtggggcaatggaccttgtcaacggagcgaagaaggtgattatcgctatgcgccacacgaacaaggggcagccgaagatcttgaagaagtgcactttaccgttgacggccaagagccaggctaacctgatcgttacggaactcggtgttattgaagttatcaacgacggcttgttattgacggagattaacaagaacacaaccattgacgaaattcgctcattaactgccgctgacctcttgatcagtaacgaattacgccctatggcagtatag

SEQ ID NO: 43 - protein, 221 aa, Clostridium acetobutylicum
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 44 - DNA, 780 bp, Clostridium acetobutylicum
atgctagaaaagttaaacatcatcaagttccgtaaggttactttcatgttaaaggacgaggtcattaagcagattagcacaccgttaacatcgccagcattccctcgtggaccttacaagttccacaaccgggaatacttcaacatcgtgtaccgcaccgacatggacgcactgcgcaaggtcgttccggaaccgcttgaaattgacgaacctctagtacgtttcgagattatggcaatgcacgacaccagtgggttaggttgctacacggaaagtggtcaggcaattccagttagcttcaacggtgtcaagggcgactacttgcacatgatgtacctggacaacgaaccggctatcgcagtaggtcgtgaattgtctgcatacccaaagaagttaggctaccctaagttattcgtcgacagcgacacattagtagggacattggactacggaaagttacgggtggcaacagcaactatgggttacaagcacaaggcgcttgacgcaaacgaggcgaaggaccagatctgccgtccgaactacatgcttaagatcattcccaactacgacggctctcctcgtatttgcgaactcattaacgcaaagattacagacgttacggttcacgaagcatggactggtcctactcggctgcagttattcgaccacgcgatggcaccactgaacgacctgccagtaaaggaaatcgtctcttcatcgcacatccttgcagacatcatccttcctcgtgcagaagtaatctacgactacttgaagtag

SEQ ID NO: 45 - protein, 259 aa, Clostridium acetobutylicum
MLEKLNIIKFRKVTFMLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 46 - DNA, 1071 bp, Lactobacillus antri
atgcgtggtttcgcgatgattggtccaaacgaaaccgggttcattgaaaaggatgatccggaaatgggtccacgggatgcgttagttaaaccactagctgttgcaccatgcacctcggatatccacacggtctacgaaaacgcaattggcccacggcacaacatgatcttaggtcacgaggcttgtggcgaaatcgttgcagtgggtgatgaagtaaaagatttcaaagtaggggacaaggttttagtaccagccgtgacccctgattggagcaaattggaaagtcaagccggttacgcttctcacagtgggggtatgcttgctggttggaaattcagcaacttcaaggatggcgtgttcgcgacccgcttccacgtgaacgatgcagatggtaacttggcgcaccttccgttgggaatggacccaggtgaagcctgtatgttaagtgatatgattccgaccggattgcacgcagatgaaatggccaacgtacaatacggcgatacagtagcagttattggtattggtcccgtgggcttgatggcccttcgtggggcagtcctgcacggtgcgggtcgcattttcgctgtcggatctcgtcccaaaaccattgaagttgcaaaggagtatggcgcaaccgacatcattgattaccaccaaggtgatattgcggaccaaatcttgaagttaacggataaccagggggttgataaagttctaattgctggtggcgacactgaacacactttcgatcaagcagcgcgtatgacgaaacccggaggtgcgatttcaaacgttgcttacttgaacggtaacgatgtcttgaagatcaaagctgcagattggggagttggcatgtcgaacattaccattgatggtgggttgatgccaggtggtcgcttgcgcatggaaaaactcgcatcattgattaccaacggtcgtcttgacccatccctaatgatcacccacaagttctacggtttcgagaagattccagatgctctggagttaatgcacaacaagcctgccgatctcatcaaacctgtcgtttactgcgatgatattgattag

SEQ ID NO: 47 - protein, 356 aa, Lactobacillus antri
MRGFAMIGPNETGFIEKDDPEMGPRDALVKPLAVAPCTSDIHTVYENAIGPRHNMILGHEACGEIVAVGDEVKDFKVGDKVLVPAVTPDWSKLESQAGYASHSGGMLAGWKFSNFKDGVFATRFHVNDADGNLAHLPLGMDPGEACMLSDMIPTGLHADEMANVQYGDTVAVIGIGPVGLMALRGAVLHGAGRIFAVGSRPKTIEVAKEYGATDIIDYHQGDIADQILKLTDNQGVDKVLIAGGDTEHTFDQAARMTKPGGAISNVAYLNGNDVLKIKAADWGVGMSNITIDGGLMPGGRLRMEKLASLITNGRLDPSLMITHKFYGFEKIPDALELMHNKPADLIKPVVYCDDID

SEQ ID NO: 48 - DNA, 1407 bp, Propionibacterium freudenreichii
atgaccatcagcccagaactaatccagcaggtcgttcgtgaaacggtgcgtgaggtcatctcacgccaggattccggcaccgatgcccccagcggcaccgacggcatcttcaccgacatgaacagtgcggtggacgccgccgatgtggcgtggcgccagtacatggactgctcgttgcgcgaccgcaaccggttcatccaggcgatccgcgacgtggccagcgagcccgacaacctggagtacatggccaccgccaccgtcgaggagacggggatgggcaatgtgccccacaagatcctcaagaatcgctacgccgccctgtacacaccgggcaccgaggacatcatcaccgaggcctggtcgggtgacgacggcctgaccaccgtcgagttctcgcccttcggcgtgatcggggcgatcacccccacgacgaatcccaccgagacggtgatcaacaacacgatcggcatgctcgcggccggcaacgcggtggtcttcagcccccatccgcgtgccaagaagatcaccctgtggctggtgcgcaagatcaaccgcgcgctcgccgctgcgggtgcgcccgccaacctggtggtgacggtcgaggagccctccatcgacaacaccaacgccatgatgtcgcacgagaaggtgcgcatgctcgtggcaaccggcggcccgggcatcgtcaaggccgtgctgtccagcggcaagaaggccatcggtgccggtgccggcaacccgccggccgtggtggatgacaccgccgacatcgccaaggcggcccgggacatcgtggacggggccagcttcgacaacaacctcccgtgcaccgccgagaaggaggtgctggcggttgattccattgccgacctgctcaagttcgagatgctcaagcacggctgcttcgagttgaaggatcgcgccgtgatggacaagctggccgccctggtgaccaagggccagcatgccaacgccgcctatgtgggcaagccggccgcccagctcgcctccgaggtgggcctgagcgcacccaaggacacgcgcctgctgatctgcgaggtgcccttcgaccatccgttcgtgcaggtggagctgatgatgccgatcctgccgatcgtgcggatgcccgatgtcgacaccgccatcgacaaggcggtggaggtggagcacggcaaccgtcacacggccgtgatgcactcgtcgaatgtcaacgcgctgaccaagatgggcaagctcatccagaccacgatcttcgtgaagaacggtccctcgtacaacggcatcggcatcgacggtgagggctttcccaccttcaccatcgccggcccgaccggtgagggcctgacctcggcccgctgcttcgcccgcaagcgtcgttgcgtgttgaagagcggtctcaacattcgc

SEQ ID NO: 49 - DNA, 1407 bp, Propionibacterium freudenreichii
atgaccatcagtccggagttaatccaacaagttgtccgcgaaactgtccgtgaagttatcagccgtcaagactctggcaccgatgcaccgtcaggtactgatggcatctttaccgatatgaatagcgctgttgatgcagcggatgttgcttggcgtcagtatatggattgtagcctgcgtgatcgcaaccgtttcattcaggctatccgcgatgtggccagtgagcctgataatctggagtatatggcgactgcgaccgttgaagaaaccgggatgggcaacgtgccacacaagattttgaagaatcgttatgcagcactgtatactccaggcaccgaagacattatcaccgaggcgtggagtggcgatgacggcttaacgaccgtagaattctcaccgtttggagtgattggggcaatcacgccaacgacaaatccgacagaaacggtgatcaacaatacgatcggcatgttagcagcaggcaacgccgtggtctttagccctcacccacgtgcgaagaagattaccctgtggctggttcgcaaaatcaaccgtgcgttggctgctgccggagcccctgcgaacctggtggtgactgttgaagaaccgtccattgacaacaccaacgccatgatgagccatgaaaaagtgcgcatgctggtggccaccggtggaccaggaattgtgaaagcagttctgagctccggcaagaaagccattggcgcaggcgcaggtaatccaccagcggtggtggacgatacggcagatatcgccaaagcagctcgcgatattgtagacggtgcgagcttcgataacaacctgccgtgtactgcggagaaagaagttctggctgttgactctattgccgatttgctgaaattcgagatgctgaaacatggctgttttgagctgaaagatcgcgcagttatggataaactggctgcgctggtgacgaaggggcagcacgcaaatgcagcgtacgtgggtaaaccggcagcccagctggcttctgaagtaggcttatctgcaccgaaggatacccgcttactgatttgcgaggttccgtttgatcatccgttcgtgcaggttgaactgatgatgccgatcttaccgattgtgcgcatgcctgacgttgacaccgcaatcgataaagcagttgaagtggaacacggcaaccgccacactgcggttatgcatagcagcaacgttaacgccctgaccaaaatggggaagctgattcagactaccattttcgtgaagaatggtccgagctacaatgggattgggatcgatggtgaaggattccctacattcacaattgccggtccaacaggtgagggtttaacctctgctcgttgctttgcccgtaaacgccgttgcgtgttaaagagtggcctgaatatccgc

SEQ ID NO: 50 - DNA, 1407 bp, Propionibacterium freudenreichii
atgaccatttctccagagctgattcaacaagtagtgcgcgagactgtgcgcgaggtgatctcgcgtcaggatagcggcactgatgcaccgtcgggcaccgatggtatctttacggatatgaacagtgcggttgatgcagccgatgtggcatggcgtcagtacatggattgcagcttgcgggaccgtaaccggttcattcaagcgattcgtgacgtggcttctgaaccggacaatctggagtatatggcgactgcaacggtagaagaaaccggtatgggcaatgttcctcataagattctgaaaaaccgctatgcagcactgtacactccaggcacggaagacatcattaccgaagcgtggagtggggatgatggcctgacgaccgttgagttttccccgttcggtgtgattggtgcgattaccccgacgacgaatccgacagagacggtgattaacaatacgattggtatgttagctgcgggtaatgcggtagtgtttagtccgcatccacgtgccaagaagattaccctgtggctggtgcgcaagatcaatcgtgctttagcagcagcgggtgccccagcgaatttggtagtaaccgtagaagaaccgtcgatcgataacacgaatgccatgatgtcgcatgaaaaggtgcgtatgttagtggccactggtggtccaggcattgtgaaagcagtgctgagtagcgggaagaaagctatcggagcaggtgccggtaaccctccagccgtggtcgacgatactgccgacatcgcgaaagcagcacgcgatattgtggatggtgcgtctttcgacaacaatttgccgtgcactgcggagaaagaggtcctggcagtggattcgattgcggatctgctgaagtttgagatgctgaaacatggttgtttcgaactgaaagaccgtgcggtgatggacaagttagccgctctggtcaccaaagggcagcatgcgaacgcagcgtacgtgggtaaaccggcagcccagctggcttctgaagtaggcttatctgcaccgaaggatacccggttactgatctgcgaagttccgtttgatcatccgttcgtccaggtcgaactgatgatgcctatcctgccgatcgtgcgcatgccggatgtggataccgcaatcgacaaagctgtggaagtagagcacggcaaccgccatactgcggttatgcatagctcaaacgttaatgcgttaaccaaaatgggcaaactgattcaaacaacgatcttcgtgaagaatggtccgtcctacaacggtattggcattgatggagagggctttccaaccttcacgatcgcaggtccgactggcgaaggtctgacaagtgcacgctgctttgcacgcaaacgccgttgtgtgctgaaatcaggcctgaacattcgt

SEQ ID NO: 51 - protein, 469 aa, Propionibacterium freudenreichii
MTISPELIQQVVRETVREVISRQDSGTDAPSGTDGIFTDMNSAVDAADVAWRQYMDCSLRDRNRFIQAIRDVASEPDNLEYMATATVEETGMGNVPHKILKNRYAALYTPGTEDIITEAWSGDDGLTTVEFSPFGVIGAITPTTNPTETVINNTIGMLAAGNAVVFSPHPRAKKITLWLVRKINRALAAAGAPANLVVTVEEPSIDNTNAMMSHEKVRMLVATGGPGIVKAVLSSGKKAIGAGAGNPPAVVDDTADIAKAARDIVDGASFDNNLPCTAEKEVLAVDSIADLLKFEMLKHGCFELKDRAVMDKLAALVTKGQHANAAYVGKPAAQLASEVGLSAPKDTRLLICEVPFDHPFVQVELMMPILPIVRMPDVDTAIDKAVEVEHGNRHTAVMHSSNVNALTKMGKLIQTTIFVKNGPSYNGIGIDGEGFPTFTIAGPTGEGLTSARCFARKRRCVLKSGLNIR

SEQ ID NO: 52 - DNA, 1392 bp, Rhodopseudomonas palustris
atggtggccaaggcgatccgcgaccacgccggaactgcgcagccgtccggtaacgccgcgacgtcgtcggcggcggtcagcgatggcgtgttcgagaccatggatgccgcggtcgaggccgctgctctggcgcagcagcaatacctgctttgctcgatgtccgaccgcgcgcgcttcgtgcagggaatccgcgacgtcatcttgaatcaggatacgctcgaaaagatgtcccgcatggcggtcgaagaaaccggcatgggcaattacgagcataagctgatcaagaaccggctagccggcgaaaagaccccgggcatcgaagatctcaccaccgacgctttcagcggcgacaacggtctgacgctggtggaatactcgccgttcggggtgatcggcgcgatcacgccgaccaccaatccgacggaaaccatcgtctgcaattcgatcggaatgctggcggccggcaacagcgtggtgttcagcccgcatccgcgcgcccggcaggtgtcgctgctgctggtccgcctgatcaaccagaagctggcagcgctcggcgcgccggaaaacctggtggtgaccgtggagaagccctcgatcgagaacaccaacgccatgatggcgcatcccaaggtgcggatgctggttgccaccggcggccccgccatcgtcaaggcggtgctgtcgaccggcaagaaggcgatcggcgccggcgccggcaatccgcccgtcgtggtcgacgaaaccgccaacatcgaaaaagccgcctgcgacatcgtcaatggctgcagcttcgacaacaacctgccctgcgtcgccgagaaggagatcatcgcagtcgcccagatcgccgattacctgatcttcaacttgaagaagaacggcgcctacgagatcaaggatccggcggttctgcagcaactgcaggacctggtgctgaccgccaagggcgggccacagaccaaatgcgtcggcaagagtgcggtgtggctgctgtcgcagatcggcatcagcgtcgacgccagcatcaagatcatcctgatggaagtgccgcgggaacatcccttcgtgcaggaagaactgatgatgccgatcctgccgctggtccgggtcgaaaccgtggatgatgcgatcgacctcgccatcgaggtggagcacgacaatcgccacaccgcgatcatgcattccaccgatgtgcgcaagctcaccaagatggccaagctgatccagaccacgatcttcgtgaagaacggcccgtcttatgcggggctcggcgccggcggcgagggatattccaccttcaccatcgcgggtcccacgggcgagggcctgacctcggcgaagtcgttcgcgcgccgccgcaaatgcgtgatggtggaagcgctcaatatccgt

SEQ ID NO: 53 - DNA, 1392 bp, Rhodopseudomonas palustris
atggtcgcaaaagcaattcgtgaccacgctggtaccgcacagccctccggcaacgcagccacgagtagtgccgctgttagtgacggggttttcgaaacaatggatgccgctgtcgaagcagcagctttggctcaacagcaatacctcctctgcagtatgtcggaccgtgcgcgattcgtacaaggtatccgtgacgtaattctgaaccaggacaccctagagaagatgtcgcggatggctgttgaagagacggggatggggaactacgagcacaagttgattaagaaccgtctcgctggcgaaaagactccaggtatcgaggacttaacgaccgatgcattctcgggtgacaacggcctcacattagttgaatactcacccttcggtgttatcggtgcgatcaccccgacgactaacccgacggaaacaatcgtctgcaactccattggtatgcttgcagcagggaactcagtagtattctcacctcacccacgtgcgcgtcaagtttccttgttgttagtacgtctgattaaccaaaaactggctgctctcggagctccagaaaacttagtagttaccgttgaaaaaccgagcattgaaaacacgaacgccatgatggcccacccaaaagtgcgcatgctggtcgcgacgggtggtccggcaatcgtcaaggcagtgttaagcacaggcaagaaagctattggtgcaggggcaggtaacccaccagtggtagttgatgagaccgctaacattgagaaagcagcctgcgatattgttaacggatgcagcttcgacaacaacctaccctgcgttgcggaaaaggagatcattgcggtggcccaaattgccgactacttgattttcaacttgaaaaagaacggtgcctacgagatcaaagacccagctgtccttcaacaattgcaagacctggtgttaactgctaagggtggcccacaaactaaatgcgtaggcaaatcagcagtatggcttctctctcagattgggatttctgttgatgctagcatcaaaatcatcttgatggaagttccccgtgaacacccgttcgttcaggaagaactaatgatgccaattcttccattagtccgcgttgaaactgtcgacgatgccattgacttagccatcgaagtagaacacgacaaccgtcacactgcaatcatgcactcaaccgacgtacgtaaactcaccaaaatggccaaattgatccaaacgactatcttcgttaagaacggtccatcatacgctggtttaggtgcaggtggggaaggatacagcactttcactattgcaggccctactggggagggattaacaagtgcaaagtcgttcgctcgtcgtcggaaatgcgtgatggtagaagccttaaacattcgc

SEQ ID NO: 54 - protein, 464 aa, Rhodopseudomonas palustris
MVAKAIRDHAGTAQPSGNAATSSAAVSDGVFETMDAAVEAAALAQQQYLLCSMSDRARFVQGIRDVILNQDTLEKMSRMAVEETGMGNYEHKLIKNRLAGEKTPGIEDLTTDAFSGDNGLTLVEYSPFGVIGAITPTTNPTETIVCNSIGMLAAGNSVVFSPHPRARQVSLLLVRLINQKLAALGAPENLVVTVEKPSIENTNAMMAHPKVRMLVATGGPAIVKAVLSTGKKAIGAGAGNPPVVVDETANIEKAACDIVNGCSFDNNLPCVAEKEIIAVAQIADYLIFNLKKNGAYEIKDPAVLQQLQDLVLTAKGGPQTKCVGKSAVWLLSQIGISVDASIKIILMEVPREHPFVQEELMMPILPLVRVETVDDAIDLAIEVEHDNRHTAIMHSTDVRKLTKMAKLIQTTIFVKNGPSYAGLGAGGEGYSTFTIAGPTGEGLTSAKSFARRRKCVMVEALNIR

SEQ ID NO: 55 - DNA, 1599 bp, Rhodobacter capsulatus
atgaaagacagcgatatcgaagacgccgtggcccgggtcttgagcggctacaccgccccgaaaagccttgaggcgacggtgaccaaggccctgacggatctggcaaaacccggcacgcagggctgtgtccgggaagccccgaagcccgccgatccgatcgatgacatcatcggcggcatcctgacgcgcgagttgggcgagaagaactgttcctgctgcaaggcgggcagctgcaccgcgcccgcaaattgcctgtcgatccccgacgatcaggccgaaaccgtgggcgacggcatctttgccacgatggatgcggccgtggaggccgccgccgaggcgcagcggcaatatctgttctgttcgatgtcggcgcgcaagcgcttcatcgacggcctccgcgaggtctttctgaaccccgcgctgctggagcggatctcgcggctggcggtcgaacagaccggcatgggcaatgtcgcgcacaagatcatcaagaaccggctggcggcggaaaagaccccgggcatcgaagacctgaccaccgaagcgcaaagcggcgatgacgggctgacgctggtcgagctgtccgcctatggcgtcatcggggcgatcacgccgacgacgaacccgaccgaaaccatcatctgcaacgcgatcgggatgttggccgcgggcaatgcggtggtcttcagcccccatccgcgcgcgcgcggcgtcagcctgctggcgatcaagctgatcaaccgcaagctggcggcgctgggggcaccggcgaaccttgtcgtcaccgtgcaggcgccctcgatcgagaacacgaatgcgatgatggcgcatcccaaggtgcggatgctggtggcgacgggcggcccggcgatcgtgaagacggtgctgtcctcgggcaagaaggcgatcggggcaggggcgggcaatccgccggtggtggttgacgaaaccgccgacattccgaaagcggcgctggatatcgtcaacggctgttcgttcgacaacaacatgccctgcgtggcggaaaaggagctgatcgcggtggcggaaatcgccgatttcctgaccgcggagctggtccgcaacggcgcgcatcggctgaccgatccggcgcagatcgccgcgctggaaaaactggtgctgaccgaaaagggcgggccgcagacgggctgcgtcggcaaatcggcggtttggttgctcgacaagatcggtgtcaaagccgcgcccgaaacccggatcatcctgatcgagacgggcaaggatcacccctttgtcgtcgaagagctgatgatgccgatcctgcctttggtgcgcgtggcatgcgtcgacgaagccattgatctggcggtcgttcttgaacacggcaaccgccataccgcgatcatgcattcgaccaatgtgcgcaagctgacgaagatggcgaagctcatccagaccacgatcttcgtgaagaacggcccctcttatgccgggcttggcgtcggcggcgagggctatgccaccttcaccatcgccgggccgacaggcgaggggctgacctctgcccgctccttcgcccgccgccgcaaatgcgtgatggtcgaagcgctgaacgtgcgc

SEQ ID NO: 56 - DNA, 1599 bp, Rhodobacter capsulatus
atgaaagatagtgatattgaagatgcggttgcgcgtgtcttatcgggttacactgcccctaaatcactagaggcaacagtaaccaaagcactcactgatttagcgaaaccagggacacaggggtgcgtacgtgaagctcctaaacctgcggaccctattgatgatatcatcggtggtatcttaacgcgtgaactaggtgagaagaattgttcatgttgcaaagcgggttcatgcacagcaccagcgaattgtctttcaattccggatgatcaagcggagacagtcggtgatggcattttcgctacgatggatgctgctgtcgaagcagctgccgaagcacaacgtcagtacctcttttgtagcatgagtgctcggaaacgctttatcgatggtcttcgcgaagtattcttgaatcctgccctgctggaacgcatctctcgcttagcagtagagcaaacaggcatgggcaatgttgcgcacaagattatcaagaatcgtttagccgcagagaaaacaccaggtatcgaagatctgaccacagaagctcaaagtggtgatgacgggttaacattagtagaattgtcagcttatggagtgatcggagctattactccaaccaccaatccgacagaaactatcatttgtaacgcaatcggtatgttagccgcaggtaatgcggttgtcttttcaccacatccacgcgctcgtggtgtatcattgttagccatcaaattgattaaccgcaagttagcagcgctgggagctccagccaatttggtcgtcacggtacaagccccttccattgaaaacacaaatgctatgatggcgcatccaaaggttcgtatgctcgtggccacaggcggtccggctatcgttaagactgtcttaagttccggcaagaaagctatcggtgctggcgcaggtaatccaccagttgtagttgatgaaactgccgacattccgaaggctgctttagacatcgtgaatggttgcagcttcgataacaatatgccttgcgtggcagaaaaagagttaatcgctgttgcggaaatcgctgactttttgacagcggaactcgttcgtaatggtgcccaccgtttaaccgatccagcacaaattgcagctttagaaaaacttgtccttacagagaaaggcggtcctcaaactggctgcgtcggcaaaagtgccgtctggttgcttgataagattggagtcaaagccgctcctgaaacccgtatcatcttgattgaaactgggaaggaccacccgttcgtcgtcgaagagttaatgatgccgattttgcctctggttcgggtggcgtgtgttgacgaagccatcgacttagctgtagttctggaacatggcaaccgtcacactgcaatcatgcactcaactaatgtacgcaagttaacaaagatggccaagttgattcaaactactatctttgtcaagaatggcccatcatatgctggcttgggtgtagggggtgaaggttacgcgacatttaccattgcgggtccaacgggtgaaggcctcacttcagcacgttcttttgcacgtcgccgtaaatgtgttatggttgaggccttgaatgtacgt

SEQ ID NO: 57 - protein, 533 aa, Rhodobacter capsulatus
MKDSDIEDAVARVLSGYTAPKSLEATVTKALTDLAKPGTQGCVREAPKPADPIDDIIGGILTRELGEKNCSCCKAGSCTAPANCLSIPDDQAETVGDGIFATMDAAVEAAAEAQRQYLFCSMSARKRFIDGLREVFLNPALLERISRLAVEQTGMGNVAHKIIKNRLAAEKTPGIEDLTTEAQSGDDGLTLVELSAYGVIGAITPTTNPTETIICNAIGMLAAGNAVVFSPHPRARGVSLLAIKLINRKLAALGAPANLVVTVQAPSIENTNAMMAHPKVRMLVATGGPAIVKTVLSSGKKAIGAGAGNPPVVVDETADIPKAALDIVNGCSFDNNMPCVAEKELIAVAEIADFLTAELVRNGAHRLTDPAQIAALEKLVLTEKGGPQTGCVGKSAVWLLDKIGVKAAPETRIILIETGKDHPFVVEELMMPILPLVRVACVDEAIDLAVVLEHGNRHTAIMHSTNVRKLTKMAKLIQTTIFVKNGPSYAGLGVGGEGYATFTIAGPTGEGLTSARSFARRRKCVMVEALNVR

SEQ ID NO: 58 - DNA, 1590 bp, Rhodospirillum rubrum
atgatgaatgacggccaaattgctgcagctgttgcgaaagtgcttgaagcctatggcgttccagccgatccaagcgctgcagcgccagcaccggcagcacctgttgcgccagcagcacccaccgctggttcagtttccgagatgattgcccgtggcattgctaaggcaagtagcgatgatcaaatcgcccaaattgttgccaaagtcgtcggagattactctgcacaagccgcaaaaccggctgttgttcctggagcagcggcatcgaccgaagctggtgatggagttttcgataccatggatgcagcggttgatgcggcagtcctggcgcagcagcagtatctgctgtgttcgatgacggatcgtcagcggtttgtggatggtattcgcgaagtcattttgcaaaaagataccctggaacttatcagtcggatggctgcagaagagactggcatgggtaactatgaacataagctaatcaaaaaccgtctggcagcagagaaaactccaggcactgaagatcttaccacagaagcattttcaggtgatgatggtttgaccttagttgagtattcaccttttggtgcgattggagcagttgctccgactacgaatcctaccgagacgattatctgtaacagtattggcatgttggcagcaggtaattctgtcattttcagtccccatccacgtgcgaccaaagtttccctacttactgttaagttgatcaatcaaaaactcgcatgtctgggtgcacctgctaacttggtagtaactgtcagcaagccgtcagtggagaataccaatgctatgatggctcatccgaaaatccgtatgttggtagctacgggtggaccaggcattgtcaaggccgttatgtctaccggcaaaaaggcaattggggcaggggctggtaatccaccggttgtggttgacgagactgcagacattgaaaaagccgcattagatatcatcaatggttgtagttttgacaacaatttgccatgtattgccgaaaaagagatcattgcagttgcgcagatcgctgactatctcatctttagtatgaagaaacaaggtgcctatcagattacagatccagccgtcttgcgcaagttacaagatcttgtcctgaccgccaaaggtggcccacaaacatcatgtgtgggaaaatcggctgtttggttacttaacaaaatcggaattgaagtcgactcctccgtgaaggtgattttgatggaagtgccgaaggaacaccctttcgtgcaagaagaacttatgatgccaatcttgcctctcgtgcgcgtttcagacgtggatgaagccattgccgttgcaatcgaagttgaacatgggaaccgtcatactgccattatgcatagtaccaacgttcgaaagttaacgaaaatggcaaaacttattcagacgaccatctttgtgaagaatggccctagttatgcaggcttaggcgtaggcggtgaagggtatacaacatttactattgcaggcccaactggggaaggacttacgagtgcaaagagctttgcacggaaacgtaaatgtgttatggttgaagcattgaatatccgc

SEQ ID NO: 59 - DNA, 1591 bp, Rhodospirillum rubrum
atgatgaatgacggccaaattgctgcagctgttgcgaaagtgcttgaagcctatggcgttccagccgatccaagcgctgcagcgccagcaccggcagcacctgttgcgccagcagcacccaccgctggttcagtttccgagatgattgcccgtggcattgctaaggcaagtagcgatgatcaaatcgcccaaattgttgccaaagtcgtcggagattactctgcacaagccgcaaaaccggctgttgttcctggagcagcggcatcgaccgaagctggtgatggagttttcgataccatggatgcagcggttgatgcggcagtcctggcgcagcagcagtatctgctgtgttcgatgacggatcgtcagcggtttgtggatggtattcgcgaagtcattttgcaaaaagataccctggaacttatcagtcggatggctgcagaagagactggcatgggtaactatgaacataagctaatcaaaaaccgtctggcagcagagaaaactccaggcactgaagatcttaccacagaagcattttcaggtgatgatggtttgaccttagttgagtattcaccttttggtgcgattggagcagttgctccgactacgaatcctaccgagacgattatctgtaacagtattggcatgttggcagcaggtaattctgtcattttcagtccccatccacgtgcgaccaaagtttccctacttactgttaagttgatcaatcaaaaactcgcatgtctgggtgcacctgctaacttggtagtaactgtcagcaagccgtcagtggagaataccaatgctatgatggctcatccgaaaatccgtatgttggtagctacgggtggaccaggcattgtcaaggccgttatgtctaccggcaaaaaggcaattggggcaggggctggtaatccaccggttgtggttgacgagactgcagacattgaaaaagccgcattagatatcatcaatggttgtagttttgacaacaatttgccatgtattgccgaaaaagagatcattgcagttgcgcagatcgctgactatctcatctttagtatgaagaaacaaggtgcctatcagattacagatccagccgtcttgcgcaagttacaagatcttgtcctgaccgccaaaggtggcccacaaacatcatgtgtgggaaaatcggctgtttggttacttaacaaaatcggaattgaagtcgactcctccgtgaaggtgattttgatggaagtgccgaaggaacaccctsttcgtgcaagaagaacttatgatgccaatcttgcctctcgtgcgcgtttcagacgtggatgaagccattgccgttgcaatcgaagttgaacatgggaaccgtcatactgccattatgcatagtaccaacgttcgaaagttaacgaaaatggcaaaacttattcagacgaccatctttgtgaagaatggccctagttatgcaggcttaggcgtaggcggtgaagggtatacaacatttactattgcaggcccaactggggaaggacttacgagtgcaaagagctttgcacggaaacgtaaatgtgttatggttgaagcattgaatatccgc

SEQ ID NO: 60 - protein, 530 aa, Rhodospirillum rubrum
MMNDGQIAAAVAKVLEAYGVPADPSAAAPAPAAPVAPAAPTAGSVSEMIARGIAKASSDDQIAQIVAKVVGDYSAQAAKPAVVPGAAASTEAGDGVFDTMDAAVDAAVLAQQQYLLCSMTDRQRFVDGIREVILQKDTLELISRMAAEETGMGNYEHKLIKNRLAAEKTPGTEDLTTEAFSGDDGLTLVEYSPFGAIGAVAPTTNPTETIICNSIGMLAAGNSVIFSPHPRATKVSLLTVKLINQKLACLGAPANLVVTVSKPSVENTNAMMAHPKIRMLVATGGPGIVKAVMSTGKKAIGAGAGNPPVVVDETADIEKAALDIINGCSFDNNLPCIAEKEIIAVAQIADYLIFSMKKQGAYQITDPAVLRKLQDLVLTAKGGPQTSCVGKSAVWLLNKIGIEVDSSVKVILMEVPKEHPFVQEELMMPILPLVRVSDVDEAIAVAIEVEHGNRHTAIMHSTNVRKLTKMAKLIQTTIFVKNGPSYAGLGVGGEGYTTFTIAGPTGEGLTSAKSFARKRKCVMVEALNIR

SEQ ID NO: 61 - DNA, 1404 bp, Eubacterium hallii
atgaatatcgatgttgaattaattgaaaaagtcgttaaaaaagtattaaacgatgttgagactggctcaagcgaaagcgaatatggatatggtatctttgatactatggacgaggcaattgaagcttccgcaaaagctcagaaagaatatatgaaccattccatggctgacagacagagatacgttgaaggtatcagagaagttgtttgtacaaaagaaaatcttgagtacatgagtaaactcgctgtagaagaaagcggaatgggtgcttatgaatataaagtaatcaaaaaccgtttagcagcagttaaatctccaggtgttgaagatttaacaacagaagctttatcaggtgatgacggacttacattagttgagtactgtccattcggtgtaattggtgctatcgctcctacaacaaaccctacagaaacagttatctgtaactccatcgcaatgcttgctggtggaaacactgtagtattcagcccacatccacgttcaaaaggcgtttccatctggttaatcaaaaaactgaacgctaaattagaagagctgggcgctcctagaaacttaatcgtaactgttaaagaaccatctatcgaaaacacaaatatcatgatgaaccatccaaaggttcgtatgcttgttgctacaggtggtcctggaatcgttaaagcagttatgtcaacaggtaagaaagctatcggagctggtgctggtaaccctccagtagtagtagatgagacagctgatatcgaaaaagcagctaaggatatcgttaacggatgttctttcgataacaaccttccatgtatcgcagagaaagaagttatcgctgtagatcagatcgctgactacttaatcttcaacatgaagaacaatggcgcatacgaagtaaaagatcctgaaatcatcgaaaagatggttgatctcgtaacaaaagacagaaagaaaccagctgttaacttcgtaggtaagagcgcacagtacatccttgacaaagttggaatcaaggttggaccagaagttaaatgtatcatcatggaagctcctaaggatcatccattcgtacagatcgagttaatgatgcctatccttcctatcgttcgtgttcctaacgtagacgaagctatcgacttcgctgtagaagtagaacatggtaacagacatacagctatgatgcattctaagaatgtagataaacttacaaagatggcaaaagaaatcgaaacaactattttcgtaaagaatggcccatcttatgcaggtatcggtgttggcggaatgggatatacaacattcacaattgccggacctacaggtgaaggtttaacatctgctaaatcattctgccgtaagagaagatgtgtattacaggacggtctccatatcagaatgaaa

SEQ ID NO: 62 - DNA, 1404 bp, Eubacterium hallii
atgaacatcgacgtagaacttatcgaaaaggttgtgaagaaggtcttaaacgacgtggaaaccgggtcaagcgaaagtgaatacggttacggtatcttcgacaccatggacgaagccatcgaggcaagcgcaaaggctcagaaggaatacatgaaccactctatggcagaccgtcagcggtacgttgagggtattcgggaagtggtgtgcacaaaggaaaaccttgaatacatgtcaaagctcgcagttgaagaaagcggcatgggtgcatacgaatacaaggttatcaagaaccgcttagccgctgtgaagagtccgggtgttgaagacctgacgacggaagcattatcaggtgacgacggattaacattagtagaatactgcccattcggagttatcggtgcaatcgcaccgacaaccaaccctactgaaactgtcatttgcaacagcattgctatgctagcagggggtaacacggtagttttctcaccacaccctcgttctaagggtgtgagcatttggttaatcaagaagctcaacgcaaagttagaggaattaggcgctccacggaacttgattgttactgttaaggaaccgagtatcgagaacacgaacattatgatgaaccacccaaaggttcgtatgctcgtcgctaccggaggtccaggcatcgttaaggctgtcatgtctactggtaagaaggcaatcggtgcaggagcaggaaacccaccagtagtcgtagacgaaaccgcagacatcgaaaaggcagcgaaggacattgttaacgggtgctcgttcgacaacaacttgccttgcatcgctgaaaaggaagtcatcgcagtagaccagattgcagactaccttatcttcaacatgaagaacaatggcgcttacgaagtcaaggacccggagattatcgagaagatggttgaccttgtgacaaaggaccgtaagaagcctgcagttaacttcgtcgggaagtcagcacagtacatcctagacaaggttggcatcaaggtaggtcccgaagtaaagtgcatcatcatggaagcacctaaggaccacccattcgtacagattgagttgatgatgcctatcttaccaattgtacgcgtccctaacgtagacgaagcaatcgacttcgcggttgaggttgaacacggaaaccgtcacactgctatgatgcactcgaagaacgtggacaagctcacaaagatggctaaggaaatcgaaacgacaatcttcgtgaagaatggtccctcctacgctggaatcggagttggcggtatgggttacaccacattcacaattgcgggtcccactggggaaggtttgacgtccgccaagagcttctgccggaaacgtcgctgcgttttacaggacggccttcacatccgtatgaag

SEQ ID NO: 63 - protein, 468 aa, Eubacterium hallii
MNIDVELIEKVVKKVLNDVETGSSESEYGYGIFDTMDEAIEASAKAQKEYMNHSMADRQRYVEGIREVVCTKENLEYMSKLAVEESGMGAYEYKVIKNRLAAVKSPGVEDLTTEALSGDDGLTLVEYCPFGVIGAIAPTTNPTETVICNSIAMLAGGNTVVFSPHPRSKGVSIWLIKKLNAKLEELGAPRNLIVTVKEPSIENTNIMMNHPKVRMLVATGGPGIVKAVMSTGKKAIGAGAGNPPVVVDETADIEKAAKDIVNGCSFDNNLPCIAEKEVIAVDQIADYLIFNMKNNGAYEVKDPEIIEKMVDLVTKDRKKPAVNFVGKSAQYILDKVGIKVGPEVKCIIMEAPKDHPFVQIELMMPILPIVRVPNVDEAIDFAVEVEHGNRHTAMMHSKNVDKLTKMAKEIETTIFVKNGPSYAGIGVGGMGYTTFTIAGPTGEGLTSAKSFCRKRRCVLQDGLHIRMK

SEQ ID NO: 64 - DNA, 1914 bp, Propionibacterium freudenreichii
atgagcagcacggatcaggggaccaaccccgccgacactgacgacctcactcccaccacactcagtctggccggggatttccccaaggccactgaggagcagtgggagcgcgaagttgagaaggtattcaaccgtggtcgtccaccggagaagcagctgaccttcgccgagtgtctgaagcgcctgacggttcacaccgtcgatggcatcgacatcgtgccgatgtaccgtccgaaggacgcgccgaagaagctgggttaccccggcgtcacccccttcacccgcggcaccacggtgcgcaacggtgacatggatgcctgggacgtgcgcgccctgcacgaggatcccgacgagaagttcacccgcaaggcgatccttgaagacctggagcgtggcgtcacctccctgttgttgcgcgttgatcccgacgcgatcgcacccgagcacctcgacgaggtcctctccgacgtcctgctggaaatgaccaaggtggaggtcttcagccgctacgaccagggtgccgccgccgaggccttgatgggcgtctacgagcgctccgacaagccggcgaaggacctggccctgaacctgggcctggatcccatcggcttcgcggccctgcagggcaccgagccggatctgaccgtgctcggtgactgggtgcgccgcctggcgaagttctcaccggactcgcgcgccgtcacgatcgacgcgaacgtctaccacaacgccggtgccggcgacgtggcagagctcgcttgggcactggccaccggcgcggagtacgtgcgcgccctggtcgaacagggcttcaacgccacagaggccttcgacacgatcaacttccgtgtcaccgccacccacgaccagttcctcacgatcgcccgtcttcgcgccctgcgcgaggcatgggcccgcatcggcgaggtctttggcgtggacgaggacaagcgcggcgctcgccagaatgcgatcaccagttggcgtgagctcacccgcgaagacccctatgtcaacatccttcgcggttcgattgccaccttctccgcctccgttggcggggccgagtcgatcacgacgctgcccttcacccaggccctcggcctgccggaggacgacttcccgctgcgcatcgcgcgcaacacgggcatcgtgctcgccgaagaggtgaacatcggccgcgtcaacgacccggccggtggctcctactacgtcgagtcgctcactcgcaccctggccgacgctgcctggaaggaattccaggaggtcgagaagctcggtggcatgtcgaaggcggtcatgaccgagcacgtcaccaaggtgctcgacgcctgcaatgccgagcgcgccaagcgcctggccaaccgcaagcagccgatcaccgcggtcagcgagttcccgatgatcggggcccgcagcatcgagaccaagccgttcccaaccgctccggcgcgcaagggcctggcctggcatcgcgattccgaggtgttcgagcagctgatggatcgctccaccagcgtctccgagcgccccaaggtgttccttgcctgcctgggcacccgtcgcgacttcggtggccgcgagggcttctccagcccggtatggcacatcgccggtatcgacaccccgcaggtcgaaggcggcaccaccgccgagatcgtcgaggcgttcaagaagtcgggcgcccaggtggccgatctctgctcgtccgccaagatctacgcgcagcagggacttgaggttgccaaggcgctcaaggccgccggcgcgaaggccctgtatctgtcgggcgccttcaaggagttcggcgatgacgccgccgaggccgagaagctgatcgacggacgcctgtacatgggcatggatgtcgtcgacaccctgtcctccacccttgatatcttgggagtcgcgaag

SEQ ID NO: 65 - DNA, 1914 bp, Propionibacterium freudenreichii
atgtcttctactgaccagggtaccaatcctgccgacaccgatgacttaacgccaacgaccctgtcattggcaggcgattttccgaaggccaccgaagaacagtgggaacgtgaagttgaaaaagttttcaaccgtggtcgtccgcctgaaaagcaattgacatttgctgagtgcttgaaacgattgacggtgcacacggtggatggtattgatattgttcccatgtatcgcccaaaagatgcaccgaagaagttaggttatccaggtgtgacaccattcactcgtggtacgacggtgcgcaacggtgatatggatgcgtgggatgtacgtgcgttgcatgaagatcctgatgaaaagttcacccggaaggcgatcctggaagacttggaacggggtgtcactagcttgttgttacgtgttgatccagatgccattgcacctgaacacttggatgaggtgctttcagatgttttacttgaaatgactaaggttgaagtcttttctcgctatgatcagggtgcagcagccgaggccttaatgggcgtatatgagcgatcagacaaaccagcgaaagacttagctttgaacttaggtttagatcccattggttttgcagccctccaagggactgaacccgatttgacggttttaggtgattgggtacgtcggctcgccaagttttcaccagattcgcgtgcagtgaccatcgatgccaacgtctatcacaatgcaggagctggggacgttgcagaacttgcgtgggcattggccaccggagcagaatatgtacgtgccctggtggaacagggctttaacgcaacagaagcattcgatacgatcaatttccgtgttacagcgactcatgatcagttcttaactattgcgcgtctacgcgcacttcgggaagcatgggcacggattggcgaagtgttcggggtcgacgaagataaacgtggtgcccgtcagaatgcaatcacctcttggcgtgaattgactcgtgaagatccatatgtgaatatcttacgcggatcgattgccacttttagtgcatcagttggtggtgcggaatcaattaccaccctaccgtttacacaggctttagggctccccgaagatgactttcccctccggattgcacgcaatactggtatcgtgttagccgaagaagtcaacattggtcgtgttaatgatccagcagggggaagttactacgtggaatcactaacccggacacttgccgacgcagcttggaaagagttccaggaagtcgaaaaacttggtgggatgtcaaaagccgtaatgactgagcatgtcaccaaagtgttagatgcttgcaacgctgaacgggcaaaacgtctcgcgaaccgtaaacagccaattaccgcagtatcggaatttccgatgattggagcacgcagtatcgaaaccaaaccgtttccaactgcccctgcacggaaggggttagcatggcatcgtgattcagaagttttcgagcagttaatggatcgttccacatctgtctcggaacgcccaaaagtttttctggcgtgcttagggactcgtcgggattttggtggtcgtgagggttttagtagccctgtgtggcacattgccggaattgatactccgcaagttgagggaggaacgactgcagagattgtcgaagccttcaagaaaagtggggctcaagttgcagatctgtgcagtagcgccaaaatctatgcacagcaggggttagaagttgcaaaagcgcttaaagctgccggtgcaaaagccttatacttatctggtgcgttcaaagagttcggtgatgatgcagcagaagcagagaagttgattgatggccgtttgtatatgggcatggatgtagttgacaccctctctagtacattggatattcttggtgtcgctaaa

SEQ ID NO: 66 - protein, 638 aa, Propionibacterium freudenreichii
MSSTDQGTNPADTDDLTPTTLSLAGDFPKATEEQWEREVEKVFNRGRPPEKQLTFAECLKRLTVHTVDGIDIVPMYRPKDAPKKLGYPGVTPFTRGTTVRNGDMDAWDVRALHEDPDEKFTRKAILEDLERGVTSLLLRVDPDAIAPEHLDEVLSDVLLEMTKVEVFSRYDQGAAAEALMGVYERSDKPAKDLALNLGLDPIGFAALQGTEPDLTVLGDWVRRLAKFSPDSRAVTIDANVYHNAGAGDVAELAWALATGAEYVRALVEQGFNATEAFDTINFRVTATHDQFLTIARLRALREAWARIGEVFGVDEDKRGARQNAITSWRELTREDPYVNILRGSIATFSASVGGAESITTLPFTQALGLPEDDFPLRIARNTGIVLAEEVNIGRVNDPAGGSYYVESLTRTLADAAWKEFQEVEKLGGMSKAVMTEHVTKVLDACNAERAKRLANRKQPITAVSEFPMIGARSIETKPFPTAPARKGLAWHRDSEVFEQLMDRSTSVSERPKVFLACLGTRRDFGGREGFSSPVWHIAGIDTPQVEGGTTAEIVEAFKKSGAQVADLCSSAKIYAQQGLEVAKALKAAGAKALYLSGAFKEFGDDAAEAEKLIDGRLYMGMDVVDTLSSTLDILGVAK

SEQ ID NO: 67 - DNA, 2187 bp, Propionibacterium freudenreichii
gtgagcactctgccccgttttgattcagttgacctgggcaatgccccggttcctgctgatgccgcacagcgcttcgaggagttggccgccaaggccggcaccgaagaggcgtgggagacggctgagcagattccggttggcaccctgttcaacgaagacgtctacaaggacatggactggctggacacctacgccggtatcccgccgttcgtccacggcccatatgcaaccatgtacgcgttccgtccctggacgattcgccagtacgccggcttctccacggccaaggagtccaacgccttctaccgccgcaaccttgcggcgggccagaagggcctgtcggttgccttcgacctgcccacccaccgcggctacgactcggacaatccccgcgtcgccggtgacgtcggcatggccggggtggccatcgactccatctatgacatgcgcgagctgttcgccggcattccgctggaccagatgagcgtgtcgatgaccatgaacggcgccgtgctgccgatcctggccctctatgtggtgaccgccgaggagcagggcgtcaagcccgagcagctcgccgggacgatccagaacgacatcctcaaggagttcatggttcgtaacacctatatctacccgccgcagccgagtatgcgaatcatctccgagatcttcgcctacacgagtgccaatatgccgaagtggaattcgatttccatttccggctaccacatgcaggaagccggcgccacggccgacatcgagatggcctacaccctggccgacggtgtcgactacatccgcgccggcgagtcggtgggcctcaatgtcgaccagttcgcgccgcgtctgtccttcttctggggcatcggcatgaacttcttcatggaggttgccaagctgcgtgccgcacgtatgttgtgggccaagctggtgcatcagttcgggccgaagaatccgaagtcgatgagcctgcgcacccactcgcagacctccggttggtcgctgaccgcccaggacgtctacaacaacgtcgtgcgtacctgcatcgaggccatggccgccacccagggccatacccagtcgctgcacacgaactcgctcgacgaggccattgccctaccgaccgatttcagcgcccgcatcgcccgtaacacccagctgttcctgcagcaggaatcgggcacgacgcgcgtgatcgacccgtggagcggctcggcatacgtcgaggagctcacctgggacctggcccgcaaggcatggggccacatccaggaggtcgagaaggtcggcggcatggccaaggccatcgaaaagggcatccccaagatgcgcattgaggaagccgccgcccgcacccaggcacgcatcgactccggccgtcagccgctgatcggcgtgaacaagtaccgcctggagcacgagccgccgctcgatgtgctcaaggttgacaactccacggtgctcgccgagcagaaggccaagctggtcaagctgcgcgccgagcgcgatcccgagaaggtcaaggccgccctcgacaagatcacctgggctgccgccaaccccgacgacaaggatccggatcgcaacctgctgaagctgtgcatcgacgctggccgcgccatggcgacggtcggcgagatgagcgacgcgctcgagaaggtcttcggacgctacaccgcccagattcgcaccatctccggtgtgtactcgaaggaagtgaagaacacgcctgaggttgaggaagcacgcgagctcgttgaggaattcgagcaggccgagggccgtcgtcctcgcatcctgctggccaagatgggccaggacggtcacgaccgtggccagaaggtcatcgccaccgcctatgccgacctcggtttcgacgtcgacgtgggcccgctgttccagaccccggaggagaccgcacgtcaggccgtcgaggccgatgtgcacgtggtgggcgtttcgtcgctcgccggcgggcatctgacgctggttccggccctgcgcaaggagctggacaagctcggacgtcccgacatcctcatcaccgtgggcggcgtgatccctgagcaggacttcgacgagctgcgtaaggacggcgccgtggagatctacacccccggcaccgtcattccggagtcggcgatctcgctggtcaagaaactgcgggcttcgctcgatgcctag

SEQ ID NO: 68 - DNA, 2184 bp, Propionibacterium freudenreichii
atgtcaactttaccacgctttgacagtgttgatctcggtaatgcacccgttccggctgatgccgcacaacggtttgaagaactggctgccaaagctggtacagaagaagcctgggaaactgccgagcaaatccctgtcggtacgttattcaacgaagatgtgtacaaagatatggattggttggatacttatgccggtattcctcctttcgtgcatggtccgtatgccacaatgtatgcgtttcgtccttggaccatccgtcaatatgcagggtttagtacagccaaagaatcgaatgccttctatcgccgaaacttagcagcgggacaaaaagggctgagtgttgcatttgacttgcccacacatcggggttatgactcagataatccccgtgttgcaggggatgtagggatggctggtgtcgcaattgatagcatctatgatatgcgggagttgtttgcaggcattccactagatcaaatgtccgtctcaatgactatgaatggagctgttttgcctattctggcgctatacgtcgttacagccgaagagcagggcgtcaagccagaacaattagcgggtacgattcaaaatgatattctcaaagagtttatggttcgtaacacgtacatctatccaccgcagccttcaatgcgcatcatttcggagatctttgcttatacgtcggccaacatgccaaaatggaactccatttccatctctggttatcatatgcaagaagcaggtgcgacagctgatattgaaatggcatacactttggcagatggtgttgattacatccgtgcaggtgaatccgttggcttgaatgtcgatcagtttgctccacgcctctccttcttttggggcatcggcatgaacttctttatggaagttgctaaacttcgcgcagctcgtatgttatgggcgaagctcgttcaccagtttggcccaaagaaccctaaatctatgtcattgcgtactcactctcaaacctcgggttggtcacttaccgcacaagatgtgtacaataacgttgtacgtacctgtattgaagcaatggcagcgacccagggccacacacaaagccttcataccaactctttggatgaagctattgctttgcccacggactttagtgcacggatcgcacgtaatactcagttgttcttacagcaagaaagcgggactactcgtgtcatcgacccatggagtgggtctgcctatgttgaggagttaacctgggatctagcacgcaaggcttggggtcatattcaggaagttgaaaaagttgggggtatggcaaaagccattgagaaagggattccgaagatgcgtattgaagaagcagcagcgcgaacccaagcccgtattgatagtggtcgtcaaccccttattggtgtcaacaagtaccgcttggaacatgagcctcccctggatgttttgaaagtagataactctaccgtgcttgcagaacagaaagctaagttggttaagttacgtgcggaacgtgacccggaaaaagtaaaagctgcattagataagattacatgggcagccgcaaaccctgacgataaagacccggatcgcaacttgttgaaactctgcatcgacgctggtcgtgccatggcaacagttggtgaaatgagcgatgcgctggaaaaagtttttggtcgctacacagcacagattcgtacgatcagtggggtctatagcaaggaagtcaaaaacactccagaagttgaagaagcgcgtgaactggtagaagaatttgaacaagcagagggacggcgtcctcgcattttgttggccaaaatgggacaggacgggcacgatcgtggtcagaaagttattgccacggcatatgcagacttaggatttgacgttgacgttggcccactttttcaaactccggaggaaactgcccgtcaagcagtagaagcggacgtgcatgtagttggtgtttcgtcactagccggtggacacttaacactagttccagcgctgcgtaaagagttagataaacttggtcgtccggacatccttatcaccgttggtggcgtgatcccagaacaagattttgacgaactacgtaaggatggtgccgtagaaatctatacaccgggtaccgttattcccgagtctgctatctcattagtcaagaaattgcgtgcaagtttggatgct

SEQ ID NO: 69 - protein, 728 aa, Propionibacterium freudenreichii
MSTLPRFDSVDLGNAPVPADAAQRFEELAAKAGTEEAWETAEQIPVGTLFNEDVYKDMDWLDTYAGIPPFVHGPYATMYAFRPWTIRQYAGFSTAKESNAFYRRNLAAGQKGLSVAFDLPTHRGYDSDNPRVAGDVGMAGVAIDSIYDMRELFAGIPLDQMSVSMTMNGAVLPILALYVVTAEEQGVKPEQLAGTIQNDILKEFMVRNTYIYPPQPSMRIISEIFAYTSANMPKWNSISISGYHMQEAGATADIEMAYTLADGVDYIRAGESVGLNVDQFAPRLSFFWGIGMNFFMEVAKLRAARMLWAKLVHQFGPKNPKSMSLRTHSQTSGWSLTAQDVYNNVVRTCIEAMAATQGHTQSLHTNSLDEAIALPTDFSARIARNTQLFLQQESGTTRVIDPWSGSAYVEELTWDLARKAWGHIQEVEKVGGMAKAIEKGIPKMRIEEAAARTQARIDSGRQPLIGVNKYRLEHEPPLDVLKVDNSTVLAEQKAKLVKLRAERDPEKVKAALDKITWAAANPDDKDPDRNLLKLCIDAGRAMATVGEMSDALEKVFGRYTAQIRTISGVYSKEVKNTPEVEEARELVEEFEQAEGRRPRILLAKMGQDGHDRGQKVIATAYADLGFDVDVGPLFQTPEETARQAVEADVHVVGVSSLAGGHLTLVPALRKELDKLGRPDILITVGGVIPEQDFDELRKDGAVEIYTPGTVIPESAISLVKKLRASLDA

SEQ ID NO: 70 - DNA, 1011 bp, Propionibacterium freudenreichii
atgcctaggccgttcaatgtccccgagcttgttgacgaggtcctcgccaacaagcgcccgggcctcgcgcgtgcgatcacgctcgtcgagtcaacactgccggctcaccggccgttggctcgcgagctgctcgccgccctgctcccccattcgggcaacgccatccgcgttggcctgacgggggtgccgggggccggcaagtcgacgttcaccgacgccatgggcgtgcggctcatcgatcgtggccacaaggtcgcggtgctggcggttgacccgtccagttcgcgcaccggcggctcgatcctgggcgaccgcacccgcatgggcaagctggctgagtccgacagcgccttcatccgcccctcgccctcggcaggccacctcggtggcgtggcgcgggcgacgcgggaggcgatgatcatcgtcgaggccgcaggctatgacacggtgatcgtcgagacggtcggtgtgggtcagtccgaagtggcggtgtcgggcatggtggataccttcctcatgcttgcgctcaccggttcgggcgaccagctgcagggcatcaagcgaggcattctggagctcgccgatgtgattgccgtgaacaaggccgacggggacaatgccggcgaggcgcgggtgaccgcgcgcgatctgtccatcgccatgaagctcatcaacgacgaggccgatgggtggcgcactccggtgctcacctgctcggcctacaccggtgatgggctggatgatgtgtggaaggccgttgtcgaacaccgtgactgggtggacaagacggtgggcctcaagcagtatcgcgccgatcagcaggtggattggatgtggtcgcagatccagagcgctgttctggattcgctgcggtccacgcccgagctgctgaagctgggccacaagctcgaacacgaggttgcggagcagcgcaccagcgccctggaggcgtcgatggagttcttgtcgacctatgccaagtcggttcccggatttgaatgggatccgtca

SEQ ID NO: 71 - DNA, 1011 bp, Propionibacterium freudenreichii
atgccacgtccgttcaatgttccagaattggtcgatgaagtcttggctaacaagcgtcctggcctagcacgcgcaattactttagtggaaagcacgttgccagcccatcgtccgttagcccgtgaattactcgcagcgttgttgccacattcggggaatgcaattcgtgtgggtttaactggcgttccaggagcaggcaagtcaacttttaccgatgcaatgggcgtacgcttgattgatcgcggacacaaagttgcagtactcgcagtagatccaagctcaagtcgtactggtgggtccattttgggtgatcggacccgtatgggcaaattggccgaatcagactcagccttcattcggccatcgccaagtgcaggtcacttgggtggtgttgcacgtgccactcgagaagctatgatcattgttgaagctgctggttacgacacagttatcgttgaaacagtgggtgtcggccagtctgaagttgcggtttctggcatggttgatacctttctcatgctagcgttaactgggagtggggaccaattgcaaggcatcaaacgtggcattcttgaattggcagatgtaattgcagtgaataaggctgatggtgacaatgcaggcgaagcacgggttactgcacgcgacttaagcattgctatgaagttgatcaatgatgaagctgacgggtggcggactcccgtgttaacctgtagcgcctacaccggtgatggtctcgatgacgtttggaaagcagttgtcgaacatcgcgactgggttgacaaaacggtgggcttgaaacaatatcgtgcagaccagcaagttgattggatgtggtcgcaaattcagagtgctgtgttagatagcctacgcagcacaccggaactgttgaagttaggccataaactagagcatgaagttgcagaacagcgtaccagtgcattggaagcatcaatggagtttctgtcaacttatgccaaatccgtaccgggttttgaatgggacccatca

SEQ ID NO: 72 - protein, 337 aa, Propionibacterium freudenreichii
MPRPFNVPELVDEVLANKRPGLARAITLVESTLPAHRPLARELLAALLPHSGNAIRVGLTGVPGAGKSTFTDAMGVRLIDRGHKVAVLAVDPSSSRTGGSILGDRTRMGKLAESDSAFIRPSPSAGHLGGVARATREAMIIVEAAGYDTVIVETVGVGQSEVAVSGMVDTFLMLALTGSGDQLQGIKRGILELADVIAVNKADGDNAGEARVTARDLSIAMKLINDEADGWRTPVLTCSAYTGDGLDDVWKAVVEHRDWVDKTVGLKQYRADQQVDWMWSQIQSAVLDSLRSTPELLKLGHKLEHEVAEQRTSALEASMEFLSTYAKSVPGFEWDPS

SEQ ID NO: 73 - DNA, 444 bp, Propionibacterium freudenreichii
atgagtaatgaggatcttttcatctgtatcgatcacgtggcatatgcgtgccccgacgccgacgaggcttccaagtactaccaggagaccttcggctggcatgagctccaccgcgaggagaacccggagcagggagtcgtcgagatcatgatggccccggctgcgaagctgaccgagcacatgacccaggttcaggtcatggccccgctcaacgacgagtcgaccgttgccaagtggcttgccaagcacaatggtcgcgccggactgcaccacatggcatggcgtgtcgatgacatcgacgccgtcagcgccaccctgcgcgagcgcggcgtgcagctgctgtacgacgagcccaagctcggcaccggcggaaaccgcatcaacttcatgcatcccaagtcgggcaagggcgtgctcatcgagctcacccagtacccgaagaac

SEQ ID NO: 74 - DNA, 444 bp, Propionibacterium freudenreichii
atgagcaacgaggacttattcatttgtattgatcatgttgcctacgcatgtcctgatgccgatgaagcgagcaagtattaccaggaaacatttgggtggcatgaactccatcgcgaagaaaaccctgaacagggtgttgtggaaatcatgatggcaccagcagccaaattgaccgaacacatgacccaagtacaggtaatggcaccgttgaatgatgaaagcacggttgcaaagtggctcgccaagcacaatgggcgtgcaggcttacatcacatggcctggcgtgtggatgatattgatgctgtttctgctaccctccgtgaacggggtgttcagttattgtacgatgagccgaagttagggactgggggaaatcgcatcaactttatgcatccaaagtctggcaagggtgtcctcattgagttaacccaatacccgaagaat

SEQ ID NO: 75 - protein, 148 aa, Propionibacterium freudenreichii
MSNEDLFICIDHVAYACPDADEASKYYQETFGWHELHREENPEQGVVEIMMAPAAKLTEHMTQVQVMAPLNDESTVAKWLAKHNGRAGLHHMAWRVDDIDAVSATLRERGVQLLYDEPKLGTGGNRINFMHPKSGKGVLIELTQYPKN

SEQ ID NO: 76 - DNA, 971 bp, Escherichia coli
tctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacgg

SEQ ID NO: 77 - DNA, 971 bp, Escherichia coli
tctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacgg

SEQ ID NO: 78 - protein, 271 aa, Escherichia coli
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 79 - DNA, 2145 bp, Escherichia coli
atgtctaacgtgcaggagtggcaacagcttgccaacaaggaattgagccgtcgggagaaaactgtcgactcgctggttcatcaaaccgcggaagggatcgccatcaagccgctgtataccgaagccgatctcgataatctggaggtgacaggtacccttcctggtttgccgccctacgttcgtggcccgcgtgccactatgtataccgcccaaccgtggaccatccgtcagtatgctggtttttcaacagcaaaagagtccaacgctttttatcgccgtaacctggccgccgggcaaaaaggtctttccgttgcgtttgaccttgccacccaccgtggctacgactccgataacccgcgcgtggcgggcgacgtcggcaaagcgggcgtcgctatcgacaccgtggaagatatgaaagtcctgttcgaccagatcccgctggataaaatgtcggtttcgatgaccatgaatggcgcagtgctaccagtactggcgttttatatcgtcgccgcagaagagcaaggtgttacacctgataaactgaccggcaccattcaaaacgatattctcaaagagtacctctgccgcaacacctatatttacccaccaaaaccgtcaatgcgcattatcgccgacatcatcgcctggtgttccggcaacatgccgcgatttaataccatcagtatcagcggttaccacatgggtgaagcgggtgccaactgcgtgcagcaggtagcatttacgctcgctgatgggattgagtacatcaaagcagcaatctctgccggactgaaaattgatgacttcgctcctcgcctgtcgttcttcttcggcatcggcatggatctgtttatgaacgtcgccatgttgcgtgcggcacgttatttatggagcgaagcggtcagtggatttggcgcacaggacccgaaatcactggcgctgcgtacccactgccagacctcaggctggagcctgactgaacaggatccgtataacaacgttatccgcaccaccattgaagcgctggctgcgacgctgggcggtactcagtcactgcataccaacgcctttgacgaagcgcttggtttgcctaccgatttctcagcacgcattgcccgcaacacccagatcatcatccaggaagaatcagaactctgccgcaccgtcgatccactggccggatcctattacattgagtcgctgaccgatcaaatcgtcaaacaagccagagctattatccaacagatcgacgaagccggtggcatggcgaaagcgatcgaagcaggtctgccaaaacgaatgatcgaagaggcctcagcgcgcgaacagtcgctgatcgaccagggcaagcgtgtcatcgttggtgtcaacaagtacaaactggatcacgaagacgaaaccgatgtacttgagatcgacaacgtgatggtgcgtaacgagcaaattgcttcgctggaacgcattcgcgccacccgtgatgatgccgccgtaaccgccgcgttgaacgccctgactcacgccgcacagcataacgaaaacctgctggctgccgctgttaatgccgctcgcgttcgcgccaccctgggtgaaatttccgatgcgctggaagtcgctttcgaccgttatctggtgccaagccagtgtgttaccggcgtgattgcgcaaagctatcatcagtctgagaaatcggcctccgagttcgatgccattgttgcgcaaacggagcagttccttgccgacaatggtcgtcgcccgcgcattctgatcgctaagatgggccaggatggacacgatcgcggcgcgaaagtgatcgccagcgcctattccgatctcggtttcgacgtagatttaagcccgatgttctctacacctgaagagatcgcccgcctggccgtagaaaacgacgttcacgtagtgggcgcatcctcactggctgccggtcataaaacgctgatcccggaactggtcgaagcgctgaaaaaatggggacgcgaagatatctgcgtggtcgcgggtggcgtcattccgccgcaggattacgccttcctgcaagagcgcggcgtggcggcgatttatggtccaggtacacctatgctcgacagtgtgcgcgacgtactgaatctgataagccagcatcatgattaa

SEQ ID NO: 80 - DNA, 2145 bp, Escherichia coli
atgtctaacgttcaagaatggcaacaattggctaacaaagagctttctcgtcgcgagaaaacggtagattcacttgttcaccaaacagcagaaggcattgcaatcaaaccgttgtatacggaggcggatttagacaacttagaagtaacaggcactttaccgggacttcctccatatgttcgtggccctcgtgcaacgatgtacactgcgcagccttggacaatccgccaatatgcgggtttctctacagcaaaggaatctaacgcattctatcgtcgtaacttggctgctggccaaaaaggactttctgtagcattcgacttagctacacatcgcggatacgactctgataacccacgtgttgcaggagatgttggtaaagctggcgtagctatcgatacggttgaagatatgaaagtactttttgatcaaattcctttagacaagatgtctgtttctatgacaatgaacggtgctgtattaccagttcttgcattctacattgttgctgcggaggaacaaggcgttacaccggacaagttaactggaacaatccagaacgacatcttgaaagaatatctttgccgtaacacgtacatctatcctcctaagccttctatgcgcatcattgcggatatcatcgcttggtgtagcggaaacatgccacgtttcaatacaatctcaatctctggatatcacatgggtgaggctggagctaactgtgtacagcaggttgcttttacattagctgacggcattgagtacatcaaagctgcaatttcagctggtttgaaaatcgatgacttcgctccacgtcttagctttttcttcggaattggtatggatttgtttatgaatgtagcaatgcttcgtgcggctcgctacttatggtctgaagctgtatctggcttcggtgctcaagacccaaaatctcttgctttgcgtactcattgtcaaactagcggatggagccttactgaacaagatccttacaacaacgttatccgtacaacaattgaagctcttgctgcaacacttggaggtactcaatctcttcatacaaacgcgtttgatgaagctcttggacttcctacagatttctctgcacgtatcgcacgtaatactcagattatcattcaagaagaatctgagctttgccgtactgttgatccacttgccggatcttactacatcgaatctcttacagatcaaattgtaaaacaggctcgtgcgattatccaacaaattgacgaagctggcggtatggctaaagctattgaagcaggcttaccgaagcgtatgattgaggaagcctctgctcgtgaacaatctcttattgatcaaggaaaacgtgttattgttggtgtaaacaagtacaaacttgaccatgaagatgaaacagacgttcttgagatcgacaacgttatggttcgcaacgaacaaatcgcttctcttgagcgcattcgcgctactcgcgatgatgctgcagtaacggctgctcttaacgcacttacacatgcggctcaacataacgaaaacttacttgcggctgctgttaatgccgcacgtgttcgtgctactcttggcgagatctctgacgcacttgaagtagctttcgaccgttacttggttccttctcagtgcgttacgggagttattgctcaatcttatcatcaaagcgagaaatcagcttctgagttcgatgctatcgttgctcaaactgaacagtttcttgcggataacggtcgccgtcctcgcattcttattgcgaaaatgggtcaagacggtcacgatcgtggtgctaaagttatcgcaagcgcttattcagacttgggcttcgacgttgacttatctcctatgttctctactcctgaagaaatcgctcgtttagctgttgaaaacgacgttcacgtagtaggagcttcttctcttgcagcgggtcacaaaactcttattcctgaattagtagaagcattgaagaaatggggtcgtgaagacatttgtgttgtagccggtggcgttattccgccacaggattacgcattccttcaagaacgtggcgtagctgcgatctatggacctggcacaccaatgcttgattctgttcgtgatgttcttaacttgatttcacaacaccacgattaa

SEQ ID NO: 81 - DNA, 996 bp, Escherichia coli
atgattaatgaagccacgctggcagaaagtattcgccgcttacgtcagggtgagcgtgccacactcgcccaggccatgacgctggtggaaagccgtcacccgcgtcatcaggcactaagtacgcagctgcttgatgccattatgccgtactgcggtaacaccctgcgactgggcgttaccggcacccccggcgcggggaaaagtacctttcttgaggcctttggcatgttgttgattcgagagggattaaaggtcgcggttattgcggtcgatcccagcagcccggtcactggcggtagcattctcggggataaaacccgcatgaatgacctggcgcgtgccgaagcggcgtttattcgcccggtaccatcctccggtcatctgggcggtgccagtcagcgagcgcgggaattaatgctgttatgcgaagcagcgggttatgacgtagtgattgtcgaaacggttggcgtcgggcagtcggaaacagaagtcgcccgcatggtggactgttttatctcgttgcaaattgccggtggcggcgatgatctgcagggcattaaaaaagggctgatggaagtggctgatctgatcgttatcaacaaagacgatggcgataaccataccaatgtcgccattgcccggcatatgtacgagagtgccctgcatattctgcgacgtaaatacgacgaatggcagccacgggttctgacttgtagcgcactggaaaaacgtggaatcgatgagatctggcacgccatcatcgacttcaaaaccgcgctaactgccagtggtcgtttacaacaagtgcggcaacaacaatcggtggaatggctgcgtaagcagaccgaagaagaagtactgaatcacctgttcgcgaatgaagatttcgatcgctattaccgccagacgcttttagcggtcaaaaacaatacgctctcaccgcgcaccggcctgcggcagctcagtgaatttatccagacgcaatattttgattaa

SEQ ID NO: 82 - DNA, 996 bp, Escherichia coli
atgatcaacgaggcaactcttgctgaatcaattcgtcgccttcgccaaggtgaacgcgctacgcttgctcaagcaatgacgcttgtagagtctcgccatcctcgccatcaggctctttcaacacaacttcttgacgcaatcatgccttattgtggcaacacgcttcgccttggtgtaactggtactcctggtgctggtaaatcaactttccttgaggctttcggaatgcttcttatccgcgaaggacttaaagttgctgtaatcgctgttgacccttcatctcctgtaactggaggttctatccttggagacaaaactcgcatgaacgaccttgctcgcgctgaagctgcgttcattcgccctgttccttcatcaggtcatcttggcggagcttctcaacgcgcacgcgaacttatgcttctttgcgaggctgctggttacgacgtagttatcgtagagacagttggtgttggacagtcagagacagaggtagcacgcatggttgactgtttcatctctcttcaaatcgctggtggtggagacgaccttcaaggcatcaagaaaggtcttatggaagtagcggaccttatcgttatcaacaaagacgacggtgacaaccacactaacgtagctattgctcgccatatgtacgagtcagctcttcatatccttcgtcgcaagtatgacgagtggcaacctcgcgttcttacttgtagcgcacttgaaaaacgcggaatcgacgaaatctggcacgctatcatcgacttcaaaacagctcttactgcttcaggtcgccttcaacaggtacgccagcagcaatctgttgaatggcttcgcaaacaaacagaagaagaggttcttaaccaccttttcgcgaacgaggactttgaccgctattaccgccaaacgcttcttgctgttaagaacaacactcttagccctcgcacaggacttcgccaactttcagagttcatccaaacacagtatttcgactaa

SEQ ID NO: 83 - DNA, 29 bp, Escherichia coli
gaagatctatggtgcaaaacctttcgcgg

SEQ ID NO: 84 - DNA, 27 bp, Escherichia coli
aaaagtactcaaccaagtcattctgag

SEQ ID NO: 85 - DNA, 182 bp, Escherichia coli
aagcttatcgatagcgctatagttgttgacagaatggacatactatgatatattgttgctatagcgtacttagctggccagcatatatgtattctataaaatactattacaaggagattttagccatggaattcgcatgcggatccgcggccgcggtacccgggcgcgcctctagaaagctt

SEQ ID NO: 86 - DNA, 182 bp, Escherichia coli
aagcttatcgatcggggtttagttgttgacagggaggcttcgttgtgataagatggtagtatagcgtacttagctggccagcatatatgtattctataaaatactattacaaggagattttagccatggaattcgcatgcggatccgcggccgcggtacccgggcgcgcctctagaaagctt

SEQ ID NO: 87 - DNA, 35 bp, Lactobacillus reuteri
agtcaagcttccatggagaaggtttacattgttgc

SEQ ID NO: 88 - DNA, 51 bp, Lactobacillus reuteri
atgcggtaccgaattcctcgagtctagactaaattttcttaagcagaaccg

SEQ ID NO: 89 - DNA, 35 bp, Artificial Sequence
  ARTIFICIAL DNA PRIMER
aagcttctcgagactattacaaggagattttagcc

SEQ ID NO: 90 - DNA, 29 bp, Artificial Sequence
  ARTIFICIAL DNA PRIMER
gaattcactattacaaggagattttagtc

SEQ ID NO: 91 - DNA, 35 bp, Artificial Sequence
  ARTIFICIAL DNA PRIMER
aagcttctcgagactattacaaggagattttagtc

SEQ ID NO: 92 - DNA, 35 bp, Artificial Sequence
  ARTIFICIAL DNA PRIMER
aagcttcggccgactattacaaggagattttagcc

SEQ ID NO: 93 - protein, 714 aa, Escherichia coli
MSNVQEWQQLANKELSRREKTVDSLVHQTAEGIAIKPLYTEADLDNLEVTGTLPGLPPYVRGPRATMYTAQPWTIRQYAGFSTAKESNAFYRRNLAAGQKGLSVAFDLATHRGYDSDNPRVAGDVGKAGVAIDTVEDMKVLFDQIPLDKMSVSMTMNGAVLPVLAFYIVAAEEQGVTPDKLTGTIQNDILKEYLCRNTYIYPPKPSMRIIADIIAWCSGNMPRFNTISISGYHMGEAGANCVQQVAFTLADGIEYIKAAISAGLKIDDFAPRLSFFFGIGMDLFMNVAMLRAARYLWSEAVSGFGAQDPKSLALRTHCQTSGWSLTEQDPYNNVIRTTIEALAATLGGTQSLHTNAFDEALGLPTDFSARIARNTQIIIQEESELCRTVDPLAGSYYIESLTDQIVKQARAIIQQIDEAGGMAKAIEAGLPKRMIEEASAREQSLIDQGKRVIVGVNKYKLDHEDETDVLEIDNVMVRNEQIASLERIRATRDDAAVTAALNALTHAAQHNENLLAAAVNAARVRATLGEISDALEVAFDRYLVPSQCVTGVIAQSYHQSEKSASEFDAIVAQTEQFLADNGRRPRILIAKMGQDGHDRGAKVIASAYSDLGFDVDLSPMFSTPEEIARLAVENDVHVVGASSLAAGHKTLIPELVEALKKWGREDICVVAGGVIPPQDYAFLQERGVAAIYGPGTPMLDSVRDVLNLISQHHD

SEQ ID NO: 94 - protein, 331 aa, Escherichia coli
MINEATLAESIRRLRQGERATLAQAMTLVESRHPRHQALSTQLLDAIMPYCGNTLRLGVTGTPGAGKSTFLEAFGMLLIREGLKVAVIAVDPSSPVTGGSILGDKTRMNDLARAEAAFIRPVPSSGHLGGASQRARELMLLCEAAGYDVVIVETVGVGQSETEVARMVDCFISLQIAGGGDDLQGIKKGLMEVADLIVINKDDGDNHTNVAIARHMYESALHILRRKYDEWQPRVLTCSALEKRGIDEIWHAIIDFKTALTASGRLQQVRQQQSVEWLRKQTEEEVLNHLFANEDFDRYYRQTLLAVKNNTLSPRTGLRQLSEFIQTQYFD

SEQ ID NO: 95 - DNA, 29 bp, Artificial Sequence
  ARTIFICIAL DNA PRIMER
ggtaccactattacaaggagattttagtc

SEQ ID NO: 96 - DNA, 30 bp, Artificial Sequence
  ARTIFICIAL DNA PRIMER
atcctctagagaaggagatataccatgcgt

SEQ ID NO: 97 - DNA, 29 bp, Artificial Sequence
  ARTIFICIAL DNA PRIMER
tgcaagcttttagcggatattcaggccac

SEQ ID NO: 98 - DNA, 30 bp, Artificial Sequence
  ARTIFICIAL DNA PRIMER
atcctctagagaaggagatataccatggcc

SEQ ID NO: 99 - DNA, 29 bp, Artificial Sequence
  ARTIFICIAL DNA PRIMER
tgcaagcttttagacctcccaggaacgca

SEQ ID NO: 100 - DNA, 30 bp, Artificial Sequence
  ARTIFICIAL DNA PRIMER
atcctctagagaaggagatataccatgaat

SEQ ID NO: 101 - DNA, 29 bp, Artificial Sequence
  ARTIFICIAL DNA PRIMER
tgcaagcttttagcccgccagcacgcaac

SEQ ID NO: 102 - DNA, 783 bp, Escherichia coli
atgtcttatcagtatgttaacgttgtcactatcaacaaagtggcggtcattgagtttaactatggccgaaaacttaatgccttaagtaaagtctttattgatgatcttatgcaggcgttaagcgatctcaaccggccggaaattcgctgtatcattttgcgcgcaccgagtggatccaaagtcttctccgcaggtcacgatattcacgaactgccgtctggcggtcgcgatccgctctcctatgatgatccattgcgtcaaatcacccgcatgatccaaaaattcccgaaaccgatcatttcgatggtggaaggtagtgtttggggtggcgcatttgaaatgatcatgagttccgatctgatcatcgccgccagtacctcaaccttctcaatgacgcctgtaaacctcggcgtcccgtataacctggtcggcattcacaacctgacccgcgacgcgggcttccacattgtcaaagagctgatttttaccgcttcgccaatcaccgcccagcgcgcgctggctgtcggcatcctcaaccatgttgtggaagtggaagaactggaagatttcaccttacaaatggcgcaccacatctctgagaaagcgccgttagccattgccgttatcaaagaagagctgcgtgtactgggcgaagcacacaccatgaactccgatgaatttgaacgtattcaggggatgcgccgcgcggtgtatgacagcgaagattaccaggaagggatgaacgctttcctcgaaaaacgtaaacctaatttcgttggtcat

SEQ ID NO: 103 - protein, 261 aa, Escherichia coli
MSYQYVNVVTINKVAVIEFNYGRKLNALSKVFIDDLMQAFSDLNRPEIRCIILRAPSGSKVFSAGHDIHELPSGGRDPLSYDDPLRQITRMIQKFPKPIISMVEGSVWGGAFEMIMSSDLIIAASTSTFSMTPVNLGVPYNLVGIHNLTRDAGFHIVKELIFTASPITAQRALAVGILNHVVEVEELEDFTLQMAHHISEKAPLAIAVIKEELRVLGEAHTMNSDEFERIQGMRRAVYDSEDYQEGMNAFLEKRKPNFVGH

SEQ ID NO: 104 - DNA, 51 bp, Artificial Sequence
  ARTIFICIAL DNA PRIMER\
caccgaattcaagaaggagatataccatgtctaacgtgcaggagtggcaac

SEQ ID NO: 105 - DNA, 55 bp, Artificial Sequence
  ARTIFICIAL DNA PRIMER
ctagtctagaaagcttgcggccgcggatccttaatcatgatgctggcttatcaga

SEQ ID NO: 106 - DNA, 59 bp, Artificial Sequence
  ARTIFICIAL DNA PRIMER
caccgaattcgcggccgcaagaaggagatataccatgattaatgaagccacgctggcag

SEQ ID NO: 107 - DNA, 57 bp, Artificial Sequence
  ARTIFICIAL DNA PRIMER
ctagtctagaaagcttggccggccggcgcgccttaatcaaaatattgcgtctggata

SEQ ID NO: 108 - DNA, 67 bp, Artificial Sequence
  ARTIFICIAL DNA PRIMER
caccgaattcggcgcgccggccggccaagaaggagatataccatgtcttatcagtatgttaacgttg

SEQ ID NO: 109 - DNA, 49 bp, Artificial Sequence
  ARTIFICIAL DNA PRIMER
ctagtctagaaagcttttaattaactaatgaccaacgaaattaggttta

SEQ ID NO: 110 - DNA, 41 bp, Artificial Sequence
  ARTIFICIAL DNA PRIMER
caccgaattcttaattaaaaggagatataccatgaccatca

SEQ ID NO: 111 - DNA, 49 bp, Artificial Sequence
  ARTIFICIAL DNA PRIMER
ctagtctagaaagcttcctgcaggttagcggatattcaggccactcttt

SEQ ID NO: 112 - DNA, 27 bp, Artificial Sequence
  ARTIFICIAL DNA PRIMER
tagtctagactcgaggaattcggtacc

SEQ ID NO: 113 - DNA, 1155 bp, Propionibacterium freudenreichii
atgaacacgcttgccgataacccggtcattatcgaagcgctgcgcacacctattgggactacgggtggtgtgtttgcggatcaaactaccactgatctagcagcgccagtcttgggtgaactcgacgctcggctgcctaccggtactggttttggcgaagttgtgttaggtaacgttcgtgggcctggcggtgatccagcacgtgttgccgctttggctgcagggattgatccagcagtaccagcgttaacattagaccgccagtgcggtagcggtatggcagcaattgagtatgcatggcaccgatgccggtcacaaccaggggtcgttgctgccggaggtgtccaagcggcaagcacacagcctattacattatggcctggccgtgatggtgaaccacccagttcgtttgaccgtgcgccatttgccccacctccgtggttagatcctgatatgggggttatggctgaccgtttagcagctgaacttcacattagtcgtgaacgtcaagatcgttatgcgctgcgctctcatacccgtgcctcaaccgcacgagatgcaggggatttcgatgcggaaatcgtgccaatcgctcgtgtacgtcgtgatcaacgtccacgctcaggttttactatggcacgcttggcacgtttcccagcggcttttcgccctggtggcacggttacggcagcaaatagttgtgggattaacgacgcagcagccgctgtgacgatggtagatgctgctacccatgcgcacttagcaactccaggcttgcgcgtgttagctgcacgtacagttggttatgatccggaccggtttgggttgggcctcgtaccggctgttcgggcagcgcttgacgacgctggtttgggtttggaccaaattgatgcattagagttcaatgaagcgtttgcggctcaagtattggcttgtgcggatgctttggatttggatgagcatcgtctctgtccacgtggcggagcaatttcgctaggtcatccttggggtgcgtccggtgccatcttacttgttcgtctctttagtcgcttggtacgtgaaggcgcaggtcgttacggtttggctgcgatttctattggtggtgggcaaggctctgctgttgttgtcgaggcagtacatccccgtgggaattag

SEQ ID NO: 114 - protein, 384 aa, Propionibacterium freudenreichii
MNTLADNPVIIEALRTPIGTTGGVFADQTTTDLAAPVLGELDARLPTGTGFGEVVLGNVRGPGGDPARVAALAAGIDPAVPALTLDRQCGSGMAAIEYAWHRCRSQPGVVAAGGVQAASTQPITLWPGRDGEPPSSFDRAPFAPPPWLDPDMGVMADRLAAELHISRERQDRYALRSHTRASTARDAGDFDAEIVPIARVRRDQRPRSGFTMARLARFPAAFRPGGTVTAANSCGINDAAAAVTMVDAATHAHLATPGLRVLAARTVGYDPDRFGLGLVPAVRAALDDAGLGLDQIDALEFNEAFAAQVLACADALDLDEHRLCPRGGAISLGHPWGASGAILLVRLFSRLVREGAGRYGLAAISIGGGQGSAVVVEAVHPRGN

SEQ ID NO: 115 - DNA, 1170 bp, Lactobacillus brevis
atggcagaagaggttgtgattgttagtgcggcacgtacccctatcggcaagtttggcaaatccttacggggattgagtgcagaacaactaggcaccattgccgctaaagcagcgattactcgtagtggcttagatcctgcgacgattcaacagaccatctttggaagcgtgctgcaagctgggcaaggccaaaacattgcgcgtcagatcgaattgaacgcaggcttaccagtcacttcgaccgcaatgaccattaaccaggtgtgtggctcatccctcaaagctattcgtttgggccagtccgccattttgatgggggatgccgatatcgttcttgtaggtggtacagaatccatgtccaatgcaccttaccttactcctcaaacccgttggggtcataagtttggtgacattacgcttacagattctctcgcccatgatggtttaacagacgcttttacggatatccctatgggaatcacggctgagaacgttgccgcacagttccacatctcacgtgccgaccaggacgcttttgcccttcgttcccaacgtcgtgccacggctgcccagcaagctaatcggtttgccgacgaaattgttcccgtaagcctgggtacaacgactctcacaactgatgaagccgtccggactaccacttcgcgtgagcaactggcagcgttgcgtccagcattcaaaagtgttggcaccgttactgcagggaacgctgcaggcttgaatgacggagccgctgcgatgattctgatgaagaaaagtactgcacgcgctgctgggattgcgtacttggcaacccttcgcggttaccaagaagtcgggattgatccagccatcatgggatatgctccggttacagctattcagcaactccttacgaaaactcagcgcagtgtgtcggatattgaccgttttgaattgaatgaggcttttgcagcccaaagtgttgctgtaggtcaagctctagcattaccagatgaccgcttgaatgttaatggtggtgccattgcactcggtcatcctctaggtgcttccggcacccgtatcgtcgttacattgctgtacgaacttttgcattccgggacccatttgggtgttgcttctatgtgcattggtggtggaatgggaatggcattgatgattgagtcgaattag

SEQ ID NO: 116 - protein, 389 aa, Lactobacillus brevis
MAEEVVIVSAARTPIGKFGKSLRGLSAEQLGTIAAKAAITRSGLDPATIQQTIFGSVLQAGQGQNIARQIELNAGLPVTSTAMTINQVCGSSLKAIRLGQSAILMGDADIVLVGGTESMSNAPYLTPQTRWGHKFGDITLTDSLAHDGLTDAFTDIPMGITAENVAAQFHISRADQDAFALRSQRRATAAQQANRFADEIVPVSLGTTTLTTDEAVRTTTSREQLAALRPAFKSVGTVTAGNAAGLNDGAAAMILMKKSTARAAGIAYLATLRGYQEVGIDPAIMGYAPVTAIQQLLTKTQRSVSDIDRFELNEAFAAQSVAVGQALALPDDRLNVNGGAIALGHPLGASGTRIVVTLLYELLHSGTHLGVASMCIGGGMGMALMIESN

SEQ ID NO: 117 - DNA, 834 bp, Lactobacillus salivarius
atgtcaagtttcgttgcgaacgaagaagaaatccggaacttcttatgtcgtcccaacatgaagaagcaggacggcattatgttcgcatacgttaccagtgccgaaaagctgactgagctcgtaccaaagccgctcaaggttgttgcacccgttatcatgggatacgtcacacacatgggcgacccaacattcgctgcaccatacgacgaagcggttctgtacgcgttcgtgagttaccaggacaagatcatgggagcatacccattcgtcttatacctttcgggtgaaggtgctgaagaagccatgatcgcaggtcgtgaaggtgcttgtatgccgaagaagttagcagacgacatcaagatcgagaagtccgaaaacaaggtccaggccaagattatccggcacggtacagaaatcttaaacttaaagtgggaagcaggtgttccgaacgacgttgaaactgtgaagaagatgttcggcagccaggtcaagttaggcgaaccaagcgaaatggcctcattcttcatcaactacgacattgaacagcaggacgacggttctaacaagttcgttaacaccgagcttatcgccacacagagcaccggtctaactactgacatggaaattgggaagatcgacatgaaacttacgagcagcgaggacgacccgctcgccgaattagaagttttgaagcctgtaggcggtgcacactacaagatggactactccgtcatgcacaagacgttgaaacttgactcacttaacccggaggaaattgccccataccttatcacaggtcactacgaccgtactttcgttactaagcaggactag

SEQ ID NO: 118 - protein, 277 aa, Lactobacillus salivarius
MSSFVANEEEIRNFLCRPNMKKQDGIMFAYVTSAEKLTELVPKPLKVVAPVIMGYVTHMGDPTFAAPYDEAVLYAFVSYQDKIMGAYPFVLYLSGEGAEEAMIAGREGACMPKKLADDIKIEKSENKVQAKIIRHGTEILNLKWEAGVPNDVETVKKMFGSQVKLGEPSEMASFFINYDIEQQDDGSNKFVNTELIATQSTGLTTDMEIGKIDMKLTSSEDDPLAELEVLKPVGGAHYKMDYSVMHKTLKLDSLNPEEIAPYLITGHYDRTFVTKQD

SEQ ID NO: 119 - DNA, 846 bp, Lactobacillus plantarum
atggctagcttcattgcttcggaccaggacgtgaagaacttcttaactgcaccgtctatgaacgaccaagaaggtattggattcgcgtacgcgactgacgaggcagttctgaaggctttgattccagcaccgctcaagttaatggcaccggttgtttgcggttacgtcgtacacatgggaaaaccgaccttctctgccccatacttggaagaatcattgttcgcattagtgagctacaaggacaaaatgatgggtgcatacccgattaacttgttacttcatggtccgggtgcagaatcaggtgttattgcagggcgtgaaggggcaggtatcccgaagaaactcgctgacgacattgaacttcgtcgcaacgacaacagtgcaactgctactgttgaacgtcacgggaaaaccctcttgaacgtcagttggacagctggagagttgaacgacccgtcgattatgaaacaattcgcaggtcaattagcgctcggcaaggaagctgaaatgaacagtttcttctacaaatacgacattgaccagcacgaagacggcactaaccacttctctaacgttcaacttgttgctacgcaattacgtagccttgccgaccaagtcgaaccagggaacctcagcatccagttagaatctacggacgacgacccgttcggtgagttgaaagtcttaaagcctctgggtgctgcttggttccacttcgacacaagtgtcatgttcaacacgctgaaactcgacgaagttgacgctgcaactactatgcctaagttattgacgggtcggtacgaccgctcattcttcaacccaaaagccgctacttacattatctag

SEQ ID NO: 120 - protein, 281 aa, Lactobacillus plantarum
MASFIASDQDVKNFLTAPSMNDQEGIGFAYATDEAVLKALIPAPLKLMAPVVCGYVVHMGKPTFSAPYLEESLFALVSYKDKMMGAYPINLLLHGPGAESGVIAGREGAGIPKKLADDIELRRNDNSATATVERHGKTLLNVSWTAGELNDPSIMKQFAGQLALGKEAEMNSFFYKYDIDQHEDGTNHFSNVQLVATQLRSLADQVEPGNLSIQLESTDDDPFGELKVLKPLGAAWFHFDTSVMFNTLKLDEVDAATTMPKLLTGRYDRSFFNPKAATYII

SEQ ID NO: 121 - DNA, 1071 bp, Lactobacillus fermentum
atgaaaggtttcgctatgcttggtcccaacaagaccggtttcattgaaaaagaagatcccaaagttggctcgcgtgatgctttggttcgtccgctggcagttgcgccatgcacaagtgatatccacaccgtattcgaagatgcattgggtcctcgggaaaacatgatcttagggcacgaggcttgtggggaaatcgtagaggttggtagcgaagttaaagatttcaaggtaggcgatcgtgtcctgatcccagccattacaccgaactggagtgatgtatactctcaagcaggttacgcaactgatagcgatggtatgttgggaggctggaaattctccaacattaaggatggtgtcttctcaactcgtattcacgttaacgatgcggacggaaacttagctttacttccaaaagagattgatccggcagaagcctgtatgctgagtgacatgattccgacgggtctgcacgcgtttgaaatggcaggagttaccttcggggacgccgtggccgtgattggcgtgggtccagttggtctcatggctttacgtggggctgttttacatggggctggacgcgtattcgcggttggttcacgggagaaaacagtagaagtggcgaagaagtatggtgccaccgacatcattgactaccacaacggtcgcattagcgaccaaattctggaagccacaaacggtcaaggtgttgataaagtattgattgcagggggtaacgccaaagatactttcgaagaagctgtgcggatgctcaaaccgggtggtagcatcggaaacgtcaactacctaaacggtcacgatgatgttgttttcaacgcaggtgactggggtgttggtatgggccacaaaacaattcacggtggcctcatgccaggtggccgtttgcgtatggaaaagttagccgcattggtagtgaatggtcggattgacccctcgctgatgattacacaccgtttcgaaggcttcgagaaaatccctgatgccatcgagctcatgcaccacaaaccagctgatttgatcaaaccggttgtcatttgtaacgatattgattag

SEQ ID NO: 122 - protein, 356 aa, Lactobacillus fermentum
MKGFAMLGPNKTGFIEKEDPKVGSRDALVRPLAVAPCTSDIHTVFEDALGPRENMILGHEACGEIVEVGSEVKDFKVGDRVLIPAITPNWSDVYSQAGYATDSDGMLGGWKFSNIKDGVFSTRIHVNDADGNLALLPKEIDPAEACMLSDMIPTGLHAFEMAGVTFGDAVAVIGVGPVGLMALRGAVLHGAGRVFAVGSREKTVEVAKKYGATDIIDYHNGRISDQILEATNGQGVDKVLIAGGNAKDTFEEAVRMLKPGGSIGNVNYLNGHDDVVFNAGDWGVGMGHKTIHGGLMPGGRLRMEKLAALVVNGRIDPSLMITHRFEGFEKIPDAIELMHHKPADLIKPVVICNDID

SEQ ID NO: 123 - DNA, 18 bp, Artificial Sequence
  ARTIFICIAL DNA PRIMER
ctgataagtgagctattc

SEQ ID NO: 124 - DNA, 17 bp, Artificial Sequence
  ARTIFICIAL DNA PRIMER
cagcacagttcattatc

SEQ ID NO: 125 - DNA, 48 bp, Artificial Sequence
  ARTIFICIAL DNA PRIMER
ccacattgaaaggggaggagaatcatgaaggaagttgtgattgcttct

SEQ ID NO: 126 - DNA, 48 bp, Artificial Sequence
  ARTIFICIAL DNA PRIMER
agtcgacgcggccgctagcacgcgttataagatgacaacggctttgat

SEQ ID NO: 127 - DNA, 1392 bp, Rhodopseudomonas palustris
atggtagcaaaagctatccgcgaccatgctggcactgcacagccgtcaggtaacgctgctacatcttctgcagcggtaagcgacggcgttttcgagacaatggacgctgcggtagaagcggctgctcttgctcaacagcaataccttctttgctcaatgtctgatcgcgctcgcttcgttcaaggcatccgcgacgtaatccttaaccaagacacacttgagaaaatgagccgcatggcagttgaggagacaggaatgggcaactacgagcataaacttatcaaaaaccgccttgctggcgagaaaacacctggcatcgaggaccttacaacagacgcattctcaggcgacaacggccttacacttgttgagtattcaccatttggcgtaattggcgcaatcactccaacaactaacccaactgagactatcgtatgtaactctatcggcatgcttgcggcaggcaactcagtagttttctctcctcaccctcgcgcacgccaggtatctcttcttcttgtacgccttatcaaccagaaacttgcagctcttggcgctccagagaaccttgtagttactgttgagaaaccttctatcgagaacactaacgctatgatggctcacccaaaagtacgcatgcttgtagcaactggcggtcctgcgatcgttaaagctgtactttctacaggcaaaaaggctatcggagctggtgctggcaatccgccagttgttgttgacgagacggctaacatcgagaaagctgcgtgtgacatcgtaaacggctgctctttcgacaacaaccttccttgcgttgcagagaaggagatcatcgcagtagctcagatcgctgactaccttatcttcaaccttaagaagaacggagcttatgagatcaaagaccctgcggtacttcaacaacttcaagaccttgtacttactgctaaaggtggccctcaaactaaatgtgtaggcaaatctgcagtttggcttctttctcaaatcggcatctctgtagacgcgtcaatcaagatcatccttatggaagtacctcgcgagcatccttttgtacaagaggagcttatgatgccaatccttcctcttgttcgcgtagagactgttgacgacgctatcgaccttgctatcgaggtagagcacgacaaccgccacacagctatcatgcactcaactgacgttcgcaaacttacgaaaatggctaaacttatccagactacaatctttgtaaagaacggtccttcttacgcaggacttggtgctggtggcgagggctattctacatttactatcgctggccctacgggtgagggacttacatcagctaagagcttcgctcgtcgtcgcaaatgcgttatggtagaggctcttaacatccgc

SEQ ID NO: 128 - DNA, 1392 bp, Propionibacterium freudenreichii
atgacaatctcacctgagcttatccaacaagtagttcgcgagacagtacgcgaggttatctcacgccaagactctggtactgatgctccgtctggtactgacggtatcttcacagacatgaactctgcggtagacgcagctgacgtagcatggcgtcagtatatggactgtagccttcgcgaccgcaaccgcttcatccaagctatccgcgacgtagcttcagagccggacaaccttgagtacatggctacggctacggtagaggagactggaatgggtaacgtacctcacaagatccttaagaaccgctatgctgctctttacactcctggcacggaagacatcatcactgaggcgtggtcaggcgacgacggtcttactactgtagagttttctcctttcggagttatcggagcaatcacacctactacgaacccaactgaaactgtaatcaacaacacaatcggtatgcttgcagctggcaacgcagttgttttctctcctcatcctcgtgcgaaaaagatcactctttggcttgttcgcaaaatcaatcgtgcgcttgctgctgctggcgctcctgcaaaccttgttgtaacagtagaggagccttctatcgacaacactaacgctatgatgtcacatgagaaagttcgcatgcttgtagctactggtggtcctggtatcgttaaagctgttctttcttcaggcaagaaagccatcggagctggtgctggcaaccctcctgctgtagtagacgacactgctgacatcgctaaagctgcacgcgacatcgtagacggagcatctttcgacaacaaccttccgtgcactgcagagaaagaggttcttgcagtagactctatcgcggaccttcttaagtttgaaatgcttaagcatggttgttttgaacttaaggatcgcgcagtaatggacaaacttgcagctcttgttacgaaaggtcaacacgctaacgcagcttacgtaggtaaacctgcggctcaacttgcttcagaggtaggactttctgcacctaaggacacacgccttcttatctgtgaggttccttttgaccacccgttcgtacaagttgagcttatgatgccgatccttcctatcgtacgcatgccggacgtagacactgctatcgacaaggctgtagaggtagagcatggaaaccgccatactgctgtaatgcactctagcaacgttaacgctcttacgaaaatgggcaaacttatccaaacaactatcttcgtaaagaacggtccgtcttacaacggaatcggtatcgacggagagggttttcctacttttacgattgctggtccgacaggtgagggtcttacgtctgcacgctgctttgctcgcaaacgtcgctgcgtacttaagtca

SEQ ID NO: 129 - DNA, 783 bp, Escherichia coli
atgtcttaccagtacgttaacgttgtaacaatcaacaaggttgctgttatcgagttcaactatggtcgcaaacttaacgcgctttcaaaagttttcatcgacgaccttatgcaagctctttctgaccttaatcgcccagagatccgctgcatcatccttcgcgcaccatctggctctaaagttttctctgctggccacgacatccacgagcttccatctggtggtcgcgacccactttcttacgacgaccctcttcgccagatcacacgcatgatccagaagttcccaaaaccgatcatcagcatggttgagggcagcgtttggggtggtgcgttcgagatgatcatgtcttctgaccttatcattgcggctagcacttcaacattctctatgactccggttaaccttggcgtaccgtataaccttgttggcatccataaccttacacgcgacgctggcttccatatcgttaaagagcttatctttactgcttctccaatcactgcacaacgtgcgcttgctgtaggcatccttaaccacgttgtagaggttgaggagcttgaggacttcacgcttcaaatggctcatcacatctctgagaaggctccacttgcgatcgctgttatcaaagaggagcttcgcgttcttggcgaggctcacactatgaactctgacgagttcgagcgcatccagggcatgcgtcgcgctgtatatgactctgaggactatcaagagggcatgaacgctttccttgagaagcgcaaaccaaactttgttggccat